(19) United States
(12) Patent Application Publication
(10) Pub. No.: US 2026/0190604 A1
(43) Pub. Date: Jul. 2, 2026

(54) HETEROCYCLIC COMPOUND, COMPOSITION FOR ORGANIC LAYER INCLUDING THE SAME AND LIGHT EMITTING DEVICE INCLUDING THE SAME

(71) Applicant: LT MATERIALS CO., LTD., Gyeonggi-do (KR)

(72) Inventors: YU JIN HEO, Gyeonggi-do (KR); JUN TAE MO, Gyeonggi-do (KR); DONG JUN KIM, Gyeonggi-do (KR); DAE HYUK CHOI, Gyeonggi-do (KR)

(21) Appl. No.: 19/433,459

(22) Filed: Dec. 26, 2025

(30) Foreign Application Priority Data
Dec. 26, 2024 (KR) 10-2024-0197016

(51) Int. Cl. H10K 50/12; C07B 59/00; C07D 405/14; C09K 11/06; H10K 85/30; H10K 85/40; H10K 85/60

(52) U.S. Cl. CPC H10K 50/12; C07B 59/004; C07D 405/14; C09K 11/06; H10K 85/324; H10K 85/342; H10K 85/40; H10K 85/622; H10K 85/626; H10K 85/633; H10K 85/654; H10K 85/6572; H10K 85/6574; H10K 85/6576; C07B 2200/05; C09K 2211/1029

(57) ABSTRACT

A heterocyclic compound according to an embodiment is represented by Formula 1. A light-emitting device includes a first electrode, a second electrode, and one or more organic layer interposed between the first electrode and the second electrode. At least one of the one or more organic layer includes the above-described heterocyclic compound.

### CROSS-REFERENCE TO RELATED APPLICATION AND CLAIM OF PRIORITY

This application claims priority to and the benefit of Korean Patent Application No. 10-2024-0197016 filed on Dec. 26, 2024, with the Korean Intellectual Property Office (KIPO), and all the benefits accruing therefrom under 35 U.S.C. § 119, the entire disclosure of which is incorporated herein by reference.

### BACKGROUND

### 1. Field of the Invention

The present disclosure relates to a heterocyclic compound and a light-emitting device including the same.

### 2. Description of the Related Art

An organic light-emitting display (OLED) device includes organic light-emitting devices having self-luminescent properties. Since the organic light-emitting display device does not require a separate light source, it may provide a wide viewing angle and a fast response speed, and may enhance contrast and brightness.

In the organic light-emitting device, an organic light-emitting layer is formed for each pixel, and the organic light-emitting layer may be interposed between opposing electrodes. Holes and electrons injected from the respective electrodes recombine in the organic light-emitting layer to generate excitons, and light may be generated through the energy release of the excitons.

Research is being conducted on materials applicable to the organic light-emitting layer to achieve high efficiency and long life organic light-emitting devices.

### SUMMARY

An object of the present disclosure is to provide a heterocyclic compound.

Another object of the present disclosure is to provide a light-emitting device including the heterocyclic compound.

The heterocyclic compound according to the present disclosure may be represented by Formula 1 below.

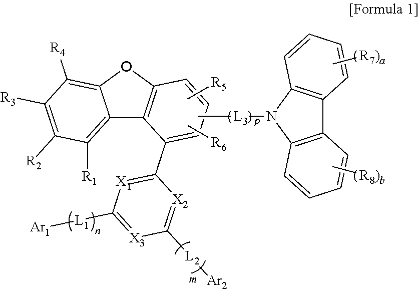

In Formula 1, X₁ to X₃ are each independently N or CR₉.

However, at least two of X₁ to X₃ are nitrogen.

R₁ to R₉ are each independently selected from the group consisting of hydrogen; deuterium; a halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, wherein adjacent groups are linked to form a substituted or unsubstituted aryl group having 6 to 60 carbon atoms; a substituted or unsubstituted heterocycloalkyl group having 2 to 60 carbon atoms; or a substituted or unsubstituted heteroaryl group having 2 to 60 carbon atoms.

R, R′ and R″ are each independently hydrogen; deuterium; a cyano group (—CN); a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms.

Ar₁ and Ar₂ are each independently a substituted or unsubstituted aryl group having 6 to 60 carbon atoms, or a substituted or unsubstituted heteroaryl group having 2 to 60 carbon atoms.

L₁ and L₂ are each independently a direct linkage, a substituted or unsubstituted arylene group having 6 to 60 carbon atoms, or a substituted or unsubstituted heteroarylene group having 2 to 60 carbon atoms.

L₃ is a substituted or unsubstituted arylene group having 6 to 60 carbon atoms, or a substituted or unsubstituted heteroarylene group having 2 to 60 carbon atoms.

However, L₃ is not a group represented by Formula 2 below.

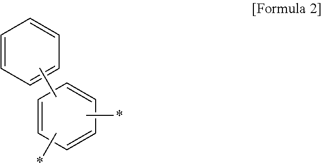

n and m are each independently 0 or an integer of 1 to 5.

p is an integer of 1 to 5.

When n, m, and p are each independently 2 or more, a plurality of L₁ groups, L₂ groups, and L₃ groups are each independently the same or different.

a and b are each independently 0 or an integer of 1 to 4.

When a and b are each independently 2 or more, a plurality of R₇ groups and R₈ groups are each independently the same or different.

* represents a bonding site.

A light-emitting device according to the present disclosure includes: a first electrode; a second electrode disposed on the first electrode; and one or more organic layers interposed between the first electrode and the second electrode. One or more of the organic layers includes the heterocyclic compound.

A composition for an organic layer of an organic light-emitting device according to the present disclosure includes: the heterocyclic compound represented by Formula 1; and a compound represented by Formula 4 below or a compound represented by Formula 5 below.

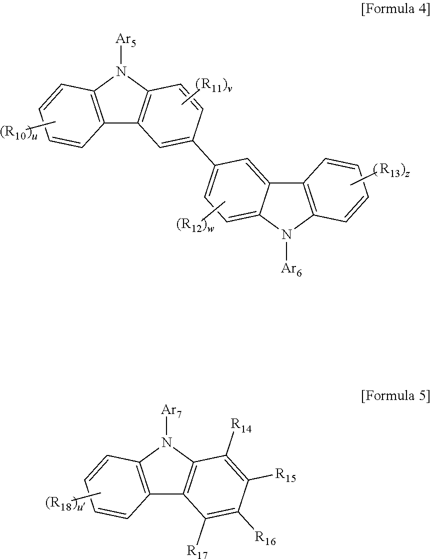

In Formula 4 and 5, two adjacent groups of R₁₄ to R₁₇ form a ring represented by

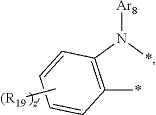

the remaining groups of R₁₀ to R₁₃, R₁₄ to R₁₇, and R₁₈ to R₁₉ that do not form a ring are each independently selected from the group consisting of hydrogen; deuterium; a halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, wherein adjacent groups are linked to form a substituted or unsubstituted aryl group having 6 to 60 carbon atoms; a substituted or unsubstituted heterocycloalkyl group having 2 to 60 carbon atoms; or a substituted or unsubstituted heteroaryl group having 2 to 60 carbon atoms,
R, R′ and R″ are each independently hydrogen; deuterium; a cyano group (—CN); a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms,
Ar₅ to Ar₈ are each independently a substituted or unsubstituted aryl group having 6 to 60 carbon atoms, or a substituted or unsubstituted heteroaryl group having 2 to 60 carbon atoms, u, u′, z and z′ are each independently 0 or an integer of 1 to 4,
v and w are each independently 0 or an integer of 1 to 3, and
* represents a bonding site.

According to exemplary embodiments, the light-emitting device may include a heterocyclic compound of one embodiment. The above-described heterocyclic compound may function, for example, as a host material to control the energy band gap and energy levels of the light-emitting layer. Therefore, the light-emitting efficiency and lifetime characteristics of the light-emitting device may be improved by the above-described heterocyclic compound.

### BRIEF DESCRIPTION OF THE DRAWINGS

The above and other objects, features and advantages of the present disclosure will be more clearly understood from the following detailed description taken in conjunction with the accompanying drawings, in which:

FIGS. 1 to 3 are schematic cross-sectional views of light-emitting devices according to embodiments of the present disclosure, respectively.

### DETAILED DESCRIPTION OF THE INVENTION

Hereinafter, exemplary embodiments of the present disclosure will be described in detail with in reference to the accompanying drawings. However, these embodiments are merely illustrative, and the present disclosure is not limited to the specific embodiments described as examples.

As used herein, when a portion is described to “include” a component, unless otherwise specified, it means that the portion does not exclude other components and may further include other components.

As used herein, the * symbol in a chemical formula represents a bonding site.

As used herein, “substitution” means that any hydrogen atom bonded to a carbon atom in a compound is replaced with another substituent. The position of substitution is not limited as long as the substituent is at a substitutable position. If two or more substitutions are made, the two or more substituents may be the same or different.

As used herein, “substituted or unsubstituted” means that at least one hydrogen atom of a compound is unsubstituted or substituted with one or more substituents selected from the group consisting of substituents, or is substituted with a substituent in which two or more of the following substituents are linked: deuterium; a halogen group; —CN; an alkyl group having 1 to 60 carbon atoms; an alkenyl group having 2 to 60 carbon atoms; an alkynyl group having 2 to 60 carbon atoms; a haloalkyl group having 1 to 60 carbon atoms; an alkoxy group having 1 to 60 carbon atoms; an aryloxy group having 6 to 60 carbon atoms; an alkylthio group having 1 to 60 carbon atoms; an arylthio group having 6 to 60 carbon atoms; an alkylsulfinyl group having 1 to 60 carbon atoms; an arylsulfinyl group having 6 to 60 carbon atoms; a cycloalkyl group having 3 to 60 carbon atoms; a heterocycloalkyl group having 2 to 60 carbon atoms; an aryl group having 6 to 60 carbon atoms; a heteroaryl group having 2 to 60 carbon atoms; —SiRR′R″; —P(═O)RR′; and —NRR′·R, R′ and R″ are each independently a substituent including at least one of hydrogen; deuterium; a halogen group; an alkyl group; an alkenyl group; an alkoxy group; a cycloalkyl group; a heterocycloalkyl group; an aryl group; and a heteroaryl group.

As used herein, when a substituent is not indicated in a chemical formula or compound structure, it means that a hydrogen atom is bonded to the carbon atom. However, since deuterium (2H) is an isotope of hydrogen, some hydrogen atoms may be deuterium.

As used herein, when a substituent is not indicated in a chemical formula or compound structure, it may mean that all positions that can be substituted are hydrogen or deuterium. In other words, deuterium is an isotope of hydrogen, and some of the hydrogen atoms may be deuterium atoms, in which case the deuterium content may be 0% to 100%.

When a substituent is not indicated in a chemical formula or compound structure, and the deuterium content is 0%, the hydrogen content is 100%, and all substituents are hydrogen, hydrogen and deuterium may nevertheless be present together in the compound unless deuterium is explicitly excluded.

Deuterium is an isotope of hydrogen, having a nucleus composed of one proton and one neutron (a deuteron). It may be represented as hydrogen-2 and its elemental symbol may be written as D or ²H.

Isotopes are atoms with the same atomic number (Z) but different mass numbers (A). Isotopes may also be defined as atoms with the same number of protons but different numbers of neutrons.

As used herein, the content T % of a specific substituent is defined as T2/T1×100=T(%), where the total number of substituents that a basic compound may have is defined as T1, and the number of the specific substituent among them is defined as T2.

In one example, a 20% deuterium content in a phenyl group represented by

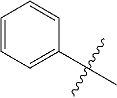 [SMILES: *c1ccccc1]

means that the total number of substituents that the phenyl group may have is 5 (T1 in the formula), and among them, the number of deuterium atoms is 1 (T2 in the formula), which may be represented as 20%. That is, cases where the deuterium content in the phenyl group is 20% may be represented by the structural formula below.

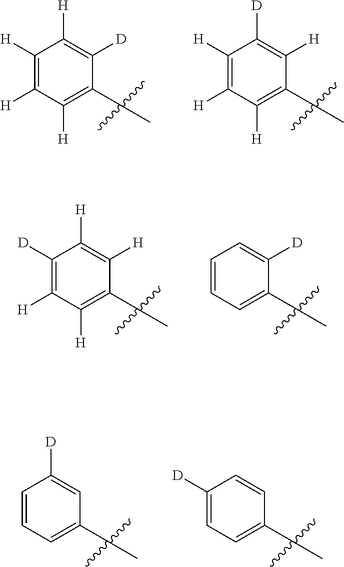 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, “a phenyl group having 0% deuterium content” may refer to a phenyl group that includes no deuterium atoms and has five hydrogen atoms.

As used herein, halogen may be fluorine, chlorine, bromine, or iodine.

As used herein, an alkyl group includes a straight or branched chain having 1 to 60 carbon atoms, and may be further substituted with one or more substituents. The alkyl group may have 1 to 60, 1 to 40, or 1 to 20 carbon atoms. Examples thereof may include a methyl group, an ethyl group, a propyl group, an n-propyl group, an isopropyl group, a butyl group, an n-butyl group, an isobutyl group, a tert-butyl group, a sec-butyl group, a 1-methylbutyl group, a 1-ethylbutyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethylpropyl group, a 1,1-dimethylpropyl group, an isohexyl group, a 2-methylpentyl group, a 4-methylhexyl group, and a 5-methylhexyl group, but are not limited thereto.

As used herein, the alkenyl group includes a straight or branched chain having 2 to 60 carbon atoms, and may be further substituted with one or more substituents. The alkenyl group may have 2 to 60, 2 to 40, or 2 to 20 carbon atoms. Examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl group, a 2-phenylvinyl group, a 2,2-diphenylvinyl group, a 2-phenyl-2-(naphthyl-1-yl)vinyl group, a 2,2-bis(diphenyl)vinyl group, a stilbenyl group, and a styrenyl group, but are not limited thereto.

As used herein, an alkynyl group includes a straight or branched chain having 2 to 60 carbon atoms, and may be further substituted with one or more substituents. The alkynyl group may have 2 to 60, 2 to 40, or 2 to 20 carbon atoms.

As used herein, a haloalkyl group refers to an alkyl group substituted with a halogen group. Examples thereof may include —CF₃ and —CF₂CF₃, but are not limited thereto.

As used herein, a cycloalkyl group includes a monocyclic or polycyclic group having 3 to 60 carbon atoms, and may be further substituted with one or more substituents. In this context, a polycyclic group refers to a group in which a cycloalkyl group is directly linked to or condensed with another cyclic group. The other cyclic group may be a cycloalkyl group, but may also be another type of cyclic group, such as a heterocycloalkyl group, an aryl group, or a heteroaryl group. The number of carbon atoms in the cycloalkyl group may be 3 to 60, 3 to 40, or 5 to 20. Examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, and a cyclooctyl group, but are not limited thereto.

As used herein, a heterocycloalkyl group includes a monocyclic or polycyclic group having 2 to 60 carbon atoms and including at least one heteroatom selected from O, S, Se, N and Si, and may be further substituted with one or more substituents. In this context, the term “polycyclic” refers to a group in which a heterocycloalkyl group is directly linked to or condensed with another ring group. The other ring group may be a heterocycloalkyl group, but may also be another type of ring group, such as a cycloalkyl group, an aryl group, or a heteroaryl group. The heterocycloalkyl group may have 2 to 60, 2 to 40, or 3 to 20 carbon atoms.

As used herein, an aryl group includes a monocyclic or polycyclic group having 6 to 60 carbon atoms, and may be further substituted with one or more substituents. In this context, the term “polycyclic” refers to a group in which an aryl group is directly linked to or condensed with another ring group. The other ring group may be an aryl group, but may also be another type of ring group, such as a cycloalkyl group, a heterocycloalkyl group, or a heteroaryl group. The aryl group may include a spiro ring structure. The aryl group may have 6 to 60, 6 to 40, or 6 to 25 carbon atoms. Specific examples of the aryl group may include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, and condensed ring groups thereof, but are not limited thereto.

As used herein, the terphenyl group may be selected from the following structures.

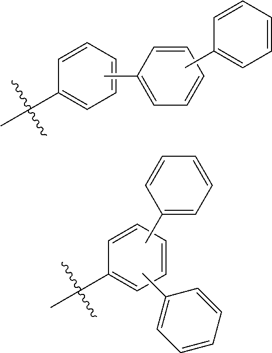

As used herein, the fluorenyl group may be substituted, and adjacent substituents may be bonded to form a ring.

The substituted fluorenyl group may be represented by the following structural formula, but is not limited thereto.

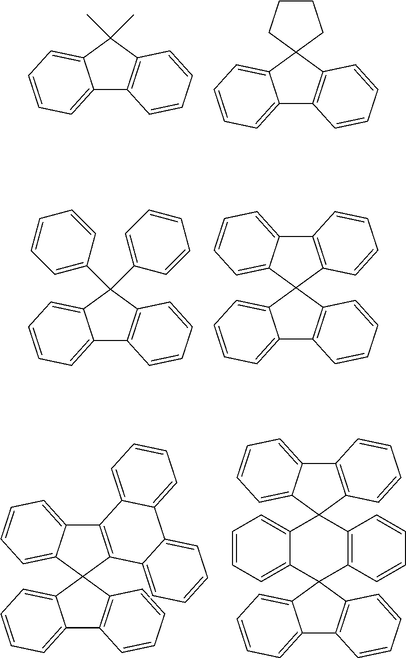

As used herein, an alkoxy group is represented by —O(R₁₀₁), and R₁₀₁ may be selected from the examples of the alkyl group described above.

As used herein, an aryloxy group is represented by —O(R₁₀₂), and R₁₀₂ may be selected from the examples of the aryl group described above.

As used herein, an alkylthio group is represented by —S(R₁₀₃), and R₁₀₃ may be selected from the examples of the alkyl group described above.

As used herein, an arylthio group is represented by —S(R₁₀₄), and R₁₀₄ may be selected from the examples of the aryl group described above.

As used herein, an alkylsulfinyl group is represented by —S(═O)> (R₁₀₅), and R₁₀₅ may be selected from the examples of the alkyl group described above.

As used herein, an arylsulfinyl group is represented by —S(═O) 2 (R₁₀₆), and R₁₀₆ may be selected from the examples of the aryl group described above.

In the present specification, a heteroaryl group includes at least one heteroatom selected from S, O, Se, N, and Si, and includes a monocyclic or polycyclic group having 2 to 60 carbon atoms, and may be further substituted by another substituent. Here, the polycyclic group refers to a group in which a heteroaryl group is directly connected to or condensed with another ring group. Here, the other ring group may be a heteroaryl group, but may also be another type of ring group, such as a cycloalkyl group, a heterocycloalkyl group, an aryl group, etc. The heteroaryl group may have 2 to 60, 2 to 40, or 3 to 25 carbon atoms. Examples of the heteroaryl group include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinozoline group, a naphthyridine group, an acridine group, a phenanthridine group, an imidazopyridine group, a diazanaphthalene group, a triazaindene group, an indole group, an indolizine group, a benzothiazole group, benzoxazole group, benzimidazole group, benzothiophene group, benzofuran group, dibenzothiophene group, dibenzofuran group, carbazole group, benzocarbazole group, dibenzocarbazole group, phenazine group, dibenzosilole group, spirobi (dibenzosilole), dihydrophenazine group, phenoxazine group, phenanthridine group, thienyl group, indolo[2,3-a]carbazole group, indolo[2,3-b]carbazole group, indoline group, 10,11-dihydro-dibenzo[b,f]azepine group, 9,10-dihydroacridine group, phenanthrazine group, phenothiathiazine group, phthalazine group, phenanthroline group, naphthobenzofuran group, naphthobenzothiophene group, benzo[c][1,2,5]thiadiazole group, Examples thereof include, but are not limited to, 2,3-dihydrobenzo[b]thiophene group, 2,3-dihydrobenzofuran group, 5,10-dihydrodibenzo[b,e][1,4]azacillin group, pyrazolo[1,5-c]quinazoline group, pyrido[1,2-b]indazole group, pyrido[1,2-a]imidazo[1,2-e]indolin group, and 5,11-dihydroindeno[1,2-b]carbazole group.

In this specification, when a substituent is a carbazole group, it means bonding to the nitrogen or carbon of the carbazole group.

In this specification, when a carbazole group is substituted, an additional substituent may be substituted on the nitrogen or carbon of the carbazole group.

In this specification, examples of a benzocarbazole group may have any of the following 5 structures:

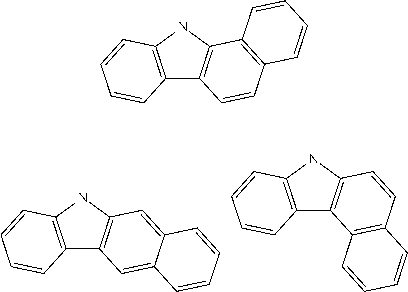

As used herein, examples of a dibenzocarbazole group may include any one of the following structures.

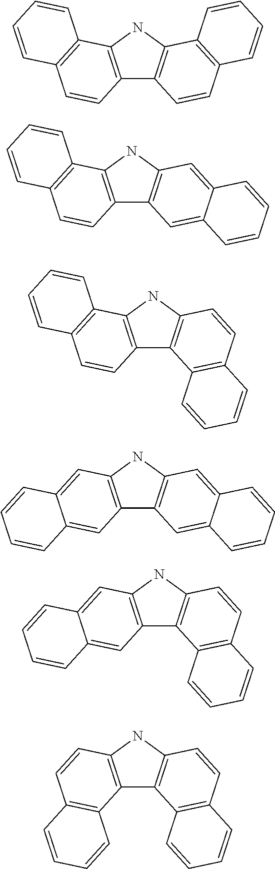

As used herein, examples of a naphthobenzofuran group may include any one of the following structures.

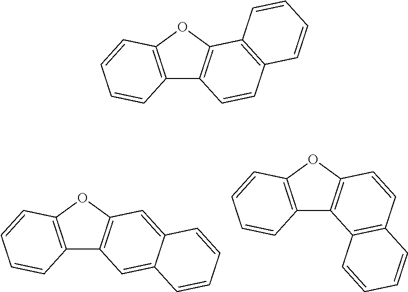

As used herein, examples of a naphthobenzothiophene group may include any one of the following structures.

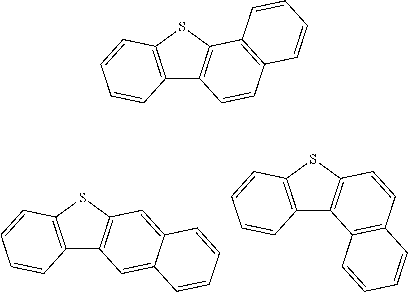

Among the substituents, —SiRR′R″ is a silyl group, which is a substituent that includes Si, and the Si atom is directly bonded as a radical. R, R′ and R″ may be the same or different, and each independently may be a substituent including at least one of hydrogen; deuterium; a halogen group, an alkyl group; an alkenyl group; an alkoxy group; a cycloalkyl group; a heterocycloalkyl group; an aryl group; and a heteroaryl group. Examples of the silyl group may include,

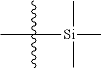 [SMILES: *[Si](C)(C)C]

(trimethylsilyl group),

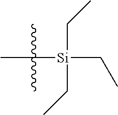 [SMILES: *[Si](CC)(CC)CC]

(triethylsilyl group),

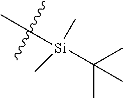 [SMILES: *[Si](C)(C)C(C)(C)C]

(t-butyldimethylsilyl group),

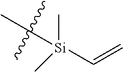 [SMILES: *[Si](C)(C)C=C]

(vinyldimethylsilyl group),

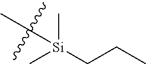 [SMILES: *[Si](C)(C)CCC]

(propyldimethylsilyl group), (triphenylsilyl group)

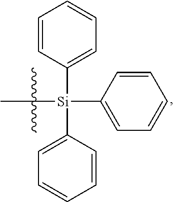 [SMILES: *[Si](c1ccccc1)(c1ccccc1)c1ccccc1]

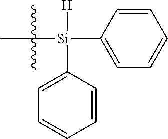 [SMILES: *[SiH](c1ccccc1)c1ccccc1]

(diphenylsilyl group), and

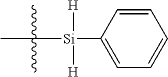 [SMILES: *[SiH2]c1ccccc1]

(phenylsilyl group), but are not limited thereto.

Among the substituents, —P(═O)RR′ is a phosphine oxide group, wherein R and R′ may be the same or different, and each independently may be a substituent including at least one of hydrogen; deuterium; a halogen group; an alkyl group; an alkenyl group; an alkoxy group; a cycloalkyl group; a heterocycloalkyl group; an aryl group; and a heteroaryl group, and in particular may be an alkyl group or an aryl group. The alkyl group and aryl group may be selected from the examples described above. For example, the phosphine oxide group may include a dimethylphosphine oxide group, a diphenylphosphine oxide group, and a dinaphthylphosphine oxide group, but is not limited thereto.

Among the substituents, —NRR′ is an amine group, wherein R and R′ may be the same or different, and each independently may be a substituent including at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂, a monoalkylamine group, a monoarylamine group, a monoheteroarylamine group, a dialkylamine group, a diarylamine group, a diheteroarylamine group, an alkylarylamine group, an alkylheteroarylamine group, and an arylheteroarylamine group, and the number of carbon atoms is not particularly limited, but is preferably 1 to 30. Examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methylanthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, and a biphenyltriphenylenylamine group, but are not limited thereto.

As used herein, an arylene group may be selected from the examples of the aryl group described above, except that it is divalent.

As used herein, a heteroarylene group may be selected from the examples of the heteroaryl group described above, except that it is divalent.

As used herein, the term “adjacent” group may refer to a substituent that is substituted on an atom directly linked to the atom substituted by the substituent, a substituent that is sterically closest to the substituent, or another substituent substituted on the atom substituted by the substituent. For example, two substituents substituted at the ortho positions of a benzene ring and two substituents substituted on the same carbon atom of an aliphatic ring may be interpreted as “adjacent” groups.

Hydrocarbon rings and heterocycles that can be formed by adjacent groups may include aliphatic hydrocarbon rings, aromatic hydrocarbon rings, aliphatic heterocycles, and aromatic heterocycles, and the structures exemplified by the above-described cycloalkyl groups, aryl groups, heterocycloalkyl groups, and heteroaryl groups may apply thereto, except that they are not monovalent.

Generally, compounds bonded to hydrogen and compounds substituted with deuterium exhibit different thermodynamic behaviors. The reason is that the mass of the deuterium atom is twice that of hydrogen. Due to this difference in atomic mass, deuterium has the characteristic of having lower vibrational energy.

In addition, the single bond dissociation energy (BDE) of carbon-deuterium is higher than that of carbon-hydrogen. Therefore, structures substituted with deuterium have increased thermal stability, resulting in improved lifetime of devices utilizing them.

When a compound is deposited on a silicon wafer, substances including deuterium tend to pack more tightly at intermolecular distance. In addition, observation of the thin film surface with an atomic force microscope (AFM) reveals that thin films formed from deuterium-containing compounds are deposited with a more uniform surface and without agglomeration.

The heterocyclic compound of Formula 1 of the present disclosure has a deuterium substitution ratio of greater than 0% and not more than 100%. When deuterium is substituted, the ground state energy is lowered compared to hydrogen-substituted compounds. Furthermore, as the carbon-deuterium bond length decreases, the molecular hard-core volume decreases. This can reduce electrical polarizability and weaken intermolecular interactions, resulting in a more stable stacking structure during device fabrication.

These characteristics create an amorphous state in the thin film, thereby lowering the crystallinity. In other words, the heterocyclic compound of Formula 1 may be effective in improving the heat resistance of OLED devices, thereby enhancing their lifetime and operating characteristics.

The heterocyclic compound according to the present disclosure may be represented by Formula 1 below.

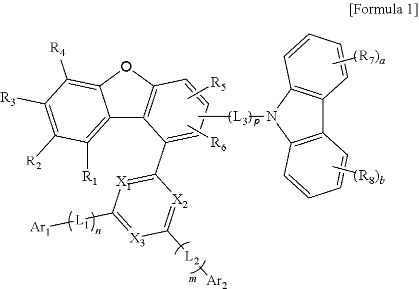

In Formula 1, X₁ to X₃ are each independently N or CR₉. However, at least two of X₁ to X₃ are nitrogen.

For example, X₁ and X₂ may be nitrogen, and X₃ may be CR₉. For example, X₁ and X₃ may be nitrogen, and X₂ may be CR₉. For example, X₂ and X₃ may be nitrogen, and X₁ may be CR₉. For example, X₁ to X₃ may each be nitrogen.

R₁ to R₉ are each independently selected from the group consisting of hydrogen; deuterium; a halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, wherein adjacent groups are linked to form a substituted or unsubstituted aryl group having 6 to 60 carbon atoms; a substituted or unsubstituted heterocycloalkyl group having 2 to 60 carbon atoms; or a substituted or unsubstituted heteroaryl group having 2 to 60 carbon atoms. R, R′ and R″ are each independently hydrogen; deuterium; a cyano group (—CN); a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms.

According to exemplary embodiments, two of R₁ to R₉ may each independently be a substituted or unsubstituted aryl group having 6 to 60 carbon atoms, or a substituted or unsubstituted heteroaryl group having 2 to 60 carbon atoms.

For example, R₇ and R₈ may each independently be a substituted or unsubstituted aryl group having 6 to 60 carbon atoms, or a substituted or unsubstituted heteroaryl group having 2 to 60 carbon atoms.

For example, R₇ may be a substituted or unsubstituted aryl group having 6 to 60 carbon atoms, or a substituted or unsubstituted heteroaryl group having 2 to 60 carbon atoms.

For example, R₈ may be a substituted or unsubstituted aryl group having 6 to 60 carbon atoms, or a substituted or unsubstituted heteroaryl group having 2 to 60 carbon atoms.

According to exemplary embodiments, R₁ to R₉ may each independently be hydrogen or deuterium. According to some embodiments, R₁ to R₉ may each be hydrogen. According to some embodiments, R₁ to R₉ may each be deuterium.

Ar₁ and Ar₂ are each independently a substituted or unsubstituted aryl group having 6 to 60 carbon atoms, or a substituted or unsubstituted heteroaryl group having 2 to 60 carbon atoms.

According to exemplary embodiments, Ar₁ and Ar₂ may each independently be a substituted or unsubstituted aryl group having 6 to 40 carbon atoms, or a substituted or unsubstituted heteroaryl group having 2 to 40 carbon atoms.

According to some embodiments, Ar₁ and Ar₂ may each independently be a substituted or unsubstituted aryl group having 6 to 20 carbon atoms, or a substituted or unsubstituted heteroaryl group having 2 to 20 carbon atoms.

According to exemplary embodiments, Ar₁ and Ar₂ are each independently an aryl group having 6 to 60 carbon atoms that is deuterated or non-deuterated, or a heteroaryl group having 2 to 60 carbon atoms that is deuterated or non-deuterated.

According to exemplary embodiments, Ar₁ and Ar₂ may each independently be an aryl group having 6 to 40 carbon atoms that is deuterated or non-deuterated, or a heteroaryl group having 2 to 40 carbon atoms that is deuterated or non-deuterated.

According to some embodiments, Ar₁ and Ar₂ may each independently be an aryl group having 6 to 20 carbon atoms that is deuterated or non-deuterated, or a heteroaryl group having 2 to 20 carbon atoms that is deuterated or non-deuterated.

For example, Ar₁ and Ar₂ may each independently be a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylenyl group, a substituted or unsubstituted anthryl group, a substituted or unsubstituted dimethylfluorenyl group, a substituted or unsubstituted diethylfluorenyl group, a substituted or unsubstituted spirobifluorenyl group, a substituted or unsubstituted furan group, a substituted or unsubstituted thiophene group, a substituted or unsubstituted benzofuran group, a substituted or unsubstituted benzothiophene group, a substituted or unsubstituted dibenzofuran group, a substituted or unsubstituted dibenzothiophene group, a substituted or unsubstituted N-carbazolyl group, a substituted or unsubstituted C-carbazolyl group, or a combination thereof.

For example, Ar₁ and Ar₂ may each independently be a deuterated or non-deuterated phenyl group, a deuterated or non-deuterated biphenyl group, a deuterated or non-deuterated terphenyl group, a deuterated or non-deuterated naphthyl group, a deuterated or non-deuterated phenanthrenyl group, a deuterated or non-deuterated triphenylenyl group, a deuterated or non-deuterated anthryl group, a deuterated or non-deuterated dimethylfluorenyl group, a deuterated or non-deuterated diethylfluorenyl group, a deuterated or non-deuterated spirobifluorenyl group, a deuterated or non-deuterated furan group, a deuterated or non-deuterated thiophene group, a deuterated or non-deuterated benzofuran group, a deuterated or non-deuterated benzothiophene group, a deuterated or non-deuterated dibenzofuran group, a deuterated or non-deuterated dibenzothiophene group, a deuterated or non-deuterated N-carbazolyl group, a deuterated or non-deuterated C-carbazolyl group, or a combination thereof.

L₁ and L₂ may each independently be a direct linkage, a substituted or unsubstituted arylene group having 6 to 60 carbon atoms, or a substituted or unsubstituted heteroarylene group having 2 to 60 carbon atoms.

According to exemplary embodiments, L₁ and L₂ may each independently be a direct linkage, a substituted or unsubstituted arylene group having 6 to 40 carbon atoms, or a substituted or unsubstituted heteroarylene group having 2 to 40 carbon atoms.

According to some embodiments, L₁ and L₂ may each independently be a direct linkage, a substituted or unsubstituted arylene group having 6 to 20 carbon atoms, or a substituted or unsubstituted heteroarylene group having 2 to 20 carbon atoms.

According to exemplary embodiments, L₁ and L₂ may each independently be a direct linkage, a deuterated or non-deuterated arylene group having 6 to 60 carbon atoms, or a deuterated or non-deuterated heteroarylene group having 2 to 60 carbon atoms.

According to exemplary embodiments, L₁ and L₂ may each independently be a direct linkage, a deuterated or non-deuterated arylene group having 6 to 40 carbon atoms, or a deuterated or non-deuterated heteroarylene group having 2 to 40 carbon atoms.

According to some embodiments, L₁ and L₂ may each independently be a direct linkage, a deuterated or non-deuterated arylene group having 6 to 20 carbon atoms, or a deuterated or non-deuterated heteroarylene group having 2 to 20 carbon atoms.

For example, L₁ and L₂ may each independently be a direct linkage, a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylenyl group, a substituted or unsubstituted anthryl group, a substituted or unsubstituted dimethylfluorenyl group, a substituted or unsubstituted diethylfluorenyl group, a substituted or unsubstituted spirobifluorenyl group, a substituted or unsubstituted furan group, a substituted or unsubstituted thiophene group, a substituted or unsubstituted benzofuran group, a substituted or unsubstituted benzothiophene group, a substituted or unsubstituted dibenzofuran group, a substituted or unsubstituted dibenzothiophene group, a substituted or unsubstituted N-carbazolyl group, a substituted or unsubstituted C-carbazolyl group, or a combination thereof.

For example, L₁ and L₂ may each independently be a direct linkage, a deuterated or non-deuterated phenyl group, a deuterated or non-deuterated biphenyl group, a deuterated or non-deuterated terphenyl group, a deuterated or non-deuterated naphthyl group, a deuterated or non-deuterated phenanthrenyl group, a deuterated or non-deuterated triphenylenyl group, a deuterated or non-deuterated anthryl group, a deuterated or non-deuterated dimethylfluorenyl group, a deuterated or non-deuterated diethylfluorenyl group, a deuterated or non-deuterated spirobifluorenyl group, a deuterated or non-deuterated furan group, a deuterated or non-deuterated thiophene group, a deuterated or non-deuterated benzofuran group, a deuterated or non-deuterated benzothiophene group, a deuterated or non-deuterated dibenzofuran group, a deuterated or non-deuterated dibenzothiophene group, a deuterated or non-deuterated N-carbazolyl group, a deuterated or non-deuterated C-carbazolyl group, or a combination thereof.

L₃ is a substituted or unsubstituted arylene group having 6 to 60 carbon atoms, or a substituted or unsubstituted heteroarylene group having 2 to 60 carbon atoms.

According to exemplary embodiments, L₃ may be a substituted or unsubstituted arylene group having 6 to 40 carbon atoms, or a substituted or unsubstituted heteroarylene group having 2 to 40 carbon atoms.

According to some embodiments, L₃ may be a substituted or unsubstituted arylene group having 6 to 20 carbon atoms, or a substituted or unsubstituted heteroarylene group having 2 to 20 carbon atoms.

According to exemplary embodiments, L₃ may be a deuterated or non-deuterated arylene group having 6 to 60 carbon atoms, or a deuterated or non-deuterated heteroarylene group having 2 to 60 carbon atoms.

According to exemplary embodiments, L₃ may be a deuterated or non-deuterated arylene group having 6 to 40 carbon atoms, or a deuterated or non-deuterated heteroarylene group having 2 to 40 carbon atoms.

According to some embodiments, L₃ may be a deuterated or non-deuterated arylene group having 6 to 20 carbon atoms, or a deuterated or non-deuterated heteroarylene group having 2 to 20 carbon atoms.

For example, L₃ may be a direct linkage, or a divalent group obtained by removing one hydrogen atom from a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylenyl group, a substituted or unsubstituted anthryl group, a substituted or unsubstituted dimethylfluorenyl group, a substituted or unsubstituted diethylfluorenyl group, a substituted or unsubstituted spirobifluorenyl group, a substituted or unsubstituted furan group, a substituted or unsubstituted thiophene group, a substituted or unsubstituted benzofuran group, a substituted or unsubstituted benzothiophene group, a substituted or unsubstituted dibenzofuran group, a substituted or unsubstituted dibenzothiophene group, a substituted or unsubstituted N-carbazolyl group, a substituted or unsubstituted C-carbazolyl group, or a combination thereof.

For example, L₃ may be a direct linkage, or a divalent group obtained by removing one hydrogen atom from a deuterated or non-deuterated phenyl group, a deuterated or non-deuterated biphenyl group, a deuterated or non-deuterated terphenyl group, a deuterated or non-deuterated naphthyl group, a deuterated or non-deuterated phenanthrenyl group, a deuterated or non-deuterated triphenylenyl group, a deuterated or non-deuterated anthryl group, a deuterated or non-deuterated dimethylfluorenyl group, a deuterated or non-deuterated diethylfluorenyl group, a deuterated or non-deuterated spirobifluorenyl group, a deuterated or non-deuterated furan group, a deuterated or non-deuterated thiophene group, a deuterated or non-deuterated benzofuran group, a deuterated or non-deuterated benzothiophene group, a deuterated or non-deuterated dibenzofuran group, a deuterated or non-deuterated dibenzothiophene group, a deuterated or non-deuterated N-carbazolyl group, a deuterated or non-deuterated C-carbazolyl group, or a combination thereof.

However, L₃ is not a group represented by Formula 2 below.

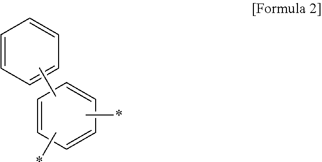

n and m are each independently 0 or an integer of 1 to 5. According to exemplary embodiments, n and m may each independently be 0, 1, or 2.

p is an integer of 1 to 5. According to exemplary embodiments, p may be 1, 2, or 3.

When n, m, and p are each independently 2 or more, a plurality of L₁ groups, L₂ groups, and L₃ groups may each independently be the same or different. For example, when n is 2 or more, a plurality of L₁ groups may be the same or different. For example, when m is 2 or more, a plurality of L₂ groups may be the same or different. For example, when pis 2 or more, a plurality of L₃ groups may be the same or different.

a and b are each independently 0 or an integer of 1 to 4. According to exemplary embodiments, a and b may each independently be 0, 1, 2, 3, or 4.

When a and b are each independently 2 or more, a plurality of R₇ groups and R₈ groups may each independently be the same or different. For example, when a is 2 or more, a plurality of R₇ groups may be the same or different. For example, when b is 2 or more, a plurality of R₈ groups may be the same or different.

When a and b are each independently 2 or more, a plurality of R₇ groups and R₈ groups may each independently be at least one of a substituted or unsubstituted aryl group having 6 to 60 carbon atoms, or a substituted or unsubstituted heteroaryl group having 2 to 60 carbon atoms. For example, when a is 2 or more, a plurality of R₇ groups may be at least one of a substituted or unsubstituted aryl group having 6 to 60 carbon atoms, or a substituted or unsubstituted heteroaryl group having 2 to 60 carbon atoms. For example, when b is 2 or more, at least one of a plurality of R₈ groups may be a substituted or unsubstituted aryl group having 6 to 60 carbon atoms, or a substituted or unsubstituted heteroaryl group having 2 to 60 carbon atoms.

* may represent a bonding site.

According to exemplary embodiments, the heterocyclic compound may be represented by any one of Formulae 1-1 to 1-3 below.

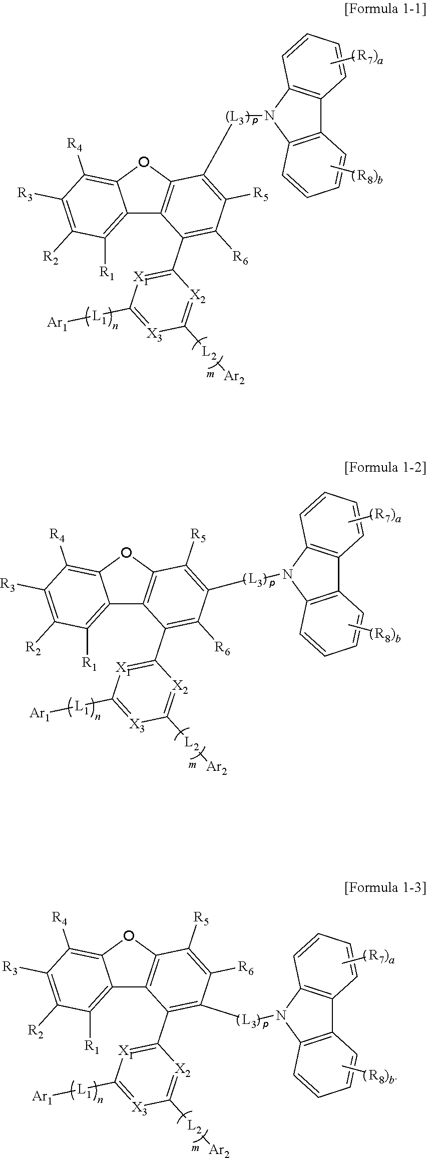

In Formulae 1-1 to 1-3, X₁ to X₃ may each independently be N or CR₉. However, at least two of X₁ to X₃ may each be nitrogen.

For example, X₁ and X₂ may be nitrogen, and X₃ may be CR₉. For example, X₁ and X₃ may be nitrogen, and X₂ may be CR₉. For example, X₂ and X₃ may be nitrogen, and X₁ may be CR₉. For example, X₁ to X₃ may each be nitrogen.

R₁ to R₉ are each independently selected from the group consisting of hydrogen; deuterium; a halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, wherein adjacent groups may be linked to form a substituted or unsubstituted aryl group having 6 to 60 carbon atoms; a substituted or unsubstituted heterocycloalkyl group having 2 to 60 carbon atoms; or a substituted or unsubstituted heteroaryl group having 2 to 60 carbon atoms. R, R′ and R″ are each independently hydrogen; deuterium; a cyano group (—CN); a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms.

According to exemplary embodiments, two of R₁ to R₉ may each independently be a substituted or unsubstituted aryl group having 6 to 60 carbon atoms, or a substituted or unsubstituted heteroaryl group having 2 to 60 carbon atoms.

For example, R₇ and R₈ may each independently be a substituted or unsubstituted aryl group having 6 to 60 carbon atoms, or a substituted or unsubstituted heteroaryl group having 2 to 60 carbon atoms.

For example, R₇ may be a substituted or unsubstituted aryl group having 6 to 60 carbon atoms, or a substituted or unsubstituted heteroaryl group having 2 to 60 carbon atoms.

For example, R₈ may be a substituted or unsubstituted aryl group having 6 to 60 carbon atoms, or a substituted or unsubstituted heteroaryl group having 2 to 60 carbon atoms.

According to exemplary embodiments, R₁ to R₉ may each independently be hydrogen or deuterium. In some embodiments, R₁ to R₉ may each be hydrogen. In some embodiments, R₁ to R₉ may each be deuterium.

Ar₁ and Ar₂ may each independently be a substituted or unsubstituted aryl group having 6 to 60 carbon atoms, or a substituted or unsubstituted heteroaryl group having 2 to 60 carbon atoms.

According to exemplary embodiments, Ar₁ and Ar₂ may each independently be a substituted or unsubstituted aryl group having 6 to 40 carbon atoms, or a substituted or unsubstituted heteroaryl group having 2 to 40 carbon atoms.

According to some embodiments, Ar₁ and Ar₂ may each independently be a substituted or unsubstituted aryl group having 6 to 20 carbon atoms, or a substituted or unsubstituted heteroaryl group having 2 to 20 carbon atoms.

According to exemplary embodiments, Ar₁ and Ar₂ may each independently be an aryl group having 6 to 60 carbon atoms that is deuterated or non-deuterated, or a heteroaryl group having 2 to 60 carbon atoms that is deuterated or non-deuterated.

According to exemplary embodiments, Ar₁ and Ar₂ may each independently be an aryl group having 6 to 40 carbon atoms that is deuterated or non-deuterated, or a heteroaryl group having 2 to 40 carbon atoms that is deuterated or non-deuterated.

According to some embodiments, Ar₁ and Ar₂ may each independently be an aryl group having 6 to 20 carbon atoms that is deuterated or non-deuterated, or a heteroaryl group having 2 to 20 carbon atoms that is deuterated or non-deuterated.

For example, Ar₁ and Ar₂ may each independently be a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylenyl group, a substituted or unsubstituted anthryl group, a substituted or unsubstituted dimethylfluorenyl group, a substituted or unsubstituted diethylfluorenyl group, a substituted or unsubstituted spirobifluorenyl group, a substituted or unsubstituted furan group, a substituted or unsubstituted thiophene group, a substituted or unsubstituted benzofuran group, a substituted or unsubstituted benzothiophene group, a substituted or unsubstituted dibenzofuran group, a substituted or unsubstituted dibenzothiophene group, a substituted or unsubstituted N-carbazolyl group, a substituted or unsubstituted C-carbazolyl group, or a combination thereof.

For example, Ar₁ and Ar₂ may each independently be a deuterated or non-deuterated phenyl group, a deuterated or non-deuterated biphenyl group, a deuterated or non-deuterated terphenyl group, a deuterated or non-deuterated naphthyl group, a deuterated or non-deuterated phenanthrenyl group, a deuterated or non-deuterated triphenylenyl group, a deuterated or non-deuterated anthryl group, a deuterated or non-deuterated dimethylfluorenyl group, a deuterated or non-deuterated diethylfluorenyl group, a deuterated or non-deuterated spirobifluorenyl group, a deuterated or non-deuterated furan group, a deuterated or non-deuterated thiophene group, a deuterated or non-deuterated benzofuran group, a deuterated or non-deuterated benzothiophene group, a deuterated or non-deuterated dibenzofuran group, a deuterated or non-deuterated dibenzothiophene group, a deuterated or non-deuterated N-carbazolyl group, a deuterated or non-deuterated C-carbazolyl group, or a combination thereof.

L₁ and L₂ may each independently be a direct linkage, a substituted or unsubstituted arylene group having 6 to 60 carbon atoms, or a substituted or unsubstituted heteroarylene group having 2 to 60 carbon atoms.

According to exemplary embodiments, L₁ and L₂ may each independently be a direct linkage, a substituted or unsubstituted arylene group having 6 to 40 carbon atoms, or a substituted or unsubstituted heteroarylene group having 2 to 40 carbon atoms.

According to some embodiments, L₁ and L₂ may each independently be a direct linkage, a substituted or unsubstituted arylene group having 6 to 20 carbon atoms, or a substituted or unsubstituted heteroarylene group having 2 to 20 carbon atoms.

According to exemplary embodiments, each independently may be a direct linkage, a deuterated or non-deuterated arylene group having 6 to 60 carbon atoms, or a deuterated or non-deuterated heteroarylene group having 2 to 60 carbon atoms.

According to exemplary embodiments, L₁ and L₂ may each independently be a direct linkage, a deuterated or non-deuterated arylene group having 6 to 40 carbon atoms, or a deuterated or non-deuterated heteroarylene group having 2 to 40 carbon atoms.

According to some embodiments, L₁ and L₂ may each independently be a direct linkage, a deuterated or non-deuterated arylene group having 6 to 20 carbon atoms, or a deuterated or non-deuterated heteroarylene group having 2 to 20 carbon atoms.

For example, L₁ and L₂ may each independently be a direct linkage, or a divalent group obtained by removing one hydrogen atom from a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylenyl group, a substituted or unsubstituted anthryl group, a substituted or unsubstituted dimethylfluorenyl group, a substituted or unsubstituted diethylfluorenyl group, a substituted or unsubstituted spirobifluorenyl group, a substituted or unsubstituted furan group, a substituted or unsubstituted thiophene group, a substituted or unsubstituted benzofuran group, a substituted or unsubstituted benzothiophene group, a substituted or unsubstituted dibenzofuran group, a substituted or unsubstituted dibenzothiophene group, a substituted or unsubstituted N-carbazolyl group, a substituted or unsubstituted C-carbazolyl group, or a combination thereof.

For example, L₁ and L₂ may each independently be a direct linkage, or a divalent group obtained by removing one hydrogen atom from a deuterated or non-deuterated phenyl group, a deuterated or non-deuterated biphenyl group, a deuterated or non-deuterated terphenyl group, a deuterated or non-deuterated naphthyl group, a deuterated or non-deuterated phenanthrenyl group, a deuterated or non-deuterated triphenylenyl group, a deuterated or non-deuterated anthryl group, a deuterated or non-deuterated dimethylfluorenyl group, a deuterated or non-deuterated diethylfluorenyl group, a deuterated or non-deuterated spirobifluorenyl group, a deuterated or non-deuterated furan group, a deuterated or non-deuterated thiophene group, a deuterated or non-deuterated benzofuran group, a deuterated or non-deuterated benzothiophene group, a deuterated or non-deuterated dibenzofuran group, a deuterated or non-deuterated dibenzothiophene group, a deuterated or non-deuterated N-carbazolyl group, a deuterated or non-deuterated C-carbazolyl group, or a combination thereof.

L₃ may be a substituted or unsubstituted arylene group having 6 to 60 carbon atoms, or a substituted or unsubstituted heteroarylene group having 2 to 60 carbon atoms.

According to exemplary embodiments, L₃ may be a substituted or unsubstituted arylene group having 6 to 40 carbon atoms, or a substituted or unsubstituted heteroarylene group having 2 to 40 carbon atoms.

In some embodiments, L₃ may be a substituted or unsubstituted arylene group having 6 to 20 carbon atoms, or a substituted or unsubstituted heteroarylene group having 2 to 20 carbon atoms.

In exemplary embodiments, L₃ may be a deuterated or non-deuterated arylene group having 6 to 60 carbon atoms, or a deuterated or non-deuterated heteroarylene group having 2 to 60 carbon atoms.

In exemplary embodiments, L₃ may be a deuterated or non-deuterated arylene group having 6 to 40 carbon atoms, or a deuterated or non-deuterated heteroarylene group having 2 to 40 carbon atoms.

In some embodiments, L₃ may be a deuterated or non-deuterated arylene group having 6 to 20 carbon atoms, or a deuterated or non-deuterated heteroarylene group having 2 to 20 carbon atoms.

For example, L₃ may be a direct linkage, or a divalent group obtained by removing one hydrogen atom from a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylenyl group, a substituted or unsubstituted anthryl group, a substituted or unsubstituted dimethylfluorenyl group, a substituted or unsubstituted diethylfluorenyl group, a substituted or unsubstituted spirobifluorenyl group, a substituted or unsubstituted furan group, a substituted or unsubstituted thiophene group, a substituted or unsubstituted benzofuran group, a substituted or unsubstituted benzothiophene group, a substituted or unsubstituted dibenzofuran group, a substituted or unsubstituted dibenzothiophene group, a substituted or unsubstituted N-carbazolyl group, a substituted or unsubstituted C-carbazolyl group, or a combination thereof.

For example, L₃ may be a direct linkage, or a divalent group obtained by removing one hydrogen atom from a deuterated or non-deuterated phenyl group, a deuterated or non-deuterated biphenyl group, a deuterated or non-deuterated terphenyl group, a deuterated or non-deuterated naphthyl group, a deuterated or non-deuterated phenanthrenyl group, a deuterated or non-deuterated triphenylenyl group, a deuterated or non-deuterated anthryl group, a deuterated or non-deuterated dimethylfluorenyl group, a deuterated or non-deuterated diethylfluorenyl group, a deuterated or non-deuterated spirobifluorenyl group, a deuterated or non-deuterated furan group, a deuterated or non-deuterated thiophene group, a deuterated or non-deuterated benzofuran group, a deuterated or non-deuterated benzothiophene group, a deuterated or non-deuterated dibenzofuran group, a deuterated or non-deuterated dibenzothiophene group, a deuterated or non-deuterated N-carbazolyl group, a deuterated or non-deuterated C-carbazolyl group, or a combination thereof.

However, L₃ may not be the group represented by Formula 2 below.

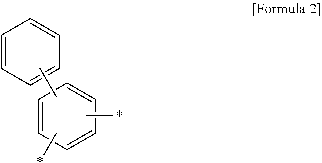

n and m are each independently 0 or an integer of 1 to 5. According to exemplary embodiments, n and m may each independently be 0, 1, or 2.

p may be an integer of 1 to 5. According to exemplary embodiments, p may be 1, 2, or 3.

When n, m, and p are each independently 2 or more, a plurality of L₁ groups, L₂ groups, and L₃ groups may each independently be the same or different. For example, when n is 2 or more, a plurality of L₁ groups may be the same or different. For example, when m is 2 or more, a plurality of L₂ groups may be the same or different. For example, when pis 2 or more, a plurality of L₃ groups may be the same or different.

a and b may each independently be 0 or an integer of 1 to 4. According to exemplary embodiments, a and b may each independently be 0, 1, 2, 3, or 4.

When a and b are each independently 2 or more, a plurality of R₇ groups and R₈ groups may each independently be the same or different. For example, when a is 2 or more, a plurality of R₇ groups may each be the same or different. For example, when b is 2 or more, a plurality of R₈ groups may each independently be the same or different.

When a and b are each independently 2 or more, a plurality of R₇ groups and R₈ groups may each independently be a substituted or unsubstituted aryl group having 6 to 60 carbon atoms, or a substituted or unsubstituted heteroaryl group having 2 to 60 carbon atoms. For example, when a is 2 or more, at least one of the plurality of R₇ groups may be a substituted or unsubstituted aryl group having 6 to 60 carbon atoms, or a substituted or unsubstituted heteroaryl group having 2 to 60 carbon atoms. For example, when b is 2 or more, at least one of the plurality of R₈ groups may be a substituted or unsubstituted aryl group having 6 to 60 carbon atoms, or a substituted or unsubstituted heteroaryl group having 2 to 60 carbon atoms.

* may represent a bonding site.

According to exemplary embodiments, the heterocyclic compound may be represented by any one of Formulae 1-4 to 1-5 below.

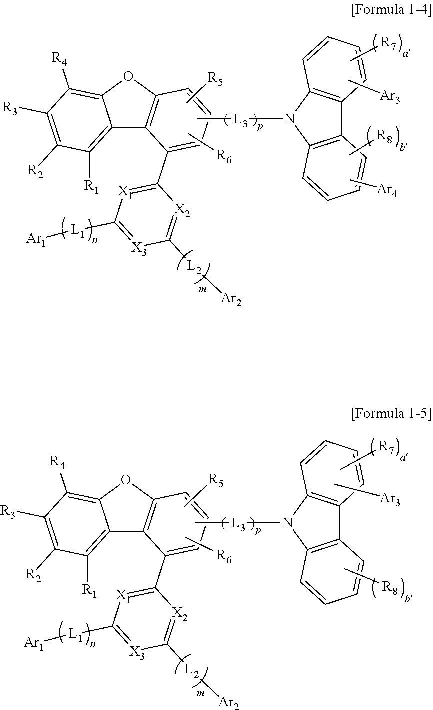

In Formulae 1-4 to 1-5, Ar₃ and Ar₄ may each independently be a substituted or unsubstituted aryl group having 6 to 60 carbon atoms, or a substituted or unsubstituted heteroaryl group having 2 to 60 carbon atoms.

According to exemplary embodiments, Ar₃ and Ar₄ may each independently be a substituted or unsubstituted aryl group having 6 to 40 carbon atoms, or a substituted or unsubstituted heteroaryl group having 2 to 40 carbon atoms.

According to some embodiments, Ar₃ and Ar₄ may each independently be a substituted or unsubstituted aryl group having 6 to 20 carbon atoms, or a substituted or unsubstituted heteroaryl group having 2 to 20 carbon atoms.

According to exemplary embodiments, Ar₃ and Ar₄ may each independently be an aryl group having 6 to 60 carbon atoms that is deuterated or non-deuterated, or a heteroaryl group having 2 to 60 carbon atoms that is deuterated or non-deuterated.

According to exemplary embodiments, Ar₃ and Ar₄ may each independently be an aryl group having 6 to 40 carbon atoms that is deuterated or non-deuterated, or a heteroaryl group having 2 to 40 carbon atoms that is deuterated or non-deuterated.

According to some embodiments, Ar₃ and Ar₄ may each independently be an aryl group having 6 to 20 carbon atoms that is deuterated or non-deuterated, or a heteroaryl group having 2 to 20 carbon atoms that is deuterated or non-deuterated.

For example, Ar₃ and Ar₄ may each independently be a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylenyl group, a substituted or unsubstituted anthryl group, a substituted or unsubstituted dimethylfluorenyl group, a substituted or unsubstituted diethylfluorenyl group, a substituted or unsubstituted spirobifluorenyl group, a substituted or unsubstituted furan group, a substituted or unsubstituted thiophene group, a substituted or unsubstituted benzofuran group, a substituted or unsubstituted benzothiophene group, a substituted or unsubstituted dibenzofuran group, a substituted or unsubstituted dibenzothiophene group, a substituted or unsubstituted N-carbazolyl group, a substituted or unsubstituted C-carbazolyl group, or a combination thereof.

For example, Ar₃ and Ar₄ may each independently be a deuterated or non-deuterated phenyl group, a deuterated or non-deuterated biphenyl group, a deuterated or non-deuterated terphenyl group, a deuterated or non-deuterated naphthyl group, a deuterated or non-deuterated phenanthrenyl group, a deuterated or non-deuterated triphenylenyl group, a deuterated or non-deuterated anthryl group, a deuterated or non-deuterated dimethylfluorenyl group, a deuterated or non-deuterated diethylfluorenyl group, a deuterated or non-deuterated spirobifluorenyl group, a deuterated or non-deuterated furan group, a deuterated or non-deuterated thiophene group, a deuterated or non-deuterated benzofuran group, a deuterated or non-deuterated benzothiophene group, a deuterated or non-deuterated dibenzofuran group, a deuterated or non-deuterated dibenzothiophene group, a deuterated or non-deuterated N-carbazolyl group, a deuterated or non-deuterated C-carbazolyl group, or a combination thereof.

a′ and b′ may each independently be 0 or an integer of 1 to 3. According to exemplary embodiments, a′ and b′ may each independently be 0, 1, or 2.

When a′ and b′ are each independently 2 or more, a plurality of R₇ groups and R₈ groups may each independently be the same or different. For example, when a′ is 2 or more, a plurality of R₇ groups may each be the same or different. For example, when b′ is 2 or more, a plurality of R₈ groups may each be the same or different.

In Formulae 1-4 and 1-5, X₁ to X₃ are each independently N or CR₉. However, at least two of X₁ to X₃ may each be nitrogen.

For example, X₁ and X₂ may be nitrogen, and X₃ may be CR₉. For example, X₁ and X₃ may be nitrogen, and X₂ may be CR₉. For example, X₂ and X₃ may be nitrogen, and X₁ may be CR₉. For example, X₁ to X₃ may each be nitrogen.

R₁ to R₉ are each independently selected from the group consisting of hydrogen; deuterium; a halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, wherein adjacent groups are linked to form a substituted or unsubstituted aryl group having 6 to 60 carbon atoms; a substituted or unsubstituted heterocycloalkyl group having 2 to 60 carbon atoms; or a substituted or unsubstituted heteroaryl group having 2 to 60 carbon atoms. R, R′ and R″ may each independently be hydrogen; deuterium; a cyano group (—CN); a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms.

According to exemplary embodiments, two of R₁ to R₉ may each independently be a substituted or unsubstituted aryl group having 6 to 60 carbon atoms, or a substituted or unsubstituted heteroaryl group having 2 to 60 carbon atoms.

For example, R₇ and R₈ may each independently be a substituted or unsubstituted aryl group having 6 to 60 carbon atoms, or a substituted or unsubstituted heteroaryl group having 2 to 60 carbon atoms.

For example, R₇ may be a substituted or unsubstituted aryl group having 6 to 60 carbon atoms, or a substituted or unsubstituted heteroaryl group having 2 to 60 carbon atoms.

For example, R₈ may be a substituted or unsubstituted aryl group having 6 to 60 carbon atoms, or a substituted or unsubstituted heteroaryl group having 2 to 60 carbon atoms.

According to exemplary embodiments, R₁ to R₉ may each independently be hydrogen or deuterium. According to some embodiments, R₁ to R₉ may each be hydrogen. According to some embodiments, R₁ to R₉ may each be deuterium.

Ar₁ and Ar₂ may each independently be a substituted or unsubstituted aryl group having 6 to 60 carbon atoms, or a substituted or unsubstituted heteroaryl group having 2 to 60 carbon atoms.

According to exemplary embodiments, Ar₁ and Ar₂ may each independently be a substituted or unsubstituted aryl group having 6 to 40 carbon atoms, or a substituted or unsubstituted heteroaryl group having 2 to 40 carbon atoms.

According to some embodiments, Ar₁ and Ar₂ may each independently be a substituted or unsubstituted aryl group having 6 to 20 carbon atoms, or a substituted or unsubstituted heteroaryl group having 2 to 20 carbon atoms.

According to exemplary embodiments, Ar₁ and Ar₂ may each independently be an aryl group having 6 to 60 carbon atoms that is deuterated or non-deuterated, or a heteroaryl group having 2 to 60 carbon atoms that is deuterated or non-deuterated.

According to exemplary embodiments, Ar₁ and Ar₂ may each independently be an aryl group having 6 to 40 carbon atoms that is deuterated or non-deuterated, or a heteroaryl group having 2 to 40 carbon atoms that is deuterated or non-deuterated.

According to some embodiments, Ar₁ and Ar₂ may each independently be an aryl group having 6 to 20 carbon atoms that is deuterated or non-deuterated, or a heteroaryl group having 2 to 20 carbon atoms that is deuterated or non-deuterated.

For example, Ar₁ and Ar₂ may each independently be a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylenyl group, a substituted or unsubstituted anthryl group, a substituted or unsubstituted dimethylfluorenyl group, a substituted or unsubstituted diethylfluorenyl group, a substituted or unsubstituted spirobifluorenyl group, a substituted or unsubstituted furan group, a substituted or unsubstituted thiophene group, a substituted or unsubstituted benzofuran group, a substituted or unsubstituted benzothiophene group, a substituted or unsubstituted dibenzofuran group, a substituted or unsubstituted dibenzothiophene group, a substituted or unsubstituted N-carbazolyl group, a substituted or unsubstituted C-carbazolyl group, or a combination thereof.

For example, Ar₁ and Ar₂ may each independently be a deuterated or non-deuterated phenyl group, a deuterated or non-deuterated biphenyl group, a deuterated or non-deuterated terphenyl group, a deuterated or non-deuterated naphthyl group, a deuterated or non-deuterated phenanthrenyl group, a deuterated or non-deuterated triphenylenyl group, a deuterated or non-deuterated anthryl group, a deuterated or non-deuterated dimethylfluorenyl group, a deuterated or non-deuterated diethylfluorenyl group, a deuterated or non-deuterated spirobifluorenyl group, a deuterated or non-deuterated furan group, a deuterated or non-deuterated thiophene group, a deuterated or non-deuterated benzofuran group, a deuterated or non-deuterated benzothiophene group, a deuterated or non-deuterated dibenzofuran group, a deuterated or non-deuterated dibenzothiophene group, a deuterated or non-deuterated N-carbazolyl group, a deuterated or non-deuterated C-carbazolyl group, or a combination thereof.

L₁ and L₂ may each independently be a direct linkage, a substituted or unsubstituted arylene group having 6 to 60 carbon atoms, or a substituted or unsubstituted heteroarylene group having 2 to 60 carbon atoms.

According to exemplary embodiments, L₁ and L₂ may each independently be a direct linkage, a substituted or unsubstituted arylene group having 6 to 40 carbon atoms, or a substituted or unsubstituted heteroarylene group having 2 to 40 carbon atoms.

According to some embodiments, L₁ and L₂ may each independently be a direct linkage, a substituted or unsubstituted arylene group having 6 to 20 carbon atoms, or a substituted or unsubstituted heteroarylene group having 2 to 20 carbon atoms.

According to exemplary embodiments, L₁ and L₂ may each independently be a direct linkage, a deuterated or non-deuterated arylene group having 6 to 60 carbon atoms, or a deuterated or non-deuterated heteroarylene group having 2 to 60 carbon atoms.

According to exemplary embodiments, L₁ and L₂ may each independently be a direct linkage, a deuterated or non-deuterated arylene group having 6 to 40 carbon atoms, or a deuterated or non-deuterated heteroarylene group having 2 to 40 carbon atoms.

According to some embodiments, L₁ and L₂ may each independently be a direct linkage, a deuterated or non-deuterated arylene group having 6 to 20 carbon atoms, or a deuterated or non-deuterated heteroarylene group having 2 to 20 carbon atoms.

For example, L₁ and L₂ may each independently be a direct linkage, or a divalent group obtained by removing one hydrogen atom from a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylenyl group, a substituted or unsubstituted anthryl group, a substituted or unsubstituted dimethylfluorenyl group, a substituted or unsubstituted diethylfluorenyl group, a substituted or unsubstituted spirobifluorenyl group, a substituted or unsubstituted furan group, a substituted or unsubstituted thiophene group, a substituted or unsubstituted benzofuran group, a substituted or unsubstituted benzothiophene group, a substituted or unsubstituted dibenzofuran group, a substituted or unsubstituted dibenzothiophene group, a substituted or unsubstituted N-carbazolyl group, a substituted or unsubstituted C-carbazolyl group, or a combination thereof.

For example, L₁ and L₂ may each independently be a direct linkage, or a divalent group obtained by removing one hydrogen atom from a deuterated or non-deuterated phenyl group, a deuterated or non-deuterated biphenyl group, a deuterated or non-deuterated terphenyl group, a deuterated or non-deuterated naphthyl group, a deuterated or non-deuterated phenanthrenyl group, a deuterated or non-deuterated triphenylenyl group, a deuterated or non-deuterated anthryl group, a deuterated or non-deuterated dimethylfluorenyl group, a deuterated or non-deuterated diethylfluorenyl group, a deuterated or non-deuterated spirobifluorenyl group, a deuterated or non-deuterated furan group, a deuterated or non-deuterated thiophene group, a deuterated or non-deuterated benzofuran group, a deuterated or non-deuterated benzothiophene group, a deuterated or non-deuterated dibenzofuran group, a deuterated or non-deuterated dibenzothiophene group, a deuterated or non-deuterated N-carbazolyl group, a deuterated or non-deuterated C-carbazolyl group, or a combination thereof.

L₃ may be a substituted or unsubstituted arylene group having 6 to 60 carbon atoms, or a substituted or unsubstituted heteroarylene group having 2 to 60 carbon atoms.

According to exemplary embodiments, L₃ may be a substituted or unsubstituted arylene group having 6 to 40 carbon atoms, or a substituted or unsubstituted heteroarylene group having 2 to 40 carbon atoms.

According to some embodiments, L₃ may be a substituted or unsubstituted arylene group having 6 to 20 carbon atoms, or a substituted or unsubstituted heteroarylene group having 2 to 20 carbon atoms.

According to exemplary embodiments, L₃ may be a deuterated or non-deuterated arylene group having 6 to 60 carbon atoms, or a deuterated or non-deuterated heteroarylene group having 2 to 60 carbon atoms.

According to exemplary embodiments, L₃ may be a deuterated or non-deuterated arylene group having 6 to 40 carbon atoms, or a deuterated or non-deuterated heteroarylene group having 2 to 40 carbon atoms.

According to some embodiments, L₃ may be a deuterated or non-deuterated arylene group having 6 to 20 carbon atoms, or a deuterated or non-deuterated heteroarylene group having 2 to 20 carbon atoms.

For example, L₃ may be a direct linkage, or a divalent group obtained by removing one hydrogen atom from a direct linkage, or a divalent group obtained by removing one hydrogen atom from a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylenyl group, a substituted or unsubstituted anthryl group, a substituted or unsubstituted dimethylfluorenyl group, a substituted or unsubstituted diethylfluorenyl group, a substituted or unsubstituted spirobifluorenyl group, a substituted or unsubstituted furan group, a substituted or unsubstituted thiophene group, a substituted or unsubstituted benzofuran group, a substituted or unsubstituted benzothiophene group, a substituted or unsubstituted dibenzofuran group, a substituted or unsubstituted dibenzothiophene group, a substituted or unsubstituted N-carbazolyl group, a substituted or unsubstituted C-carbazolyl group, or a combination thereof.

For example, L₃ may be a direct linkage, or a divalent group obtained by removing one hydrogen atom from a deuterated or non-deuterated phenyl group, a deuterated or non-deuterated biphenyl group, a deuterated or non-deuterated terphenyl group, a deuterated or non-deuterated naphthyl group, a deuterated or non-deuterated phenanthrenyl group, a deuterated or non-deuterated triphenylenyl group, a deuterated or non-deuterated anthryl group, a deuterated or non-deuterated dimethylfluorenyl group, a deuterated or non-deuterated diethylfluorenyl group, a deuterated or non-deuterated spirobifluorenyl group, a deuterated or non-deuterated furan group, a deuterated or non-deuterated thiophene group, a deuterated or non-deuterated benzofuran group, a deuterated or non-deuterated benzothiophene group, a deuterated or non-deuterated dibenzofuran group, a deuterated or non-deuterated dibenzothiophene group, a deuterated or non-deuterated N-carbazolyl group, a deuterated or non-deuterated C-carbazolyl group, or a combination thereof.

However, L₃ may not be the group represented by Formula 2 below.

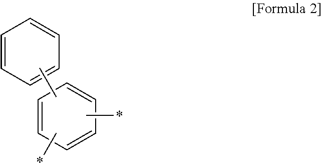

n and m may each independently be 0 or an integer of 1 to 5. According to exemplary embodiments, n and m may each independently be 0, 1, or 2.

p may be an integer of 1 to 5. According to exemplary embodiments, p may be 1, 2, or 3.

When n, m, and p are each independently 2 or more, a plurality of L₁ groups, L₂ groups, and L₃ groups may each independently be the same or different. For example, when n is 2 or more, a plurality of L₁ groups may be the same or different. For example, when m is 2 or more, a plurality of L₂ groups may be the same or different. For example, when pis 2 or more, a plurality of L₃ groups may be the same or different.

b may be 0 or an integer of 1 to 4. According to exemplary embodiments, b may be 0, 1, 2, 3, or 4.

When b is 2 or more, a plurality of R₈ groups may be the same or different.

When b is 2 or more, at least one of the a plurality of R₈ groups may be a substituted or unsubstituted aryl group having 6 to 60 carbon atoms, or a substituted or unsubstituted heteroaryl group having 2 to 60 carbon atoms.

* may represent a bonding site.

According to exemplary embodiments, in Formula 1, L₃ may be represented by one of Formulae 3-1 to 3-8 below.

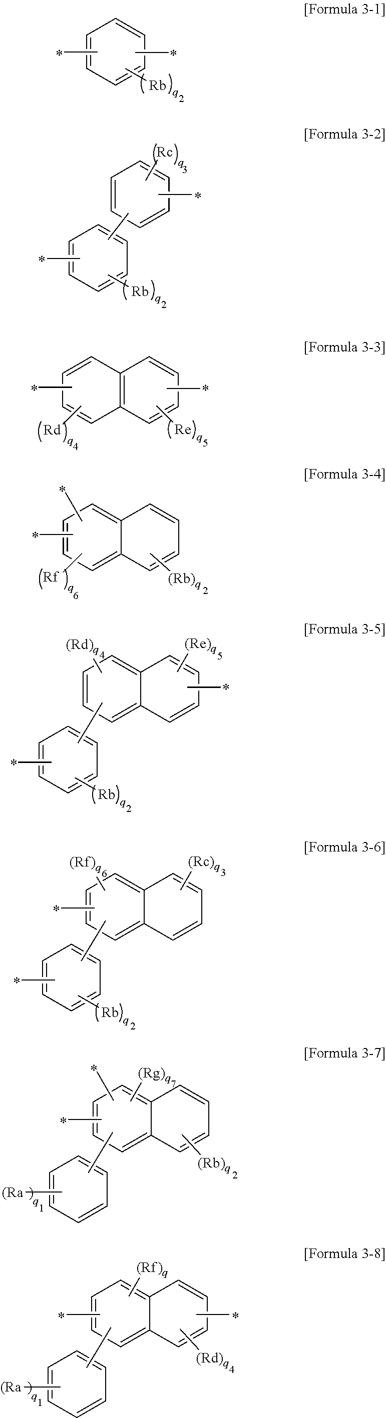

In Formulae 3-1 to 3-8, Rb may be hydrogen or deuterium, and
Ra, Rc, Rd, Re, Rf, and Rg are each independently selected from the group consisting of hydrogen; deuterium; a halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, wherein adjacent groups may be linked to form a substituted or unsubstituted aryl group having 6 to 60 carbon atoms; a substituted or unsubstituted heterocycloalkyl group having 2 to 60 carbon atoms; or a substituted or unsubstituted heteroaryl group having 2 to 60 carbon atoms.

q1 may be 0 or an integer of 1 to 5. According to exemplary embodiments, q1 may be 0, 1, or 2.

q₂ and q₃ may each independently be 0 or an integer of 1 to 4. According to exemplary embodiments, q₂ may be 0, 1, or 2. According to exemplary embodiments, q₃ may be 0, 1, or 2. q₄ and q₅ may each independently be 0 or an integer of 1 to 3. According to exemplary embodiments, q₄ may be 0, 1, or 2. According to exemplary embodiments, q₅ may be 0, 1, or 2.

q₆ may be 0, 1, or 2. According to exemplary embodiments, q₆ may be 0 or 1.

q₇ may be 0 or 1. According to exemplary embodiments, q₇ may be 0 or 1.

When q₁, q₂, q₃, 94, 95, and q₆ are each independently 2 or more, a plurality of Ra groups, Rb groups, Rc groups, Rd groups, Re groups, and Rf groups may each independently be the same or different. For example, when q₁ is 2 or more, a plurality of Ra groups may be the same or different. For example, when q₂ is 2 or more, a plurality of Rb groups may be the same or different. For example, when q₃ is 2 or more, a plurality of Rc groups may be the same or different. For example, when q₄ is 2 or more, a plurality of Rd groups may be the same or different. For example, when q₅ is 2 or more, a plurality of Re groups may be the same or different. For example, when q₆ is 2 or more, a plurality of Rf groups may be the same or different.

* may represent a bonding site.

According to exemplary embodiments, in Formula 1, Ar₁ and Ar₂ may each independently be a substituted or unsubstituted aryl group having 6 to 40 carbon atoms, or a substituted or unsubstituted heteroaryl group having 2 to 40 carbon atoms.

According to some embodiments, in Formula 1, Ar₁ and Ar₂ may each independently be a substituted or unsubstituted aryl group having 6 to 20 carbon atoms, or a substituted or unsubstituted heteroaryl group having 2 to 20 carbon atoms.

According to exemplary embodiments, in Formula 1, Ar₁ and Ar₂ may each independently be an aryl group having 6 to 40 carbon atoms that is deuterated or non-deuterated, or a heteroaryl group having 2 to 40 carbon atoms that is deuterated or non-deuterated.

In some embodiments, in Formula 1, Ar₁ and Ar₂ independently represent an aryl group having 6 to 20 carbon atoms that is deuterated or non-deuterated, or a heteroaryl group having 2 to 20 carbon atoms that is deuterated or non-deuterated.

In exemplary embodiments, in Formula 1, Ar₁ and Ar₂ may be the same or different.

In exemplary embodiments, the heterocyclic compound may have a deuterium content of 0, greater than 0 and not more than 100%.

In exemplary embodiments, the deuterium content of the compound of Formula 1 may be 0%, or 5% to 100%.

In exemplary embodiments, the deuterium content of the compound of Formula 1 may be 0%, or 10% to 100%.

According to exemplary embodiments, the deuterium content of the compound of Formula 1 may be 0%, or 15% to 100%.

According to exemplary embodiments, the deuterium content of the compound of Formula 1 may be 0%, or 20% to 100%.

According to exemplary embodiments, the deuterium content of the compound of Formula 1 may be 0%, or 25% to 100%.

According to exemplary embodiments, the deuterium content of the compound of Formula 1 may be 0%, or 30% to 100%.

According to exemplary embodiments, the deuterium content of the compound of Formula 1 may be 0%, or 50% to 100%.

According to exemplary embodiments, the deuterium content of the compound of Formula 1 may be 0%, or 70% to 100%.

According to exemplary embodiments, the deuterium content of the compound of Formula 1 may be 0%, or 90% to 100%.

According to exemplary embodiments, the deuterium content of the compound of Formula 1 may be 0%.

According to exemplary embodiments, the deuterium content of the compound of Formula 1 may be 30% to 100%.

According to exemplary embodiments, the deuterium content of the compound of Formula 1 may be 50% to 100%.

According to exemplary embodiments, the deuterium content of the compound of Formula 1 may be 70% to 100%.

According to exemplary embodiments, the deuterium content of the compound of Formula 1 may be 90% to 100%.

According to exemplary embodiments, the heterocyclic compound may be represented by any one of formulae below:

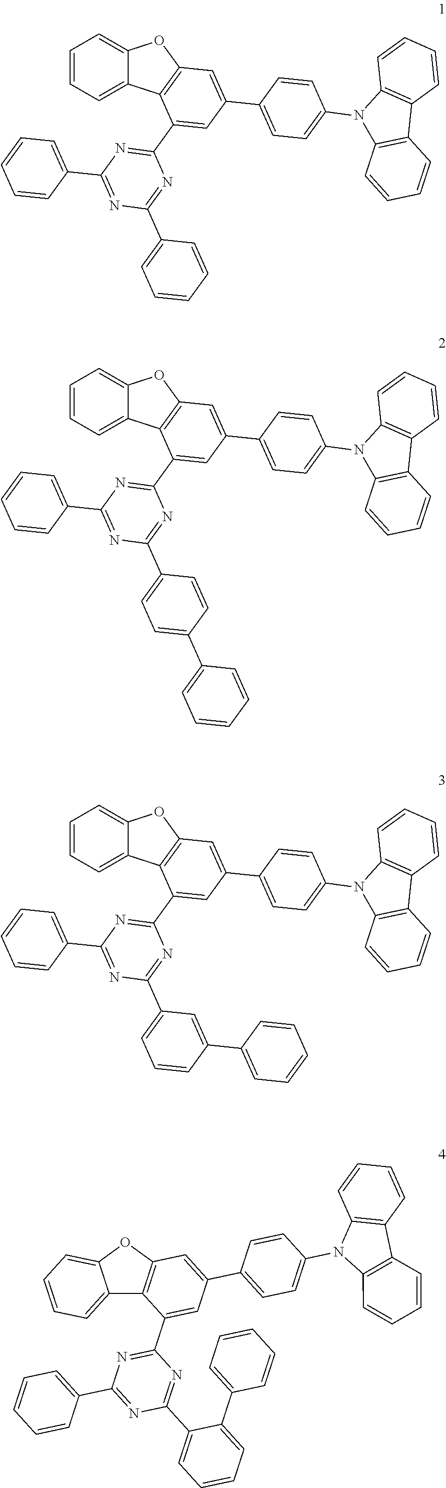 [SMILES: c1ccc(-c2nc(-c3ccccc3)nc(-c3cc(-c4ccc(-n5c6ccccc6c6ccccc65)cc4)cc4oc5ccccc5c34)n2)cc1 ; c1ccc(-c2ccc(-c3nc(-c4ccccc4)nc(-c4cc(-c5ccc(-n6c7ccccc7c7ccccc76)cc5)cc5oc6ccccc6c45)n3)cc2)cc1 ; c1ccc(-c2cccc(-c3nc(-c4ccccc4)nc(-c4cc(-c5ccc(-n6c7ccccc7c7ccccc76)cc5)cc5oc6ccccc6c45)n3)c2)cc1 ; c1ccc(-c2nc(-c3ccccc3-c3ccccc3)nc(-c3cc(-c4ccc(-n5c6ccccc6c6ccccc65)cc4)cc4oc5ccccc5c34)n2)cc1]

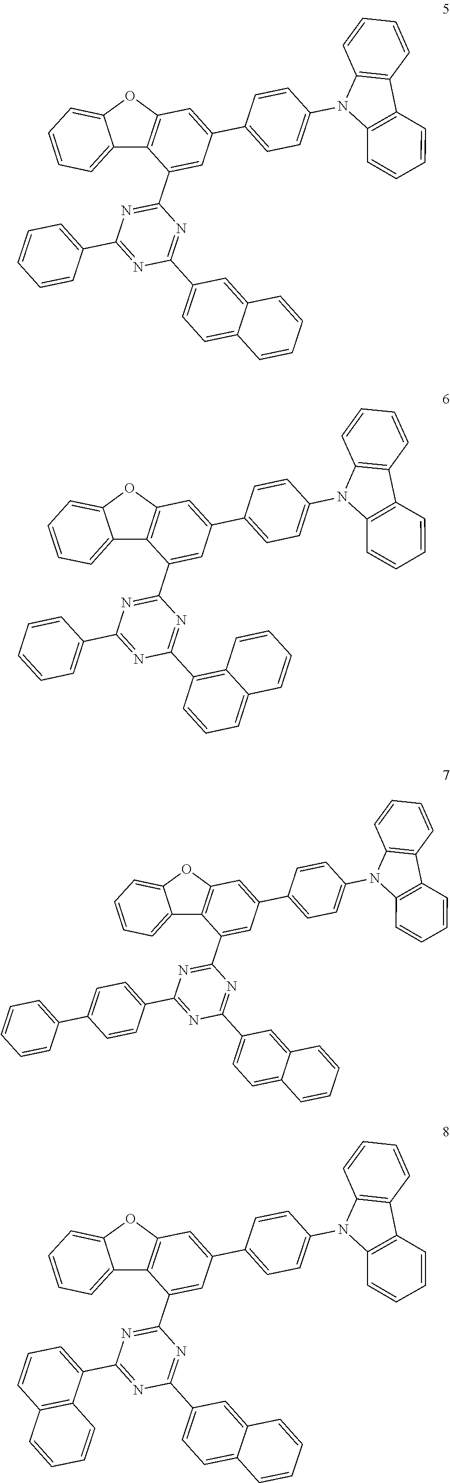 [SMILES: c1ccc(-c2nc(-c3ccc4ccccc4c3)nc(-c3cc(-c4ccc(-n5c6ccccc6c6ccccc65)cc4)cc4oc5ccccc5c34)n2)cc1 ; c1ccc(-c2nc(-c3cccc4ccccc34)nc(-c3cc(-c4ccc(-n5c6ccccc6c6ccccc65)cc4)cc4oc5ccccc5c34)n2)cc1 ; c1ccc(-c2ccc(-c3nc(-c4ccc5ccccc5c4)nc(-c4cc(-c5ccc(-n6c7ccccc7c7ccccc76)cc5)cc5oc6ccccc6c45)n3)cc2)cc1 ; c1ccc2cc(-c3nc(-c4cccc5ccccc45)nc(-c4cc(-c5ccc(-n6c7ccccc7c7ccccc76)cc5)cc5oc6ccccc6c45)n3)ccc2c1]

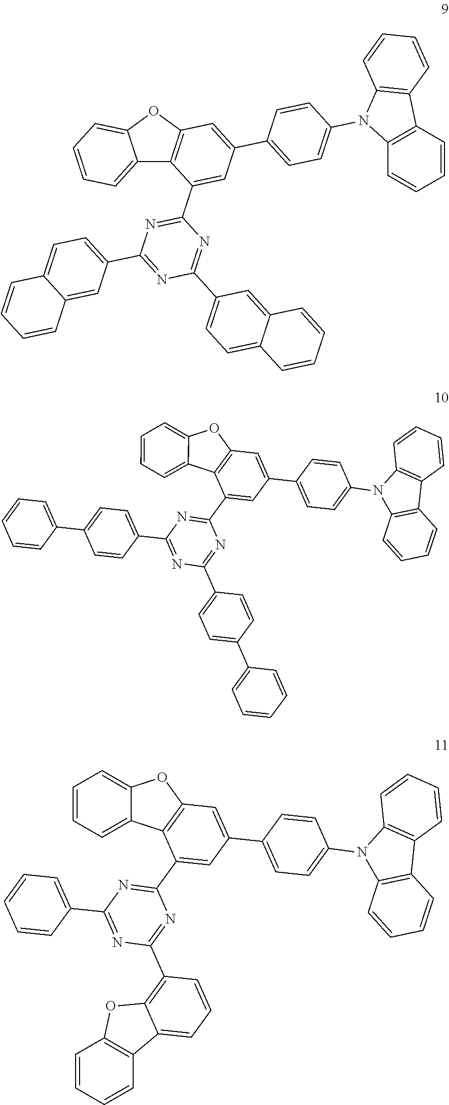 [SMILES: c1ccc2cc(-c3nc(-c4ccc5ccccc5c4)nc(-c4cc(-c5ccc(-n6c7ccccc7c7ccccc76)cc5)cc5oc6ccccc6c45)n3)ccc2c1 ; c1ccc(-c2ccc(-c3nc(-c4ccc(-c5ccccc5)cc4)nc(-c4cc(-c5ccc(-n6c7ccccc7c7ccccc76)cc5)cc5oc6ccccc6c45)n3)cc2)cc1 ; c1ccc(-c2nc(-c3cccc4c3oc3ccccc34)nc(-c3cc(-c4ccc(-n5c6ccccc6c6ccccc65)cc4)cc4oc5ccccc5c34)n2)cc1]

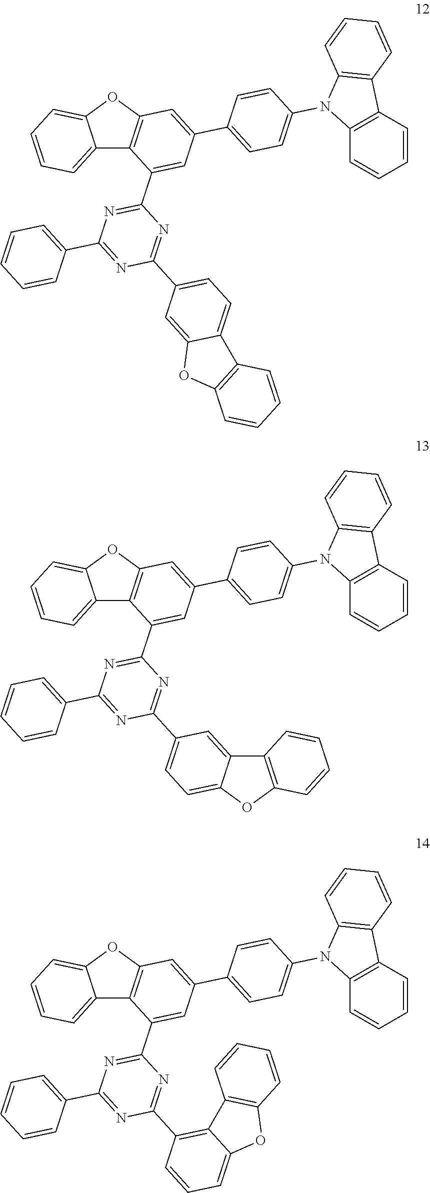 [SMILES: c1ccc(-c2nc(-c3ccc4c(c3)oc3ccccc34)nc(-c3cc(-c4ccc(-n5c6ccccc6c6ccccc65)cc4)cc4oc5ccccc5c34)n2)cc1 ; c1ccc(-c2nc(-c3ccc4oc5ccccc5c4c3)nc(-c3cc(-c4ccc(-n5c6ccccc6c6ccccc65)cc4)cc4oc5ccccc5c34)n2)cc1 ; c1ccc(-c2nc(-c3cccc4oc5ccccc5c34)nc(-c3cc(-c4ccc(-n5c6ccccc6c6ccccc65)cc4)cc4oc5ccccc5c34)n2)cc1]

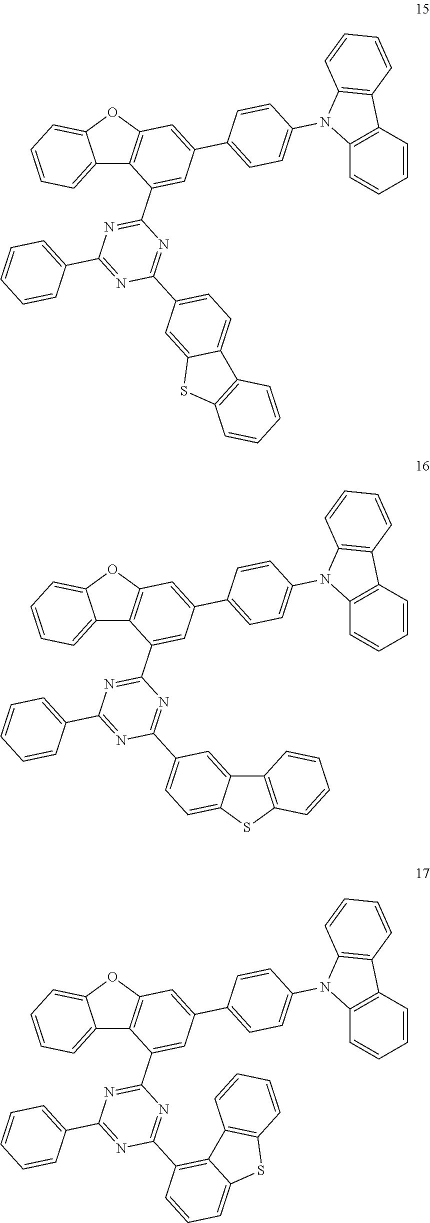 [SMILES: c1ccc(-c2nc(-c3ccc4c(c3)sc3ccccc34)nc(-c3cc(-c4ccc(-n5c6ccccc6c6ccccc65)cc4)cc4oc5ccccc5c34)n2)cc1 ; c1ccc(-c2nc(-c3ccc4sc5ccccc5c4c3)nc(-c3cc(-c4ccc(-n5c6ccccc6c6ccccc65)cc4)cc4oc5ccccc5c34)n2)cc1 ; c1ccc(-c2nc(-c3cc(-c4ccc(-n5c6ccccc6c6ccccc65)cc4)cc4oc5ccccc5c34)nc(-c3cccc4sc5ccccc5c34)n2)cc1]

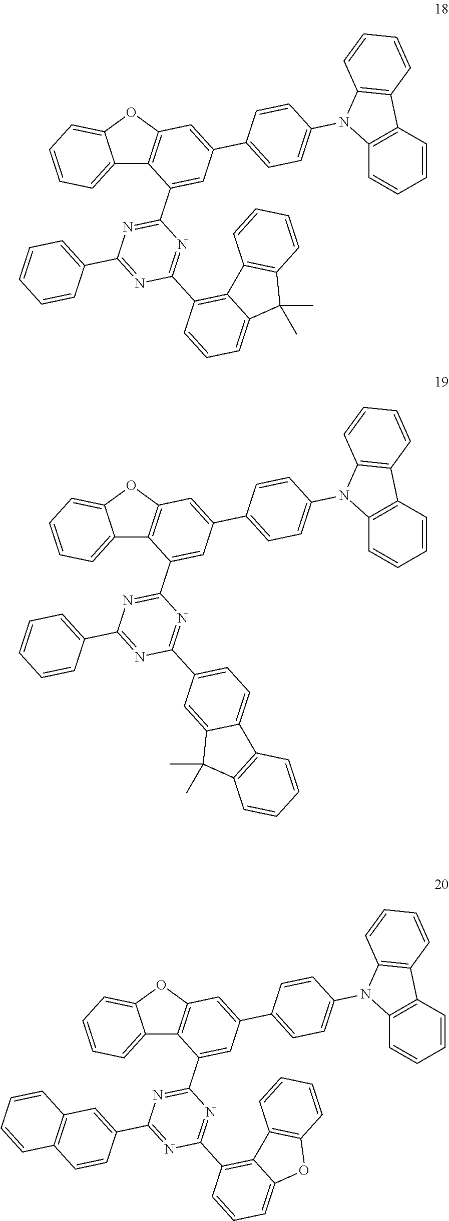 [SMILES: CC1(C)c2ccccc2-c2c(-c3nc(-c4ccccc4)nc(-c4cc(-c5ccc(-n6c7ccccc7c7ccccc76)cc5)cc5oc6ccccc6c45)n3)cccc21 ; CC1(C)c2ccccc2-c2ccc(-c3nc(-c4ccccc4)nc(-c4cc(-c5ccc(-n6c7ccccc7c7ccccc76)cc5)cc5oc6ccccc6c45)n3)cc21 ; c1ccc2cc(-c3nc(-c4cccc5oc6ccccc6c45)nc(-c4cc(-c5ccc(-n6c7ccccc7c7ccccc76)cc5)cc5oc6ccccc6c45)n3)ccc2c1]

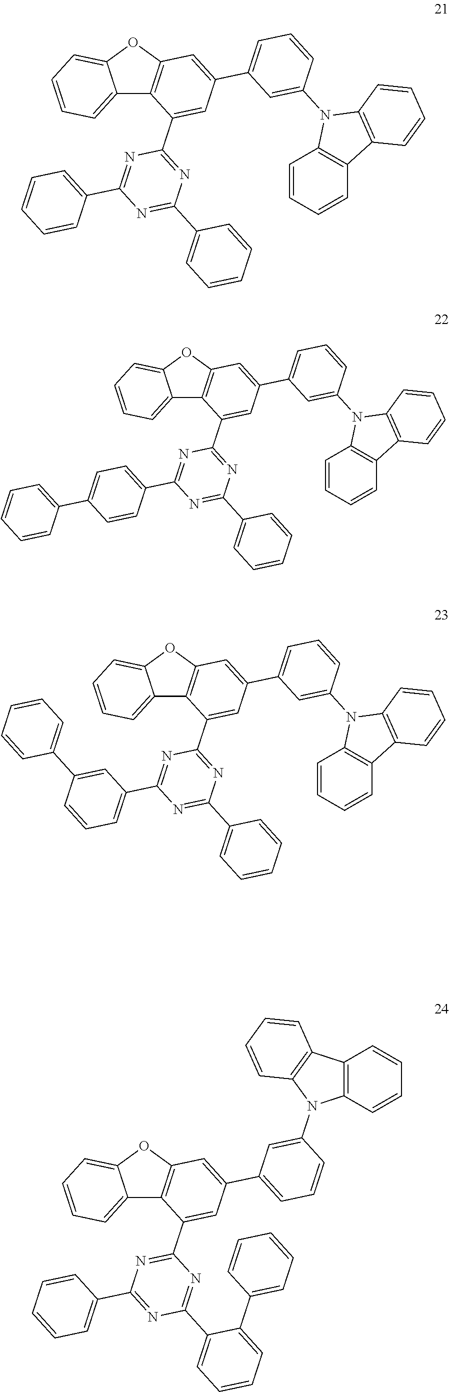 [SMILES: c1ccc(-c2nc(-c3ccccc3)nc(-c3cc(-c4cccc(-n5c6ccccc6c6ccccc65)c4)cc4oc5ccccc5c34)n2)cc1 ; c1ccc(-c2ccc(-c3nc(-c4ccccc4)nc(-c4cc(-c5cccc(-n6c7ccccc7c7ccccc76)c5)cc5oc6ccccc6c45)n3)cc2)cc1 ; c1ccc(-c2cccc(-c3nc(-c4ccccc4)nc(-c4cc(-c5cccc(-n6c7ccccc7c7ccccc76)c5)cc5oc6ccccc6c45)n3)c2)cc1 ; c1ccc(-c2nc(-c3ccccc3-c3ccccc3)nc(-c3cc(-c4cccc(-n5c6ccccc6c6ccccc65)c4)cc4oc5ccccc5c34)n2)cc1]

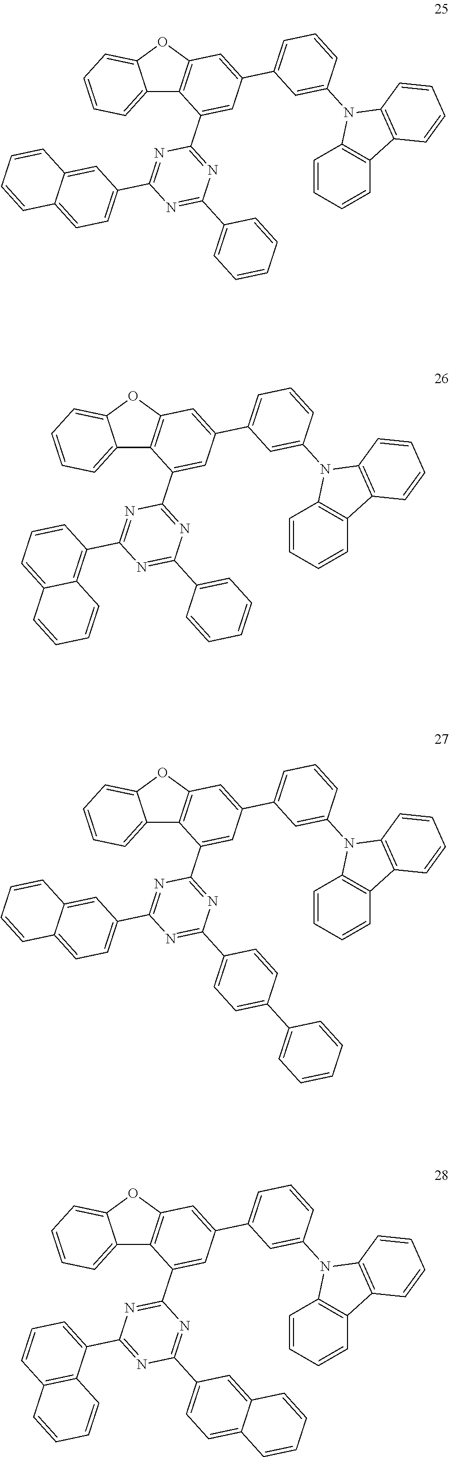 [SMILES: c1ccc(-c2nc(-c3ccc4ccccc4c3)nc(-c3cc(-c4cccc(-n5c6ccccc6c6ccccc65)c4)cc4oc5ccccc5c34)n2)cc1 ; c1ccc(-c2nc(-c3cccc4ccccc34)nc(-c3cc(-c4cccc(-n5c6ccccc6c6ccccc65)c4)cc4oc5ccccc5c34)n2)cc1 ; c1ccc(-c2ccc(-c3nc(-c4ccc5ccccc5c4)nc(-c4cc(-c5cccc(-n6c7ccccc7c7ccccc76)c5)cc5oc6ccccc6c45)n3)cc2)cc1 ; c1cc(-c2cc(-c3nc(-c4ccc5ccccc5c4)nc(-c4cccc5ccccc45)n3)c3c(c2)oc2ccccc23)cc(-n2c3ccccc3c3ccccc32)c1]

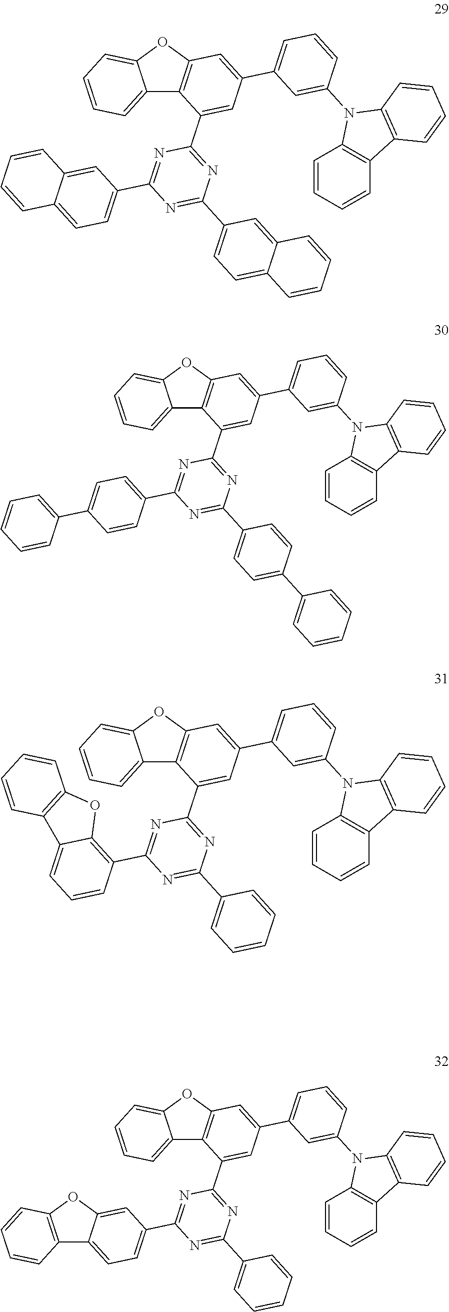 [SMILES: c1cc(-c2cc(-c3nc(-c4ccc5ccccc5c4)nc(-c4ccc5ccccc5c4)n3)c3c(c2)oc2ccccc23)cc(-n2c3ccccc3c3ccccc32)c1 ; c1ccc(-c2ccc(-c3nc(-c4ccc(-c5ccccc5)cc4)nc(-c4cc(-c5cccc(-n6c7ccccc7c7ccccc76)c5)cc5oc6ccccc6c45)n3)cc2)cc1 ; c1ccc(-c2nc(-c3cccc4c3oc3ccccc34)nc(-c3cc(-c4cccc(-n5c6ccccc6c6ccccc65)c4)cc4oc5ccccc5c34)n2)cc1 ; c1ccc(-c2nc(-c3ccc4c(c3)oc3ccccc34)nc(-c3cc(-c4cccc(-n5c6ccccc6c6ccccc65)c4)cc4oc5ccccc5c34)n2)cc1]

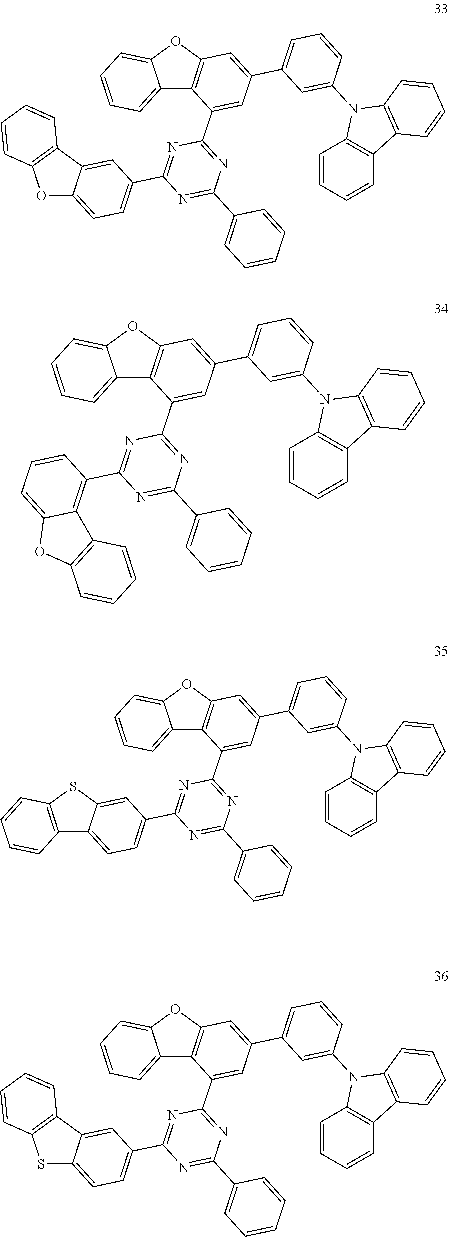 [SMILES: c1ccc(-c2nc(-c3ccc4oc5ccccc5c4c3)nc(-c3cc(-c4cccc(-n5c6ccccc6c6ccccc65)c4)cc4oc5ccccc5c34)n2)cc1 ; c1ccc(-c2nc(-c3cccc4oc5ccccc5c34)nc(-c3cc(-c4cccc(-n5c6ccccc6c6ccccc65)c4)cc4oc5ccccc5c34)n2)cc1 ; c1ccc(-c2nc(-c3ccc4c(c3)sc3ccccc34)nc(-c3cc(-c4cccc(-n5c6ccccc6c6ccccc65)c4)cc4oc5ccccc5c34)n2)cc1 ; c1ccc(-c2nc(-c3ccc4sc5ccccc5c4c3)nc(-c3cc(-c4cccc(-n5c6ccccc6c6ccccc65)c4)cc4oc5ccccc5c34)n2)cc1]

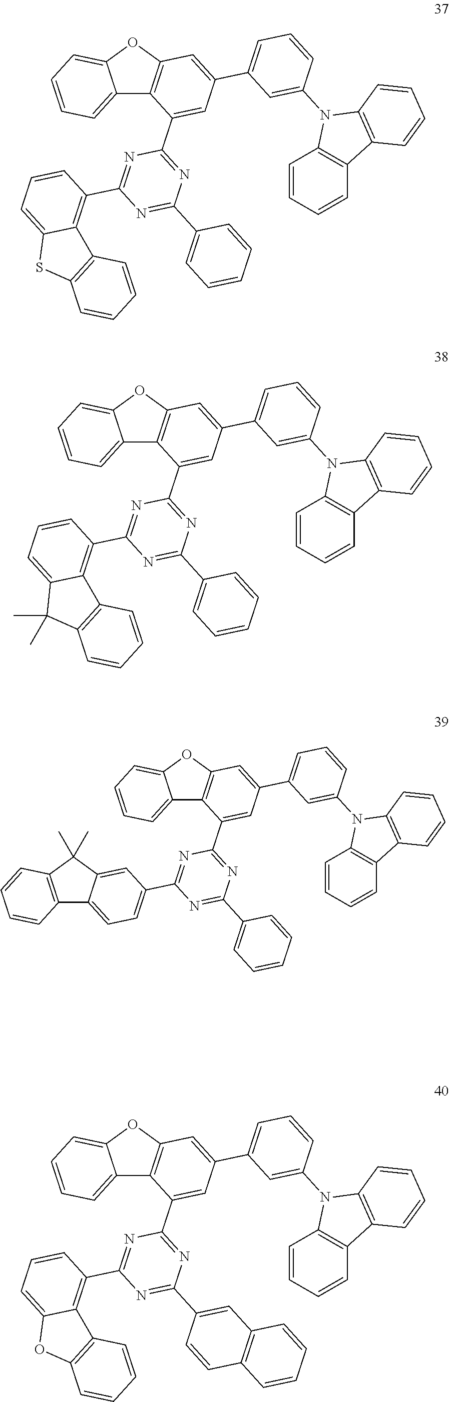 [SMILES: c1ccc(-c2nc(-c3cc(-c4cccc(-n5c6ccccc6c6ccccc65)c4)cc4oc5ccccc5c34)nc(-c3cccc4sc5ccccc5c34)n2)cc1 ; CC1(C)c2ccccc2-c2c(-c3nc(-c4ccccc4)nc(-c4cc(-c5cccc(-n6c7ccccc7c7ccccc76)c5)cc5oc6ccccc6c45)n3)cccc21 ; CC1(C)c2ccccc2-c2ccc(-c3nc(-c4ccccc4)nc(-c4cc(-c5cccc(-n6c7ccccc7c7ccccc76)c5)cc5oc6ccccc6c45)n3)cc21 ; c1cc(-c2cc(-c3nc(-c4ccc5ccccc5c4)nc(-c4cccc5oc6ccccc6c45)n3)c3c(c2)oc2ccccc23)cc(-n2c3ccccc3c3ccccc32)c1]

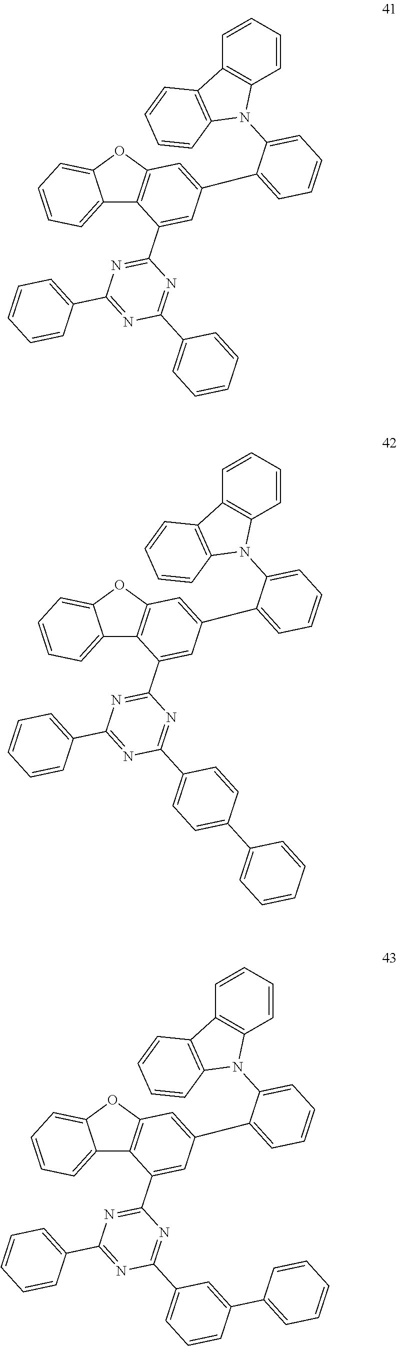 [SMILES: c1ccc(-c2nc(-c3ccccc3)nc(-c3cc(-c4ccccc4-n4c5ccccc5c5ccccc54)cc4oc5ccccc5c34)n2)cc1 ; c1ccc(-c2ccc(-c3nc(-c4ccccc4)nc(-c4cc(-c5ccccc5-n5c6ccccc6c6ccccc65)cc5oc6ccccc6c45)n3)cc2)cc1 ; c1ccc(-c2cccc(-c3nc(-c4ccccc4)nc(-c4cc(-c5ccccc5-n5c6ccccc6c6ccccc65)cc5oc6ccccc6c45)n3)c2)cc1]

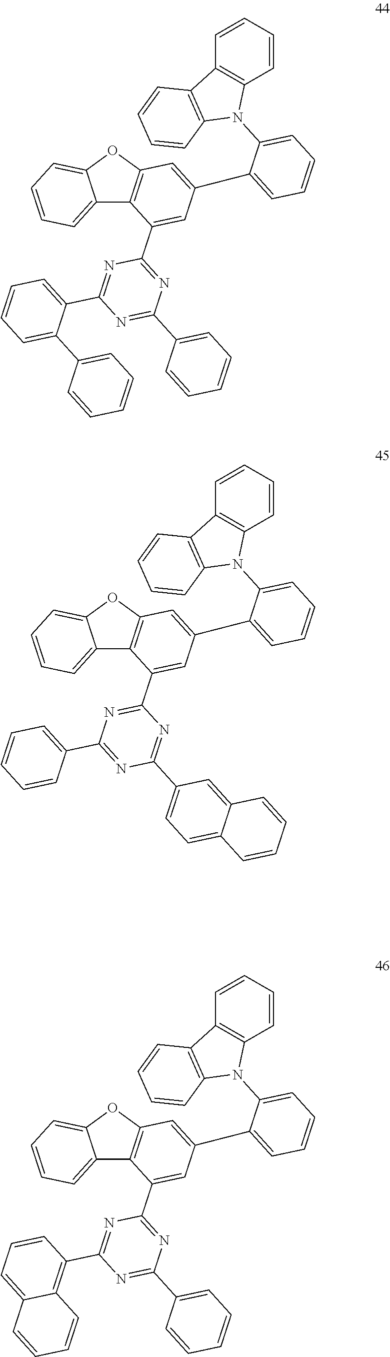 [SMILES: c1ccc(-c2nc(-c3ccccc3-c3ccccc3)nc(-c3cc(-c4ccccc4-n4c5ccccc5c5ccccc54)cc4oc5ccccc5c34)n2)cc1 ; c1ccc(-c2nc(-c3ccc4ccccc4c3)nc(-c3cc(-c4ccccc4-n4c5ccccc5c5ccccc54)cc4oc5ccccc5c34)n2)cc1 ; c1ccc(-c2nc(-c3cccc4ccccc34)nc(-c3cc(-c4ccccc4-n4c5ccccc5c5ccccc54)cc4oc5ccccc5c34)n2)cc1]

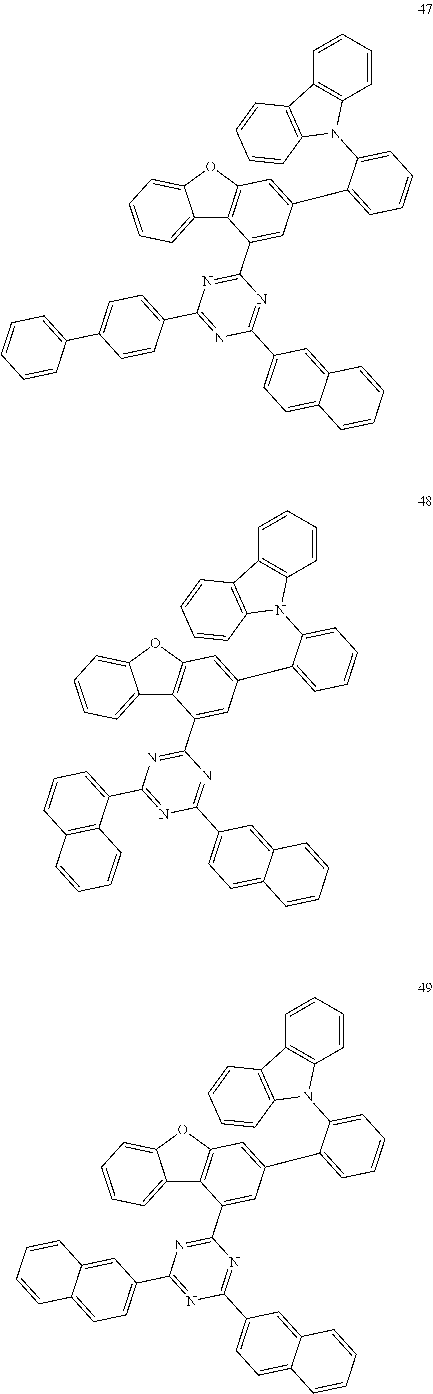 [SMILES: c1ccc(-c2ccc(-c3nc(-c4ccc5ccccc5c4)nc(-c4cc(-c5ccccc5-n5c6ccccc6c6ccccc65)cc5oc6ccccc6c45)n3)cc2)cc1 ; c1ccc(-n2c3ccccc3c3ccccc32)c(-c2cc(-c3nc(-c4ccc5ccccc5c4)nc(-c4cccc5ccccc45)n3)c3c(c2)oc2ccccc23)c1 ; c1ccc(-n2c3ccccc3c3ccccc32)c(-c2cc(-c3nc(-c4ccc5ccccc5c4)nc(-c4ccc5ccccc5c4)n3)c3c(c2)oc2ccccc23)c1]

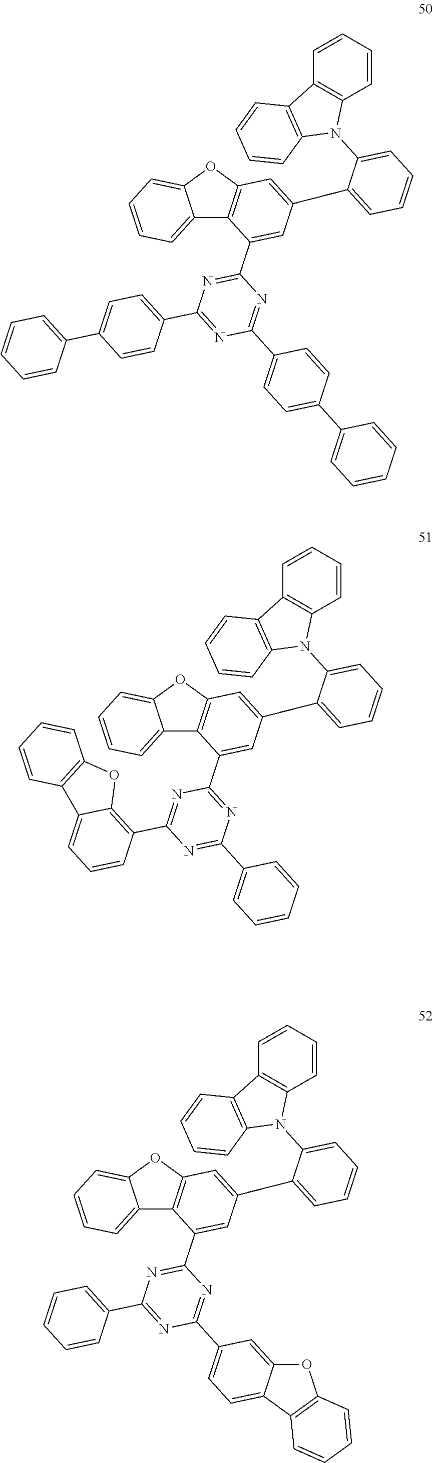 [SMILES: c1ccc(-c2ccc(-c3nc(-c4ccc(-c5ccccc5)cc4)nc(-c4cc(-c5ccccc5-n5c6ccccc6c6ccccc65)cc5oc6ccccc6c45)n3)cc2)cc1 ; c1ccc(-c2nc(-c3cccc4c3oc3ccccc34)nc(-c3cc(-c4ccccc4-n4c5ccccc5c5ccccc54)cc4oc5ccccc5c34)n2)cc1 ; c1ccc(-c2nc(-c3ccc4c(c3)oc3ccccc34)nc(-c3cc(-c4ccccc4-n4c5ccccc5c5ccccc54)cc4oc5ccccc5c34)n2)cc1]

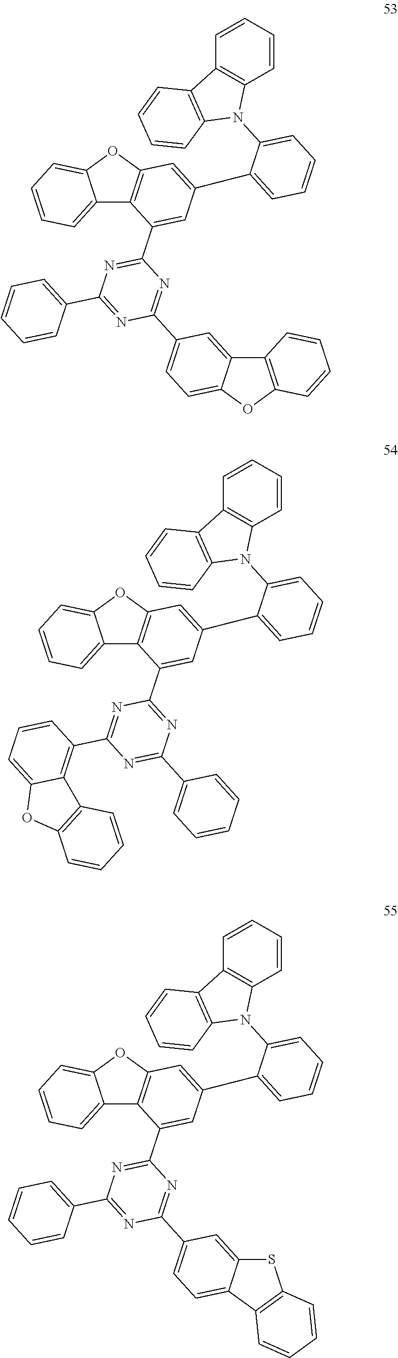 [SMILES: c1ccc(-c2nc(-c3ccc4oc5ccccc5c4c3)nc(-c3cc(-c4ccccc4-n4c5ccccc5c5ccccc54)cc4oc5ccccc5c34)n2)cc1 ; c1ccc(-c2nc(-c3cccc4oc5ccccc5c34)nc(-c3cc(-c4ccccc4-n4c5ccccc5c5ccccc54)cc4oc5ccccc5c34)n2)cc1 ; c1ccc(-c2nc(-c3ccc4c(c3)sc3ccccc34)nc(-c3cc(-c4ccccc4-n4c5ccccc5c5ccccc54)cc4oc5ccccc5c34)n2)cc1]

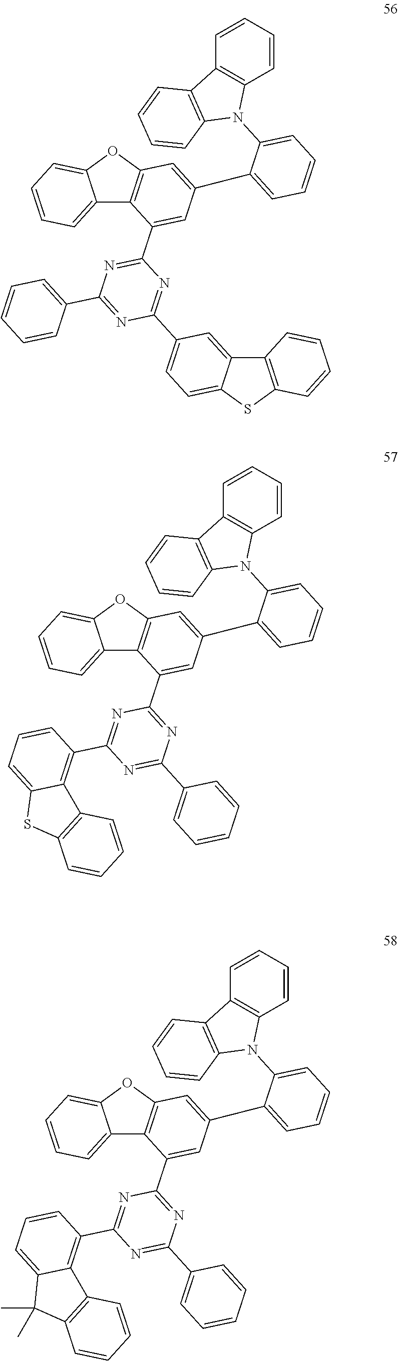 [SMILES: c1ccc(-c2nc(-c3ccc4sc5ccccc5c4c3)nc(-c3cc(-c4ccccc4-n4c5ccccc5c5ccccc54)cc4oc5ccccc5c34)n2)cc1 ; c1ccc(-c2nc(-c3cc(-c4ccccc4-n4c5ccccc5c5ccccc54)cc4oc5ccccc5c34)nc(-c3cccc4sc5ccccc5c34)n2)cc1 ; CC1(C)c2ccccc2-c2c(-c3nc(-c4ccccc4)nc(-c4cc(-c5ccccc5-n5c6ccccc6c6ccccc65)cc5oc6ccccc6c45)n3)cccc21]

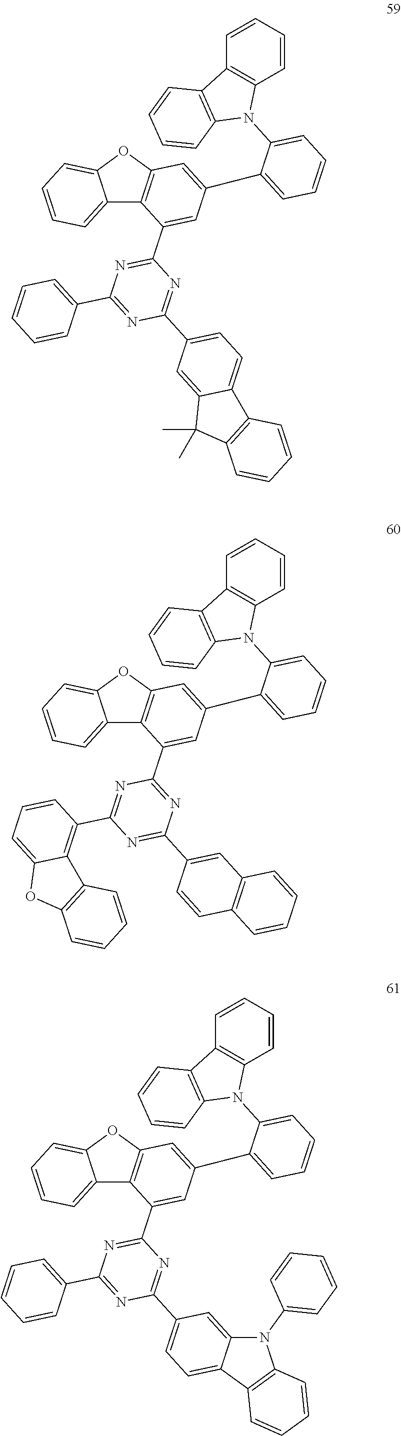 [SMILES: CC1(C)c2ccccc2-c2ccc(-c3nc(-c4ccccc4)nc(-c4cc(-c5ccccc5-n5c6ccccc6c6ccccc65)cc5oc6ccccc6c45)n3)cc21 ; c1ccc(-n2c3ccccc3c3ccccc32)c(-c2cc(-c3nc(-c4ccc5ccccc5c4)nc(-c4cccc5oc6ccccc6c45)n3)c3c(c2)oc2ccccc23)c1 ; c1ccc(-c2nc(-c3ccc4c5ccccc5n(-c5ccccc5)c4c3)nc(-c3cc(-c4ccccc4-n4c5ccccc5c5ccccc54)cc4oc5ccccc5c34)n2)cc1]

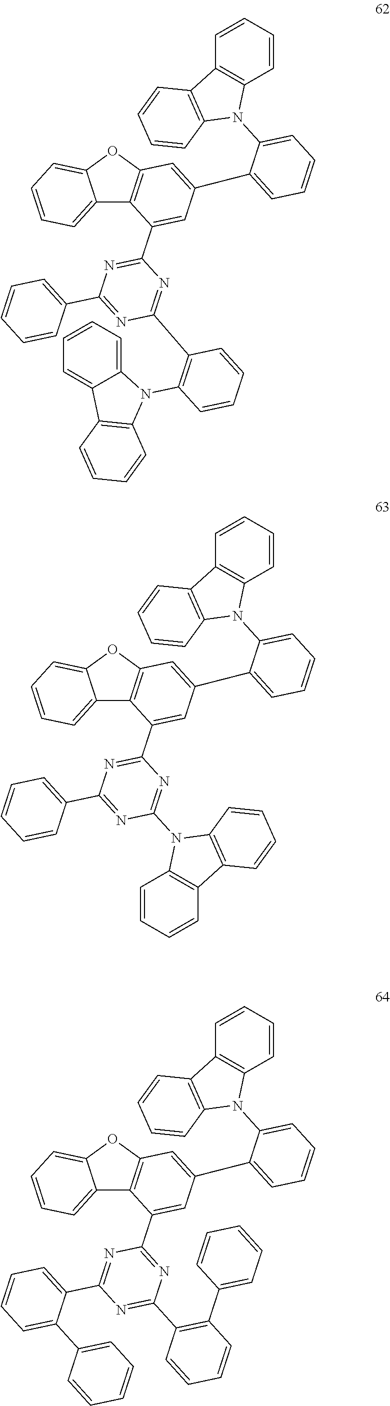 [SMILES: c1ccc(-c2nc(-c3ccccc3-n3c4ccccc4c4ccccc43)nc(-c3cc(-c4ccccc4-n4c5ccccc5c5ccccc54)cc4oc5ccccc5c34)n2)cc1 ; c1ccc(-c2nc(-c3cc(-c4ccccc4-n4c5ccccc5c5ccccc54)cc4oc5ccccc5c34)nc(-n3c4ccccc4c4ccccc43)n2)cc1 ; c1ccc(-c2ccccc2-c2nc(-c3ccccc3-c3ccccc3)nc(-c3cc(-c4ccccc4-n4c5ccccc5c5ccccc54)cc4oc5ccccc5c34)n2)cc1]

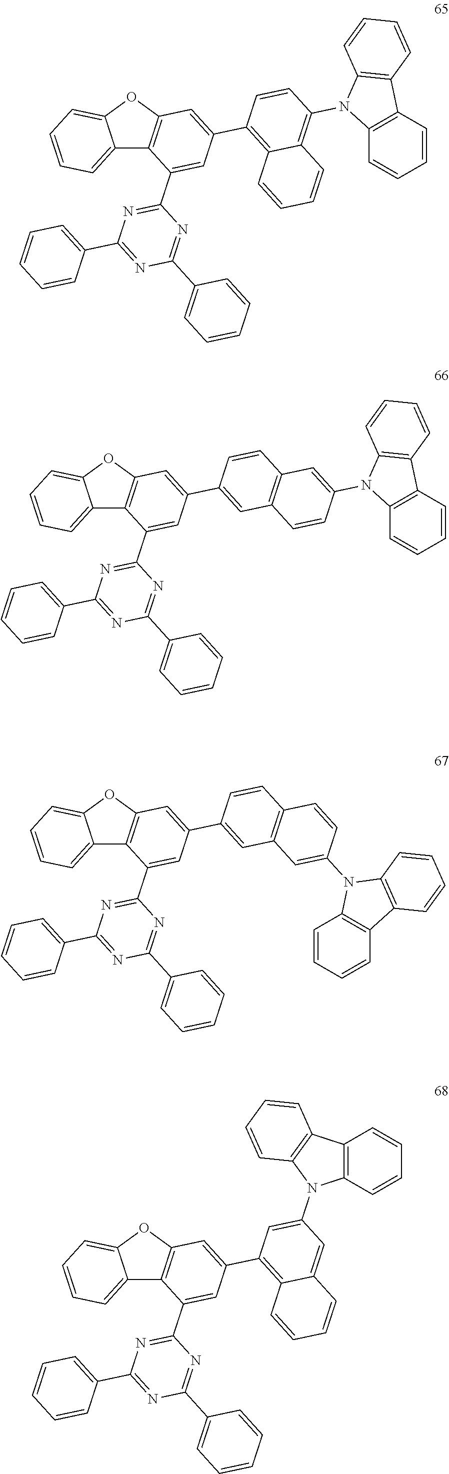 [SMILES: c1ccc(-c2nc(-c3ccccc3)nc(-c3cc(-c4ccc(-n5c6ccccc6c6ccccc65)c5ccccc45)cc4oc5ccccc5c34)n2)cc1 ; c1ccc(-c2nc(-c3ccccc3)nc(-c3cc(-c4ccc5cc(-n6c7ccccc7c7ccccc76)ccc5c4)cc4oc5ccccc5c34)n2)cc1 ; c1ccc(-c2nc(-c3ccccc3)nc(-c3cc(-c4ccc5ccc(-n6c7ccccc7c7ccccc76)cc5c4)cc4oc5ccccc5c34)n2)cc1 ; c1ccc(-c2nc(-c3ccccc3)nc(-c3cc(-c4cc(-n5c6ccccc6c6ccccc65)cc5ccccc45)cc4oc5ccccc5c34)n2)cc1]

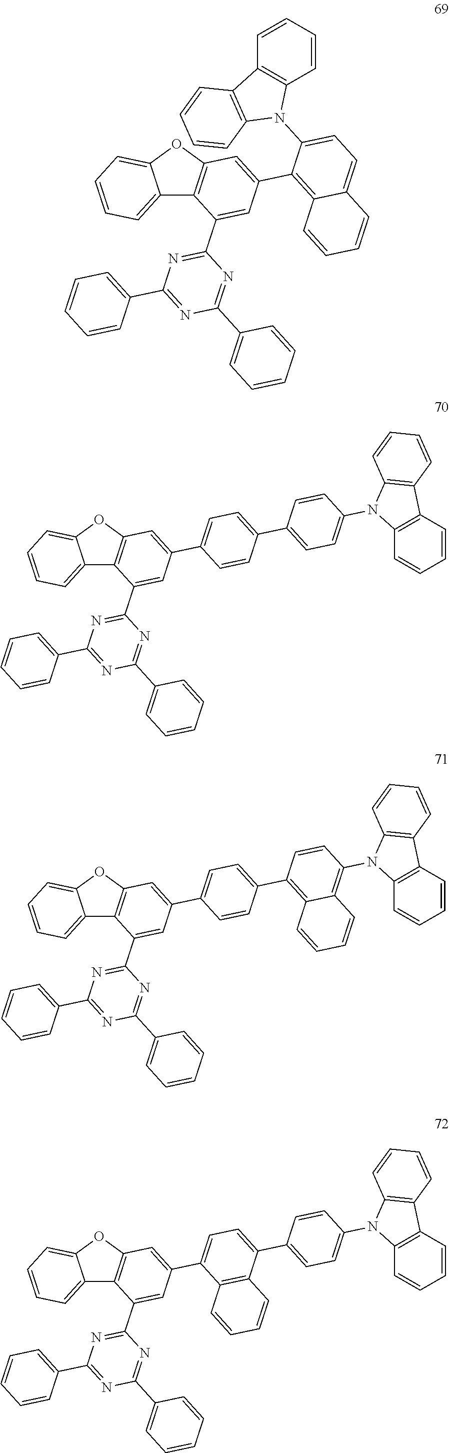 [SMILES: c1ccc(-c2nc(-c3ccccc3)nc(-c3cc(-c4c(-n5c6ccccc6c6ccccc65)ccc5ccccc45)cc4oc5ccccc5c34)n2)cc1 ; c1ccc(-c2nc(-c3ccccc3)nc(-c3cc(-c4ccc(-c5ccc(-n6c7ccccc7c7ccccc76)cc5)cc4)cc4oc5ccccc5c34)n2)cc1 ; c1ccc(-c2nc(-c3ccccc3)nc(-c3cc(-c4ccc(-c5ccc(-n6c7ccccc7c7ccccc76)c6ccccc56)cc4)cc4oc5ccccc5c34)n2)cc1 ; c1ccc(-c2nc(-c3ccccc3)nc(-c3cc(-c4ccc(-c5ccc(-n6c7ccccc7c7ccccc76)cc5)c5ccccc45)cc4oc5ccccc5c34)n2)cc1]

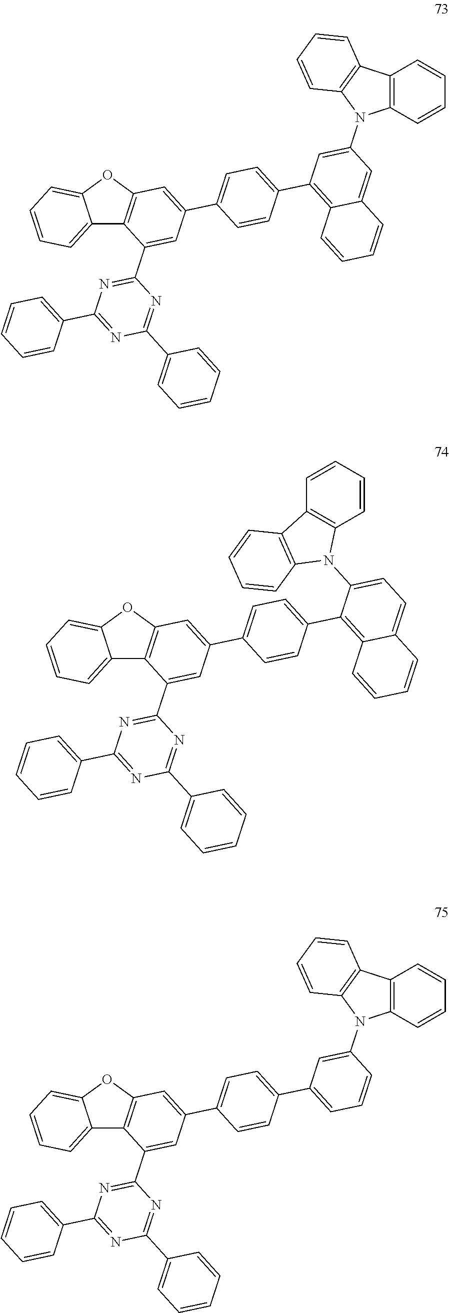 [SMILES: c1ccc(-c2nc(-c3ccccc3)nc(-c3cc(-c4ccc(-c5cc(-n6c7ccccc7c7ccccc76)cc6ccccc56)cc4)cc4oc5ccccc5c34)n2)cc1 ; c1ccc(-c2nc(-c3ccccc3)nc(-c3cc(-c4ccc(-c5c(-n6c7ccccc7c7ccccc76)ccc6ccccc56)cc4)cc4oc5ccccc5c34)n2)cc1 ; c1ccc(-c2nc(-c3ccccc3)nc(-c3cc(-c4ccc(-c5cccc(-n6c7ccccc7c7ccccc76)c5)cc4)cc4oc5ccccc5c34)n2)cc1]

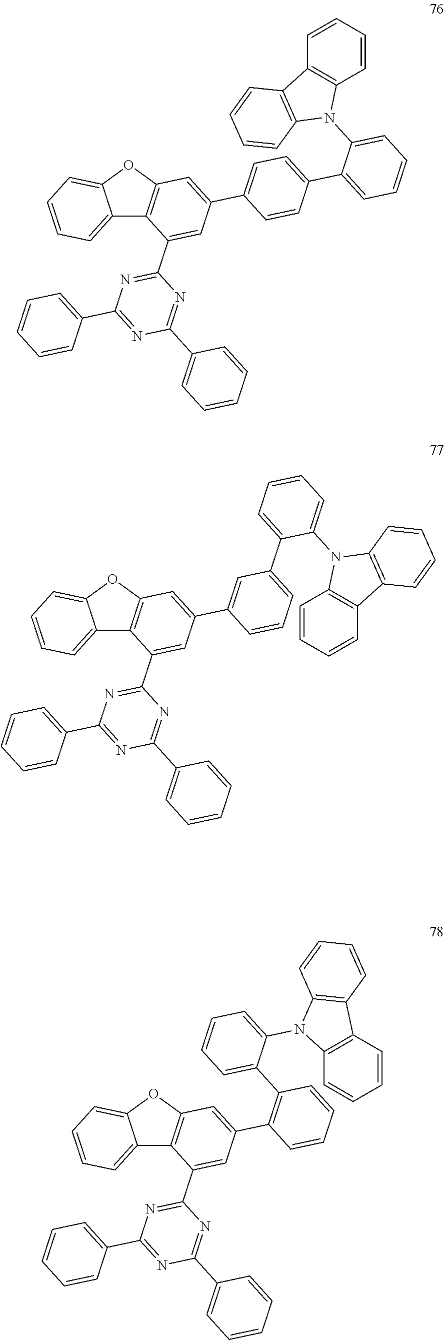 [SMILES: c1ccc(-c2nc(-c3ccccc3)nc(-c3cc(-c4ccc(-c5ccccc5-n5c6ccccc6c6ccccc65)cc4)cc4oc5ccccc5c34)n2)cc1 ; c1ccc(-c2nc(-c3ccccc3)nc(-c3cc(-c4cccc(-c5ccccc5-n5c6ccccc6c6ccccc65)c4)cc4oc5ccccc5c34)n2)cc1 ; c1ccc(-c2nc(-c3ccccc3)nc(-c3cc(-c4ccccc4-c4ccccc4-n4c5ccccc5c5ccccc54)cc4oc5ccccc5c34)n2)cc1]

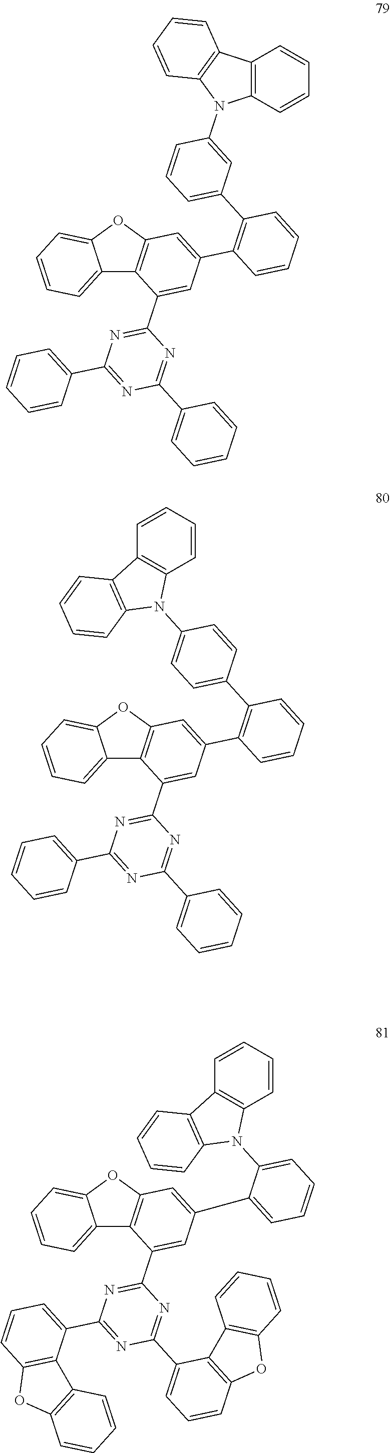 [SMILES: c1ccc(-c2nc(-c3ccccc3)nc(-c3cc(-c4ccccc4-c4cccc(-n5c6ccccc6c6ccccc65)c4)cc4oc5ccccc5c34)n2)cc1 ; c1ccc(-c2nc(-c3ccccc3)nc(-c3cc(-c4ccccc4-c4ccc(-n5c6ccccc6c6ccccc65)cc4)cc4oc5ccccc5c34)n2)cc1 ; c1ccc(-n2c3ccccc3c3ccccc32)c(-c2cc(-c3nc(-c4cccc5oc6ccccc6c45)nc(-c4cccc5oc6ccccc6c45)n3)c3c(c2)oc2ccccc23)c1]

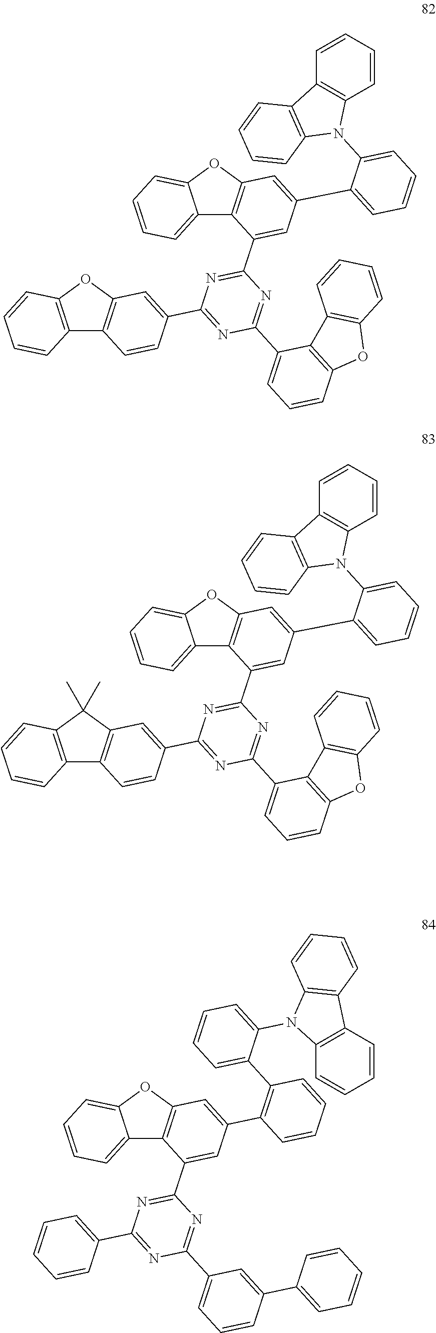 [SMILES: c1ccc(-n2c3ccccc3c3ccccc32)c(-c2cc(-c3nc(-c4ccc5c(c4)oc4ccccc45)nc(-c4cccc5oc6ccccc6c45)n3)c3c(c2)oc2ccccc23)c1 ; CC1(C)c2ccccc2-c2ccc(-c3nc(-c4cccc5oc6ccccc6c45)nc(-c4cc(-c5ccccc5-n5c6ccccc6c6ccccc65)cc5oc6ccccc6c45)n3)cc21 ; c1ccc(-c2cccc(-c3nc(-c4ccccc4)nc(-c4cc(-c5ccccc5-c5ccccc5-n5c6ccccc6c6ccccc65)cc5oc6ccccc6c45)n3)c2)cc1]

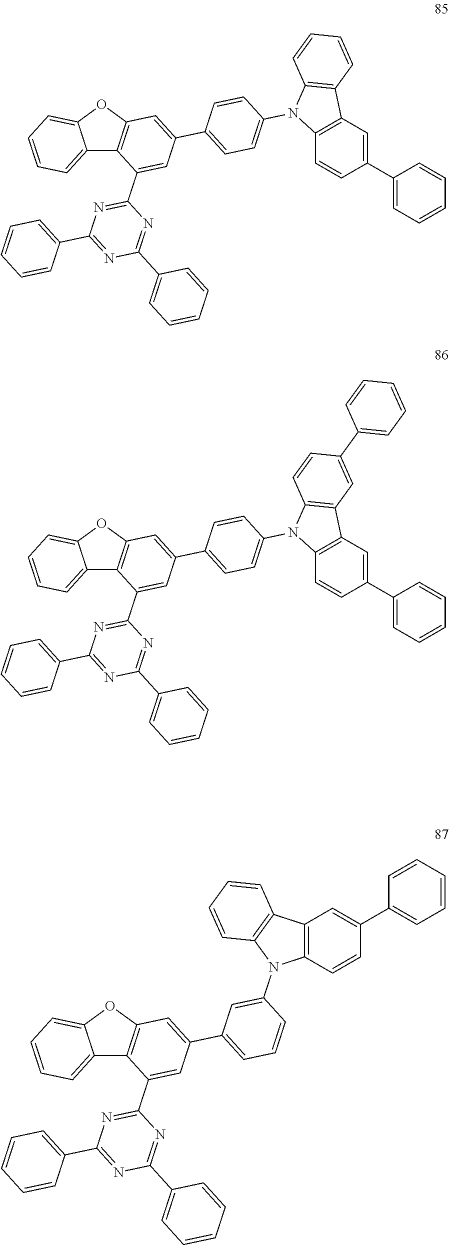 [SMILES: c1ccc(-c2ccc3c(c2)c2ccccc2n3-c2ccc(-c3cc(-c4nc(-c5ccccc5)nc(-c5ccccc5)n4)c4c(c3)oc3ccccc34)cc2)cc1 ; c1ccc(-c2ccc3c(c2)c2cc(-c4ccccc4)ccc2n3-c2ccc(-c3cc(-c4nc(-c5ccccc5)nc(-c5ccccc5)n4)c4c(c3)oc3ccccc34)cc2)cc1 ; c1ccc(-c2ccc3c(c2)c2ccccc2n3-c2cccc(-c3cc(-c4nc(-c5ccccc5)nc(-c5ccccc5)n4)c4c(c3)oc3ccccc34)c2)cc1]

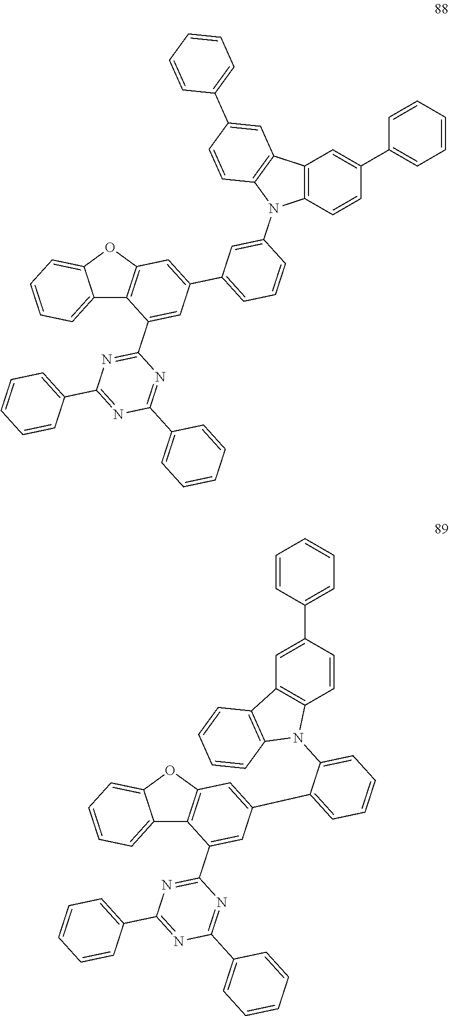 [SMILES: c1ccc(-c2ccc3c(c2)c2cc(-c4ccccc4)ccc2n3-c2cccc(-c3cc(-c4nc(-c5ccccc5)nc(-c5ccccc5)n4)c4c(c3)oc3ccccc34)c2)cc1 ; c1ccc(-c2ccc3c(c2)c2ccccc2n3-c2ccccc2-c2cc(-c3nc(-c4ccccc4)nc(-c4ccccc4)n3)c3c(c2)oc2ccccc23)cc1]

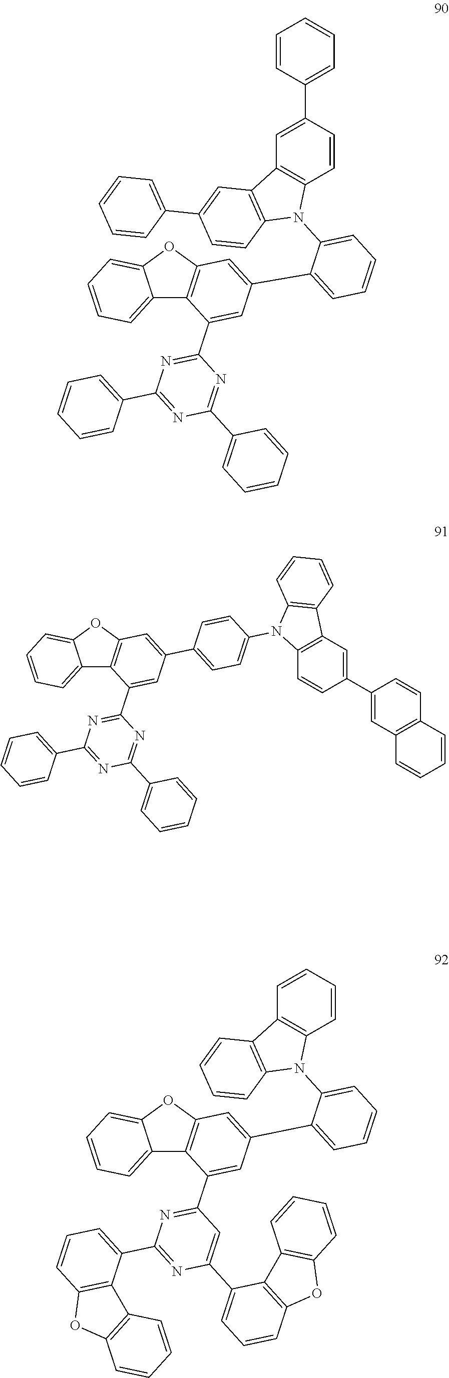 [SMILES: c1ccc(-c2ccc3c(c2)c2cc(-c4ccccc4)ccc2n3-c2ccccc2-c2cc(-c3nc(-c4ccccc4)nc(-c4ccccc4)n3)c3c(c2)oc2ccccc23)cc1 ; c1ccc(-c2nc(-c3ccccc3)nc(-c3cc(-c4ccc(-n5c6ccccc6c6cc(-c7ccc8ccccc8c7)ccc65)cc4)cc4oc5ccccc5c34)n2)cc1 ; c1ccc(-n2c3ccccc3c3ccccc32)c(-c2cc(-c3cc(-c4cccc5oc6ccccc6c45)nc(-c4cccc5oc6ccccc6c45)n3)c3c(c2)oc2ccccc23)c1]

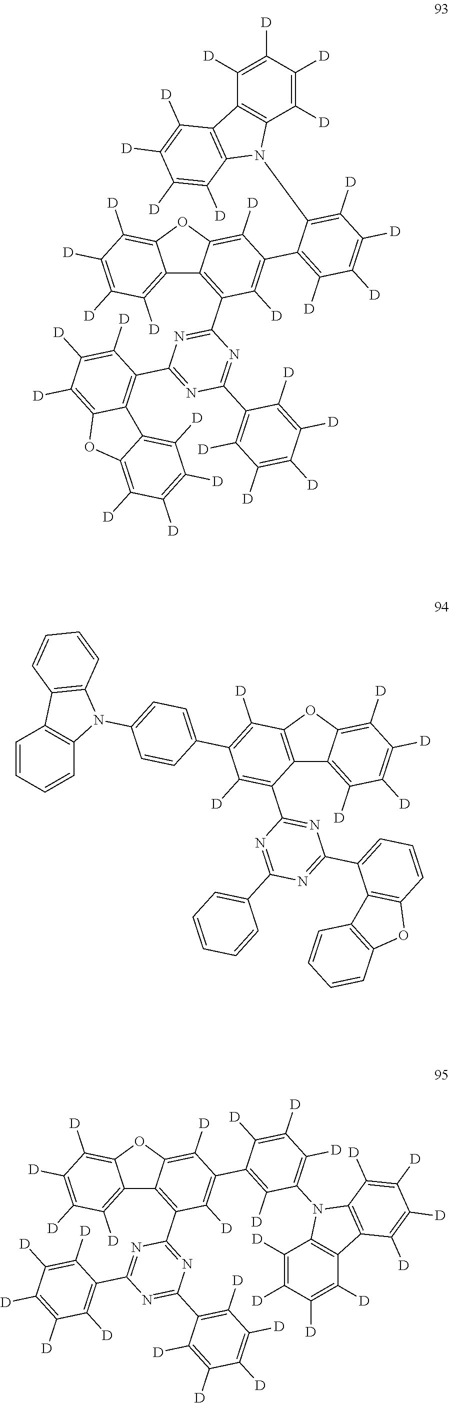

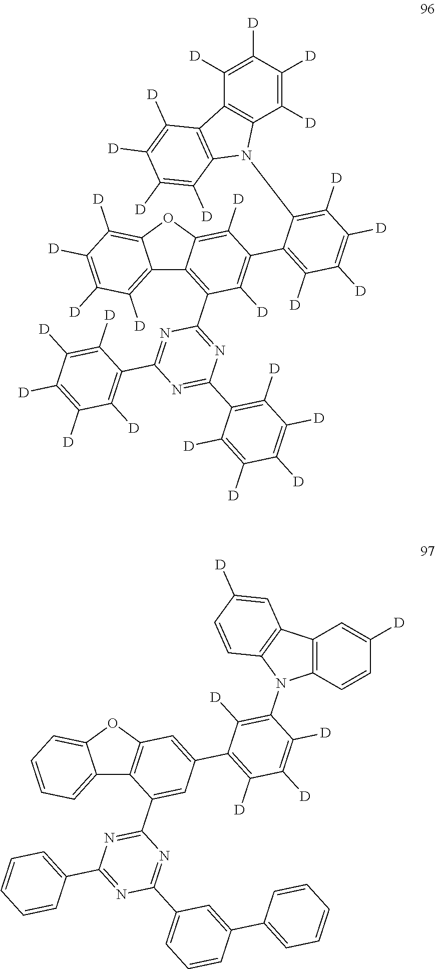 [SMILES: [2H]c1c([2H])c([2H])c(-c2nc(-c3c([2H])c([2H])c([2H])c([2H])c3[2H])nc(-c3c([2H])c(-c4c([2H])c([2H])c([2H])c([2H])c4-n4c5c([2H])c([2H])c([2H])c([2H])c5c5c([2H])c([2H])c([2H])c([2H])c54)c([2H])c4oc5c([2H])c([2H])c([2H])c([2H])c5c34)n2)c([2H])c1[2H] ; [2H]c1ccc2c(c1)c1cc([2H])ccc1n2-c1c([2H])c([2H])c([2H])c(-c2cc(-c3nc(-c4ccccc4)nc(-c4cccc(-c5ccccc5)c4)n3)c3c(c2)oc2ccccc23)c1[2H]]

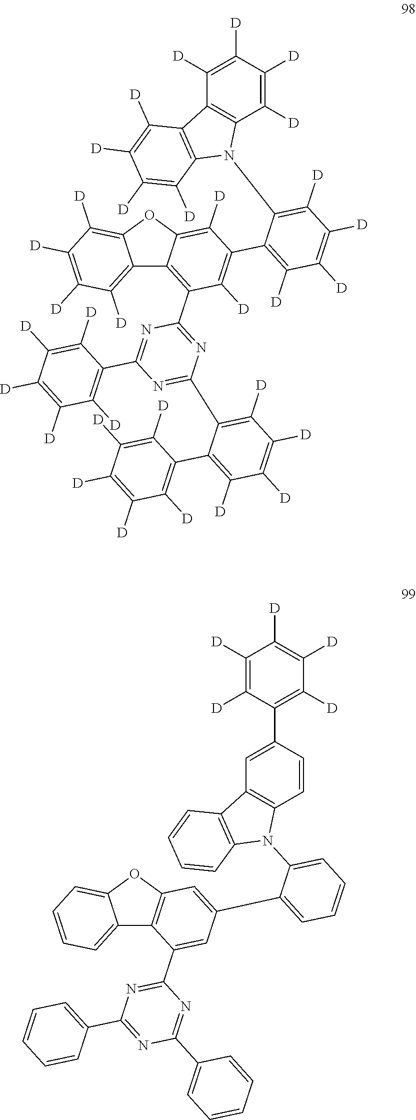 [SMILES: [2H]c1c([2H])c([2H])c(-c2nc(-c3c([2H])c([2H])c([2H])c([2H])c3-c3c([2H])c([2H])c([2H])c([2H])c3[2H])nc(-c3c([2H])c(-c4c([2H])c([2H])c([2H])c([2H])c4-n4c5c([2H])c([2H])c([2H])c([2H])c5c5c([2H])c([2H])c([2H])c([2H])c54)c([2H])c4oc5c([2H])c([2H])c([2H])c([2H])c5c34)n2)c([2H])c1[2H] ; [2H]c1c([2H])c([2H])c(-c2ccc3c(c2)c2ccccc2n3-c2ccccc2-c2cc(-c3nc(-c4ccccc4)nc(-c4ccccc4)n3)c3c(c2)oc2ccccc23)c([2H])c1[2H]]

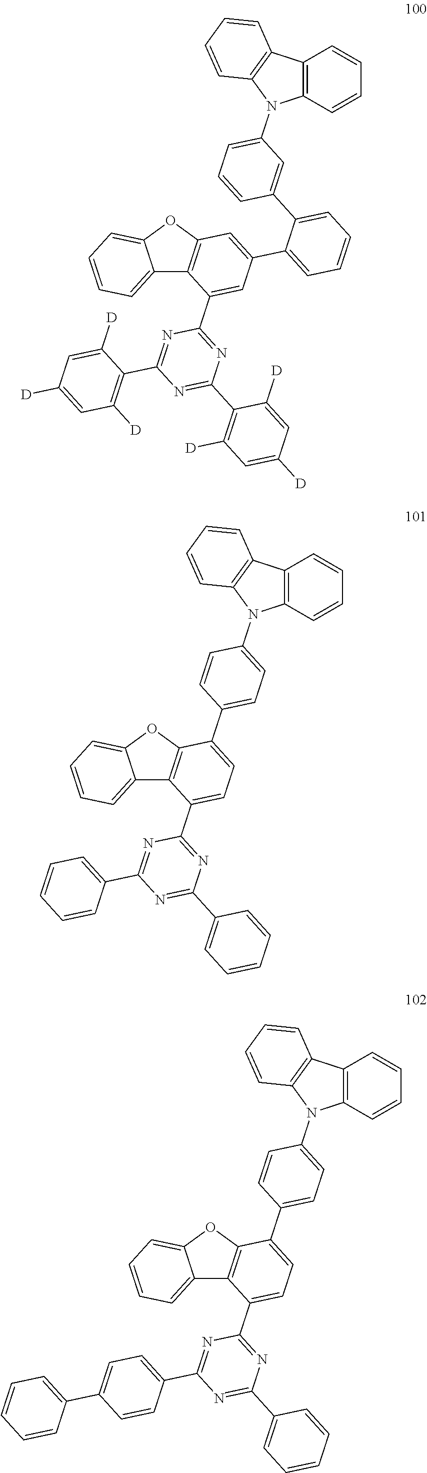 [SMILES: [2H]c1cc([2H])c(-c2nc(-c3c([2H])cc([2H])cc3[2H])nc(-c3cc(-c4ccccc4-c4cccc(-n5c6ccccc6c6ccccc65)c4)cc4oc5ccccc5c34)n2)c([2H])c1 ; c1ccc(-c2nc(-c3ccccc3)nc(-c3ccc(-c4ccc(-n5c6ccccc6c6ccccc65)cc4)c4oc5ccccc5c34)n2)cc1 ; c1ccc(-c2ccc(-c3nc(-c4ccccc4)nc(-c4ccc(-c5ccc(-n6c7ccccc7c7ccccc76)cc5)c5oc6ccccc6c45)n3)cc2)cc1]

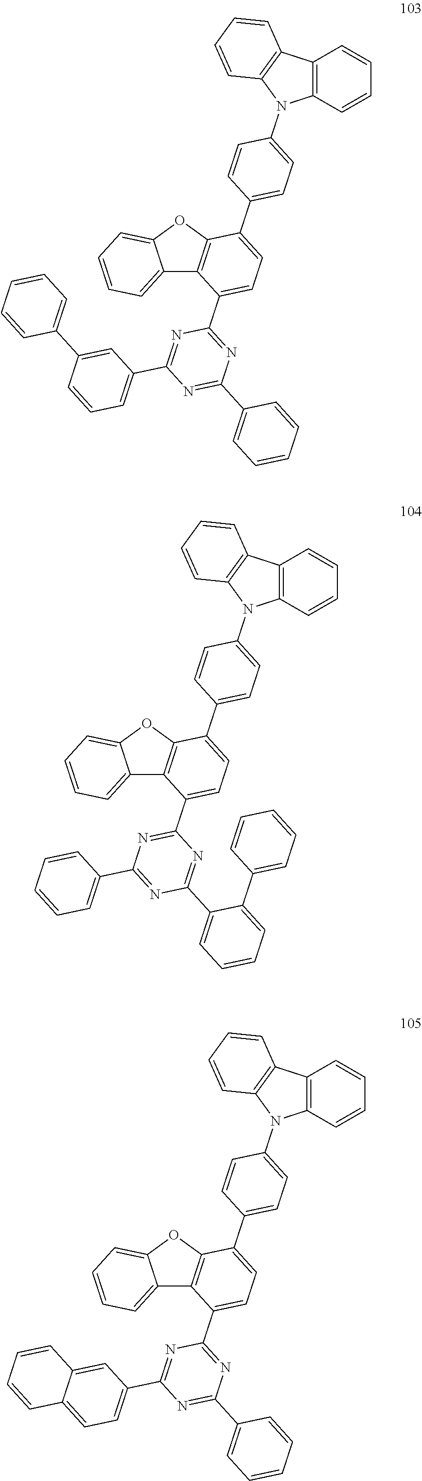 [SMILES: c1ccc(-c2cccc(-c3nc(-c4ccccc4)nc(-c4ccc(-c5ccc(-n6c7ccccc7c7ccccc76)cc5)c5oc6ccccc6c45)n3)c2)cc1 ; c1ccc(-c2nc(-c3ccccc3-c3ccccc3)nc(-c3ccc(-c4ccc(-n5c6ccccc6c6ccccc65)cc4)c4oc5ccccc5c34)n2)cc1 ; c1ccc(-c2nc(-c3ccc4ccccc4c3)nc(-c3ccc(-c4ccc(-n5c6ccccc6c6ccccc65)cc4)c4oc5ccccc5c34)n2)cc1]

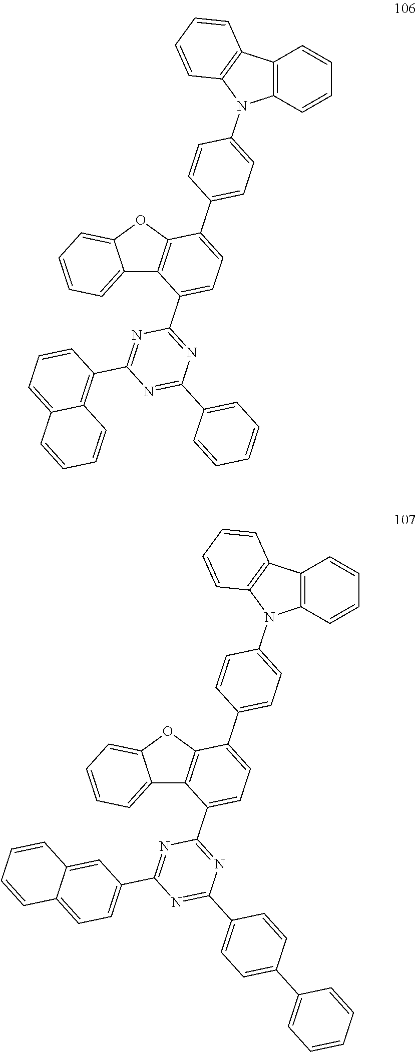 [SMILES: c1ccc(-c2nc(-c3cccc4ccccc34)nc(-c3ccc(-c4ccc(-n5c6ccccc6c6ccccc65)cc4)c4oc5ccccc5c34)n2)cc1 ; c1ccc(-c2ccc(-c3nc(-c4ccc5ccccc5c4)nc(-c4ccc(-c5ccc(-n6c7ccccc7c7ccccc76)cc5)c5oc6ccccc6c45)n3)cc2)cc1]

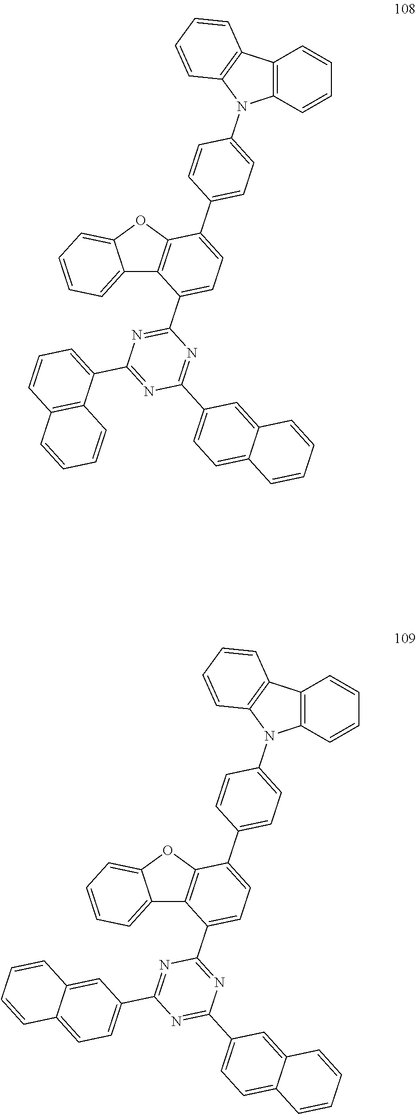 [SMILES: c1ccc2cc(-c3nc(-c4cccc5ccccc45)nc(-c4ccc(-c5ccc(-n6c7ccccc7c7ccccc76)cc5)c5oc6ccccc6c45)n3)ccc2c1 ; c1ccc2cc(-c3nc(-c4ccc5ccccc5c4)nc(-c4ccc(-c5ccc(-n6c7ccccc7c7ccccc76)cc5)c5oc6ccccc6c45)n3)ccc2c1]

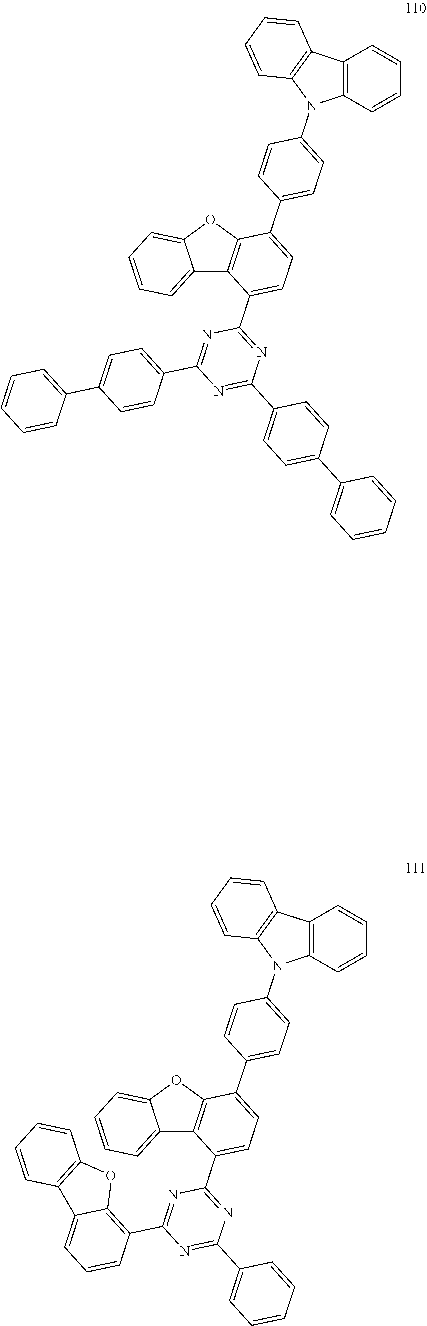 [SMILES: c1ccc(-c2ccc(-c3nc(-c4ccc(-c5ccccc5)cc4)nc(-c4ccc(-c5ccc(-n6c7ccccc7c7ccccc76)cc5)c5oc6ccccc6c45)n3)cc2)cc1 ; c1ccc(-c2nc(-c3cccc4c3oc3ccccc34)nc(-c3ccc(-c4ccc(-n5c6ccccc6c6ccccc65)cc4)c4oc5ccccc5c34)n2)cc1]

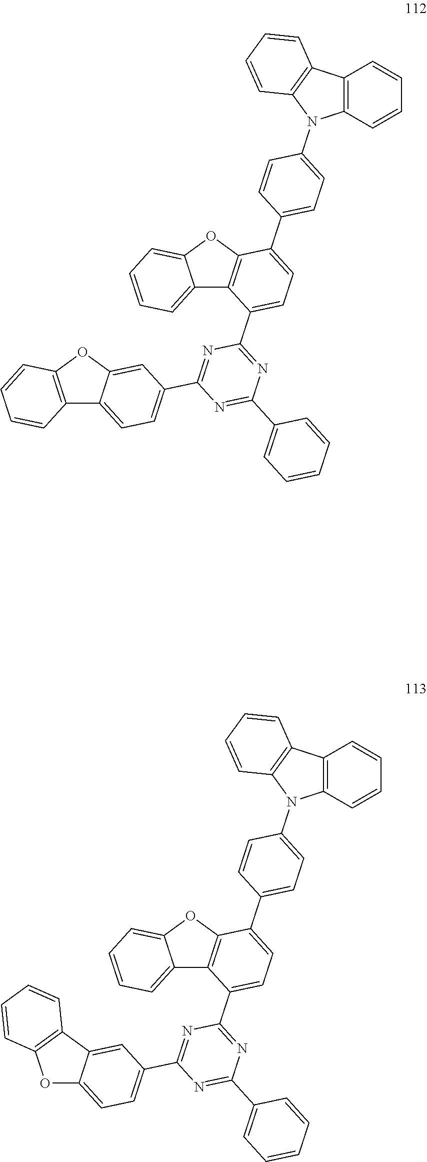 [SMILES: c1ccc(-c2nc(-c3ccc4c(c3)oc3ccccc34)nc(-c3ccc(-c4ccc(-n5c6ccccc6c6ccccc65)cc4)c4oc5ccccc5c34)n2)cc1 ; c1ccc(-c2nc(-c3ccc4oc5ccccc5c4c3)nc(-c3ccc(-c4ccc(-n5c6ccccc6c6ccccc65)cc4)c4oc5ccccc5c34)n2)cc1]

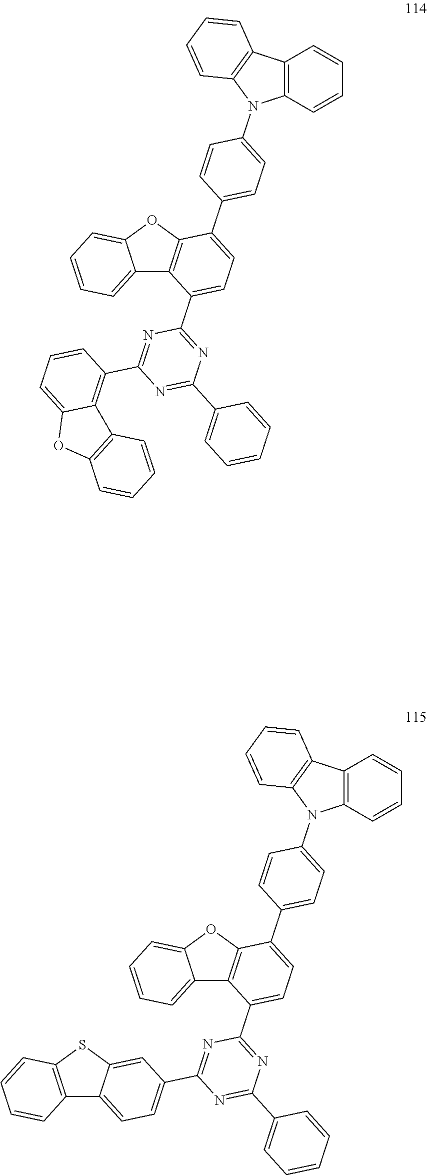 [SMILES: c1ccc(-c2nc(-c3cccc4oc5ccccc5c34)nc(-c3ccc(-c4ccc(-n5c6ccccc6c6ccccc65)cc4)c4oc5ccccc5c34)n2)cc1 ; c1ccc(-c2nc(-c3ccc4c(c3)sc3ccccc34)nc(-c3ccc(-c4ccc(-n5c6ccccc6c6ccccc65)cc4)c4oc5ccccc5c34)n2)cc1]

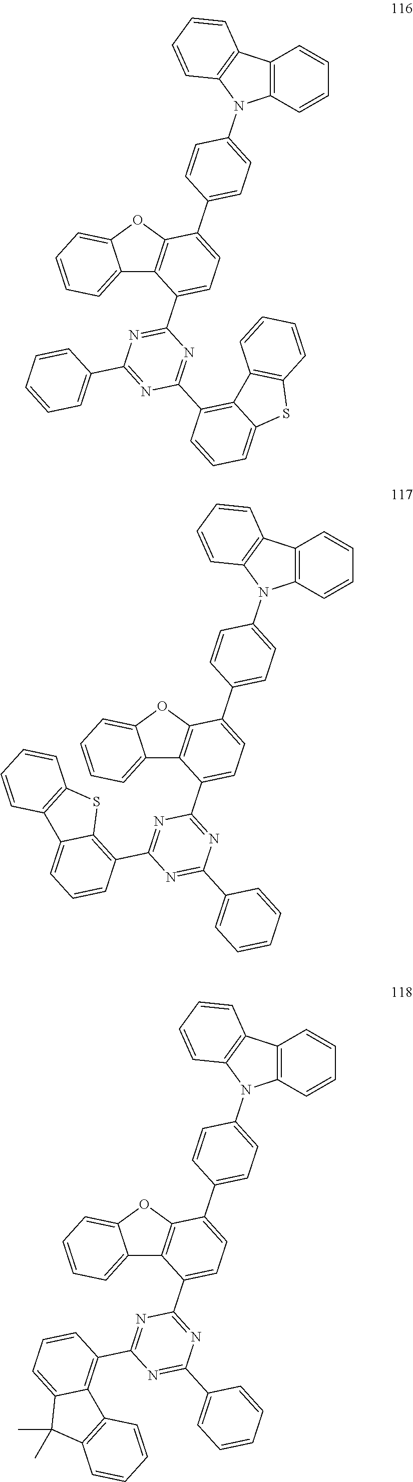

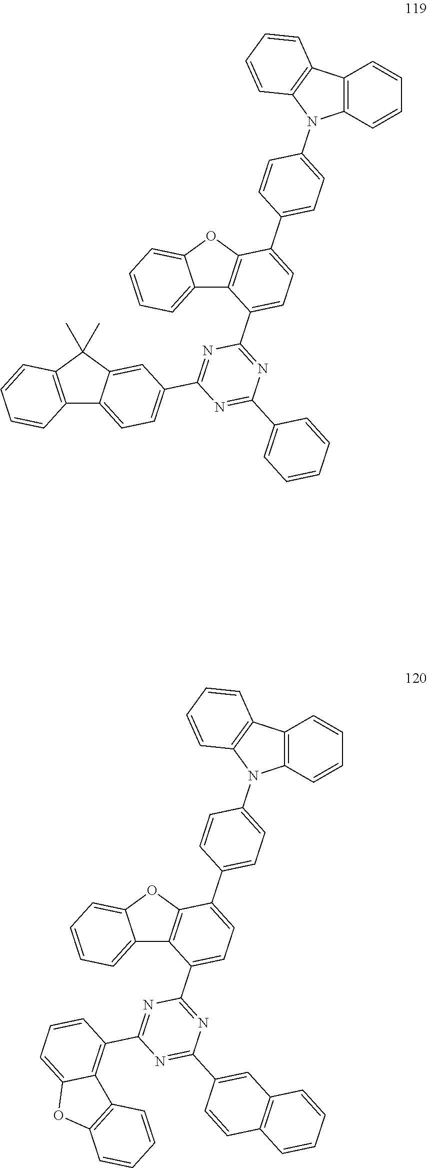 [SMILES: CC1(C)c2ccccc2-c2ccc(-c3nc(-c4ccccc4)nc(-c4ccc(-c5ccc(-n6c7ccccc7c7ccccc76)cc5)c5oc6ccccc6c45)n3)cc21 ; c1ccc2cc(-c3nc(-c4cccc5oc6ccccc6c45)nc(-c4ccc(-c5ccc(-n6c7ccccc7c7ccccc76)cc5)c5oc6ccccc6c45)n3)ccc2c1]

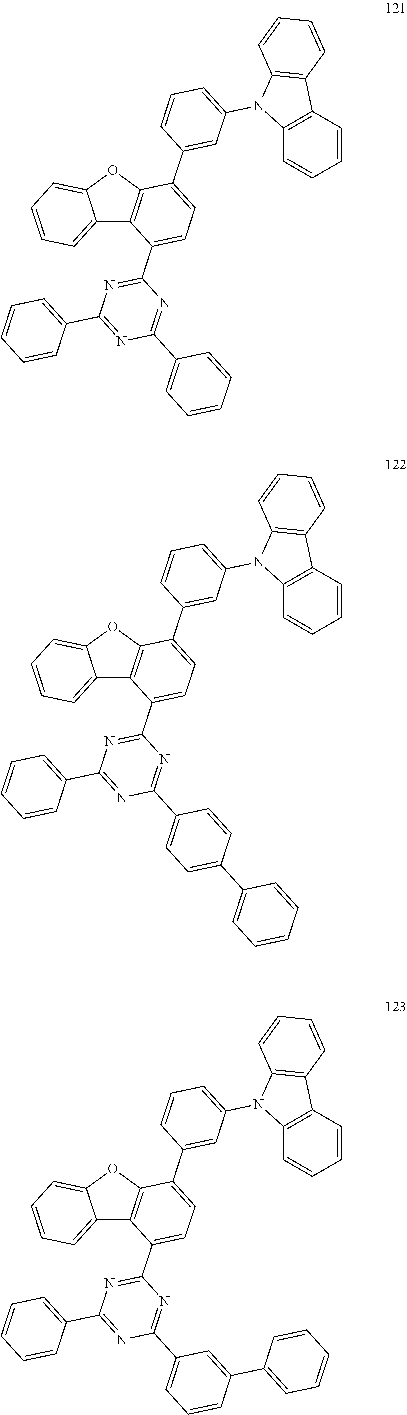 [SMILES: c1ccc(-c2nc(-c3ccccc3)nc(-c3ccc(-c4cccc(-n5c6ccccc6c6ccccc65)c4)c4oc5ccccc5c34)n2)cc1 ; c1ccc(-c2ccc(-c3nc(-c4ccccc4)nc(-c4ccc(-c5cccc(-n6c7ccccc7c7ccccc76)c5)c5oc6ccccc6c45)n3)cc2)cc1 ; c1ccc(-c2cccc(-c3nc(-c4ccccc4)nc(-c4ccc(-c5cccc(-n6c7ccccc7c7ccccc76)c5)c5oc6ccccc6c45)n3)c2)cc1]

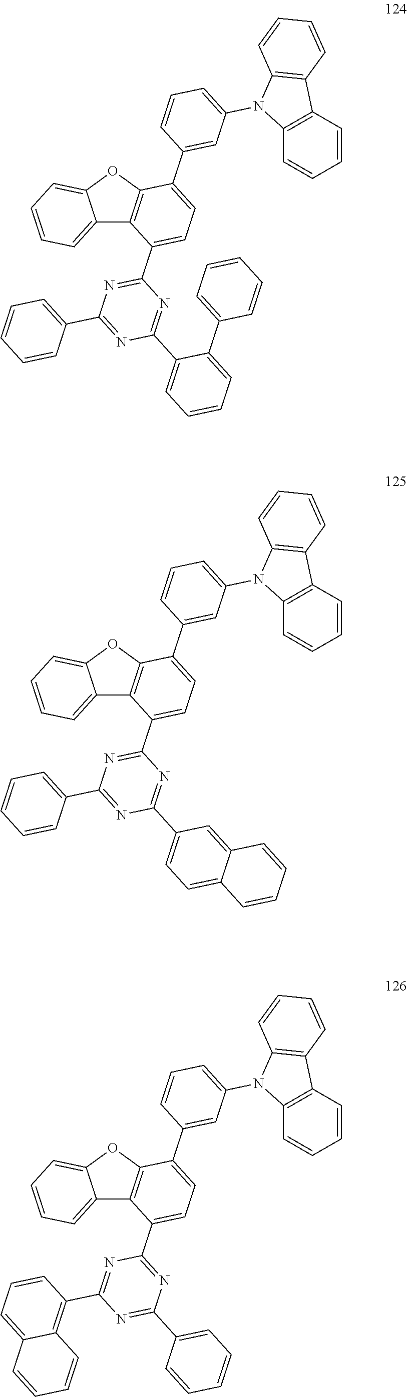 [SMILES: c1ccc(-c2nc(-c3ccccc3-c3ccccc3)nc(-c3ccc(-c4cccc(-n5c6ccccc6c6ccccc65)c4)c4oc5ccccc5c34)n2)cc1 ; c1ccc(-c2nc(-c3ccc4ccccc4c3)nc(-c3ccc(-c4cccc(-n5c6ccccc6c6ccccc65)c4)c4oc5ccccc5c34)n2)cc1 ; c1ccc(-c2nc(-c3cccc4ccccc34)nc(-c3ccc(-c4cccc(-n5c6ccccc6c6ccccc65)c4)c4oc5ccccc5c34)n2)cc1]

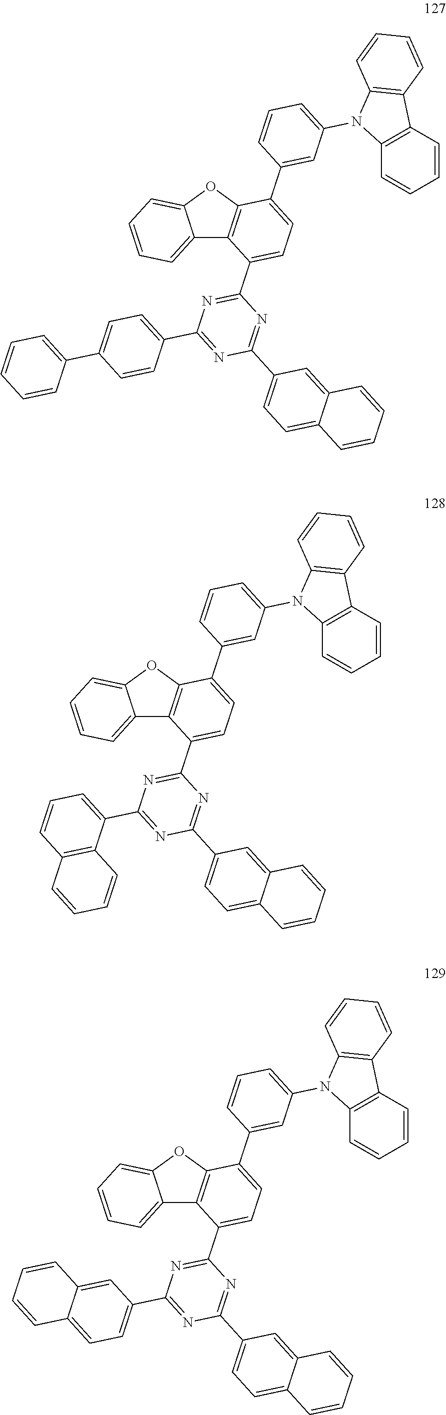 [SMILES: c1ccc(-c2ccc(-c3nc(-c4ccc5ccccc5c4)nc(-c4ccc(-c5cccc(-n6c7ccccc7c7ccccc76)c5)c5oc6ccccc6c45)n3)cc2)cc1 ; c1cc(-c2ccc(-c3nc(-c4ccc5ccccc5c4)nc(-c4cccc5ccccc45)n3)c3c2oc2ccccc23)cc(-n2c3ccccc3c3ccccc32)c1 ; c1cc(-c2ccc(-c3nc(-c4ccc5ccccc5c4)nc(-c4ccc5ccccc5c4)n3)c3c2oc2ccccc23)cc(-n2c3ccccc3c3ccccc32)c1]

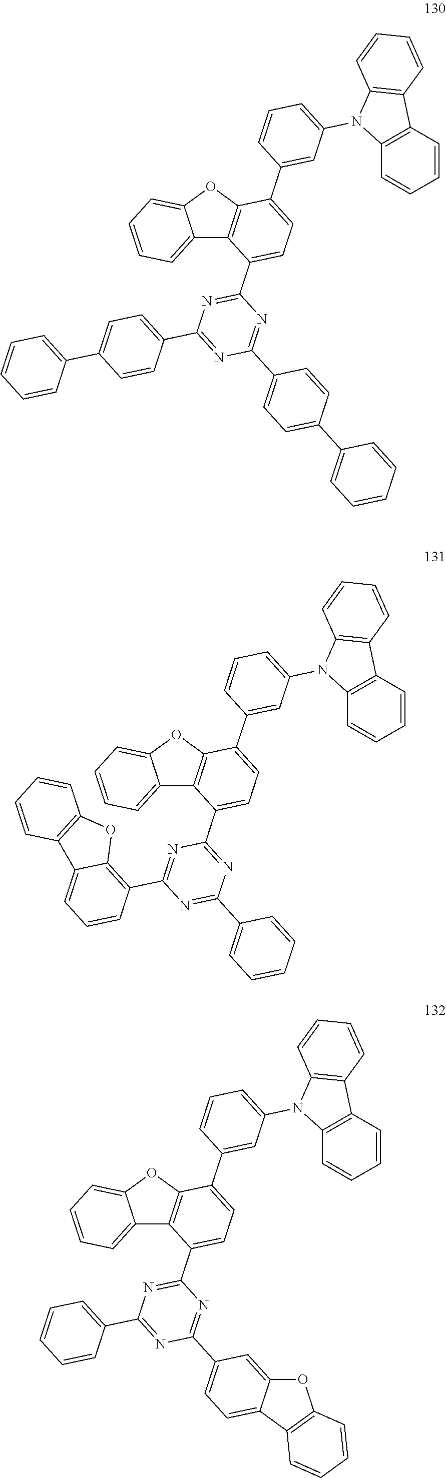 [SMILES: c1ccc(-c2ccc(-c3nc(-c4ccc(-c5ccccc5)cc4)nc(-c4ccc(-c5cccc(-n6c7ccccc7c7ccccc76)c5)c5oc6ccccc6c45)n3)cc2)cc1 ; c1ccc(-c2nc(-c3cccc4c3oc3ccccc34)nc(-c3ccc(-c4cccc(-n5c6ccccc6c6ccccc65)c4)c4oc5ccccc5c34)n2)cc1 ; c1ccc(-c2nc(-c3ccc4c(c3)oc3ccccc34)nc(-c3ccc(-c4cccc(-n5c6ccccc6c6ccccc65)c4)c4oc5ccccc5c34)n2)cc1]

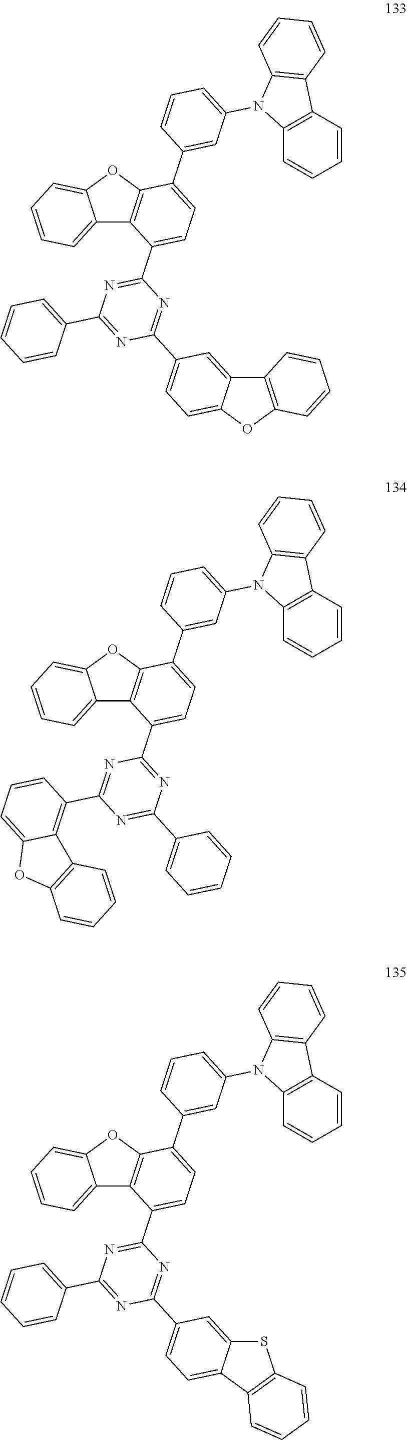 [SMILES: c1ccc(-c2nc(-c3ccc4oc5ccccc5c4c3)nc(-c3ccc(-c4cccc(-n5c6ccccc6c6ccccc65)c4)c4oc5ccccc5c34)n2)cc1 ; c1ccc(-c2nc(-c3cccc4oc5ccccc5c34)nc(-c3ccc(-c4cccc(-n5c6ccccc6c6ccccc65)c4)c4oc5ccccc5c34)n2)cc1 ; c1ccc(-c2nc(-c3ccc4c(c3)sc3ccccc34)nc(-c3ccc(-c4cccc(-n5c6ccccc6c6ccccc65)c4)c4oc5ccccc5c34)n2)cc1]

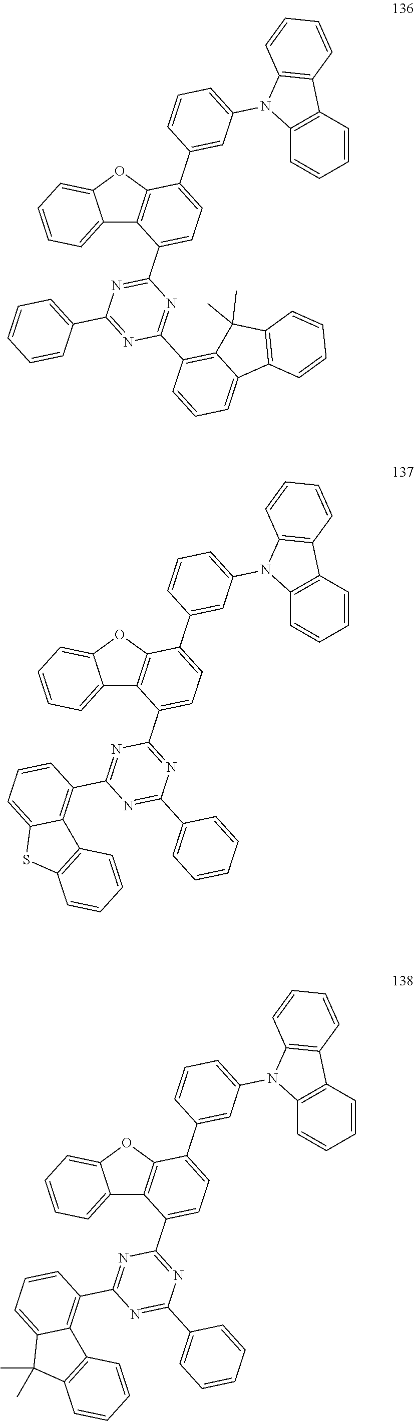 [SMILES: CC1(C)c2ccccc2-c2cccc(-c3nc(-c4ccccc4)nc(-c4ccc(-c5cccc(-n6c7ccccc7c7ccccc76)c5)c5oc6ccccc6c45)n3)c21 ; c1ccc(-c2nc(-c3cccc4sc5ccccc5c34)nc(-c3ccc(-c4cccc(-n5c6ccccc6c6ccccc65)c4)c4oc5ccccc5c34)n2)cc1 ; CC1(C)c2ccccc2-c2c(-c3nc(-c4ccccc4)nc(-c4ccc(-c5cccc(-n6c7ccccc7c7ccccc76)c5)c5oc6ccccc6c45)n3)cccc21]

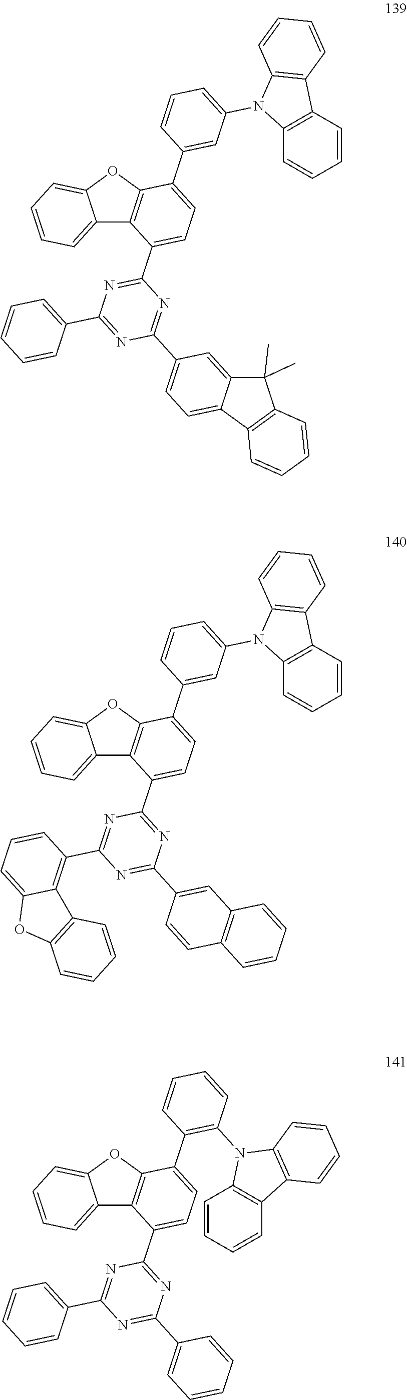 [SMILES: CC1(C)c2ccccc2-c2ccc(-c3nc(-c4ccccc4)nc(-c4ccc(-c5cccc(-n6c7ccccc7c7ccccc76)c5)c5oc6ccccc6c45)n3)cc21 ; c1cc(-c2ccc(-c3nc(-c4ccc5ccccc5c4)nc(-c4cccc5oc6ccccc6c45)n3)c3c2oc2ccccc23)cc(-n2c3ccccc3c3ccccc32)c1 ; c1ccc(-c2nc(-c3ccccc3)nc(-c3ccc(-c4ccccc4-n4c5ccccc5c5ccccc54)c4oc5ccccc5c34)n2)cc1]

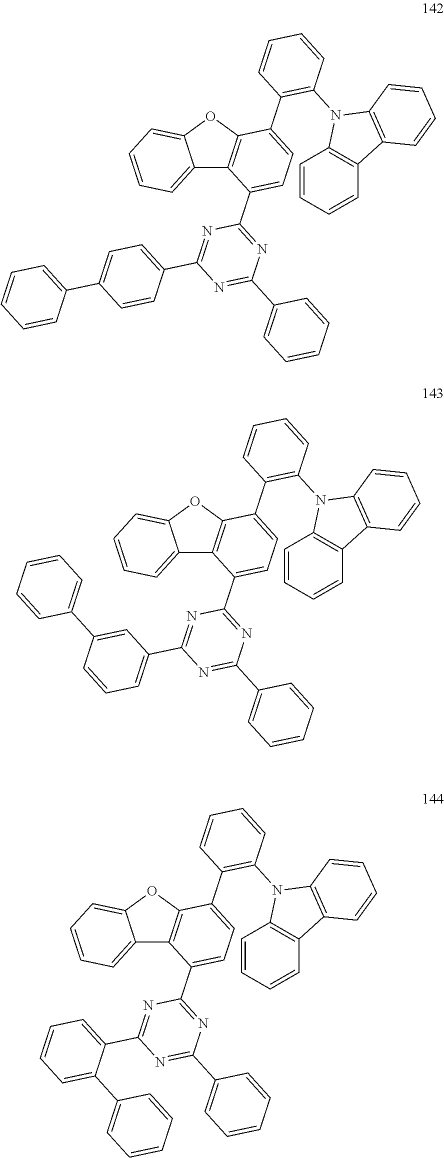 [SMILES: c1ccc(-c2ccc(-c3nc(-c4ccccc4)nc(-c4ccc(-c5ccccc5-n5c6ccccc6c6ccccc65)c5oc6ccccc6c45)n3)cc2)cc1 ; c1ccc(-c2cccc(-c3nc(-c4ccccc4)nc(-c4ccc(-c5ccccc5-n5c6ccccc6c6ccccc65)c5oc6ccccc6c45)n3)c2)cc1 ; c1ccc(-c2nc(-c3ccccc3-c3ccccc3)nc(-c3ccc(-c4ccccc4-n4c5ccccc5c5ccccc54)c4oc5ccccc5c34)n2)cc1]

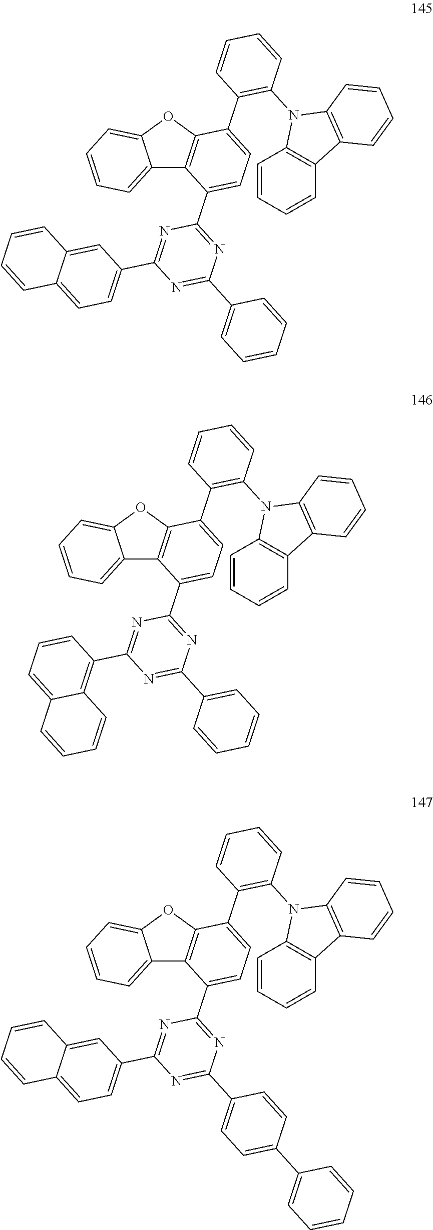 [SMILES: c1ccc(-c2nc(-c3ccc4ccccc4c3)nc(-c3ccc(-c4ccccc4-n4c5ccccc5c5ccccc54)c4oc5ccccc5c34)n2)cc1 ; c1ccc(-c2nc(-c3cccc4ccccc34)nc(-c3ccc(-c4ccccc4-n4c5ccccc5c5ccccc54)c4oc5ccccc5c34)n2)cc1 ; c1ccc(-c2ccc(-c3nc(-c4ccc5ccccc5c4)nc(-c4ccc(-c5ccccc5-n5c6ccccc6c6ccccc65)c5oc6ccccc6c45)n3)cc2)cc1]

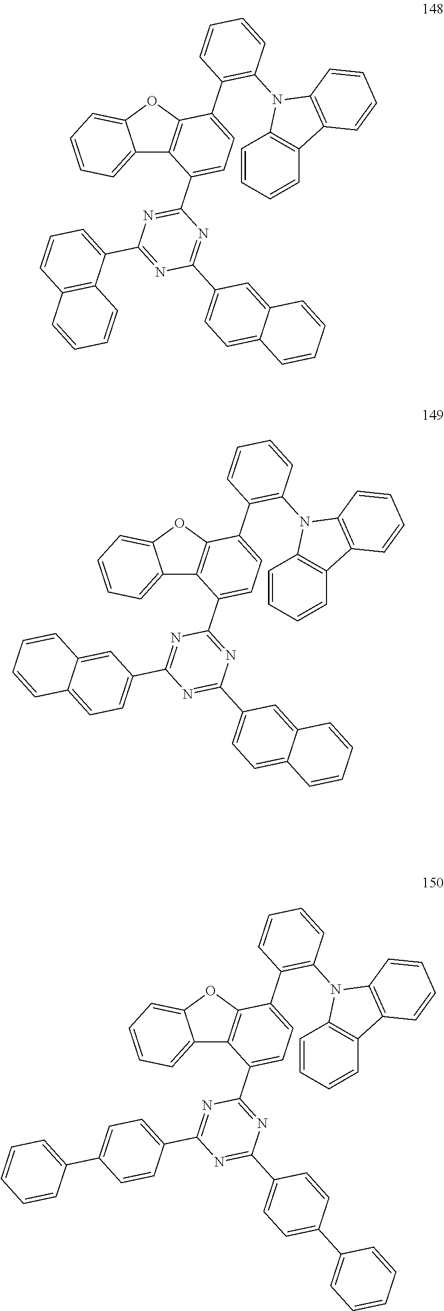 [SMILES: c1ccc(-n2c3ccccc3c3ccccc32)c(-c2ccc(-c3nc(-c4ccc5ccccc5c4)nc(-c4cccc5ccccc45)n3)c3c2oc2ccccc23)c1 ; c1ccc(-n2c3ccccc3c3ccccc32)c(-c2ccc(-c3nc(-c4ccc5ccccc5c4)nc(-c4ccc5ccccc5c4)n3)c3c2oc2ccccc23)c1 ; c1ccc(-c2ccc(-c3nc(-c4ccc(-c5ccccc5)cc4)nc(-c4ccc(-c5ccccc5-n5c6ccccc6c6ccccc65)c5oc6ccccc6c45)n3)cc2)cc1]

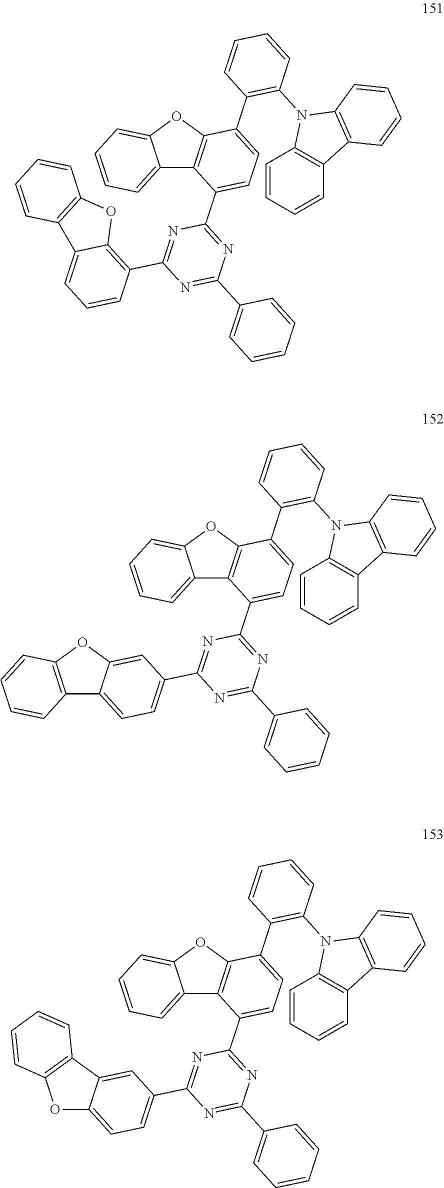 [SMILES: c1ccc(-c2nc(-c3cccc4c3oc3ccccc34)nc(-c3ccc(-c4ccccc4-n4c5ccccc5c5ccccc54)c4oc5ccccc5c34)n2)cc1 ; c1ccc(-c2nc(-c3ccc4c(c3)oc3ccccc34)nc(-c3ccc(-c4ccccc4-n4c5ccccc5c5ccccc54)c4oc5ccccc5c34)n2)cc1 ; c1ccc(-c2nc(-c3ccc4oc5ccccc5c4c3)nc(-c3ccc(-c4ccccc4-n4c5ccccc5c5ccccc54)c4oc5ccccc5c34)n2)cc1]

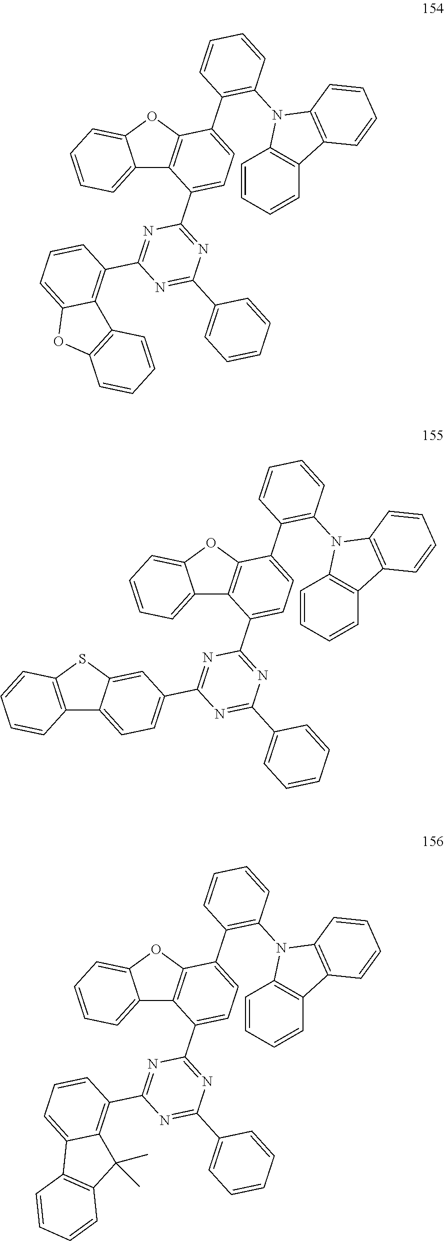 [SMILES: c1ccc(-c2nc(-c3cccc4oc5ccccc5c34)nc(-c3ccc(-c4ccccc4-n4c5ccccc5c5ccccc54)c4oc5ccccc5c34)n2)cc1 ; c1ccc(-c2nc(-c3ccc4c(c3)sc3ccccc34)nc(-c3ccc(-c4ccccc4-n4c5ccccc5c5ccccc54)c4oc5ccccc5c34)n2)cc1 ; CC1(C)c2ccccc2-c2cccc(-c3nc(-c4ccccc4)nc(-c4ccc(-c5ccccc5-n5c6ccccc6c6ccccc65)c5oc6ccccc6c45)n3)c21]

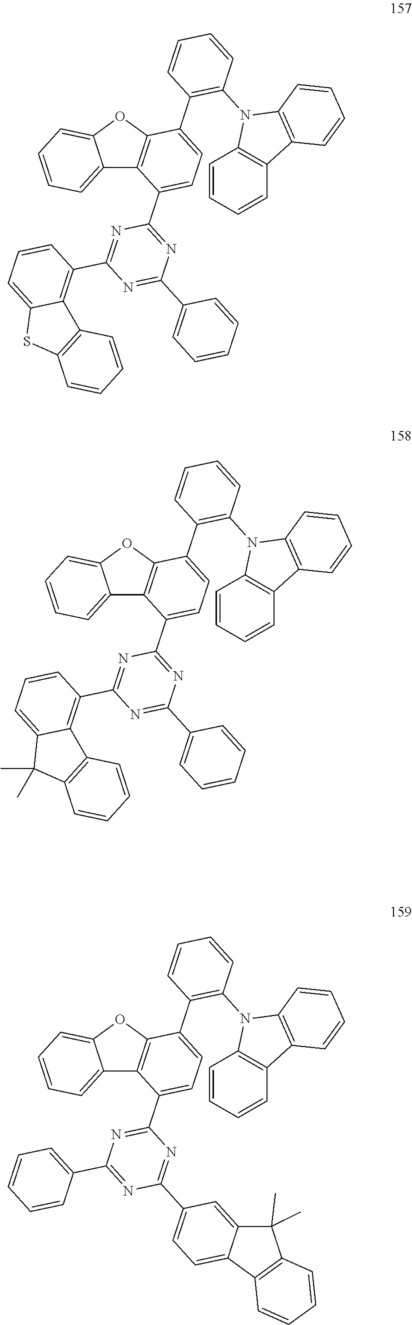 [SMILES: c1ccc(-c2nc(-c3cccc4sc5ccccc5c34)nc(-c3ccc(-c4ccccc4-n4c5ccccc5c5ccccc54)c4oc5ccccc5c34)n2)cc1 ; CC1(C)c2ccccc2-c2c(-c3nc(-c4ccccc4)nc(-c4ccc(-c5ccccc5-n5c6ccccc6c6ccccc65)c5oc6ccccc6c45)n3)cccc21 ; CC1(C)c2ccccc2-c2ccc(-c3nc(-c4ccccc4)nc(-c4ccc(-c5ccccc5-n5c6ccccc6c6ccccc65)c5oc6ccccc6c45)n3)cc21]

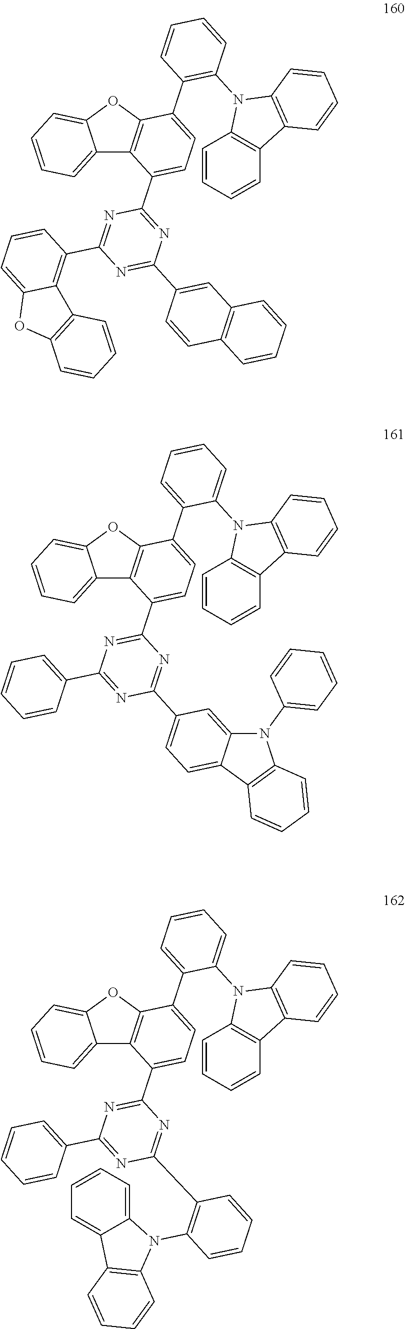 [SMILES: c1ccc(-n2c3ccccc3c3ccccc32)c(-c2ccc(-c3nc(-c4ccc5ccccc5c4)nc(-c4cccc5oc6ccccc6c45)n3)c3c2oc2ccccc23)c1 ; c1ccc(-c2nc(-c3ccc4c5ccccc5n(-c5ccccc5)c4c3)nc(-c3ccc(-c4ccccc4-n4c5ccccc5c5ccccc54)c4oc5ccccc5c34)n2)cc1 ; c1ccc(-c2nc(-c3ccccc3-n3c4ccccc4c4ccccc43)nc(-c3ccc(-c4ccccc4-n4c5ccccc5c5ccccc54)c4oc5ccccc5c34)n2)cc1]

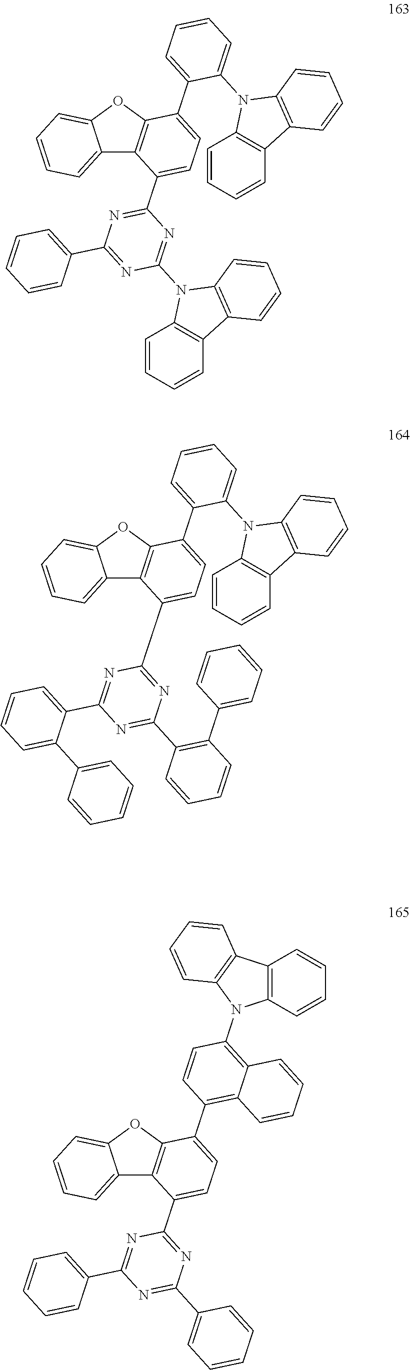 [SMILES: c1ccc(-c2nc(-c3ccc(-c4ccccc4-n4c5ccccc5c5ccccc54)c4oc5ccccc5c34)nc(-n3c4ccccc4c4ccccc43)n2)cc1 ; c1ccc(-c2ccccc2-c2nc(-c3ccccc3-c3ccccc3)nc(-c3ccc(-c4ccccc4-n4c5ccccc5c5ccccc54)c4oc5ccccc5c34)n2)cc1 ; c1ccc(-c2nc(-c3ccccc3)nc(-c3ccc(-c4ccc(-n5c6ccccc6c6ccccc65)c5ccccc45)c4oc5ccccc5c34)n2)cc1]

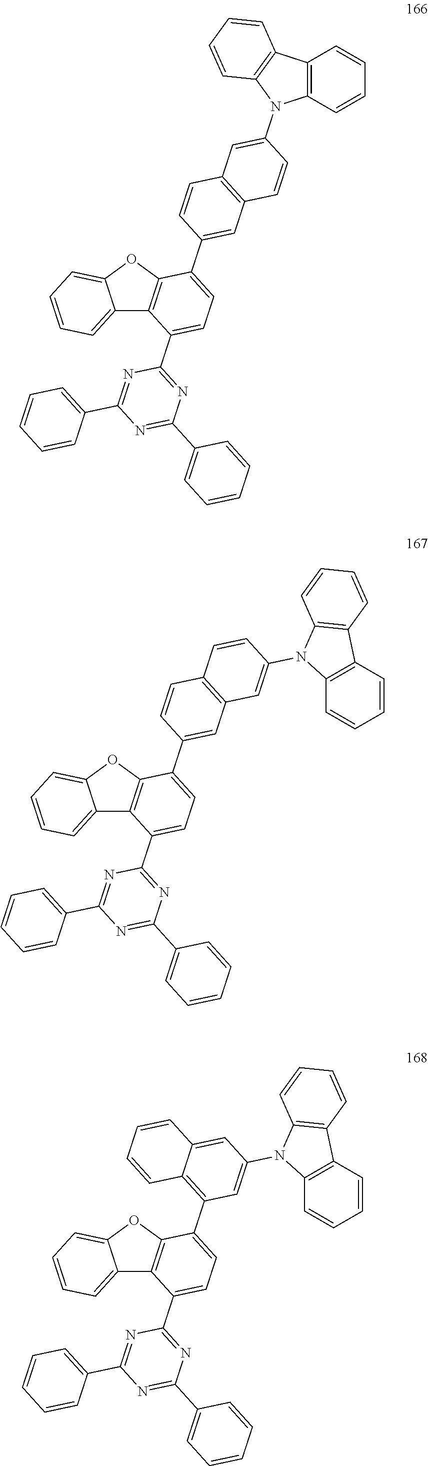 [SMILES: c1ccc(-c2nc(-c3ccccc3)nc(-c3ccc(-c4ccc5cc(-n6c7ccccc7c7ccccc76)ccc5c4)c4oc5ccccc5c34)n2)cc1 ; c1ccc(-c2nc(-c3ccccc3)nc(-c3ccc(-c4ccc5ccc(-n6c7ccccc7c7ccccc76)cc5c4)c4oc5ccccc5c34)n2)cc1 ; c1ccc(-c2nc(-c3ccccc3)nc(-c3ccc(-c4cc(-n5c6ccccc6c6ccccc65)cc5ccccc45)c4oc5ccccc5c34)n2)cc1]

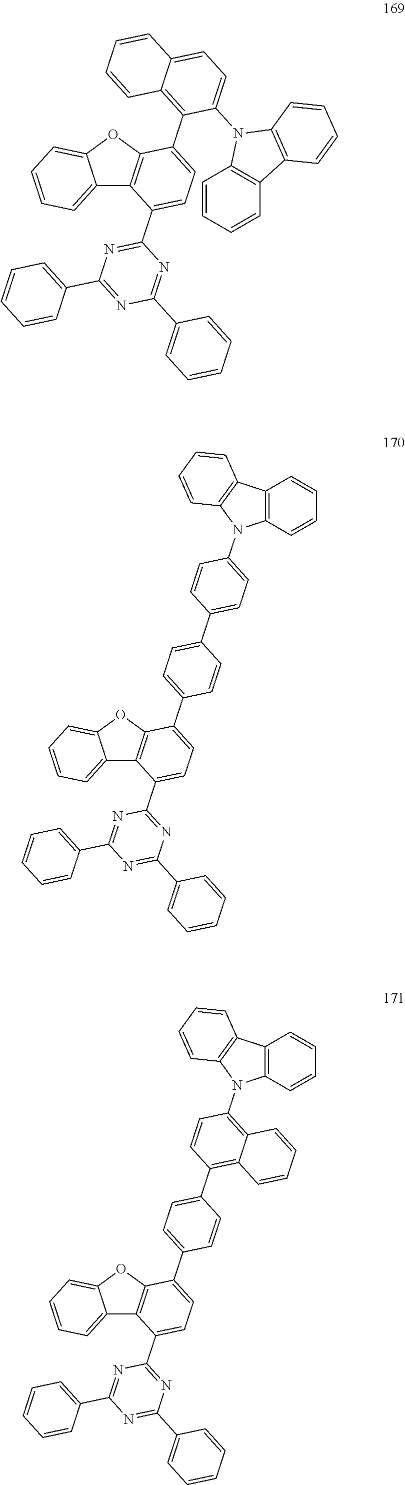 [SMILES: c1ccc(-c2nc(-c3ccccc3)nc(-c3ccc(-c4c(-n5c6ccccc6c6ccccc65)ccc5ccccc45)c4oc5ccccc5c34)n2)cc1 ; c1ccc(-c2nc(-c3ccccc3)nc(-c3ccc(-c4ccc(-c5ccc(-n6c7ccccc7c7ccccc76)cc5)cc4)c4oc5ccccc5c34)n2)cc1 ; c1ccc(-c2nc(-c3ccccc3)nc(-c3ccc(-c4ccc(-c5ccc(-n6c7ccccc7c7ccccc76)c6ccccc56)cc4)c4oc5ccccc5c34)n2)cc1]

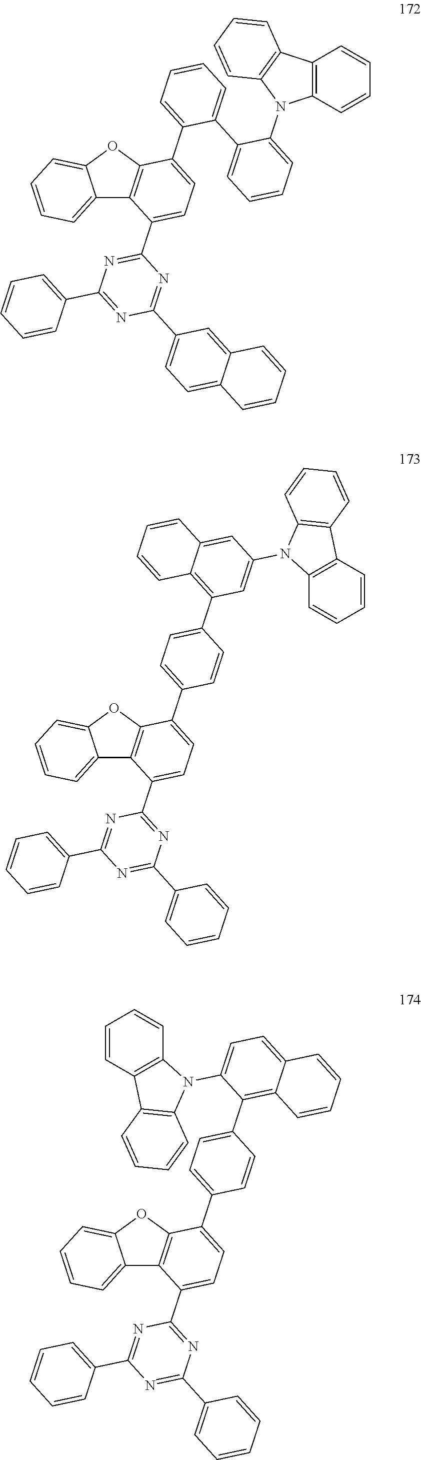 [SMILES: c1ccc(-c2nc(-c3ccc4ccccc4c3)nc(-c3ccc(-c4ccccc4-c4ccccc4-n4c5ccccc5c5ccccc54)c4oc5ccccc5c34)n2)cc1 ; c1ccc(-c2nc(-c3ccccc3)nc(-c3ccc(-c4ccc(-c5cc(-n6c7ccccc7c7ccccc76)cc6ccccc56)cc4)c4oc5ccccc5c34)n2)cc1 ; c1ccc(-c2nc(-c3ccccc3)nc(-c3ccc(-c4ccc(-c5c(-n6c7ccccc7c7ccccc76)ccc6ccccc56)cc4)c4oc5ccccc5c34)n2)cc1]

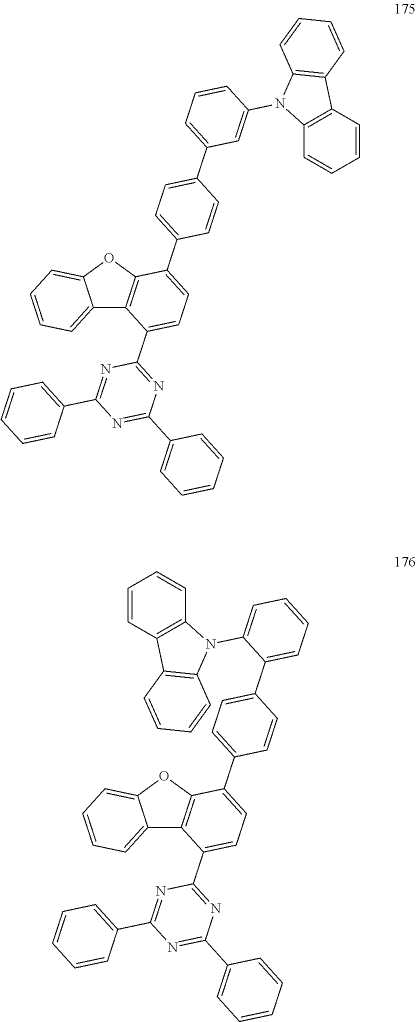 [SMILES: c1ccc(-c2nc(-c3ccccc3)nc(-c3ccc(-c4ccc(-c5cccc(-n6c7ccccc7c7ccccc76)c5)cc4)c4oc5ccccc5c34)n2)cc1 ; c1ccc(-c2nc(-c3ccccc3)nc(-c3ccc(-c4ccc(-c5ccccc5-n5c6ccccc6c6ccccc65)cc4)c4oc5ccccc5c34)n2)cc1]

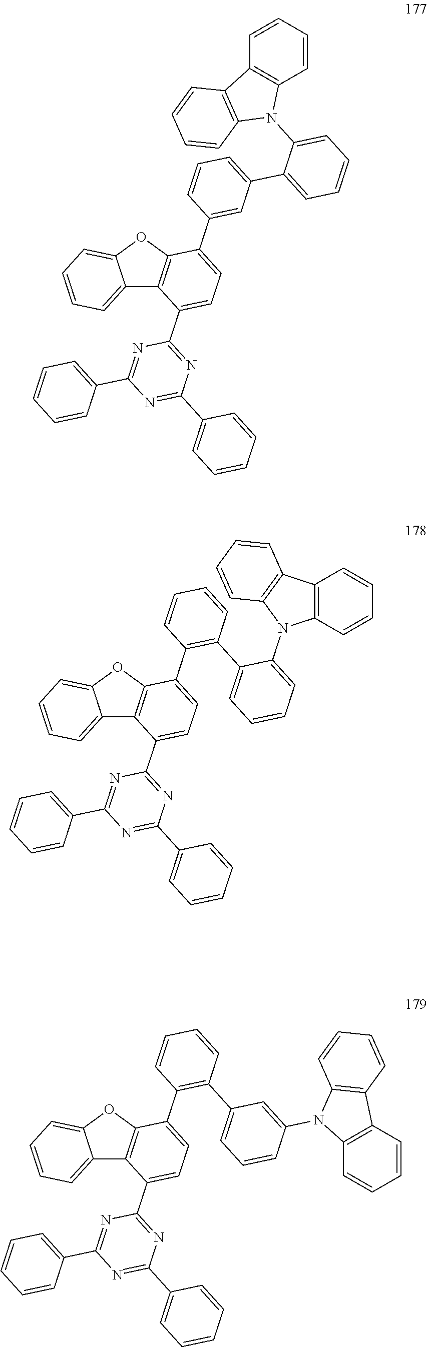 [SMILES: c1ccc(-c2nc(-c3ccccc3)nc(-c3ccc(-c4cccc(-c5ccccc5-n5c6ccccc6c6ccccc65)c4)c4oc5ccccc5c34)n2)cc1 ; c1ccc(-c2nc(-c3ccccc3)nc(-c3ccc(-c4ccccc4-c4ccccc4-n4c5ccccc5c5ccccc54)c4oc5ccccc5c34)n2)cc1 ; c1ccc(-c2nc(-c3ccccc3)nc(-c3ccc(-c4ccccc4-c4cccc(-n5c6ccccc6c6ccccc65)c4)c4oc5ccccc5c34)n2)cc1]

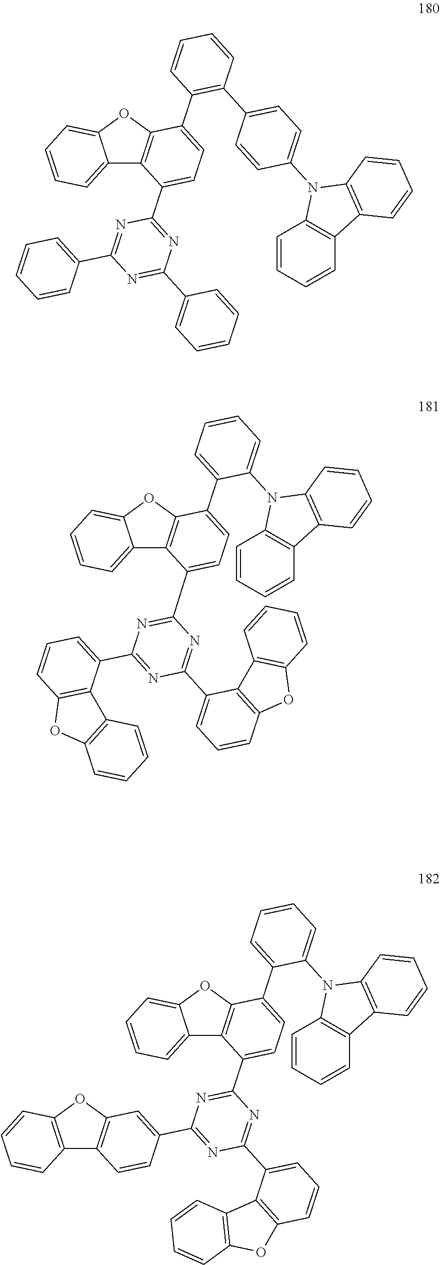 [SMILES: c1ccc(-c2nc(-c3ccccc3)nc(-c3ccc(-c4ccccc4-c4ccc(-n5c6ccccc6c6ccccc65)cc4)c4oc5ccccc5c34)n2)cc1 ; c1ccc(-n2c3ccccc3c3ccccc32)c(-c2ccc(-c3nc(-c4cccc5oc6ccccc6c45)nc(-c4cccc5oc6ccccc6c45)n3)c3c2oc2ccccc23)c1 ; c1ccc(-n2c3ccccc3c3ccccc32)c(-c2ccc(-c3nc(-c4ccc5c(c4)oc4ccccc45)nc(-c4cccc5oc6ccccc6c45)n3)c3c2oc2ccccc23)c1]

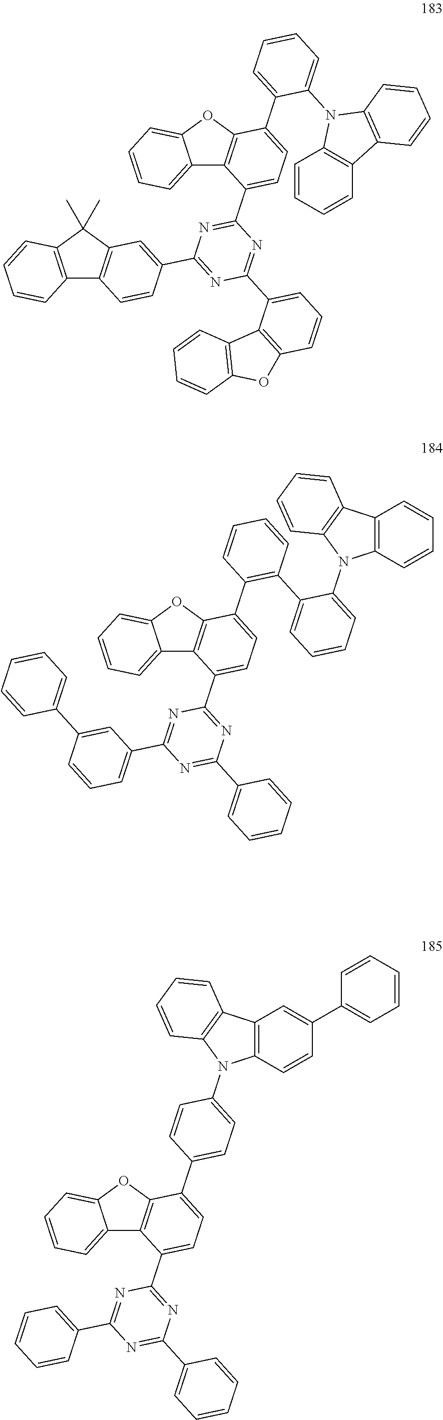 [SMILES: CC1(C)c2ccccc2-c2ccc(-c3nc(-c4cccc5oc6ccccc6c45)nc(-c4ccc(-c5ccccc5-n5c6ccccc6c6ccccc65)c5oc6ccccc6c45)n3)cc21 ; c1ccc(-c2cccc(-c3nc(-c4ccccc4)nc(-c4ccc(-c5ccccc5-c5ccccc5-n5c6ccccc6c6ccccc65)c5oc6ccccc6c45)n3)c2)cc1 ; c1ccc(-c2ccc3c(c2)c2ccccc2n3-c2ccc(-c3ccc(-c4nc(-c5ccccc5)nc(-c5ccccc5)n4)c4c3oc3ccccc34)cc2)cc1]

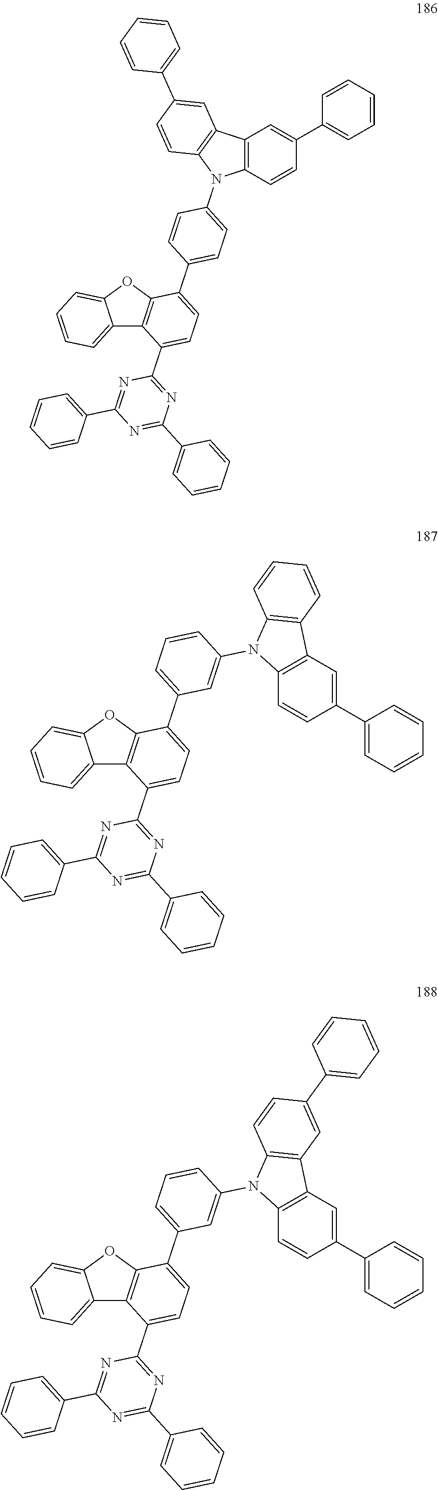 [SMILES: c1ccc(-c2ccc3c(c2)c2cc(-c4ccccc4)ccc2n3-c2ccc(-c3ccc(-c4nc(-c5ccccc5)nc(-c5ccccc5)n4)c4c3oc3ccccc34)cc2)cc1 ; c1ccc(-c2ccc3c(c2)c2ccccc2n3-c2cccc(-c3ccc(-c4nc(-c5ccccc5)nc(-c5ccccc5)n4)c4c3oc3ccccc34)c2)cc1 ; c1ccc(-c2ccc3c(c2)c2cc(-c4ccccc4)ccc2n3-c2cccc(-c3ccc(-c4nc(-c5ccccc5)nc(-c5ccccc5)n4)c4c3oc3ccccc34)c2)cc1]

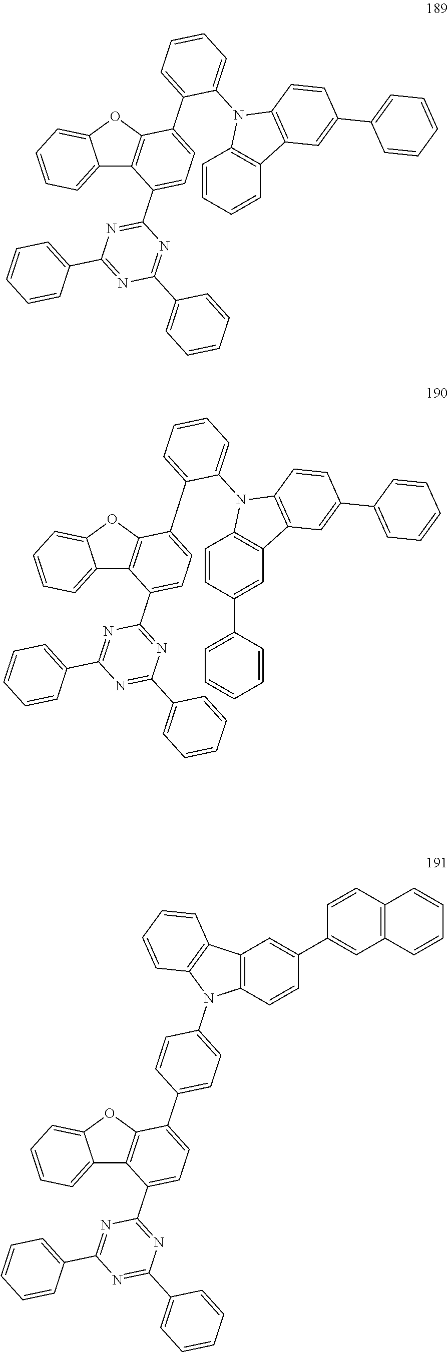 [SMILES: c1ccc(-c2ccc3c(c2)c2ccccc2n3-c2ccccc2-c2ccc(-c3nc(-c4ccccc4)nc(-c4ccccc4)n3)c3c2oc2ccccc23)cc1 ; c1ccc(-c2ccc3c(c2)c2cc(-c4ccccc4)ccc2n3-c2ccccc2-c2ccc(-c3nc(-c4ccccc4)nc(-c4ccccc4)n3)c3c2oc2ccccc23)cc1 ; c1ccc(-c2nc(-c3ccccc3)nc(-c3ccc(-c4ccc(-n5c6ccccc6c6cc(-c7ccc8ccccc8c7)ccc65)cc4)c4oc5ccccc5c34)n2)cc1]

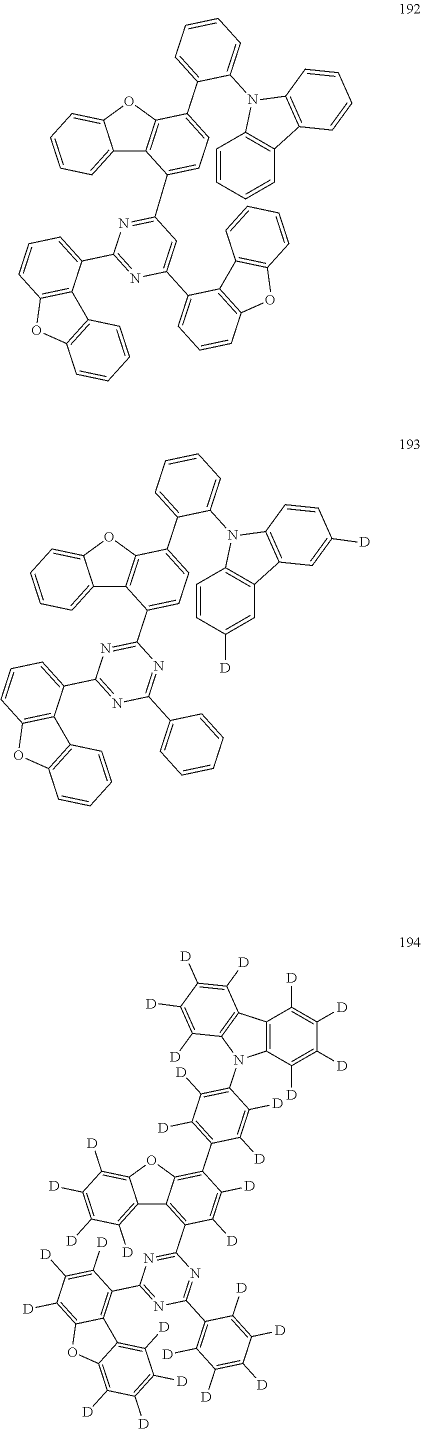 [SMILES: c1ccc(-n2c3ccccc3c3ccccc32)c(-c2ccc(-c3cc(-c4cccc5oc6ccccc6c45)nc(-c4cccc5oc6ccccc6c45)n3)c3c2oc2ccccc23)c1 ; [2H]c1ccc2c(c1)c1cc([2H])ccc1n2-c1ccccc1-c1ccc(-c2nc(-c3ccccc3)nc(-c3cccc4oc5ccccc5c34)n2)c2c1oc1ccccc12 ; [2H]c1c([2H])c([2H])c(-c2nc(-c3c([2H])c([2H])c([2H])c4oc5c([2H])c([2H])c([2H])c([2H])c5c34)nc(-c3c([2H])c([2H])c(-c4c([2H])c([2H])c(-n5c6c([2H])c([2H])c([2H])c([2H])c6c6c([2H])c([2H])c([2H])c([2H])c65)c([2H])c4[2H])c4oc5c([2H])c([2H])c([2H])c([2H])c5c34)n2)c([2H])c1[2H]]

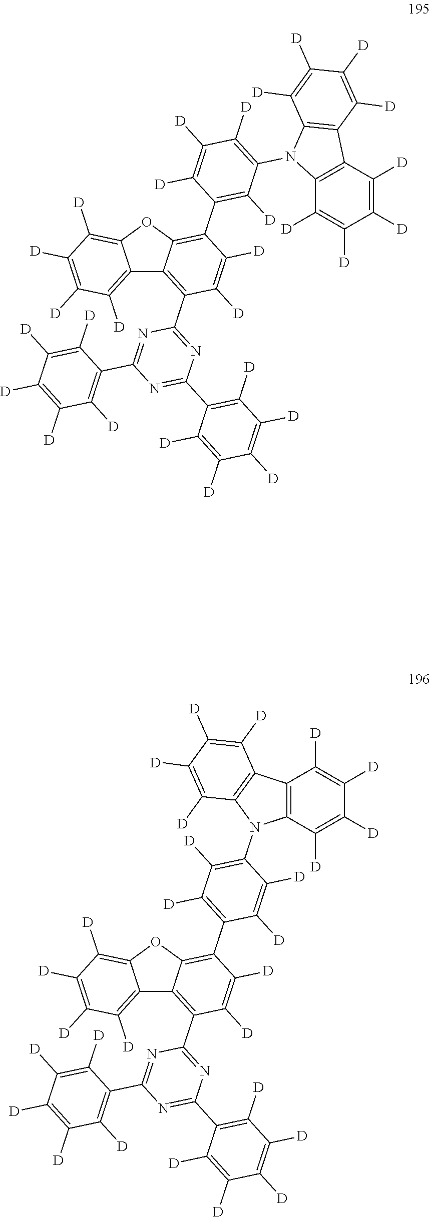 [SMILES: [2H]c1c([2H])c([2H])c(-c2nc(-c3c([2H])c([2H])c([2H])c([2H])c3[2H])nc(-c3c([2H])c([2H])c(-c4c([2H])c([2H])c([2H])c(-n5c6c([2H])c([2H])c([2H])c([2H])c6c6c([2H])c([2H])c([2H])c([2H])c65)c4[2H])c4oc5c([2H])c([2H])c([2H])c([2H])c5c34)n2)c([2H])c1[2H] ; [2H]c1c([2H])c([2H])c(-c2nc(-c3c([2H])c([2H])c([2H])c([2H])c3[2H])nc(-c3c([2H])c([2H])c(-c4c([2H])c([2H])c(-n5c6c([2H])c([2H])c([2H])c([2H])c6c6c([2H])c([2H])c([2H])c([2H])c65)c([2H])c4[2H])c4oc5c([2H])c([2H])c([2H])c([2H])c5c34)n2)c([2H])c1[2H]]

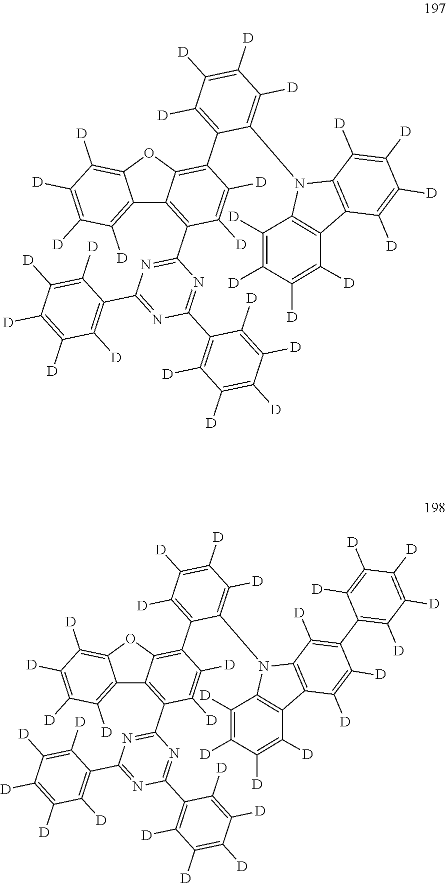

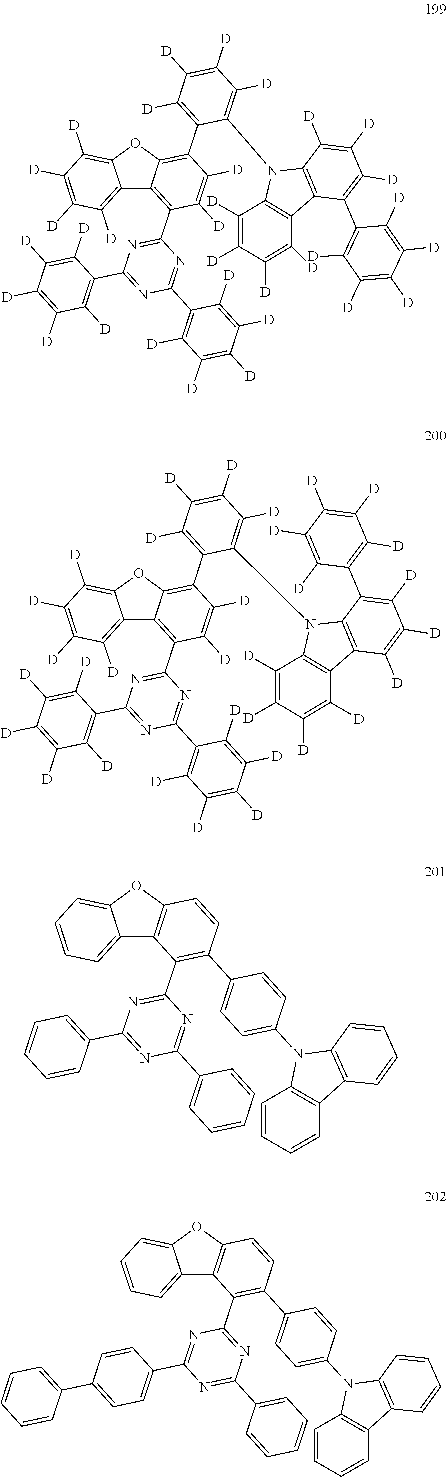

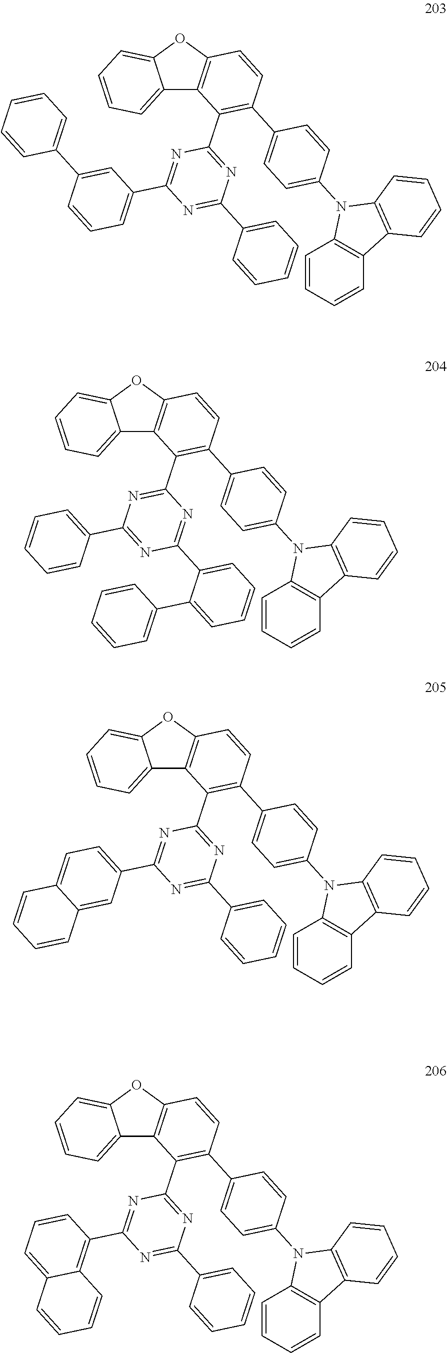 [SMILES: c1ccc(-c2cccc(-c3nc(-c4ccccc4)nc(-c4c(-c5ccc(-n6c7ccccc7c7ccccc76)cc5)ccc5oc6ccccc6c45)n3)c2)cc1 ; c1ccc(-c2nc(-c3ccccc3-c3ccccc3)nc(-c3c(-c4ccc(-n5c6ccccc6c6ccccc65)cc4)ccc4oc5ccccc5c34)n2)cc1 ; c1ccc(-c2nc(-c3ccc4ccccc4c3)nc(-c3c(-c4ccc(-n5c6ccccc6c6ccccc65)cc4)ccc4oc5ccccc5c34)n2)cc1 ; c1ccc(-c2nc(-c3cccc4ccccc34)nc(-c3c(-c4ccc(-n5c6ccccc6c6ccccc65)cc4)ccc4oc5ccccc5c34)n2)cc1]

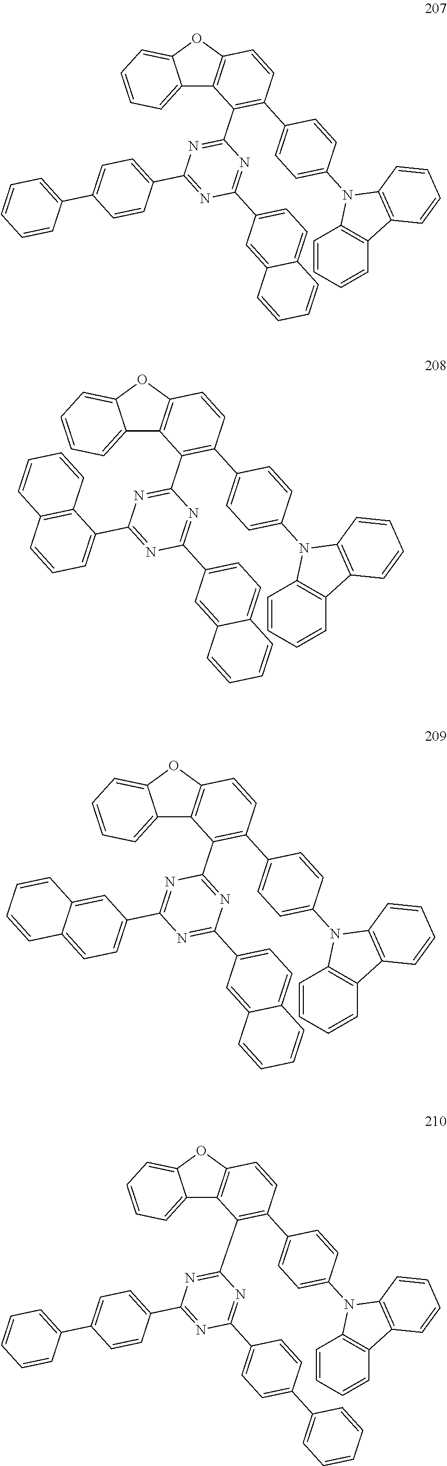 [SMILES: c1ccc(-c2ccc(-c3nc(-c4ccc5ccccc5c4)nc(-c4c(-c5ccc(-n6c7ccccc7c7ccccc76)cc5)ccc5oc6ccccc6c45)n3)cc2)cc1 ; c1ccc2cc(-c3nc(-c4cccc5ccccc45)nc(-c4c(-c5ccc(-n6c7ccccc7c7ccccc76)cc5)ccc5oc6ccccc6c45)n3)ccc2c1 ; c1ccc2cc(-c3nc(-c4ccc5ccccc5c4)nc(-c4c(-c5ccc(-n6c7ccccc7c7ccccc76)cc5)ccc5oc6ccccc6c45)n3)ccc2c1 ; c1ccc(-c2ccc(-c3nc(-c4ccc(-c5ccccc5)cc4)nc(-c4c(-c5ccc(-n6c7ccccc7c7ccccc76)cc5)ccc5oc6ccccc6c45)n3)cc2)cc1]

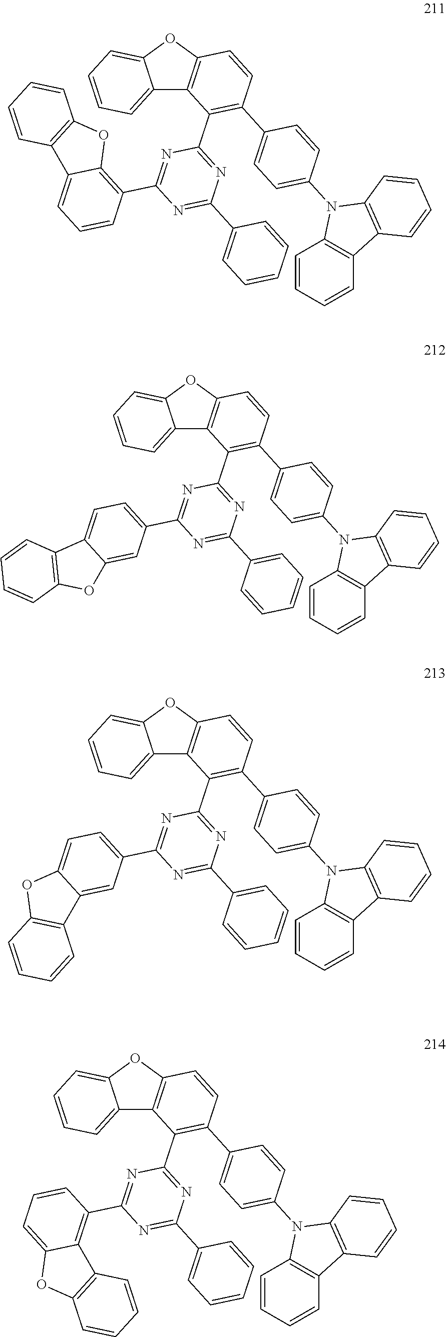 [SMILES: c1ccc(-c2nc(-c3cccc4c3oc3ccccc34)nc(-c3c(-c4ccc(-n5c6ccccc6c6ccccc65)cc4)ccc4oc5ccccc5c34)n2)cc1 ; c1ccc(-c2nc(-c3ccc4c(c3)oc3ccccc34)nc(-c3c(-c4ccc(-n5c6ccccc6c6ccccc65)cc4)ccc4oc5ccccc5c34)n2)cc1 ; c1ccc(-c2nc(-c3ccc4oc5ccccc5c4c3)nc(-c3c(-c4ccc(-n5c6ccccc6c6ccccc65)cc4)ccc4oc5ccccc5c34)n2)cc1 ; c1ccc(-c2nc(-c3cccc4oc5ccccc5c34)nc(-c3c(-c4ccc(-n5c6ccccc6c6ccccc65)cc4)ccc4oc5ccccc5c34)n2)cc1]

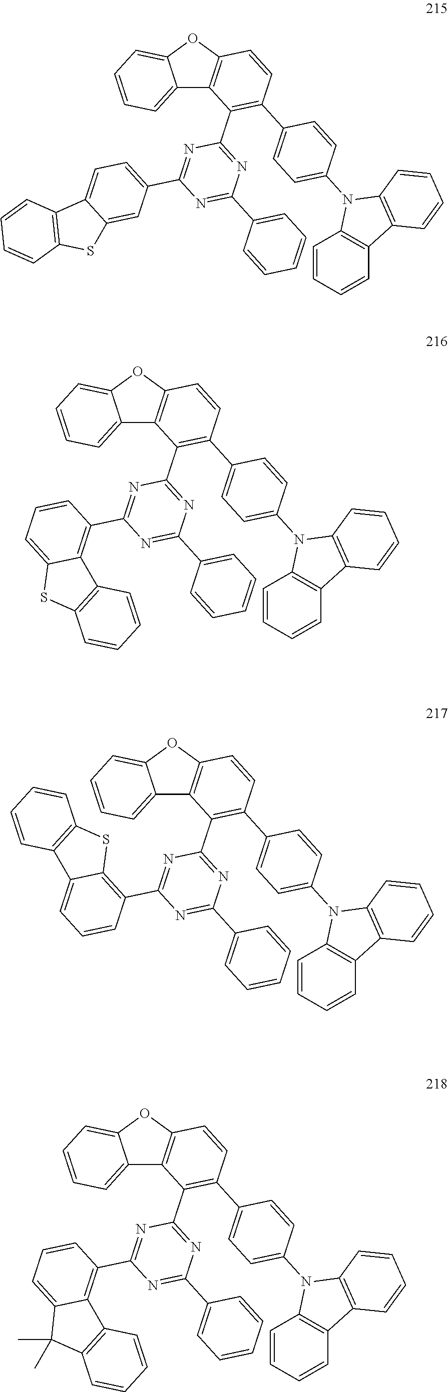 [SMILES: c1ccc(-c2nc(-c3ccc4c(c3)sc3ccccc34)nc(-c3c(-c4ccc(-n5c6ccccc6c6ccccc65)cc4)ccc4oc5ccccc5c34)n2)cc1 ; c1ccc(-c2nc(-c3c(-c4ccc(-n5c6ccccc6c6ccccc65)cc4)ccc4oc5ccccc5c34)nc(-c3cccc4sc5ccccc5c34)n2)cc1 ; c1ccc(-c2nc(-c3cccc4c3sc3ccccc34)nc(-c3c(-c4ccc(-n5c6ccccc6c6ccccc65)cc4)ccc4oc5ccccc5c34)n2)cc1 ; CC1(C)c2ccccc2-c2c(-c3nc(-c4ccccc4)nc(-c4c(-c5ccc(-n6c7ccccc7c7ccccc76)cc5)ccc5oc6ccccc6c45)n3)cccc21]

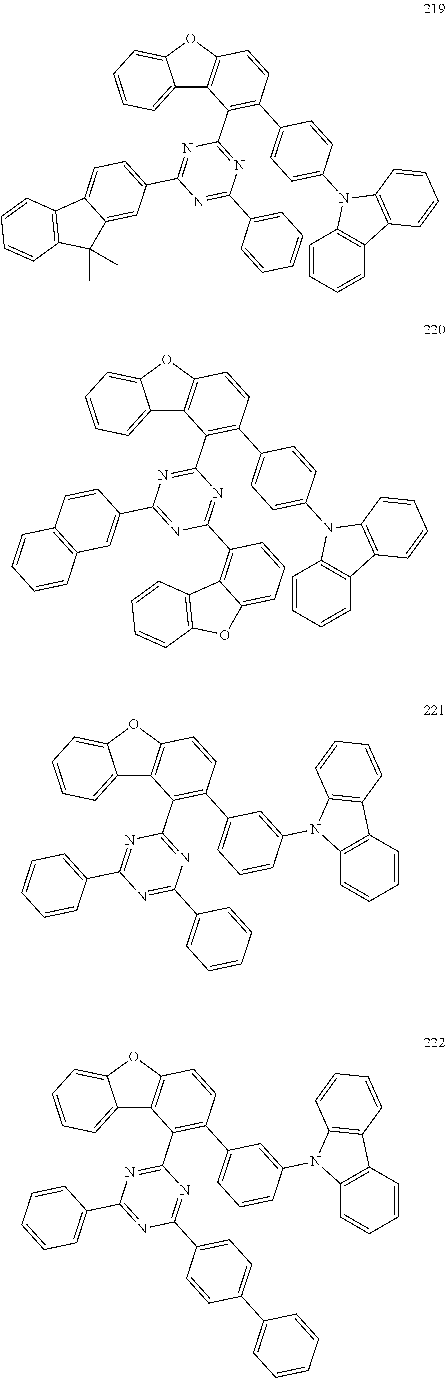 [SMILES: CC1(C)c2ccccc2-c2ccc(-c3nc(-c4ccccc4)nc(-c4c(-c5ccc(-n6c7ccccc7c7ccccc76)cc5)ccc5oc6ccccc6c45)n3)cc21 ; c1ccc2cc(-c3nc(-c4cccc5oc6ccccc6c45)nc(-c4c(-c5ccc(-n6c7ccccc7c7ccccc76)cc5)ccc5oc6ccccc6c45)n3)ccc2c1 ; c1ccc(-c2nc(-c3ccccc3)nc(-c3c(-c4cccc(-n5c6ccccc6c6ccccc65)c4)ccc4oc5ccccc5c34)n2)cc1 ; c1ccc(-c2ccc(-c3nc(-c4ccccc4)nc(-c4c(-c5cccc(-n6c7ccccc7c7ccccc76)c5)ccc5oc6ccccc6c45)n3)cc2)cc1]

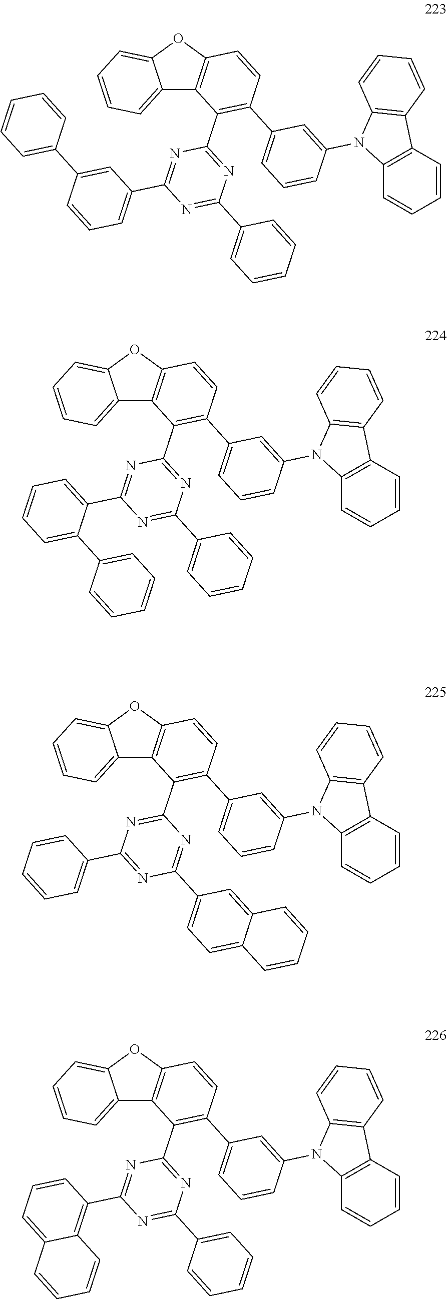 [SMILES: c1ccc(-c2cccc(-c3nc(-c4ccccc4)nc(-c4c(-c5cccc(-n6c7ccccc7c7ccccc76)c5)ccc5oc6ccccc6c45)n3)c2)cc1 ; c1ccc(-c2nc(-c3ccccc3-c3ccccc3)nc(-c3c(-c4cccc(-n5c6ccccc6c6ccccc65)c4)ccc4oc5ccccc5c34)n2)cc1 ; c1ccc(-c2nc(-c3ccc4ccccc4c3)nc(-c3c(-c4cccc(-n5c6ccccc6c6ccccc65)c4)ccc4oc5ccccc5c34)n2)cc1 ; c1ccc(-c2nc(-c3cccc4ccccc34)nc(-c3c(-c4cccc(-n5c6ccccc6c6ccccc65)c4)ccc4oc5ccccc5c34)n2)cc1]

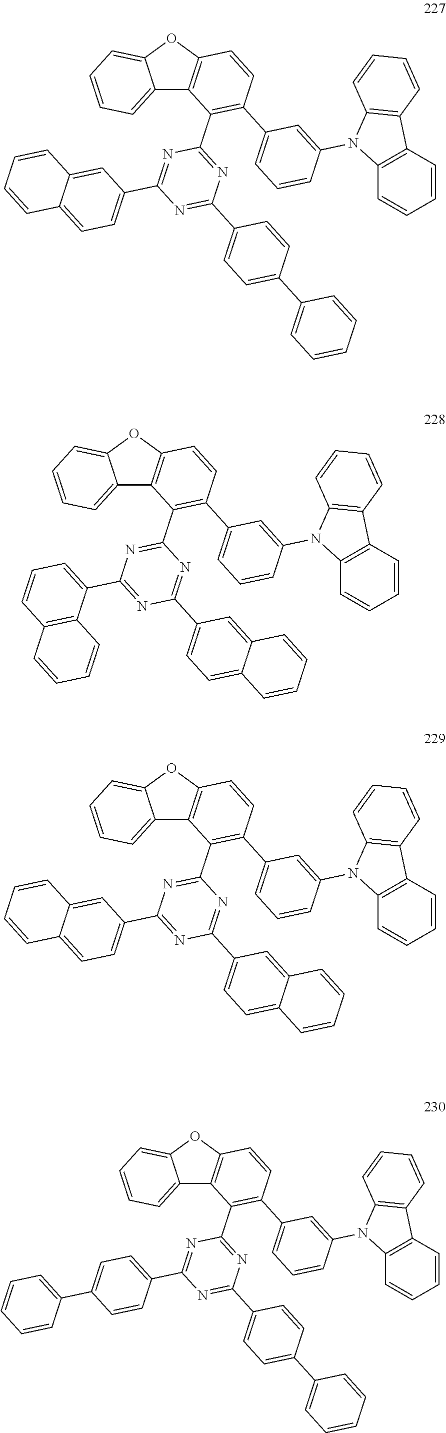 [SMILES: c1ccc(-c2ccc(-c3nc(-c4ccc5ccccc5c4)nc(-c4c(-c5cccc(-n6c7ccccc7c7ccccc76)c5)ccc5oc6ccccc6c45)n3)cc2)cc1 ; c1cc(-c2ccc3oc4ccccc4c3c2-c2nc(-c3ccc4ccccc4c3)nc(-c3cccc4ccccc34)n2)cc(-n2c3ccccc3c3ccccc32)c1 ; c1cc(-c2ccc3oc4ccccc4c3c2-c2nc(-c3ccc4ccccc4c3)nc(-c3ccc4ccccc4c3)n2)cc(-n2c3ccccc3c3ccccc32)c1 ; c1ccc(-c2ccc(-c3nc(-c4ccc(-c5ccccc5)cc4)nc(-c4c(-c5cccc(-n6c7ccccc7c7ccccc76)c5)ccc5oc6ccccc6c45)n3)cc2)cc1]

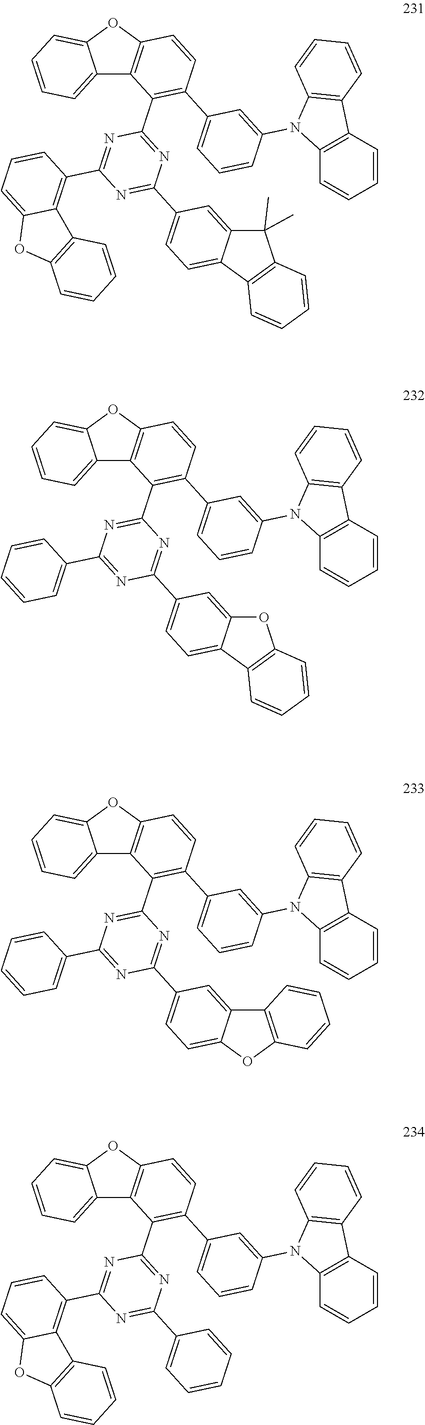 [SMILES: CC1(C)c2ccccc2-c2ccc(-c3nc(-c4cccc5oc6ccccc6c45)nc(-c4c(-c5cccc(-n6c7ccccc7c7ccccc76)c5)ccc5oc6ccccc6c45)n3)cc21 ; c1ccc(-c2nc(-c3ccc4c(c3)oc3ccccc34)nc(-c3c(-c4cccc(-n5c6ccccc6c6ccccc65)c4)ccc4oc5ccccc5c34)n2)cc1 ; c1ccc(-c2nc(-c3ccc4oc5ccccc5c4c3)nc(-c3c(-c4cccc(-n5c6ccccc6c6ccccc65)c4)ccc4oc5ccccc5c34)n2)cc1 ; c1ccc(-c2nc(-c3cccc4oc5ccccc5c34)nc(-c3c(-c4cccc(-n5c6ccccc6c6ccccc65)c4)ccc4oc5ccccc5c34)n2)cc1]

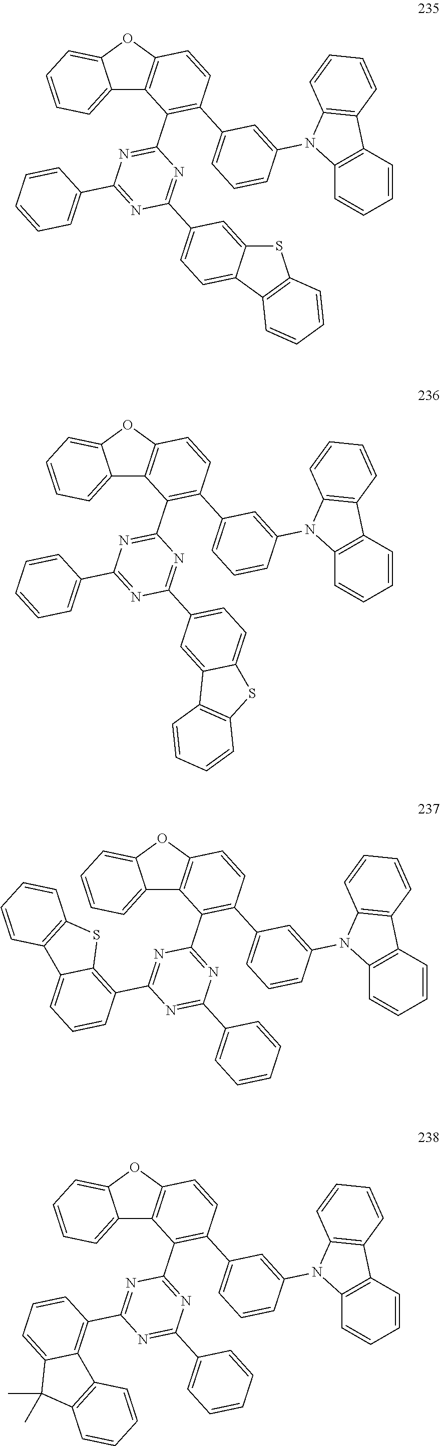 [SMILES: c1ccc(-c2nc(-c3ccc4c(c3)sc3ccccc34)nc(-c3c(-c4cccc(-n5c6ccccc6c6ccccc65)c4)ccc4oc5ccccc5c34)n2)cc1 ; c1ccc(-c2nc(-c3ccc4sc5ccccc5c4c3)nc(-c3c(-c4cccc(-n5c6ccccc6c6ccccc65)c4)ccc4oc5ccccc5c34)n2)cc1 ; c1ccc(-c2nc(-c3cccc4c3sc3ccccc34)nc(-c3c(-c4cccc(-n5c6ccccc6c6ccccc65)c4)ccc4oc5ccccc5c34)n2)cc1 ; CC1(C)c2ccccc2-c2c(-c3nc(-c4ccccc4)nc(-c4c(-c5cccc(-n6c7ccccc7c7ccccc76)c5)ccc5oc6ccccc6c45)n3)cccc21]

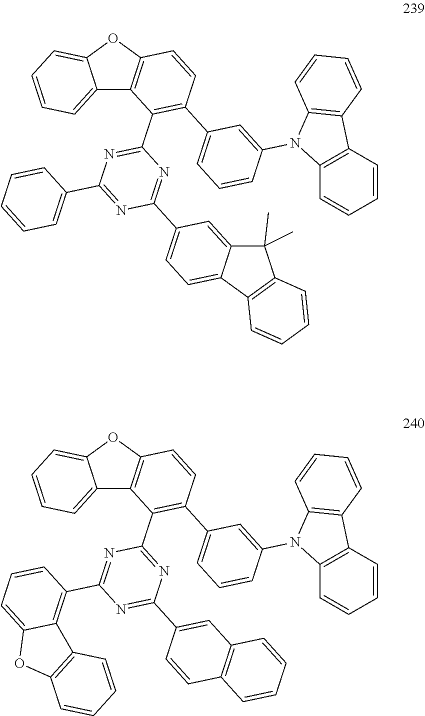 [SMILES: CC1(C)c2ccccc2-c2ccc(-c3nc(-c4ccccc4)nc(-c4c(-c5cccc(-n6c7ccccc7c7ccccc76)c5)ccc5oc6ccccc6c45)n3)cc21 ; c1cc(-c2ccc3oc4ccccc4c3c2-c2nc(-c3ccc4ccccc4c3)nc(-c3cccc4oc5ccccc5c34)n2)cc(-n2c3ccccc3c3ccccc32)c1]

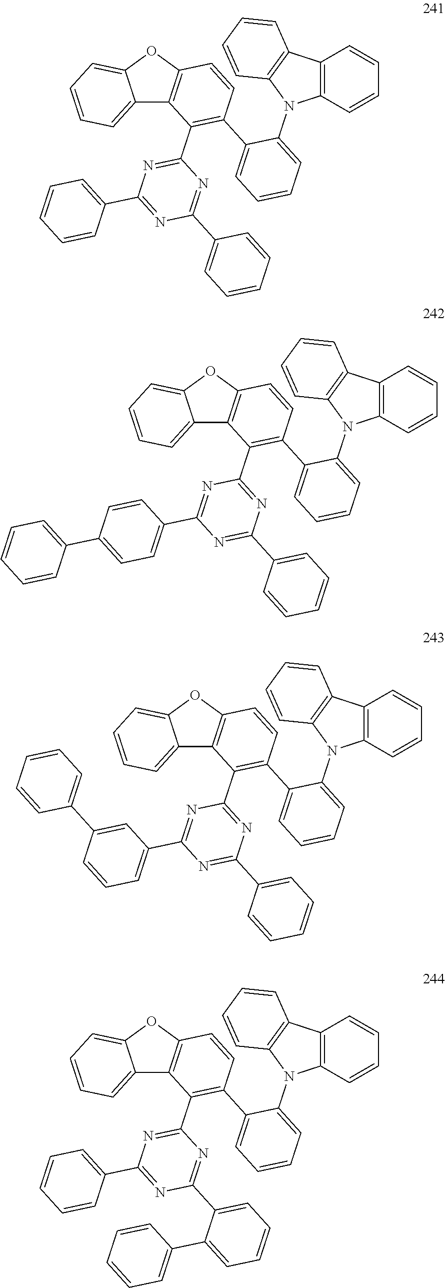

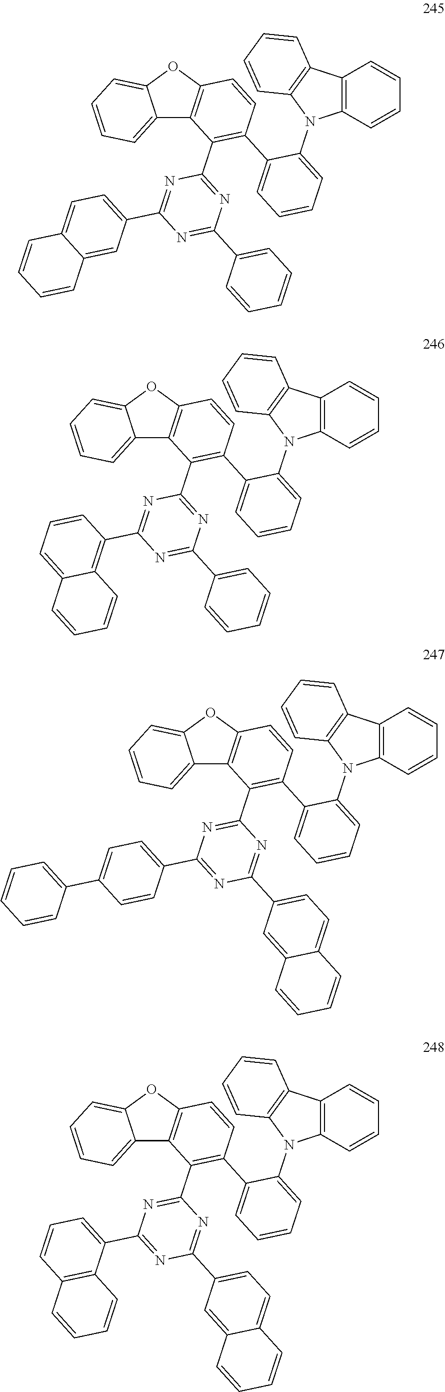

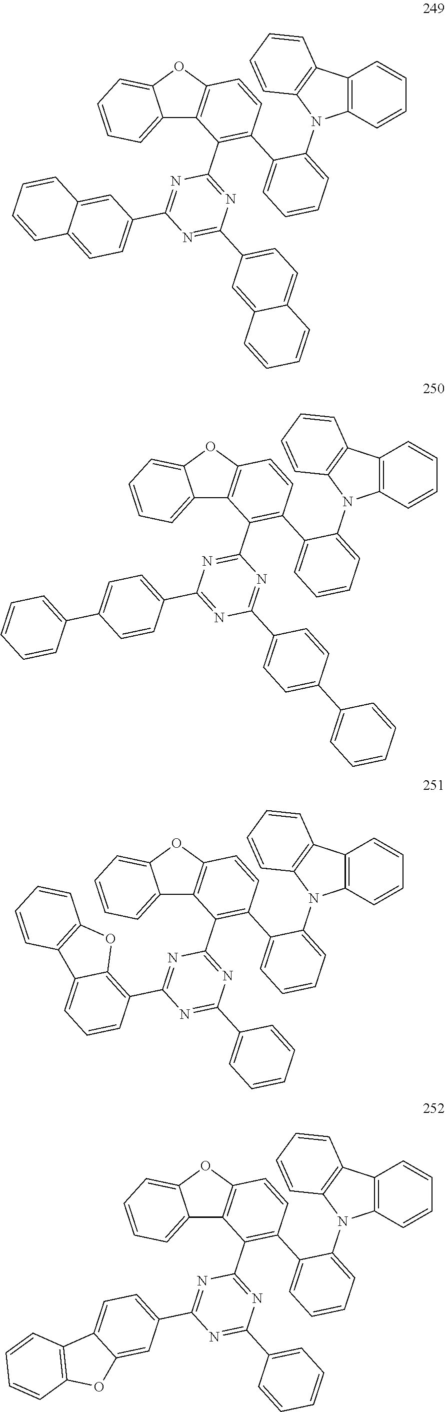 [SMILES: c1ccc(-n2c3ccccc3c3ccccc32)c(-c2ccc3oc4ccccc4c3c2-c2nc(-c3ccc4ccccc4c3)nc(-c3ccc4ccccc4c3)n2)c1 ; c1ccc(-c2ccc(-c3nc(-c4ccc(-c5ccccc5)cc4)nc(-c4c(-c5ccccc5-n5c6ccccc6c6ccccc65)ccc5oc6ccccc6c45)n3)cc2)cc1 ; c1ccc(-c2nc(-c3cccc4c3oc3ccccc34)nc(-c3c(-c4ccccc4-n4c5ccccc5c5ccccc54)ccc4oc5ccccc5c34)n2)cc1 ; c1ccc(-c2nc(-c3ccc4c(c3)oc3ccccc34)nc(-c3c(-c4ccccc4-n4c5ccccc5c5ccccc54)ccc4oc5ccccc5c34)n2)cc1]

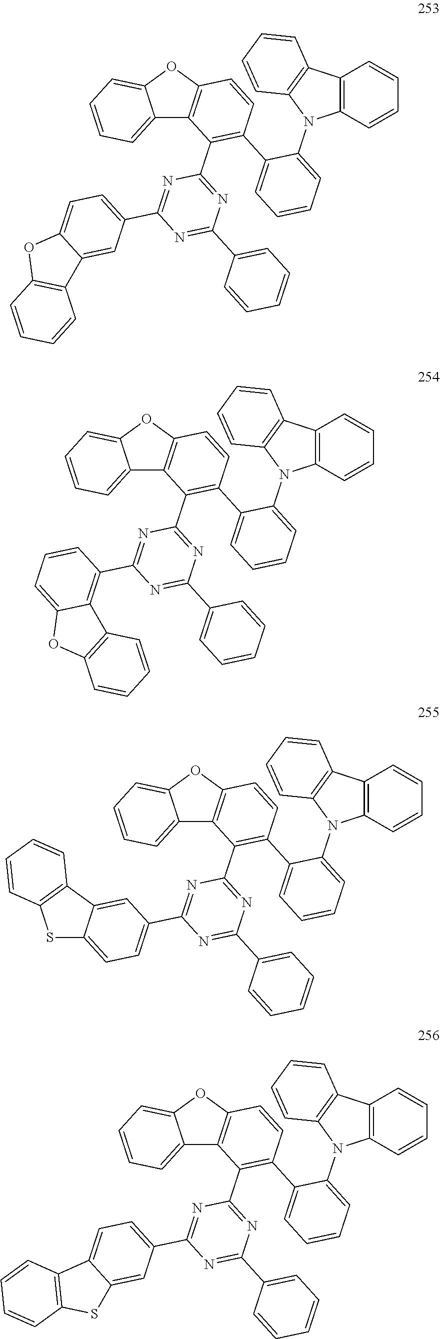 [SMILES: c1ccc(-c2nc(-c3ccc4oc5ccccc5c4c3)nc(-c3c(-c4ccccc4-n4c5ccccc5c5ccccc54)ccc4oc5ccccc5c34)n2)cc1 ; c1ccc(-c2nc(-c3cccc4oc5ccccc5c34)nc(-c3c(-c4ccccc4-n4c5ccccc5c5ccccc54)ccc4oc5ccccc5c34)n2)cc1 ; c1ccc(-c2nc(-c3ccc4sc5ccccc5c4c3)nc(-c3c(-c4ccccc4-n4c5ccccc5c5ccccc54)ccc4oc5ccccc5c34)n2)cc1 ; c1ccc(-c2nc(-c3ccc4c(c3)sc3ccccc34)nc(-c3c(-c4ccccc4-n4c5ccccc5c5ccccc54)ccc4oc5ccccc5c34)n2)cc1]

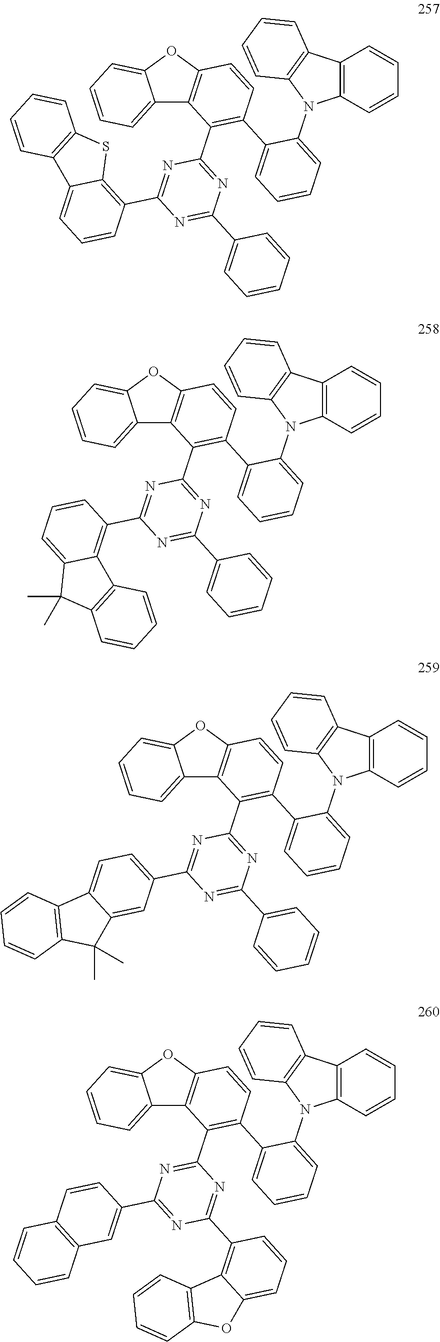

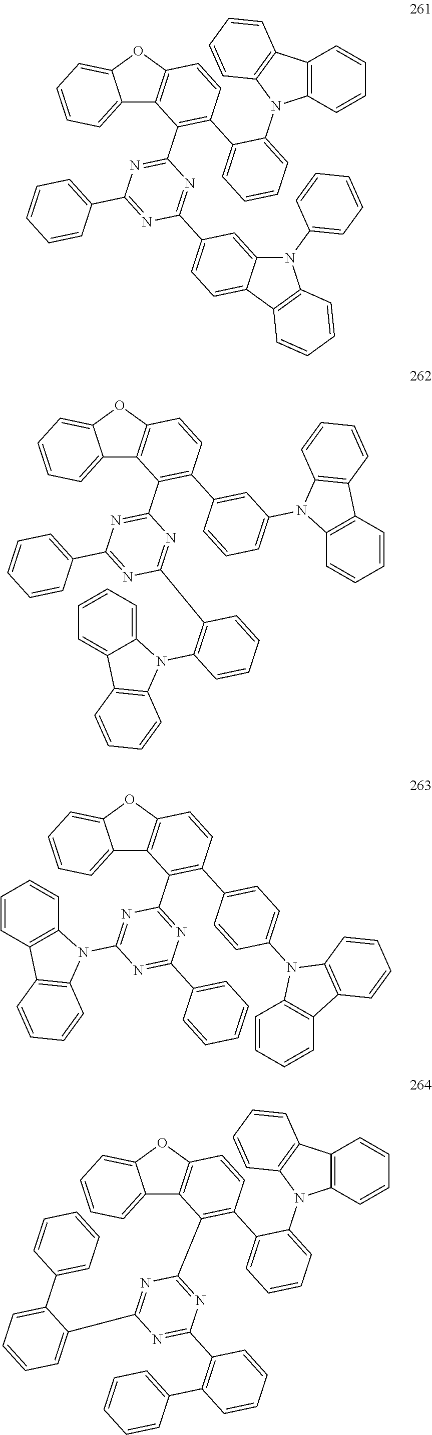 [SMILES: c1ccc(-c2nc(-c3ccc4c5ccccc5n(-c5ccccc5)c4c3)nc(-c3c(-c4ccccc4-n4c5ccccc5c5ccccc54)ccc4oc5ccccc5c34)n2)cc1 ; c1ccc(-c2nc(-c3ccccc3-n3c4ccccc4c4ccccc43)nc(-c3c(-c4cccc(-n5c6ccccc6c6ccccc65)c4)ccc4oc5ccccc5c34)n2)cc1 ; c1ccc(-c2nc(-c3c(-c4ccc(-n5c6ccccc6c6ccccc65)cc4)ccc4oc5ccccc5c34)nc(-n3c4ccccc4c4ccccc43)n2)cc1 ; c1ccc(-c2ccccc2-c2nc(-c3ccccc3-c3ccccc3)nc(-c3c(-c4ccccc4-n4c5ccccc5c5ccccc54)ccc4oc5ccccc5c34)n2)cc1]

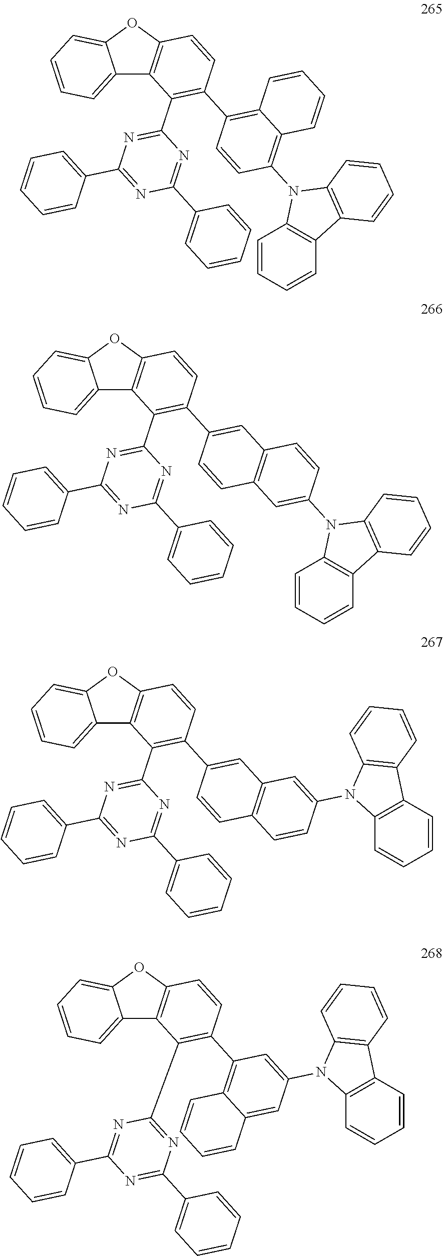 [SMILES: c1ccc(-c2nc(-c3ccccc3)nc(-c3c(-c4ccc(-n5c6ccccc6c6ccccc65)c5ccccc45)ccc4oc5ccccc5c34)n2)cc1 ; c1ccc(-c2nc(-c3ccccc3)nc(-c3c(-c4ccc5cc(-n6c7ccccc7c7ccccc76)ccc5c4)ccc4oc5ccccc5c34)n2)cc1 ; c1ccc(-c2nc(-c3ccccc3)nc(-c3c(-c4ccc5ccc(-n6c7ccccc7c7ccccc76)cc5c4)ccc4oc5ccccc5c34)n2)cc1 ; c1ccc(-c2nc(-c3ccccc3)nc(-c3c(-c4cc(-n5c6ccccc6c6ccccc65)cc5ccccc45)ccc4oc5ccccc5c34)n2)cc1]

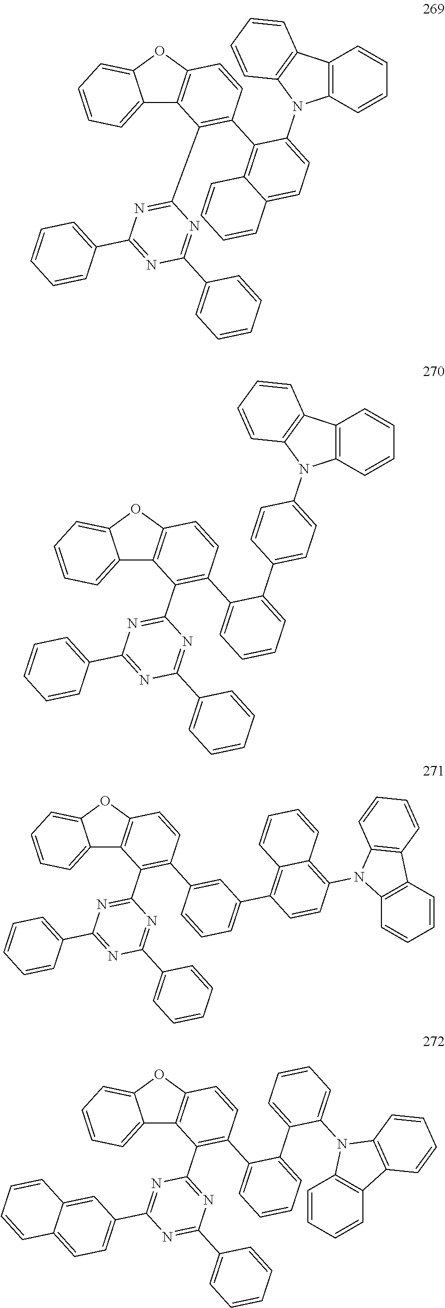 [SMILES: c1ccc(-c2nc(-c3ccccc3)nc(-c3c(-c4c(-n5c6ccccc6c6ccccc65)ccc5ccccc45)ccc4oc5ccccc5c34)n2)cc1 ; c1ccc(-c2nc(-c3ccccc3)nc(-c3c(-c4ccccc4-c4ccc(-n5c6ccccc6c6ccccc65)cc4)ccc4oc5ccccc5c34)n2)cc1 ; c1ccc(-c2nc(-c3ccccc3)nc(-c3c(-c4cccc(-c5ccc(-n6c7ccccc7c7ccccc76)c6ccccc56)c4)ccc4oc5ccccc5c34)n2)cc1 ; c1ccc(-c2nc(-c3ccc4ccccc4c3)nc(-c3c(-c4ccccc4-c4ccccc4-n4c5ccccc5c5ccccc54)ccc4oc5ccccc5c34)n2)cc1]

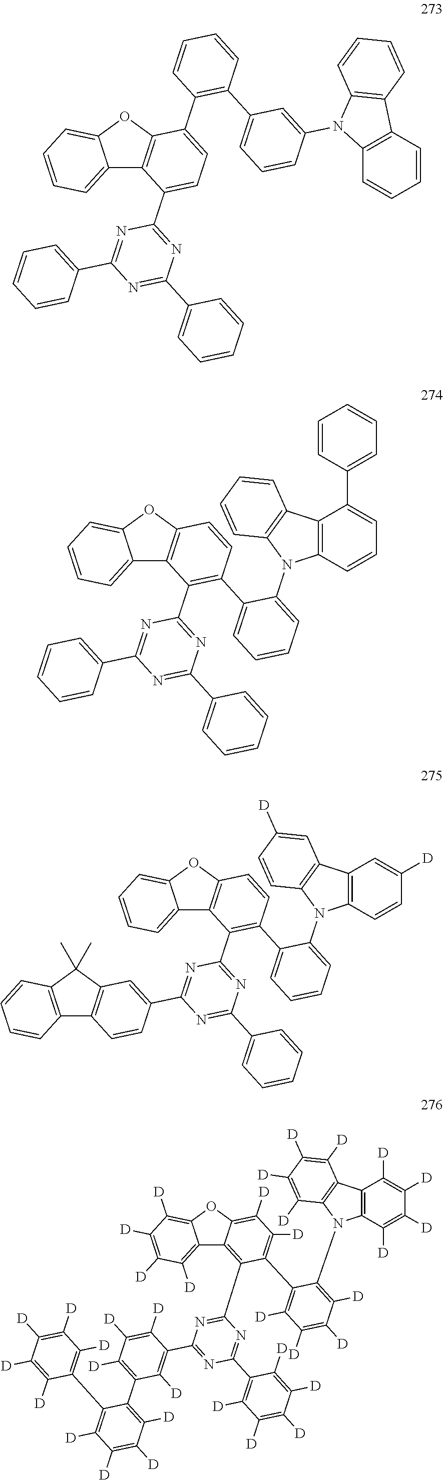

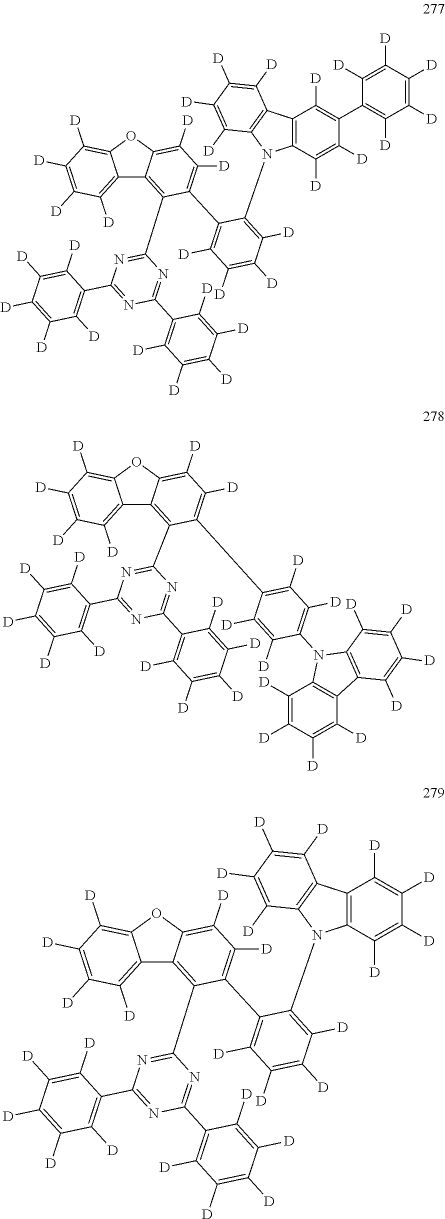

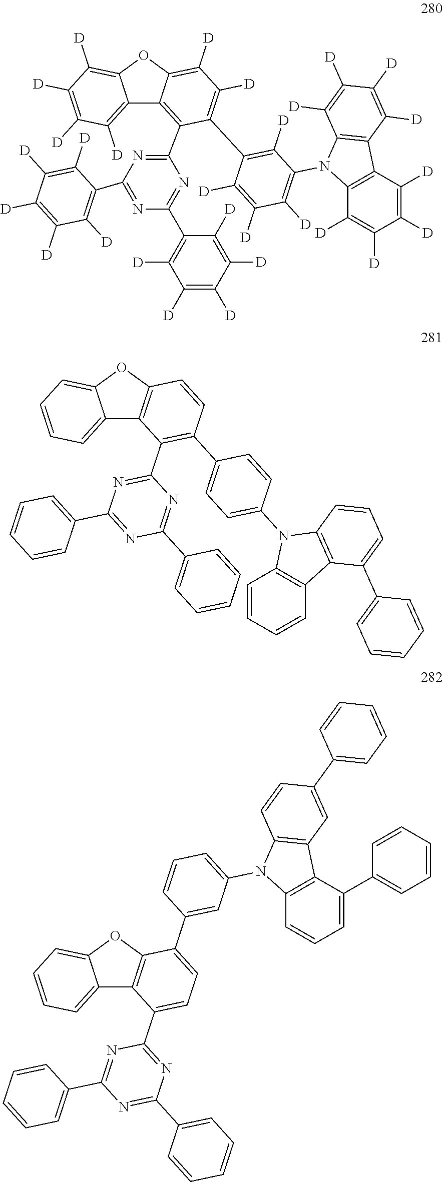 [SMILES: [2H]c1c([2H])c([2H])c(-c2nc(-c3c([2H])c([2H])c([2H])c([2H])c3[2H])nc(-c3c(-c4c([2H])c([2H])c([2H])c(-n5c6c([2H])c([2H])c([2H])c([2H])c6c6c([2H])c([2H])c([2H])c([2H])c65)c4[2H])c([2H])c([2H])c4oc5c([2H])c([2H])c([2H])c([2H])c5c34)n2)c([2H])c1[2H] ; c1ccc(-c2nc(-c3ccccc3)nc(-c3c(-c4ccc(-n5c6ccccc6c6c(-c7ccccc7)cccc65)cc4)ccc4oc5ccccc5c34)n2)cc1 ; c1ccc(-c2ccc3c(c2)c2c(-c4ccccc4)cccc2n3-c2cccc(-c3ccc(-c4nc(-c5ccccc5)nc(-c5ccccc5)n4)c4c3oc3ccccc34)c2)cc1]

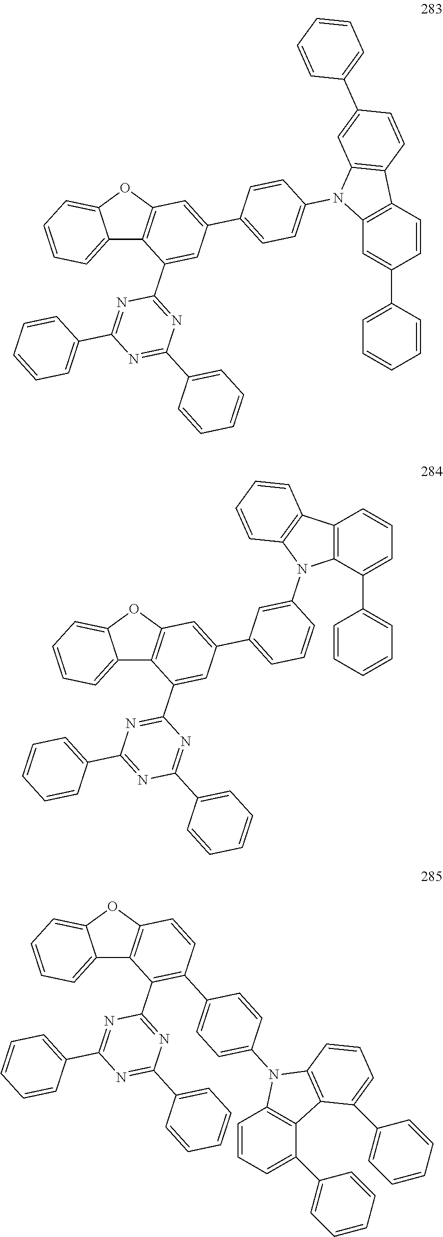 [SMILES: c1ccc(-c2ccc3c4ccc(-c5ccccc5)cc4n(-c4ccc(-c5cc(-c6nc(-c7ccccc7)nc(-c7ccccc7)n6)c6c(c5)oc5ccccc56)cc4)c3c2)cc1 ; c1ccc(-c2nc(-c3ccccc3)nc(-c3cc(-c4cccc(-n5c6ccccc6c6cccc(-c7ccccc7)c65)c4)cc4oc5ccccc5c34)n2)cc1 ; c1ccc(-c2nc(-c3ccccc3)nc(-c3c(-c4ccc(-n5c6cccc(-c7ccccc7)c6c6c(-c7ccccc7)cccc65)cc4)ccc4oc5ccccc5c34)n2)cc1]

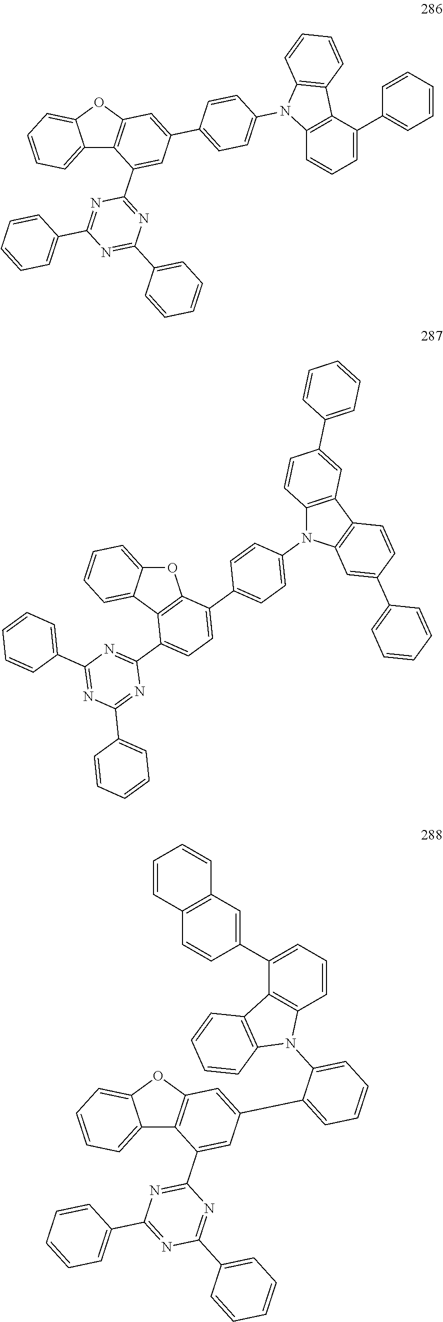 [SMILES: c1ccc(-c2nc(-c3ccccc3)nc(-c3cc(-c4ccc(-n5c6ccccc6c6c(-c7ccccc7)cccc65)cc4)cc4oc5ccccc5c34)n2)cc1 ; c1ccc(-c2ccc3c(c2)c2ccc(-c4ccccc4)cc2n3-c2ccc(-c3ccc(-c4nc(-c5ccccc5)nc(-c5ccccc5)n4)c4c3oc3ccccc34)cc2)cc1 ; c1ccc(-c2nc(-c3ccccc3)nc(-c3cc(-c4ccccc4-n4c5ccccc5c5c(-c6ccc7ccccc7c6)cccc54)cc4oc5ccccc5c34)n2)cc1]

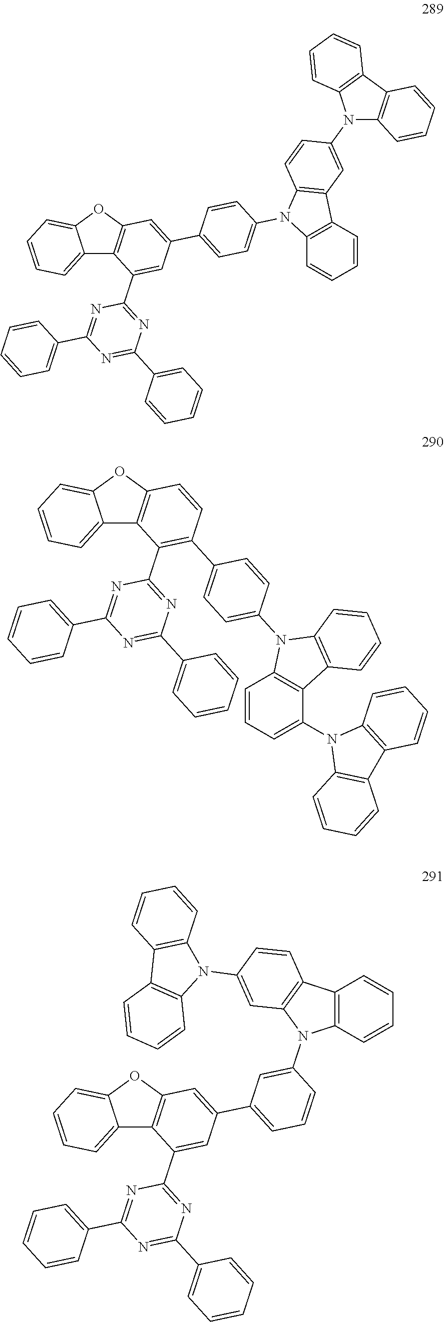 [SMILES: c1ccc(-c2nc(-c3ccccc3)nc(-c3cc(-c4ccc(-n5c6ccccc6c6cc(-n7c8ccccc8c8ccccc87)ccc65)cc4)cc4oc5ccccc5c34)n2)cc1 ; c1ccc(-c2nc(-c3ccccc3)nc(-c3c(-c4ccc(-n5c6ccccc6c6c(-n7c8ccccc8c8ccccc87)cccc65)cc4)ccc4oc5ccccc5c34)n2)cc1 ; c1ccc(-c2nc(-c3ccccc3)nc(-c3cc(-c4cccc(-n5c6ccccc6c6ccc(-n7c8ccccc8c8ccccc87)cc65)c4)cc4oc5ccccc5c34)n2)cc1]

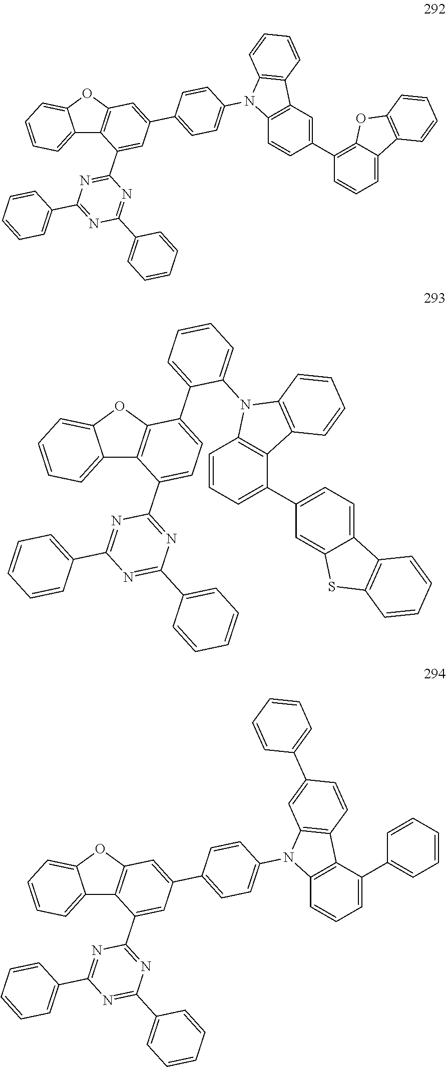 [SMILES: c1ccc(-c2nc(-c3ccccc3)nc(-c3cc(-c4ccc(-n5c6ccccc6c6cc(-c7cccc8c7oc7ccccc78)ccc65)cc4)cc4oc5ccccc5c34)n2)cc1 ; c1ccc(-c2nc(-c3ccccc3)nc(-c3ccc(-c4ccccc4-n4c5ccccc5c5c(-c6ccc7c(c6)sc6ccccc67)cccc54)c4oc5ccccc5c34)n2)cc1 ; c1ccc(-c2ccc3c4c(-c5ccccc5)cccc4n(-c4ccc(-c5cc(-c6nc(-c7ccccc7)nc(-c7ccccc7)n6)c6c(c5)oc5ccccc56)cc4)c3c2)cc1]

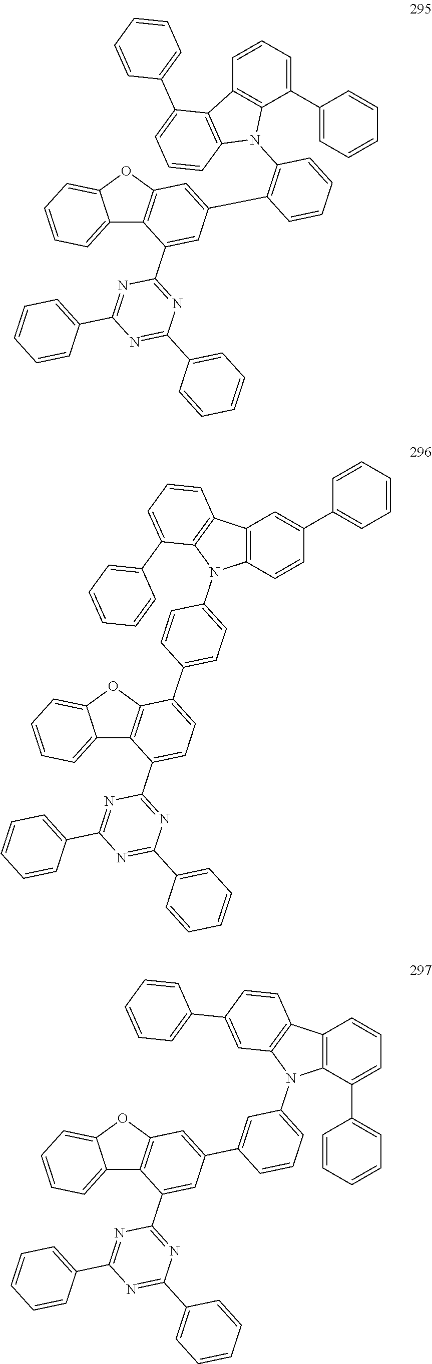 [SMILES: c1ccc(-c2nc(-c3ccccc3)nc(-c3cc(-c4ccccc4-n4c5cccc(-c6ccccc6)c5c5cccc(-c6ccccc6)c54)cc4oc5ccccc5c34)n2)cc1 ; c1ccc(-c2ccc3c(c2)c2cccc(-c4ccccc4)c2n3-c2ccc(-c3ccc(-c4nc(-c5ccccc5)nc(-c5ccccc5)n4)c4c3oc3ccccc34)cc2)cc1 ; c1ccc(-c2ccc3c4cccc(-c5ccccc5)c4n(-c4cccc(-c5cc(-c6nc(-c7ccccc7)nc(-c7ccccc7)n6)c6c(c5)oc5ccccc56)c4)c3c2)cc1]

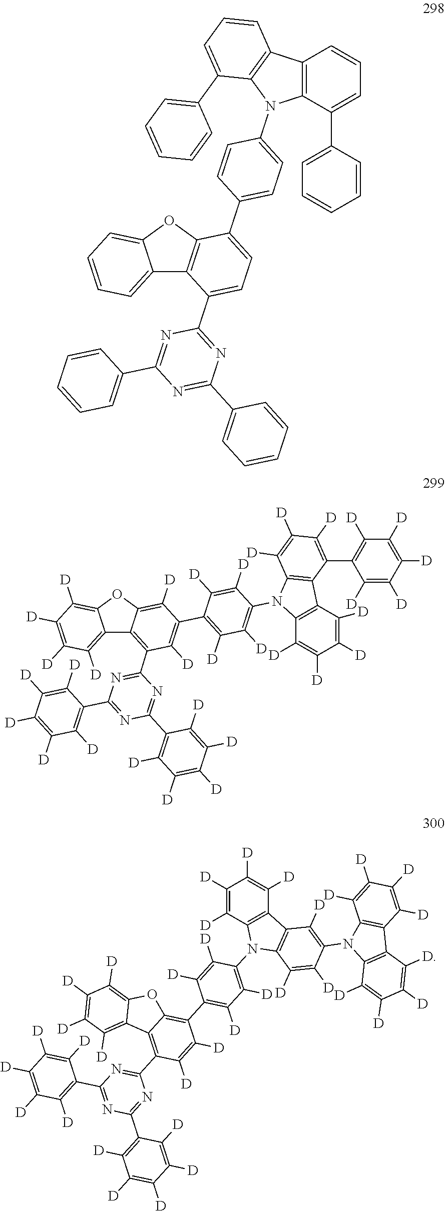

FIGS. 1 to 3 illustrate the stacking order of electrodes and organic layers of a light-emitting device according to an embodiment of the present application. However, the scope of the present application is not intended to be limited to these drawings, and the structure of organic light-emitting devices known in the art may also be applied to the present application.

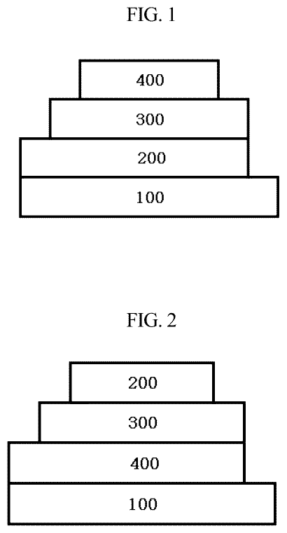

Referring to FIG. 1, a light-emitting device is illustrated, in which a first electrode (anode, 200), an organic layer 300, and a second electrode (cathode, 400) are sequentially stacked on a substrate 100. However, it is not limited to this structure, and as shown in FIG. 2, an organic light-emitting device may be implemented in which the cathode 400, the organic layer 300, and the anode 200 are sequentially stacked on the substrate.

In an exemplary embodiment, the first electrode may be an anode, and the second electrode may be a cathode. Alternatively, the first electrode may be a cathode, and the second electrode may be an anode.

According to exemplary embodiments, materials with a relatively high work function may be used as the anode material, such as transparent conductive oxides, metals, or conductive polymers. Examples of the anode material may include metals such as vanadium, chromium, copper, zinc, and gold, or an alloy thereof, metal oxides such as zinc oxide, indium oxide, indium tin oxide (ITO), and indium zinc oxide (IZO); combinations of metals and oxides such as ZnO:Al or SnO₂:Sb; and conductive polymers such as poly(3-methylthiophene), poly[3,4-(ethylene-1,2-dioxy)thiophene](PEDOT), polypyrrole and polyaniline, but are not limited thereto.

Materials with a relatively low work function may be used as cathode materials, such as metals, metal oxides, or conductive polymers. Examples of cathode materials may include metals such as magnesium, calcium, sodium, potassium, titanium, indium, yttrium, lithium, gadolinium, aluminum, silver, tin, and lead, or their alloys; and multilayer materials such as LiF/Al or LiO₂/Al, but are not limited thereto.

The light-emitting device may include one or more organic layers 300. Each of the plurality of organic layers 300 may independently have a single-layer or multilayer structure.

In an exemplary embodiment, at least one of the organic layers 300 may include a heterocyclic compound represented by Formula 1 above.

In an exemplary embodiment, at least one of the organic layers 300 may include two or more heterocyclic compounds represented by Formula 1 above.

As used herein, the specific details of the heterocyclic compound represented by Formula 1 are the same as those described above.

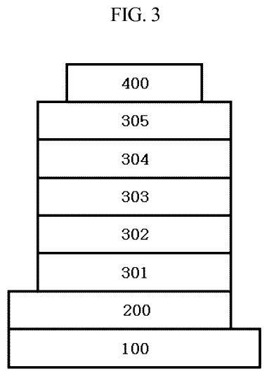

FIG. 3 is a cross-sectional view illustrating an exemplary light-emitting device in which the organic layer has a multilayer structure.

The light-emitting device shown in FIG. 3 may include a hole injection layer 301, a hole transport layer 302, a light-emitting layer 303, an electron transport layer 304, and an electron injection layer 305. However, the scope of the present application is not limited to this stacked structure, and layers other than the light-emitting layer may be omitted, and other functional layers may be added.

The light-emitting layer 303 may include a host material that is excited by holes and electrons, and a dopant material that increases light-emitting efficiency through energy absorption and emission.

In one embodiment, the light-emitting layer 303 may be independently patterned for each red light-emitting device (Pr), green light-emitting device (Pg), and blue light-emitting device (Pb), thereby emitting different colors of light for each element. For example, the light-emitting layer 303 may be patterned into a red light-emitting layer, a green light-emitting layer, and a blue light-emitting layer for each element.

In one embodiment, the light-emitting layer 303 may not be patterned for each light-emitting device, but may be provided in common to a plurality of light-emitting devices. For example, the light-emitting layer 303 may emit white light, and the color of each element may be implemented through a color filter.

The host material may include a phosphorescent host, a fluorescent host, or a combination thereof. For example, the host material may include ADN (9,10-di(2-naphthyl) anthracene), MADN (2-methyl-9,10-bis(naphthalen-2-yl) anthracene), TBADN (9,10-di-(2-naphthyl)-2-t-butyl-anthracene), CBP (4,4′-bis(N-carbazolyl)-1,1′-biphenyl), mCP(1,3-di-9-carbazolylbenzene), TCP(1,3,5-tri(carbazol-9-yl)benzene), DPEPO(bis[2-(diphenylphosphino)phenyl]ether oxide), PPF (2,8-bis(diphenylphosphoryl)dibenzo[b,d]furan), TCTA (4,4′,4″-tris(carbazol-9-yl)-triphenylamine), CP1 (hexaphenylcyclotriphosphazene), UGH2 (1,4-bis(triphenylsilyl)benzene), DPSiO₃ (hexaphenylcyclotrisiloxane), DPSiO₄ (octaphenylcyclotetrasiloxane), or a combination thereof.

The dopant material may include a phosphorescent dopant, a fluorescent dopant, or a combination thereof. For example, the dopant material may include a metal complex containing iridium (Ir), platinum (Pt), osmium (Os), gold (Au), titanium (Ti), zirconium (Zr), hafnium (Hf), europium (Eu), terbium (Tb), or thulium (Tm), or BCzVB (1,4-bis[2-(3-N-ethylcarbazolyl)vinyl]benzene), DPAVB (4-(di-p-tolylamino)-4′-[(di-p-tolylamino) styryl]stilbene), N-BDAVBi (N-(4-((E)-2-(6-((E)-4-(diphenylamino) styryl) naphthalen-2-yl)vinyl)phenyl)-N-phenylbenzenamine), DPAVBi (4,4′-bis[2-(4-(N,N-diphenylamino)phenyl)vinyl]biphenyl), TBP(2,5,8,11-tetra-t-butylperylene), or a combination thereof.

In one embodiment, the host material may include a heterocyclic compound represented by Formula 1 above. For example, the heterocyclic compound may serve as a red phosphorescent dopant.

By including the heterocyclic compound as the host material, the charge transport properties within the light-emitting device and the stability of the light-emitting layer 303 may be further improved, thereby increasing the light-emitting efficiency and lifetime of the light-emitting device. Accordingly, the light-emitting properties may be improved without increasing the operating voltage.

In some embodiments, the content of the dopant material in the light-emitting layer 303 may be about 0.01 parts by weight or more, about 1 part by weight or more, or about 2 parts by weight or more, and may be about 15 parts by weight or less, about 10 parts by weight or less, or about 8 parts by weight or less, based on 100 parts by weight of the host material. Within this range, the formation and emission energy of excitons may be increased, and the light-emitting efficiency and stability may be further improved.

In some embodiments, the organic layer 300 may further include a compound represented by Formula 4 below or a compound represented by Formula 5 below.

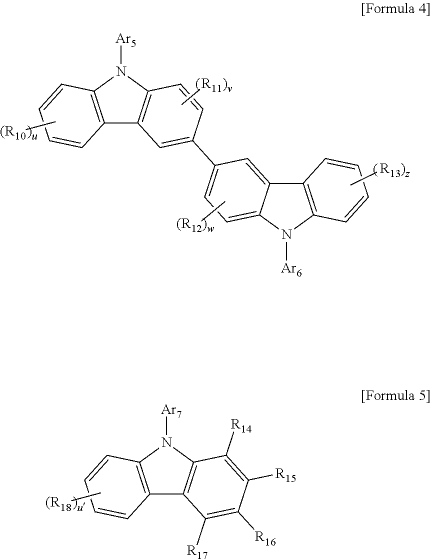

In Formulae 4 and 5, two adjacent groups of R₁₄ to R₁₇ may form a ring represented by

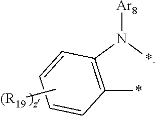

The remaining groups among R₁₀ to R₁₃, R₁₄ to R₁₇, and R₁₈ to R₁₉ that do not form a ring may each independently be selected from the group consisting of hydrogen; deuterium; a halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, wherein adjacent groups may be linked to form a substituted or unsubstituted aryl group having 6 to 60 carbon atoms; a substituted or unsubstituted heterocycloalkyl group having 2 to 60 carbon atoms; or a substituted or unsubstituted heteroaryl group having 2 to 60 carbon atoms.

R, R′R″ may each independently be hydrogen; deuterium; a cyano group (—CN); a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms.

According to exemplary embodiments, the remaining groups among R₁₀ to R₁₃, R₁₄ to R₁₇, and R₁₈ to R₁₉ that do not form a ring may each independently be hydrogen or deuterium. According to some embodiments, the remaining groups among R₁₀ to R₁₃, R₁₄ to R₁₇, and R₁₈ to R₁₉ that do not form a ring may be hydrogen. According to some embodiments, the remaining groups among R₁₀ to R₁₃, R₁₄ to R₁₇, and R₁₈ to R₁₉ that do not form a ring may be deuterium.

Ar₅ to Ar₈ may each independently be a substituted or unsubstituted aryl group having 6 to 60 carbon atoms, or a substituted or unsubstituted heteroaryl group having 2 to 60 carbon atoms.

According to exemplary embodiments, Ar₅ to Ar₈ may each independently be a substituted or unsubstituted aryl group having 6 to 40 carbon atoms, or a substituted or unsubstituted heteroaryl group having 2 to 40 carbon atoms.

According to some embodiments, Ar₅ to Ar₈ may each independently be a substituted or unsubstituted aryl group having 6 to 20 carbon atoms, or a substituted or unsubstituted heteroaryl group having 2 to 20 carbon atoms.

According to exemplary embodiments, Ar₅ to Ar₈ may each independently be an aryl group having 6 to 60 carbon atoms that is deuterated or non-deuterated, or a heteroaryl group having 2 to 60 carbon atoms that is deuterated or non-deuterated.

According to exemplary embodiments, Ar₅ to Ar₈ may each independently be an aryl group having 6 to 40 carbon atoms that is deuterated or non-deuterated, or a heteroaryl group having 2 to 40 carbon atoms that is deuterated or non-deuterated.

According to some embodiments, Ar₅ to Ar₈ may each independently be an aryl group having 6 to 20 carbon atoms that is deuterated or non-deuterated, or a heteroaryl group having 2 to 20 carbon atoms that is deuterated or non-deuterated.

For example, Ar₅ and Ar₆ may each independently be a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylenyl group, a substituted or unsubstituted anthryl group, a substituted or unsubstituted dimethylfluorenyl group, a substituted or unsubstituted diethylfluorenyl group, a substituted or unsubstituted spirobifluorenyl group, a substituted or unsubstituted furan group, a substituted or unsubstituted thiophene group, a substituted or unsubstituted benzofuran group, a substituted or unsubstituted benzothiophene group, a substituted or unsubstituted dibenzofuran group, a substituted or unsubstituted dibenzothiophene group, a substituted or unsubstituted N-carbazolyl group, a substituted or unsubstituted C-carbazolyl group, or a combination thereof.

For example, Ar₅ and Ar₆ may each independently be a deuterated or non-deuterated phenyl group, a deuterated or non-deuterated biphenyl group, a deuterated or non-deuterated terphenyl group, a deuterated or non-deuterated naphthyl group, a deuterated or non-deuterated phenanthrenyl group, a deuterated or non-deuterated triphenylenyl group, a deuterated or non-deuterated anthryl group, a deuterated or non-deuterated dimethylfluorenyl group, a deuterated or non-deuterated diethylfluorenyl group, a deuterated or non-deuterated spirobifluorenyl group, a deuterated or non-deuterated furan group, a deuterated or non-deuterated thiophene group, a deuterated or non-deuterated benzofuran group, a deuterated or non-deuterated benzothiophene group, a deuterated or non-deuterated dibenzofuran group, a deuterated or non-deuterated dibenzothiophene group, a deuterated or non-deuterated N-carbazolyl group, a deuterated or non-deuterated C-carbazolyl group, or a combination thereof.

u, u′, z, and z′ may each independently be 0 or an integer of 1 to 4. In exemplary embodiments, u,
u′, z, and z′ may each independently be 0, 1, or 2.

v and w may each independently be 0 or an integer of 1 to 3. In exemplary embodiments, v and w may each independently be 0, 1, or 2.

* may represent a bonding site.

In exemplary embodiments, the compound represented by Formula 5 may be represented by any one of Formulae 5-1 to 5-5 below.

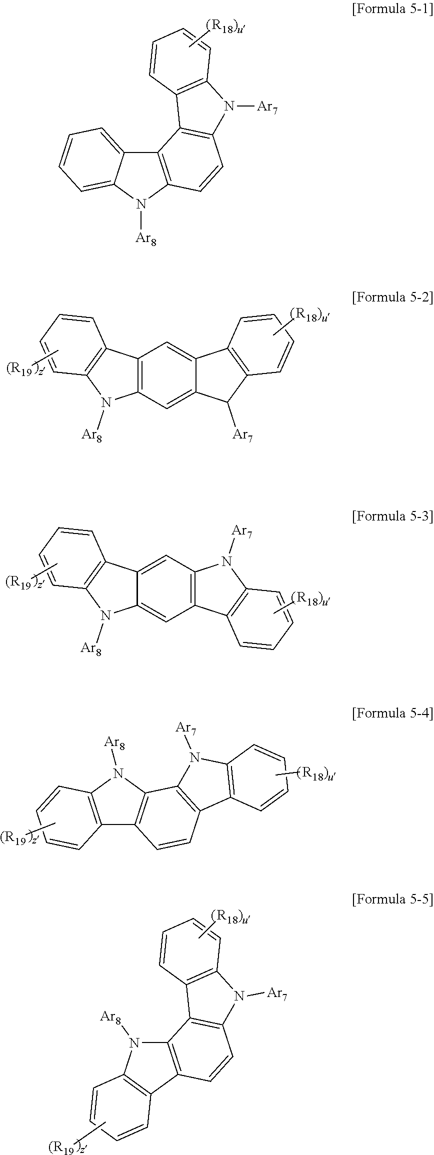

In Formulae 5-1 to 5-5, R₁₈ and R₁₉ may each independently be selected from the group consisting of hydrogen; deuterium; a halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, wherein adjacent groups may be linked to form a substituted or unsubstituted aryl group having 6 to 60 carbon atoms; a substituted or unsubstituted heterocycloalkyl group having 2 to 60 carbon atoms; or a substituted or unsubstituted heteroaryl group having 2 to 60 carbon atoms.

R, R′, R″ may each independently be hydrogen; deuterium; a cyano group (—CN); a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms.

According to exemplary embodiments, R₁₈ and R₁₉ may each independently be hydrogen or deuterium. According to some embodiments, R₁₈ and R₁₉ may be hydrogen. According to some embodiments, R₁₈ and R 19 may be deuterium.

Ar₇ and Ar₅ may each independently be a substituted or unsubstituted aryl group having 6 to 60 carbon atoms, or a substituted or unsubstituted heteroaryl group having 2 to 60 carbon atoms.

According to exemplary embodiments, Ar₇ and Ar₈ may each independently be a substituted or unsubstituted aryl group having 6 to 40 carbon atoms, or a substituted or unsubstituted heteroaryl group having 2 to 40 carbon atoms.

According to some embodiments, Ar₇ and Ar₈ may each independently be a substituted or unsubstituted aryl group having 6 to 20 carbon atoms, or a substituted or unsubstituted heteroaryl group having 2 to 20 carbon atoms.

According to exemplary embodiments, Ar₇ and Ar₈ may each independently be an aryl group having 6 to 60 carbon atoms that is deuterated or non-deuterated, or a heteroaryl group having 2 to 60 carbon atoms that is deuterated or non-deuterated.

According to exemplary embodiments, Ar₇ and Ar₈ may each independently be an aryl group having 6 to 40 carbon atoms that is deuterated or non-deuterated, or a heteroaryl group having 2 to 40 carbon atoms that is deuterated or non-deuterated.

According to some embodiments, Ar₇ and Ar₈ may each independently be an aryl group having 6 to 20 carbon atoms that is deuterated or non-deuterated, or a heteroaryl group having 2 to 20 carbon atoms that is deuterated or non-deuterated.

For example, Ar₇ and Ar₈ may each independently be a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylenyl group, a substituted or unsubstituted anthryl group, a substituted or unsubstituted dimethylfluorenyl group, a substituted or unsubstituted diethylfluorenyl group, a substituted or unsubstituted spirobifluorenyl group, a substituted or unsubstituted furan group, a substituted or unsubstituted thiophene group, a substituted or unsubstituted benzofuran group, a substituted or unsubstituted benzothiophene group, a substituted or unsubstituted dibenzofuran group, a substituted or unsubstituted dibenzothiophene group, a substituted or unsubstituted N-carbazolyl group, a substituted or unsubstituted C-carbazolyl group, or a combination thereof.

For example, Ar₇ and Ar₈ may each independently be a deuterated or non-deuterated phenyl group, a deuterated or non-deuterated biphenyl group, a deuterated or non-deuterated terphenyl group, a deuterated or non-deuterated naphthyl group, a deuterated or non-deuterated phenanthrenyl group, a deuterated or non-deuterated triphenylenyl group, a deuterated or non-deuterated anthryl group, a deuterated or non-deuterated dimethylfluorenyl group, a deuterated or non-deuterated diethylfluorenyl group, a deuterated or non-deuterated spirobifluorenyl group, a deuterated or non-deuterated furan group, a deuterated or non-deuterated thiophene group, a deuterated or non-deuterated benzofuran group, a deuterated or non-deuterated benzothiophene group, a deuterated or non-deuterated dibenzofuran group, a deuterated or non-deuterated dibenzothiophene group, a deuterated or non-deuterated N-carbazolyl group, a deuterated or non-deuterated C-carbazolyl group, or a combination thereof.

u′ and z′ may each independently be 0 or an integer of 1 to 4. According to exemplary embodiments, u′ and z′ may each independently be 0, 1, or 2.

When u′ and z′ are each independently 2 or more, a plurality of R₁₈ groups and R₁₉ groups may each independently be the same or different. For example, when u′ is 2 or more, a plurality of R₁₈ groups may be the same or different. For example, when z′ is 2 or more, a plurality of R₁₉ groups may be the same or different.

According to exemplary embodiments, the deuterium content of the compound of Formula 4 may be 0%, or greater than 0 and not more than 100%.

According to exemplary embodiments, the deuterium content of the compound of Formula 4 may be 0%, or 5% to 100%.

According to exemplary embodiments, the deuterium content of the compound of Formula 4 may be 0%, or 10% to 100%.

According to exemplary embodiments, the deuterium content of the compound of Formula 4 may be 0%, or 15% to 100%.

According to exemplary embodiments, the deuterium content of the compound of Formula 4 may be 0%, or 20% to 100%.

According to exemplary embodiments, the deuterium content of the compound of Formula 4 may be 0%, or 25% to 100%.

According to exemplary embodiments, the deuterium content of the compound of Formula 4 may be 0%, or 30% to 100%.

According to exemplary embodiments, the deuterium content of the compound of Formula 4 may be 0%, or 50% to 100%.

According to exemplary embodiments, the deuterium content of the compound of Formula 4 may be 0%, or 70% to 100%.

According to exemplary embodiments, the deuterium content of the compound of Formula 4 may be 0%, or 90% to 100%.

According to exemplary embodiments, the deuterium content of the compound of Formula 4 may be 0%.

According to exemplary embodiments, the deuterium content of the compound of Formula 4 may be 30% to 100%.

According to exemplary embodiments, the deuterium content of the compound of Formula 4 may be 50% to 100%.

According to exemplary embodiments, the deuterium content of the compound of Formula 4 may be 70% to 100%.

According to exemplary embodiments, the deuterium content of the compound of Formula 4 may be 90% to 100%.

According to exemplary embodiments, the deuterium content of the compound of Formula 5 may be 0%, or greater than 0 and not more than 100%.

According to exemplary embodiments, the deuterium content of the compound of Formula 5 may be 0%, or 5% to 100%.

According to exemplary embodiments, the deuterium content of the compound of Formula 5 may be 0%, or 10% to 100%.

According to exemplary embodiments, the deuterium content of the compound of Formula 5 may be 0%, or 15% to 100%.

According to exemplary embodiments, the deuterium content of the compound of Formula 5 may be 0%, or 20% to 100%.

In exemplary embodiments, the deuterium content of the compound of Formula 5 may be 0%, or 25% to 100%.

In exemplary embodiments, the deuterium content of the compound of Formula 5 may be 0%, or 30% to 100%.

In exemplary embodiments, the deuterium content of the compound of Formula 5 may be 0%, or 50% to 100%.

In exemplary embodiments, the deuterium content of the compound of Formula 5 may be 0%, or 70% to 100%.

In exemplary embodiments, the deuterium content of the compound of Formula 5 may be 0%, or 90% to 100%.

In exemplary embodiments, the deuterium content of the compound of Formula 5 may be 0%.

According to exemplary embodiments, the deuterium content of the compound of Formula 5 may be 30% to 100%.

According to exemplary embodiments, the deuterium content of the compound of Formula 5 may be 50% to 100%.

According to exemplary embodiments, the deuterium content of the compound of Formula 5 may be 70% to 100%.

According to exemplary embodiments, the deuterium content of the compound of Formula 5 may be 90% to 100%.

The compound of Formula 4 may be represented by any one of formulae below:

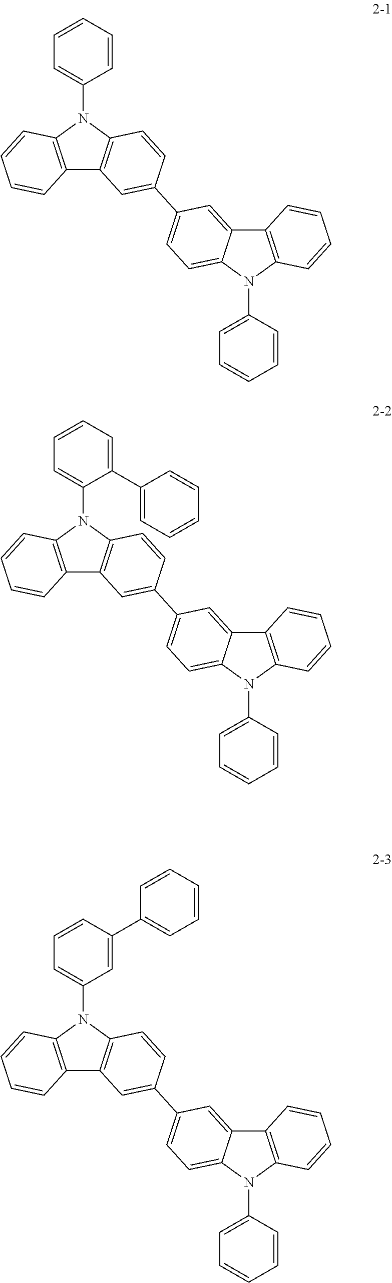 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1]

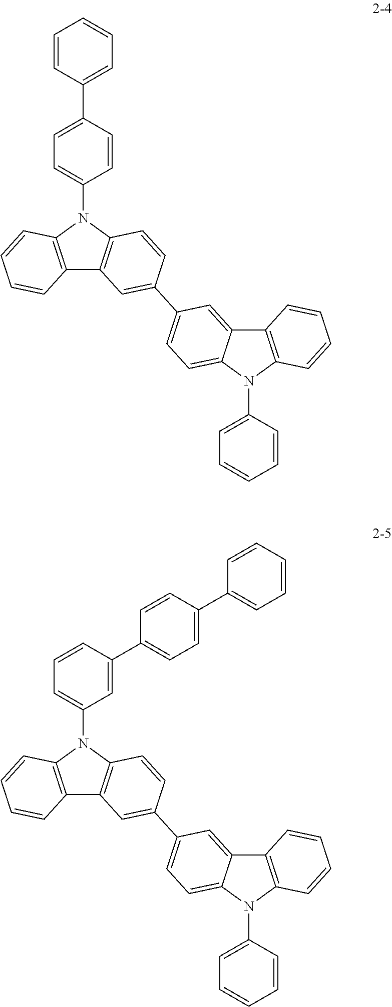 [SMILES: c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1]

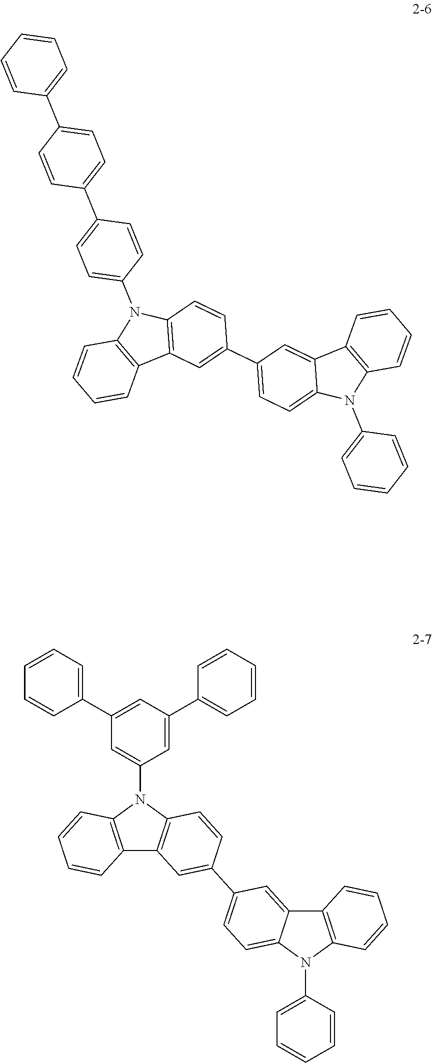 [SMILES: c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

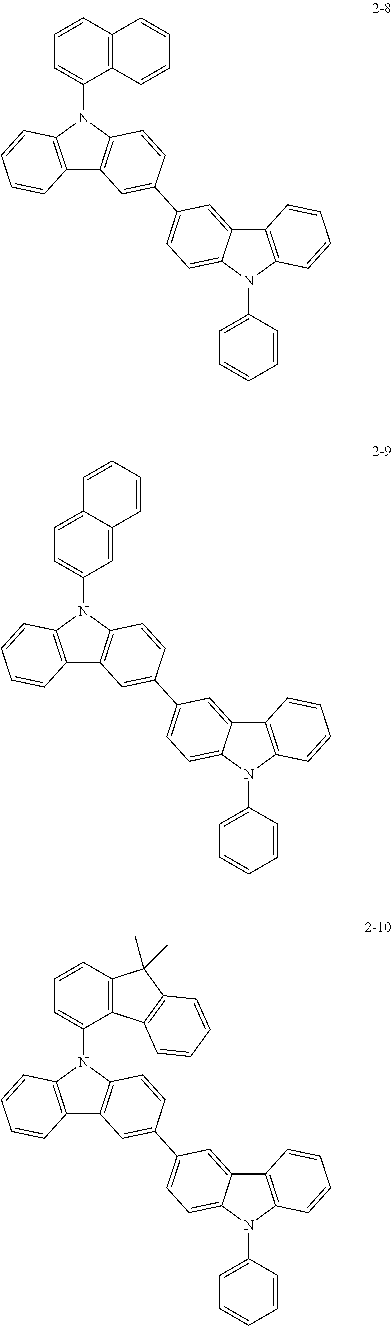 [SMILES: c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21]

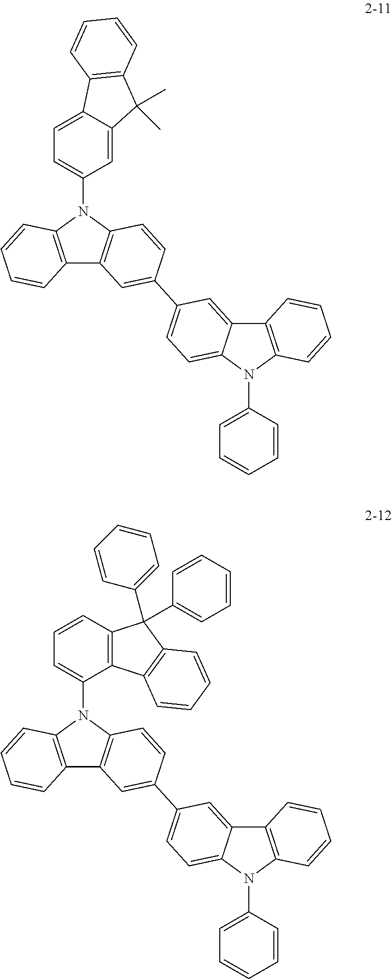 [SMILES: CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

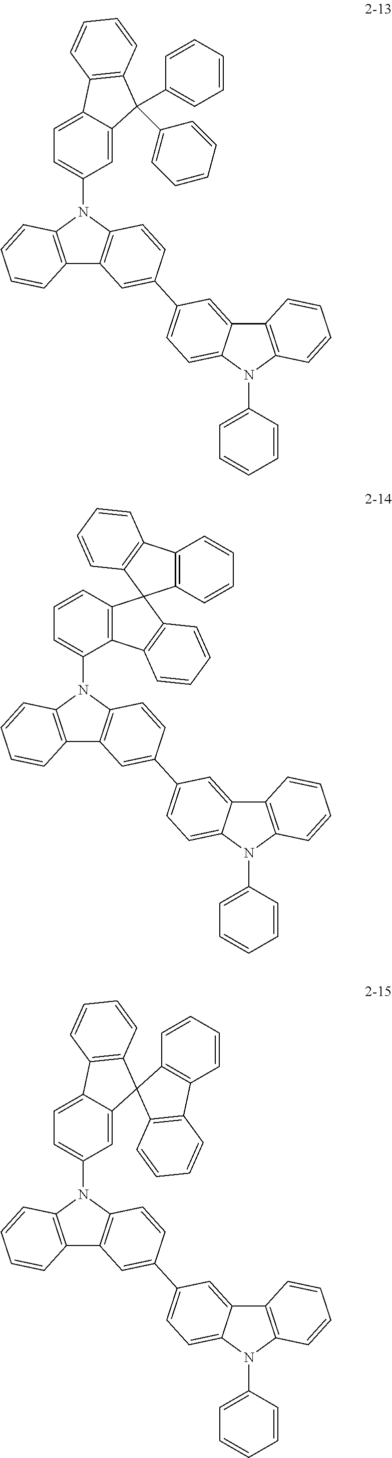 [SMILES: c1ccc(-n2c3ccccc3c3cc(-c4ccc5c(c4)c4ccccc4n5-c4ccc5c(c4)C(c4ccccc4)(c4ccccc4)c4ccccc4-5)ccc32)cc1 ; c1ccc(-n2c3ccccc3c3cc(-c4ccc5c(c4)c4ccccc4n5-c4cccc5c4-c4ccccc4C54c5ccccc5-c5ccccc54)ccc32)cc1 ; c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1]

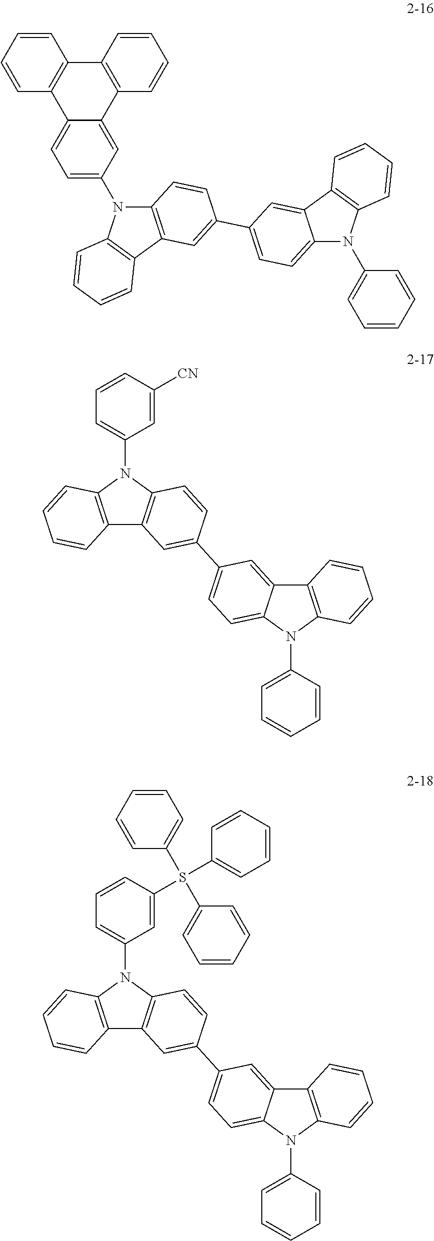 [SMILES: c1ccc(-n2c3ccccc3c3cc(-c4ccc5c(c4)c4ccccc4n5-c4ccc5c6ccccc6c6ccccc6c5c4)ccc32)cc1 ; N#Cc1cccc(-n2c3ccccc3c3cc(-c4ccc5c(c4)c4ccccc4n5-c4ccccc4)ccc32)c1 ; c1ccc(-n2c3ccccc3c3cc(-c4ccc5c(c4)c4ccccc4n5-c4cccc(S(c5ccccc5)(c5ccccc5)c5ccccc5)c4)ccc32)cc1]

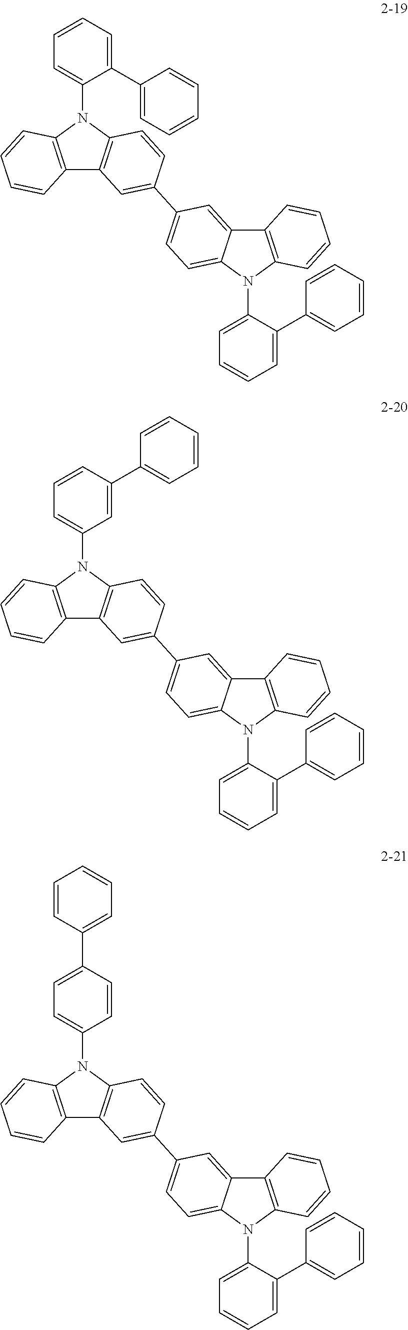 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5ccccc5-c5ccccc5)ccc43)cc2)cc1]

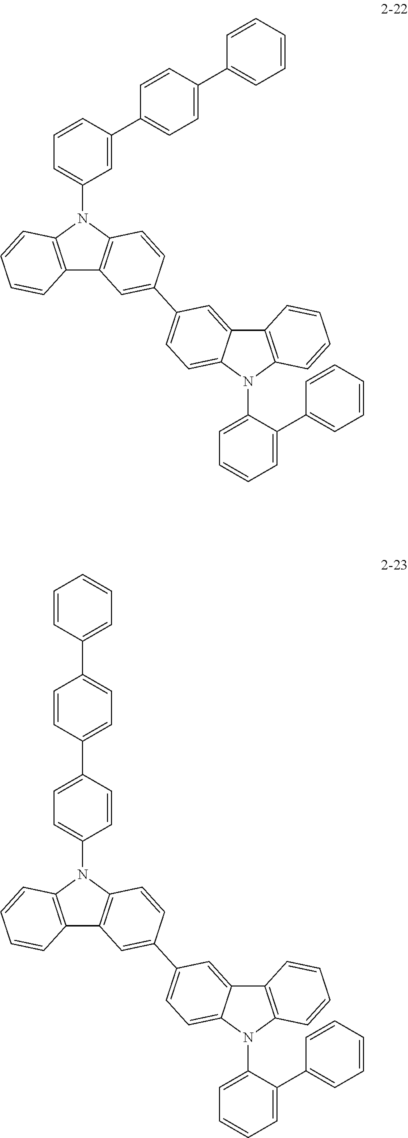 [SMILES: c1ccc(-c2ccc(-c3cccc(-n4c5ccccc5c5cc(-c6ccc7c(c6)c6ccccc6n7-c6ccccc6-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6-c6ccccc6)ccc54)cc3)cc2)cc1]

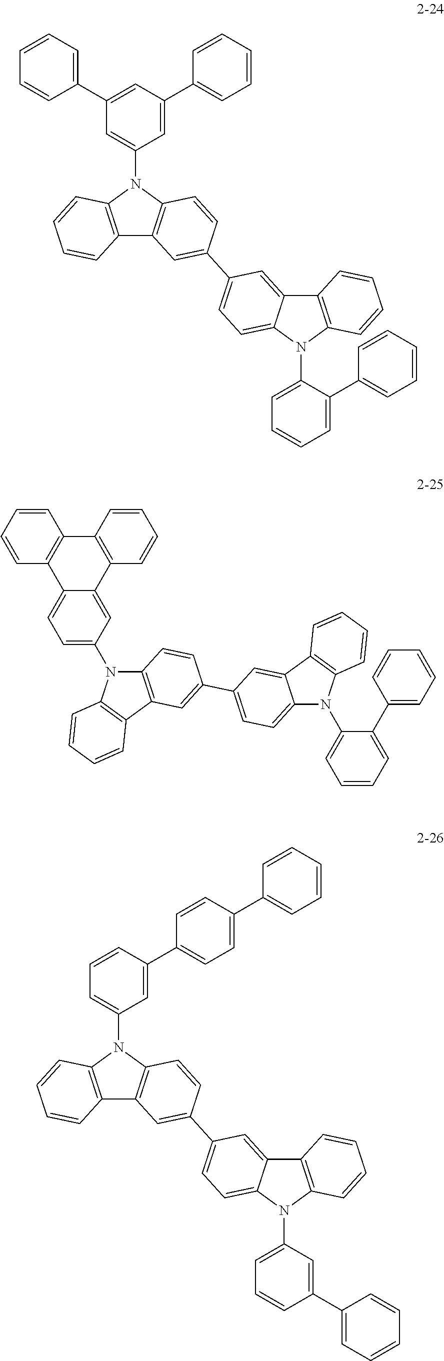 [SMILES: c1ccc(-c2cc(-c3ccccc3)cc(-n3c4ccccc4c4cc(-c5ccc6c(c5)c5ccccc5n6-c5ccccc5-c5ccccc5)ccc43)c2)cc1 ; c1ccc(-c2ccccc2-n2c3ccccc3c3cc(-c4ccc5c(c4)c4ccccc4n5-c4ccc5c6ccccc6c6ccccc6c5c4)ccc32)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1]

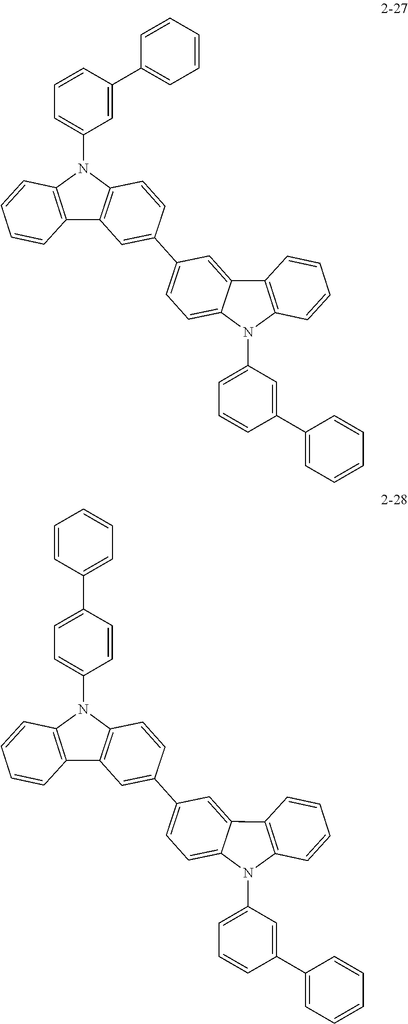 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

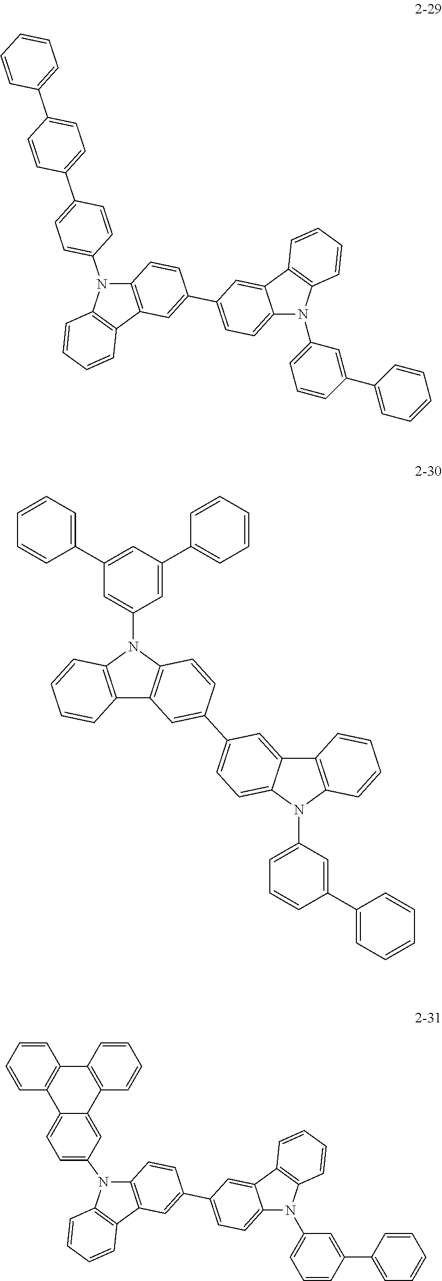 [SMILES: c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccccc4c4cc(-c5ccc6c(c5)c5ccccc5n6-c5ccc6c7ccccc7c7ccccc7c6c5)ccc43)c2)cc1]

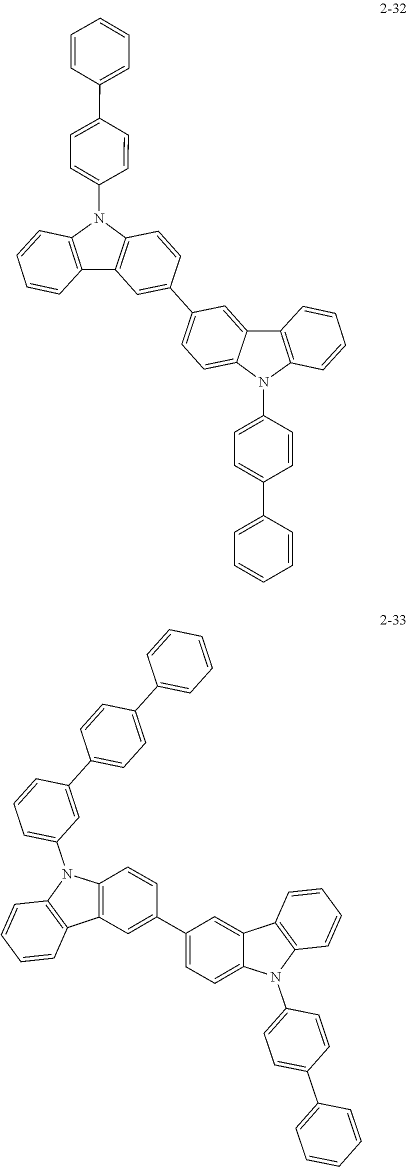 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1]

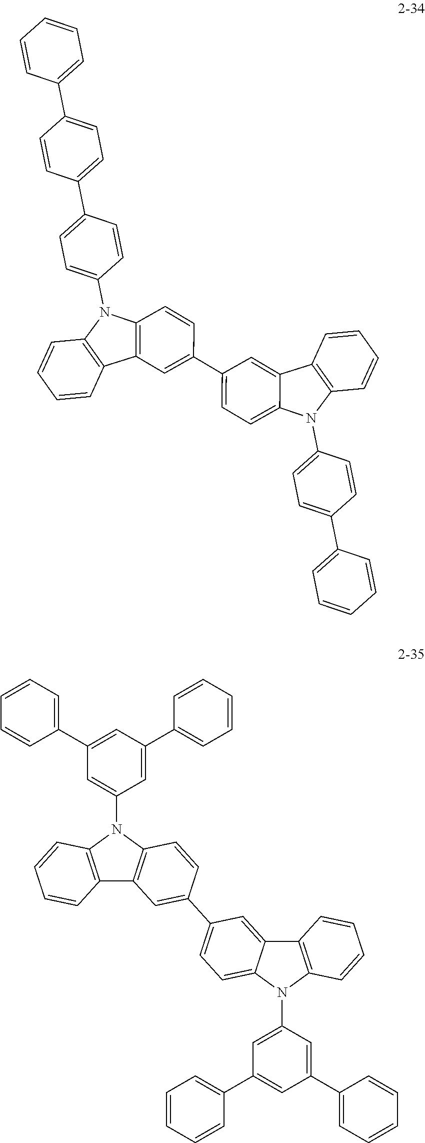 [SMILES: c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1]

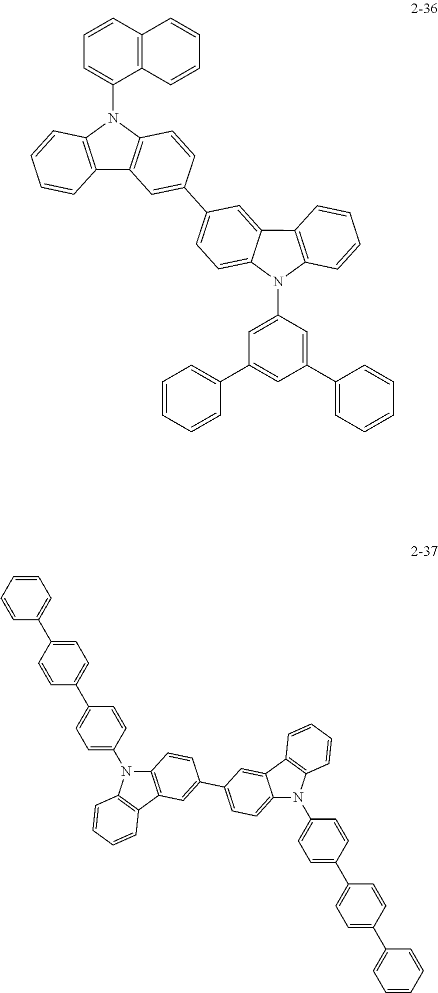 [SMILES: c1ccc(-c2cc(-c3ccccc3)cc(-n3c4ccccc4c4cc(-c5ccc6c(c5)c5ccccc5n6-c5cccc6ccccc56)ccc43)c2)cc1 ; c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1]

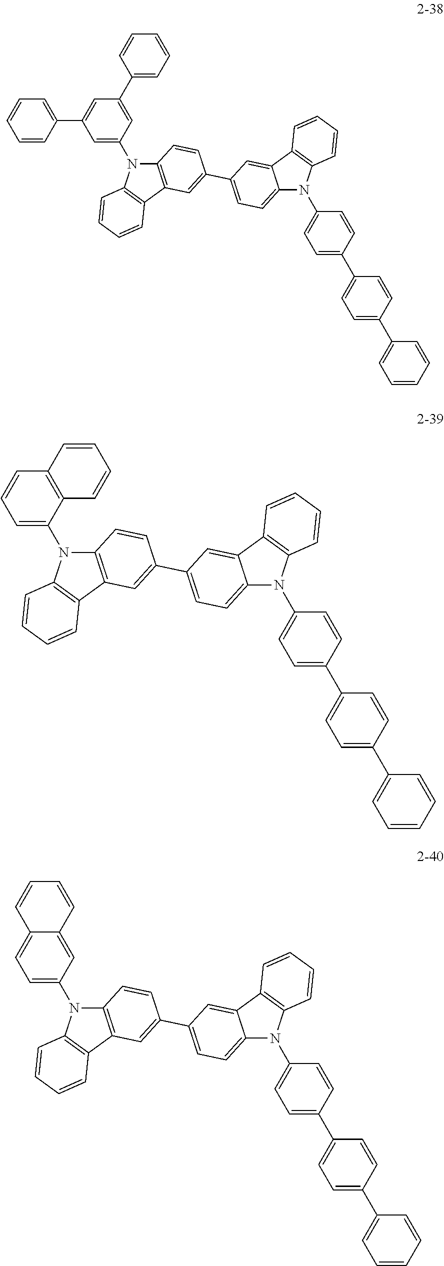 [SMILES: c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1]

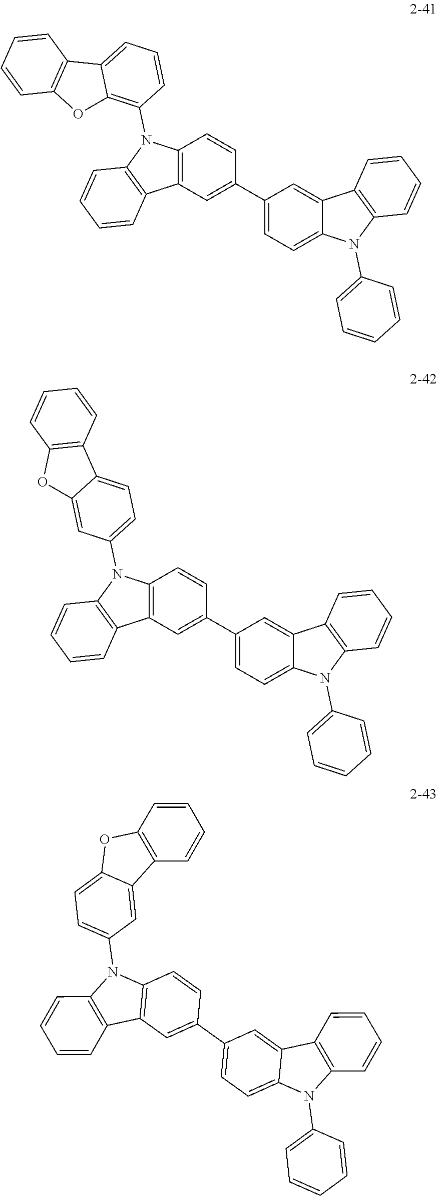 [SMILES: c1ccc(-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4ccc5c(c4)oc4ccccc45)ccc32)cc1 ; c1ccc(-n2c3ccccc3c3cc(-c4ccc5c(c4)c4ccccc4n5-c4ccc5oc6ccccc6c5c4)ccc32)cc1]

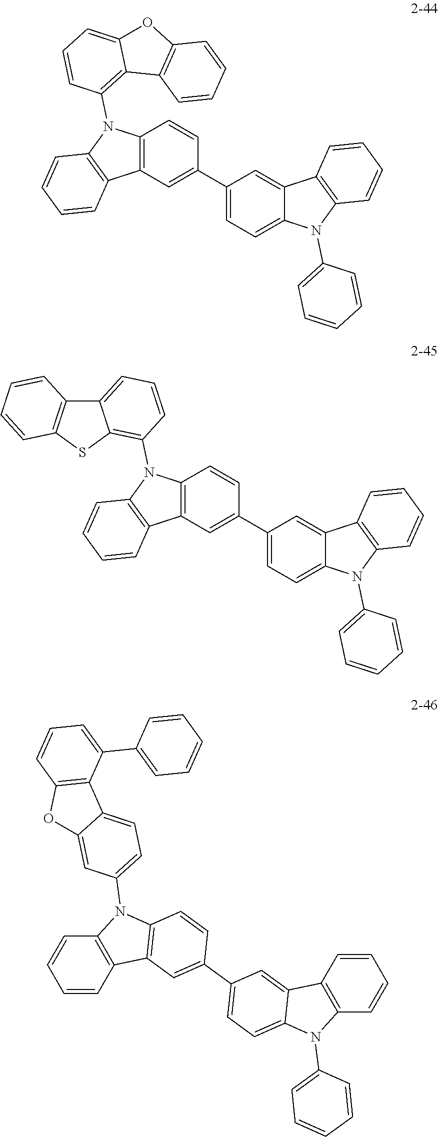 [SMILES: c1ccc(-n2c3ccccc3c3cc(-c4ccc5c(c4)c4ccccc4n5-c4cccc5oc6ccccc6c45)ccc32)cc1 ; c1ccc(-n2c3ccccc3c3cc(-c4ccc5c(c4)c4ccccc4n5-c4cccc5c4sc4ccccc45)ccc32)cc1 ; c1ccc(-c2cccc3oc4cc(-n5c6ccccc6c6cc(-c7ccc8c(c7)c7ccccc7n8-c7ccccc7)ccc65)ccc4c23)cc1]

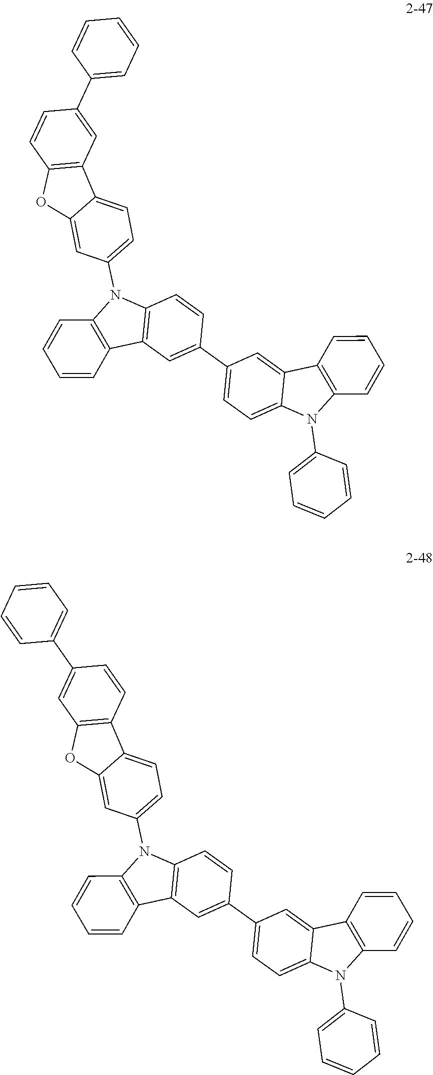 [SMILES: c1ccc(-c2ccc3oc4cc(-n5c6ccccc6c6cc(-c7ccc8c(c7)c7ccccc7n8-c7ccccc7)ccc65)ccc4c3c2)cc1 ; c1ccc(-c2ccc3c(c2)oc2cc(-n4c5ccccc5c5cc(-c6ccc7c(c6)c6ccccc6n7-c6ccccc6)ccc54)ccc23)cc1]

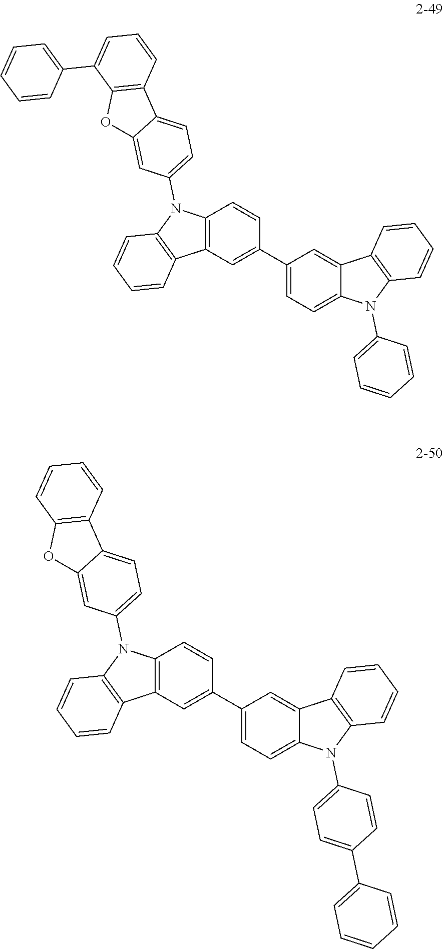 [SMILES: c1ccc(-c2cccc3c2oc2cc(-n4c5ccccc5c5cc(-c6ccc7c(c6)c6ccccc6n7-c6ccccc6)ccc54)ccc23)cc1 ; c1ccc(-c2ccc(-n3c4ccccc4c4cc(-c5ccc6c(c5)c5ccccc5n6-c5ccc6c(c5)oc5ccccc56)ccc43)cc2)cc1]

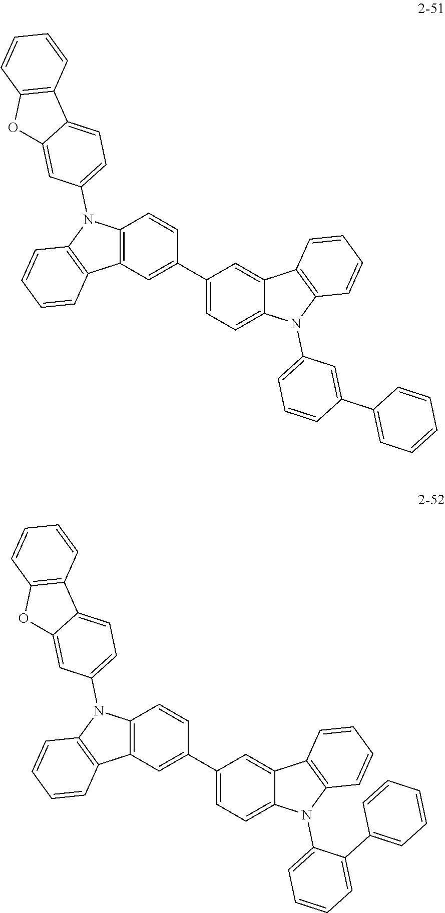 [SMILES: c1ccc(-c2cccc(-n3c4ccccc4c4cc(-c5ccc6c(c5)c5ccccc5n6-c5ccc6c(c5)oc5ccccc56)ccc43)c2)cc1 ; c1ccc(-c2ccccc2-n2c3ccccc3c3cc(-c4ccc5c(c4)c4ccccc4n5-c4ccc5c(c4)oc4ccccc45)ccc32)cc1]

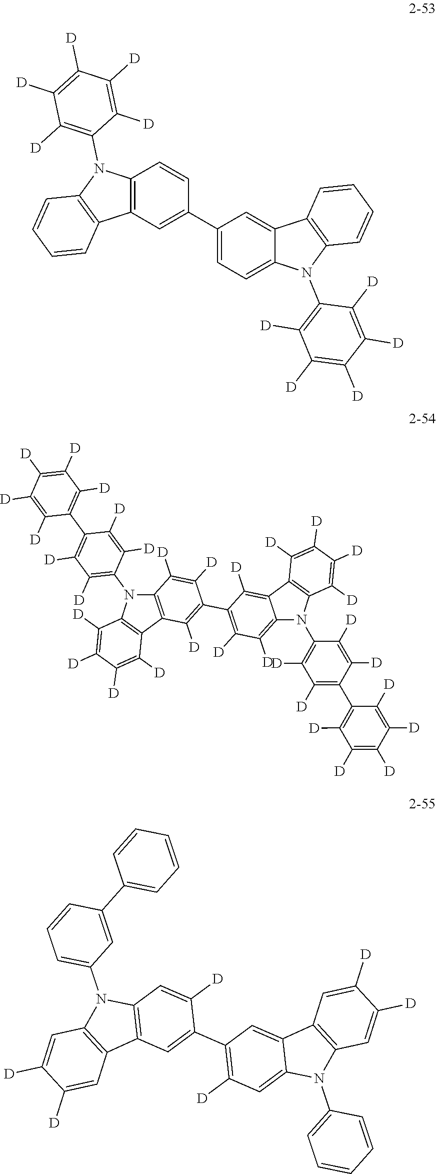 [SMILES: [2H]c1c([2H])c([2H])c(-n2c3ccccc3c3cc(-c4ccc5c(c4)c4ccccc4n5-c4c([2H])c([2H])c([2H])c([2H])c4[2H])ccc32)c([2H])c1[2H] ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c(-c6c([2H])c([2H])c([2H])c([2H])c6[2H])c([2H])c5[2H])c([2H])c([2H])c43)c([2H])c2[2H])c([2H])c1[2H] ; [2H]c1cc2c3cc(-c4cc5c6cc([2H])c([2H])cc6n(-c6cccc(-c7ccccc7)c6)c5cc4[2H])c([2H])cc3n(-c3ccccc3)c2cc1[2H]]

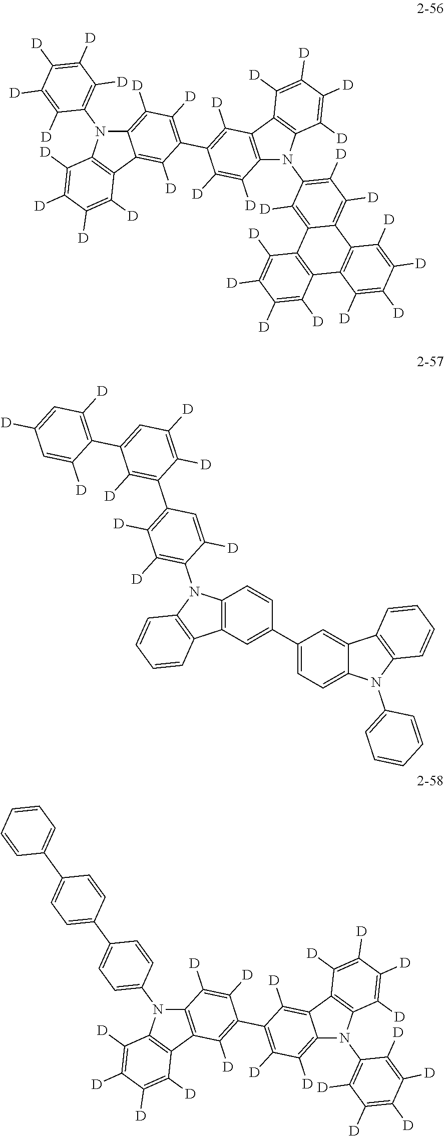

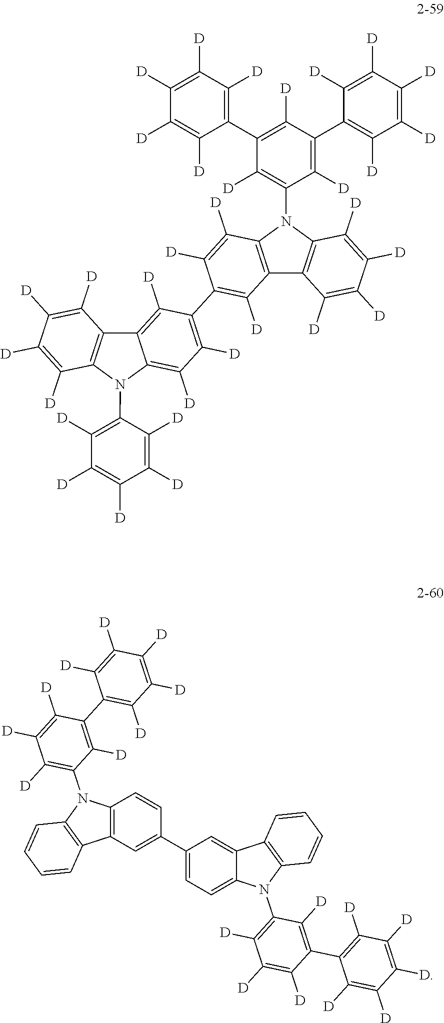

The compound of Formula 5 may be represented by any one of the formulae below.

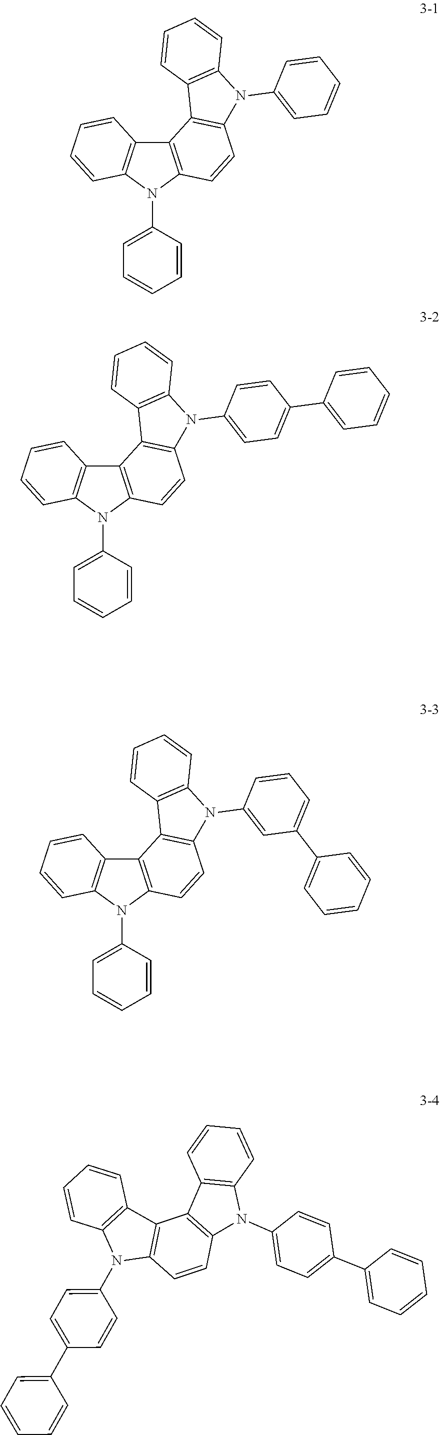 [SMILES: c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccc(-c7ccccc7)cc6)c5ccc43)cc2)cc1]

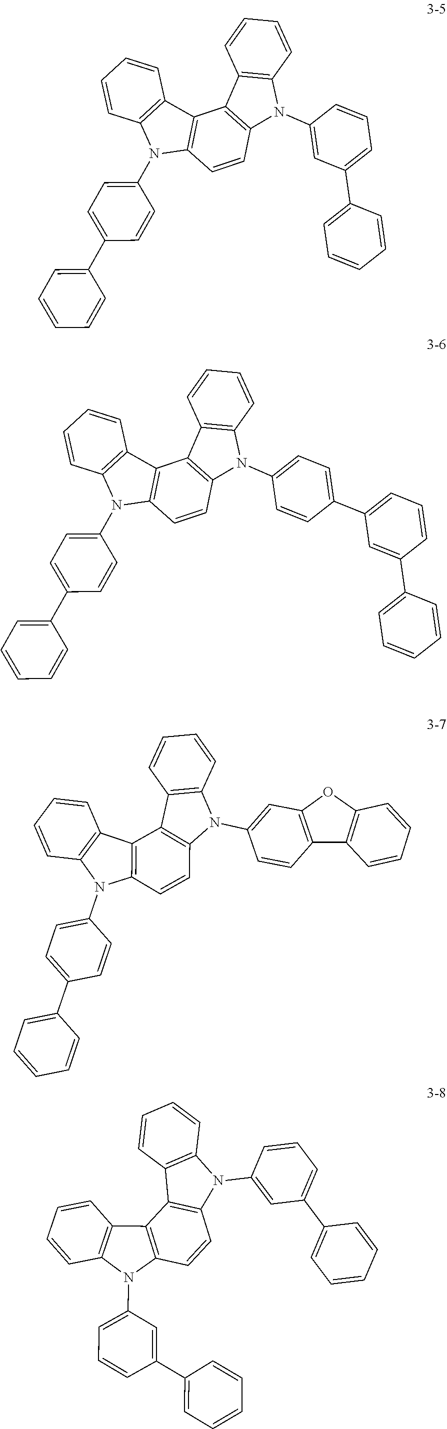 [SMILES: c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6ccc(-c7cccc(-c8ccccc8)c7)cc6)c5ccc43)cc2)cc1 ; c1ccc(-c2ccc(-n3c4ccccc4c4c5c6ccccc6n(-c6ccc7c(c6)oc6ccccc67)c5ccc43)cc2)cc1 ; c1ccc(-c2cccc(-n3c4ccccc4c4c5c6ccccc6n(-c6cccc(-c7ccccc7)c6)c5ccc43)c2)cc1]

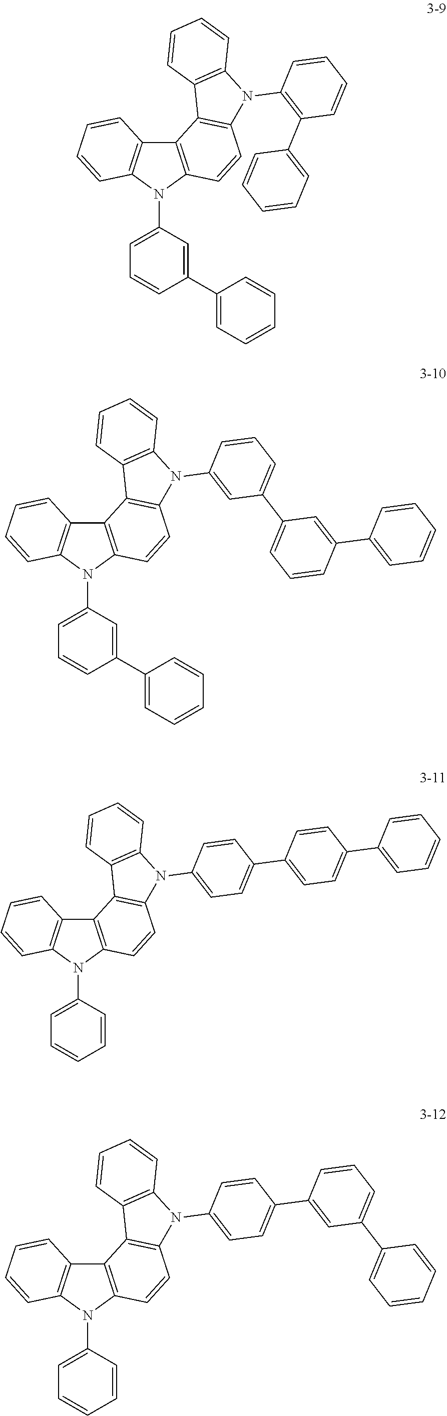 [SMILES: c1ccc(-c2cccc(-n3c4ccccc4c4c5c6ccccc6n(-c6ccccc6-c6ccccc6)c5ccc43)c2)cc1 ; c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2ccc(-c3ccc(-n4c5ccccc5c5c6c7ccccc7n(-c7ccccc7)c6ccc54)cc3)cc2)cc1 ; c1ccc(-c2cccc(-c3ccc(-n4c5ccccc5c5c6c7ccccc7n(-c7ccccc7)c6ccc54)cc3)c2)cc1]

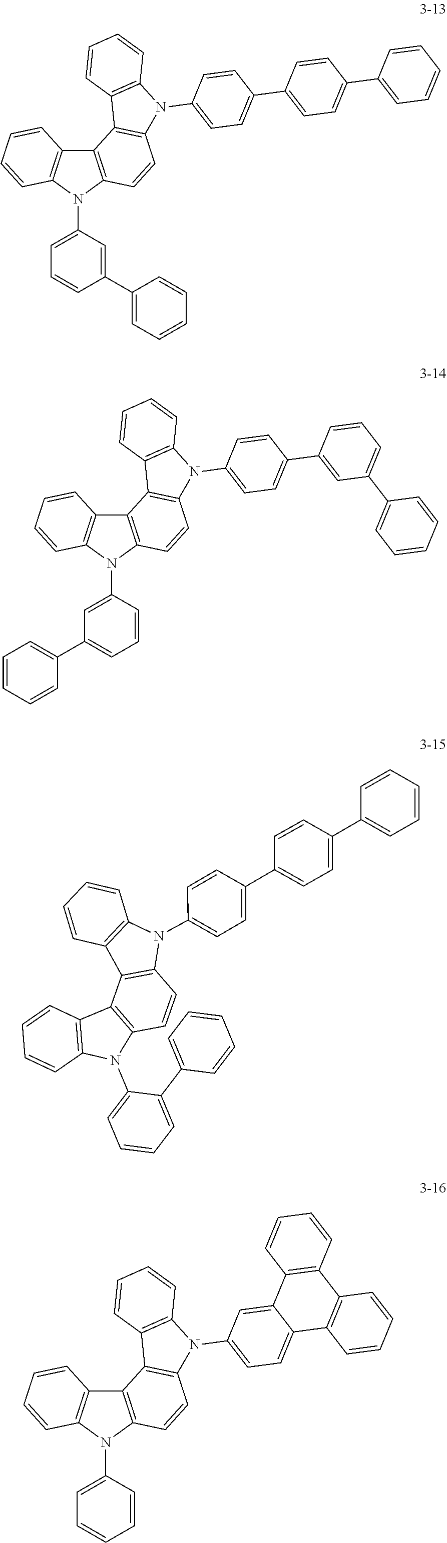 [SMILES: c1ccc(-c2ccc(-c3ccc(-n4c5ccccc5c5c6c7ccccc7n(-c7cccc(-c8ccccc8)c7)c6ccc54)cc3)cc2)cc1 ; c1ccc(-c2cccc(-c3ccc(-n4c5ccccc5c5c6c7ccccc7n(-c7cccc(-c8ccccc8)c7)c6ccc54)cc3)c2)cc1 ; c1ccc(-c2ccc(-c3ccc(-n4c5ccccc5c5c6c7ccccc7n(-c7ccccc7-c7ccccc7)c6ccc54)cc3)cc2)cc1 ; c1ccc(-n2c3ccccc3c3c4c5ccccc5n(-c5ccc6c7ccccc7c7ccccc7c6c5)c4ccc32)cc1]

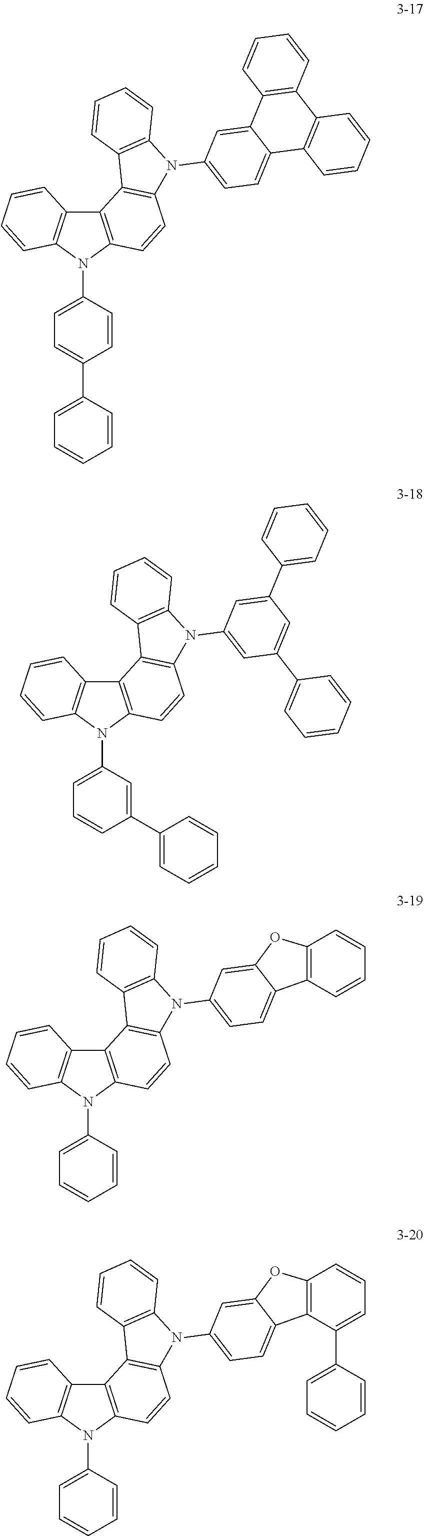 [SMILES: c1ccc(-c2ccc(-n3c4ccccc4c4c5c6ccccc6n(-c6ccc7c8ccccc8c8ccccc8c7c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1 ; c1ccc(-n2c3ccccc3c3c4c5ccccc5n(-c5ccc6c(c5)oc5ccccc56)c4ccc32)cc1 ; c1ccc(-c2cccc3oc4cc(-n5c6ccccc6c6c7c8ccccc8n(-c8ccccc8)c7ccc65)ccc4c23)cc1]

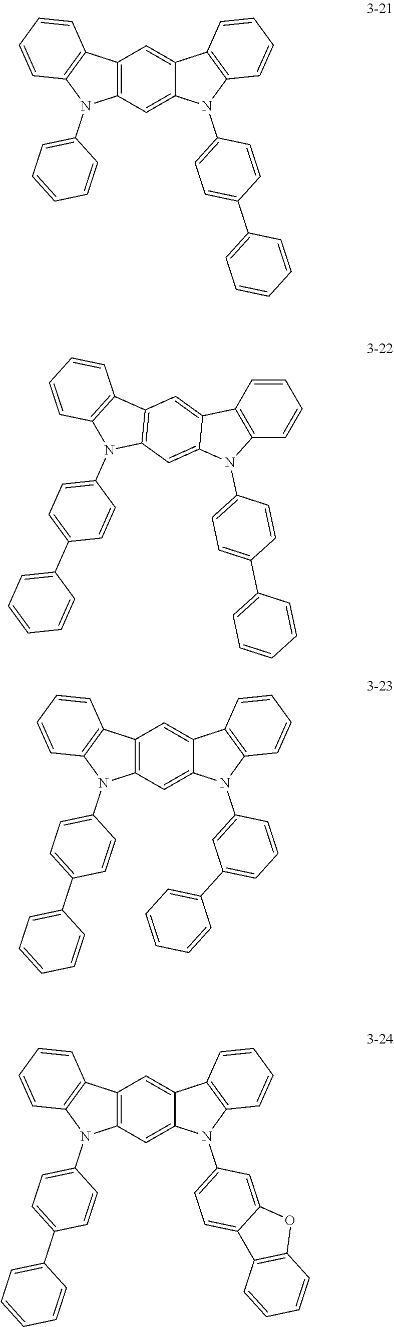

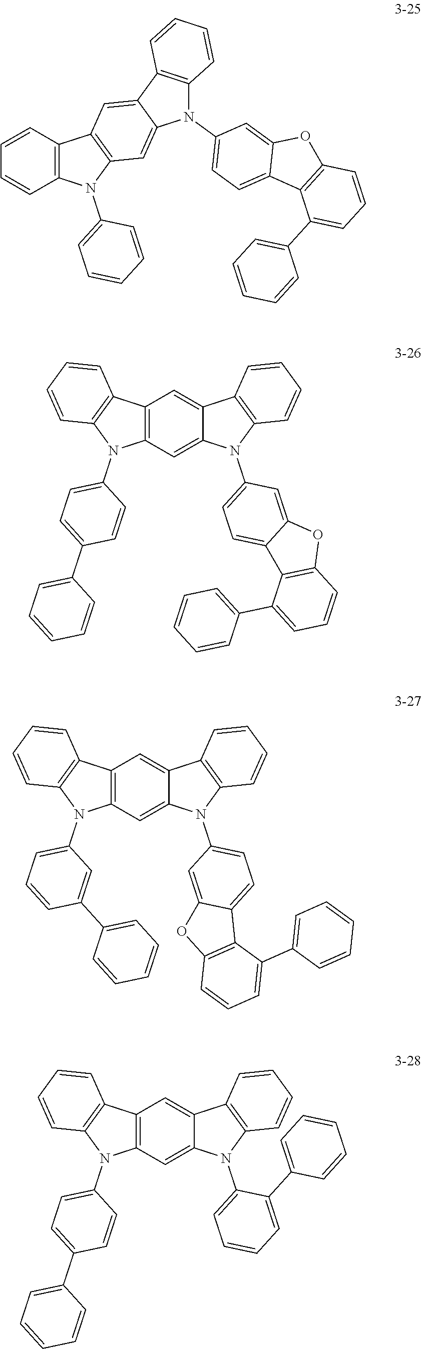 [SMILES: c1ccc(-c2cccc3oc4cc(-n5c6ccccc6c6cc7c8ccccc8n(-c8ccccc8)c7cc65)ccc4c23)cc1 ; c1ccc(-c2ccc(-n3c4ccccc4c4cc5c6ccccc6n(-c6ccc7c(c6)oc6cccc(-c8ccccc8)c67)c5cc43)cc2)cc1 ; c1ccc(-c2cccc(-n3c4ccccc4c4cc5c6ccccc6n(-c6ccc7c(c6)oc6cccc(-c8ccccc8)c67)c5cc43)c2)cc1 ; c1ccc(-c2ccc(-n3c4ccccc4c4cc5c6ccccc6n(-c6ccccc6-c6ccccc6)c5cc43)cc2)cc1]

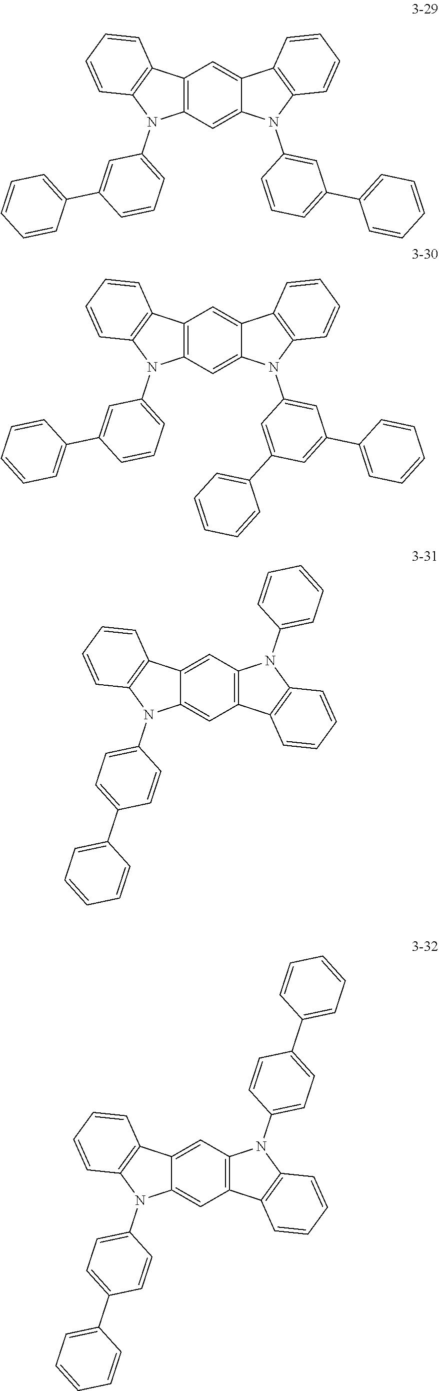 [SMILES: c1ccc(-c2cccc(-n3c4ccccc4c4cc5c6ccccc6n(-c6cccc(-c7ccccc7)c6)c5cc43)c2)cc1 ; c1ccc(-c2cccc(-n3c4ccccc4c4cc5c6ccccc6n(-c6cc(-c7ccccc7)cc(-c7ccccc7)c6)c5cc43)c2)cc1 ; c1ccc(-c2ccc(-n3c4ccccc4c4cc5c(cc43)c3ccccc3n5-c3ccccc3)cc2)cc1 ; c1ccc(-c2ccc(-n3c4ccccc4c4cc5c(cc43)c3ccccc3n5-c3ccc(-c4ccccc4)cc3)cc2)cc1]

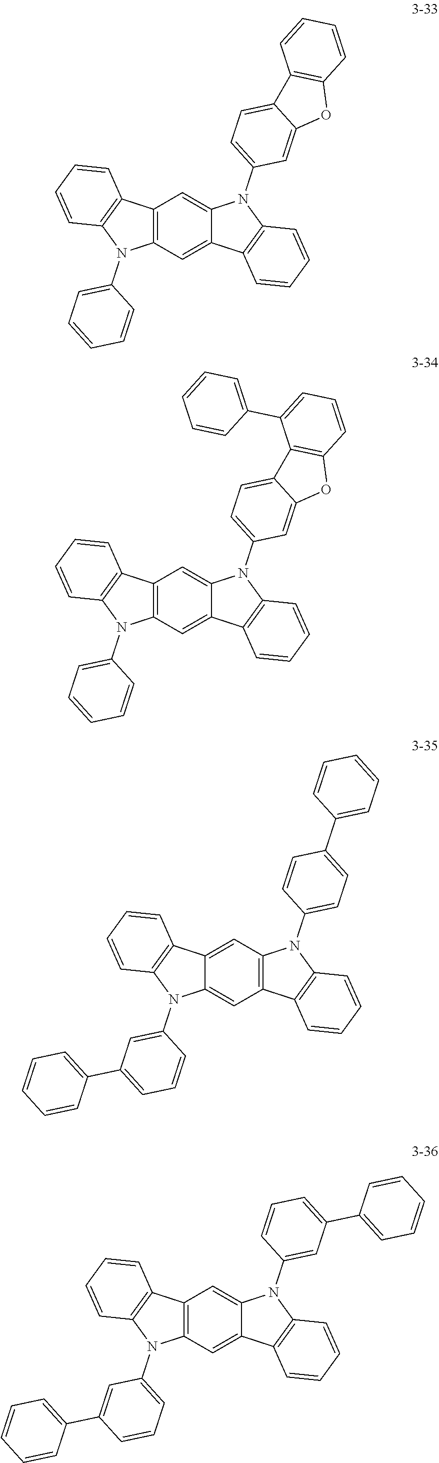 [SMILES: c1ccc(-n2c3ccccc3c3cc4c(cc32)c2ccccc2n4-c2ccc3c(c2)oc2ccccc23)cc1 ; c1ccc(-c2cccc3oc4cc(-n5c6ccccc6c6cc7c(cc65)c5ccccc5n7-c5ccccc5)ccc4c23)cc1 ; c1ccc(-c2ccc(-n3c4ccccc4c4cc5c(cc43)c3ccccc3n5-c3cccc(-c4ccccc4)c3)cc2)cc1 ; c1ccc(-c2cccc(-n3c4ccccc4c4cc5c(cc43)c3ccccc3n5-c3cccc(-c4ccccc4)c3)c2)cc1]

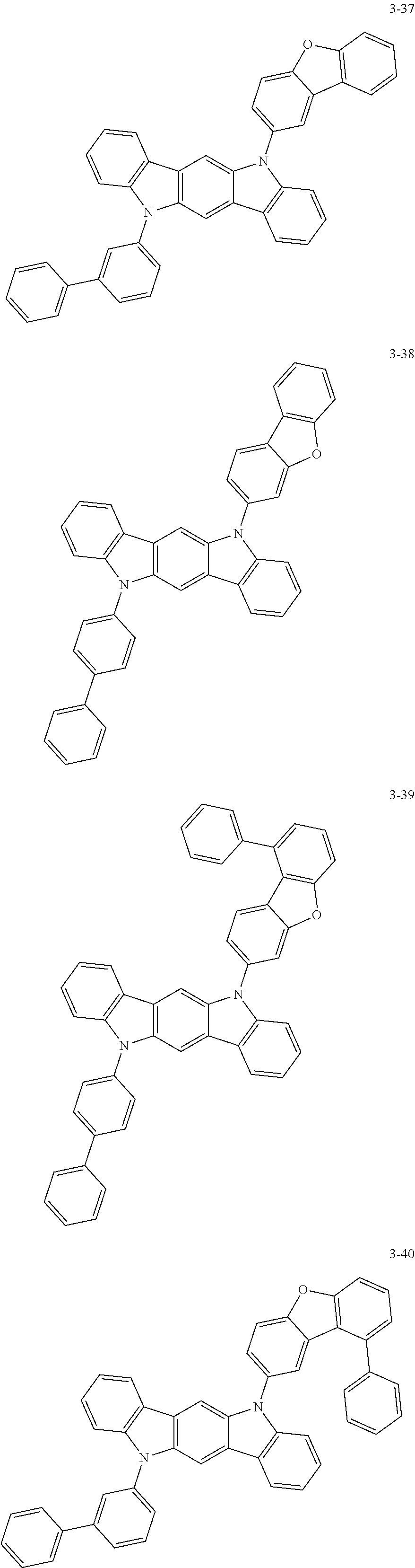 [SMILES: c1ccc(-c2cccc(-n3c4ccccc4c4cc5c(cc43)c3ccccc3n5-c3ccc4oc5ccccc5c4c3)c2)cc1 ; c1ccc(-c2ccc(-n3c4ccccc4c4cc5c(cc43)c3ccccc3n5-c3ccc4c(c3)oc3ccccc34)cc2)cc1 ; c1ccc(-c2ccc(-n3c4ccccc4c4cc5c(cc43)c3ccccc3n5-c3ccc4c(c3)oc3cccc(-c5ccccc5)c34)cc2)cc1 ; c1ccc(-c2cccc(-n3c4ccccc4c4cc5c(cc43)c3ccccc3n5-c3ccc4oc5cccc(-c6ccccc6)c5c4c3)c2)cc1]

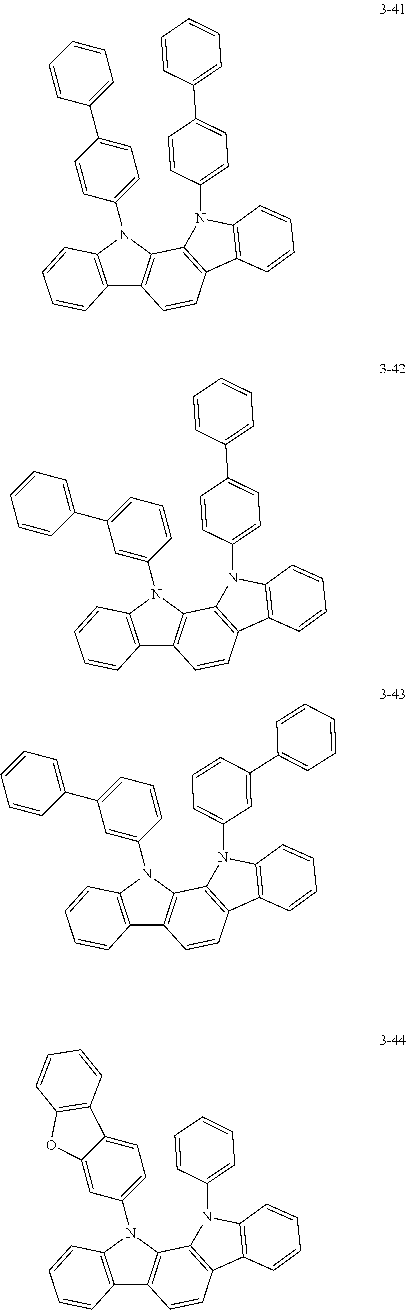 [SMILES: c1ccc(-c2ccc(-n3c4ccccc4c4ccc5c6ccccc6n(-c6ccc(-c7ccccc7)cc6)c5c43)cc2)cc1 ; c1ccc(-c2ccc(-n3c4ccccc4c4ccc5c6ccccc6n(-c6cccc(-c7ccccc7)c6)c5c43)cc2)cc1 ; c1ccc(-c2cccc(-n3c4ccccc4c4ccc5c6ccccc6n(-c6cccc(-c7ccccc7)c6)c5c43)c2)cc1 ; c1ccc(-n2c3ccccc3c3ccc4c5ccccc5n(-c5ccc6c(c5)oc5ccccc56)c4c32)cc1]

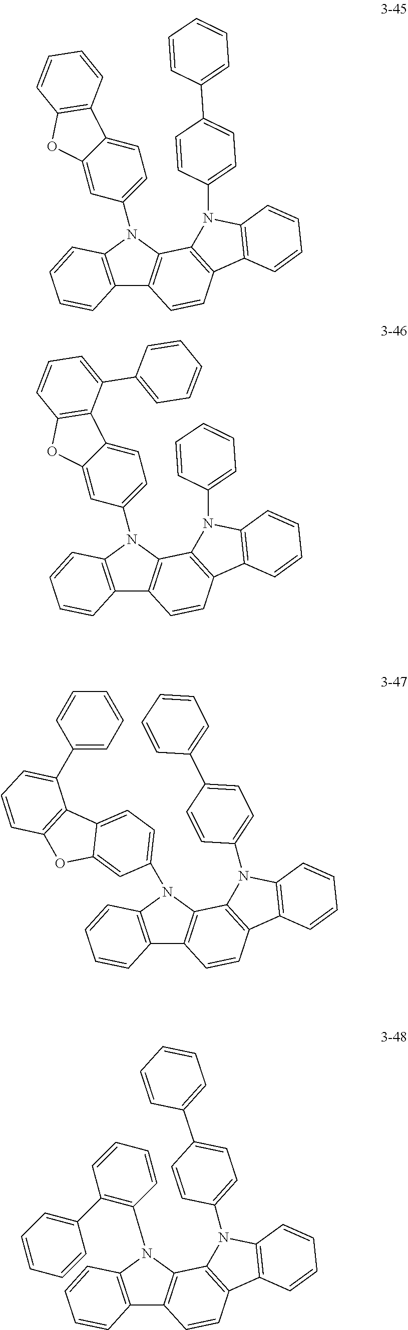

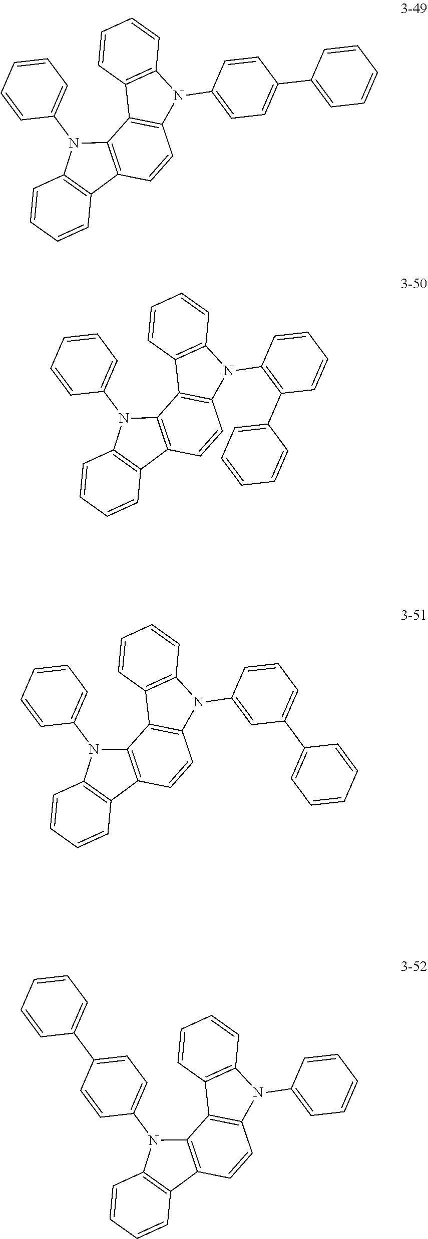 [SMILES: c1ccc(-c2ccc(-n3c4ccccc4c4c3ccc3c5ccccc5n(-c5ccccc5)c34)cc2)cc1 ; c1ccc(-c2ccccc2-n2c3ccccc3c3c2ccc2c4ccccc4n(-c4ccccc4)c23)cc1 ; c1ccc(-c2cccc(-n3c4ccccc4c4c3ccc3c5ccccc5n(-c5ccccc5)c34)c2)cc1 ; c1ccc(-c2ccc(-n3c4ccccc4c4ccc5c(c6ccccc6n5-c5ccccc5)c43)cc2)cc1]

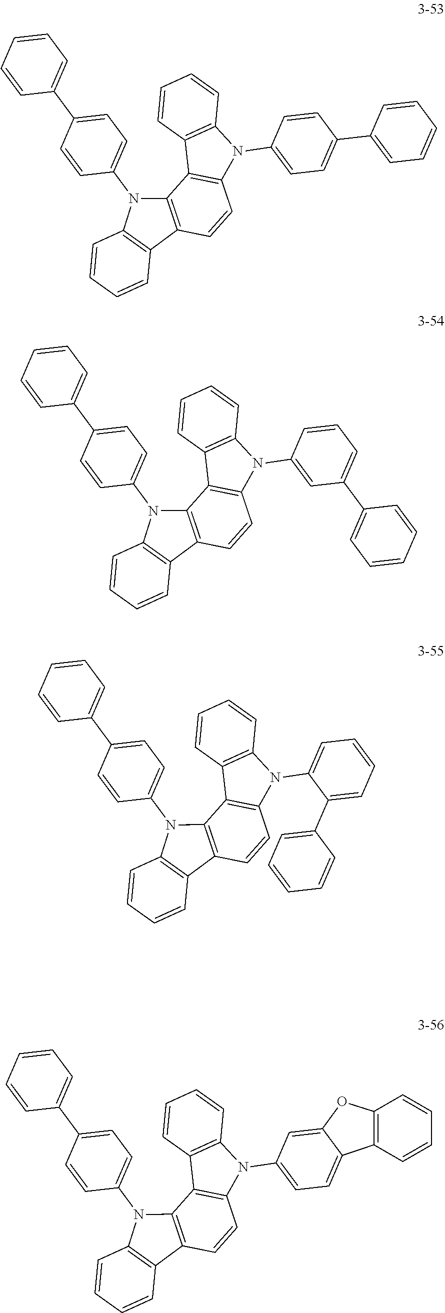 [SMILES: c1ccc(-c2ccc(-n3c4ccccc4c4c3ccc3c5ccccc5n(-c5ccc(-c6ccccc6)cc5)c34)cc2)cc1 ; c1ccc(-c2ccc(-n3c4ccccc4c4ccc5c(c6ccccc6n5-c5cccc(-c6ccccc6)c5)c43)cc2)cc1 ; c1ccc(-c2ccc(-n3c4ccccc4c4ccc5c(c6ccccc6n5-c5ccccc5-c5ccccc5)c43)cc2)cc1 ; c1ccc(-c2ccc(-n3c4ccccc4c4ccc5c(c6ccccc6n5-c5ccc6c(c5)oc5ccccc56)c43)cc2)cc1]

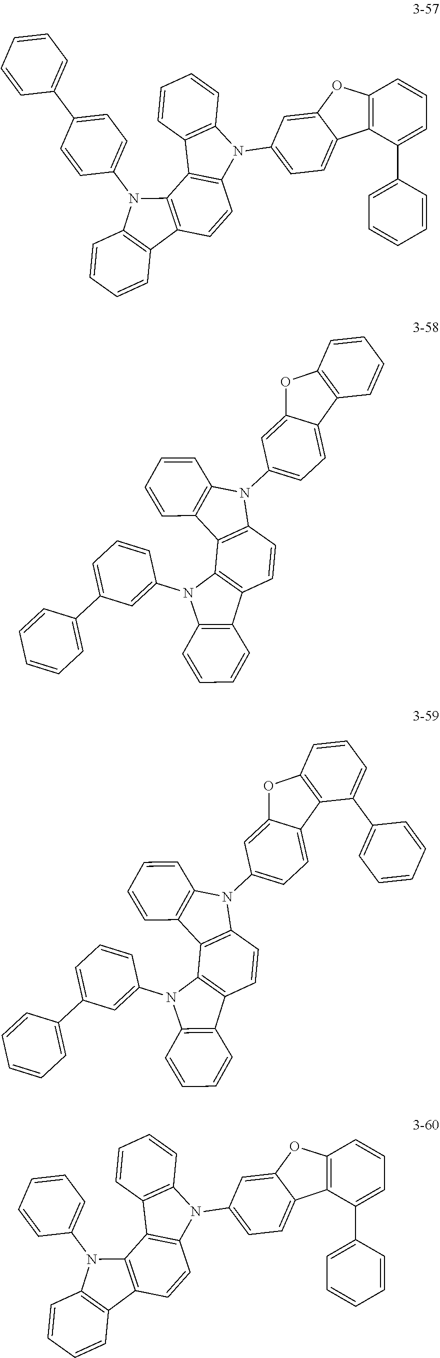 [SMILES: c1ccc(-c2ccc(-n3c4ccccc4c4ccc5c(c6ccccc6n5-c5ccc6c(c5)oc5cccc(-c7ccccc7)c56)c43)cc2)cc1 ; c1ccc(-c2cccc(-n3c4ccccc4c4ccc5c(c6ccccc6n5-c5ccc6c(c5)oc5ccccc56)c43)c2)cc1 ; c1ccc(-c2cccc(-n3c4ccccc4c4ccc5c(c6ccccc6n5-c5ccc6c(c5)oc5cccc(-c7ccccc7)c56)c43)c2)cc1 ; c1ccc(-c2cccc3oc4cc(-n5c6ccccc6c6c5ccc5c7ccccc7n(-c7ccccc7)c56)ccc4c23)cc1]

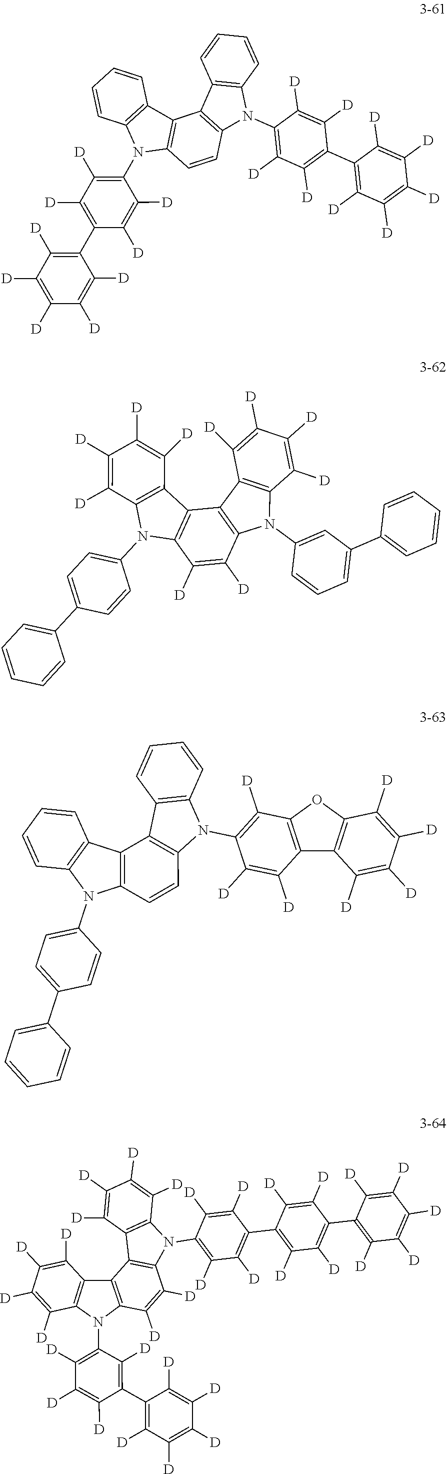

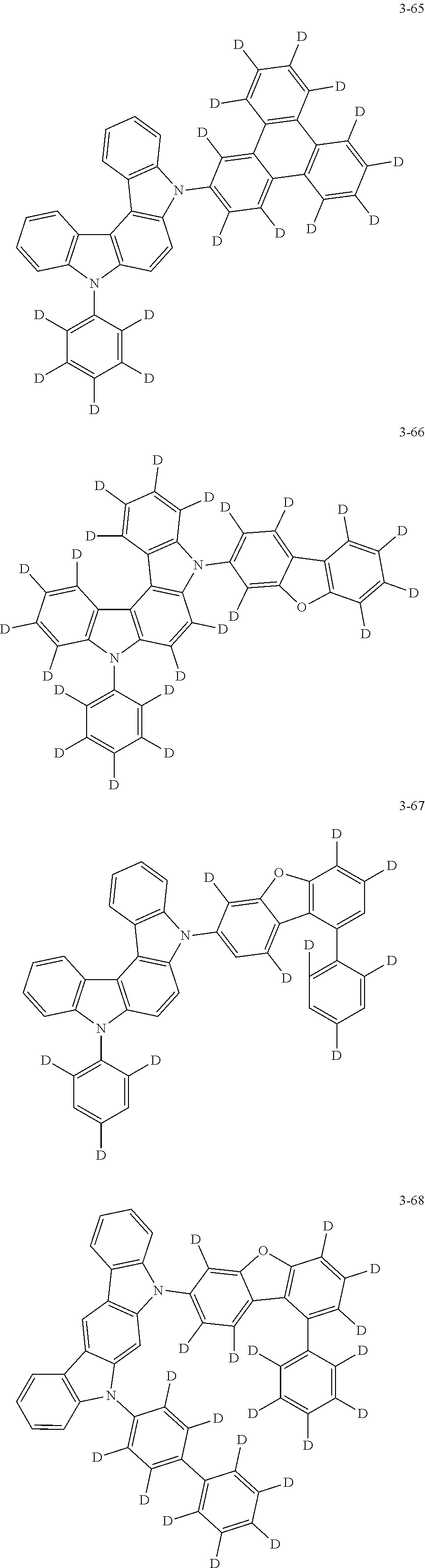

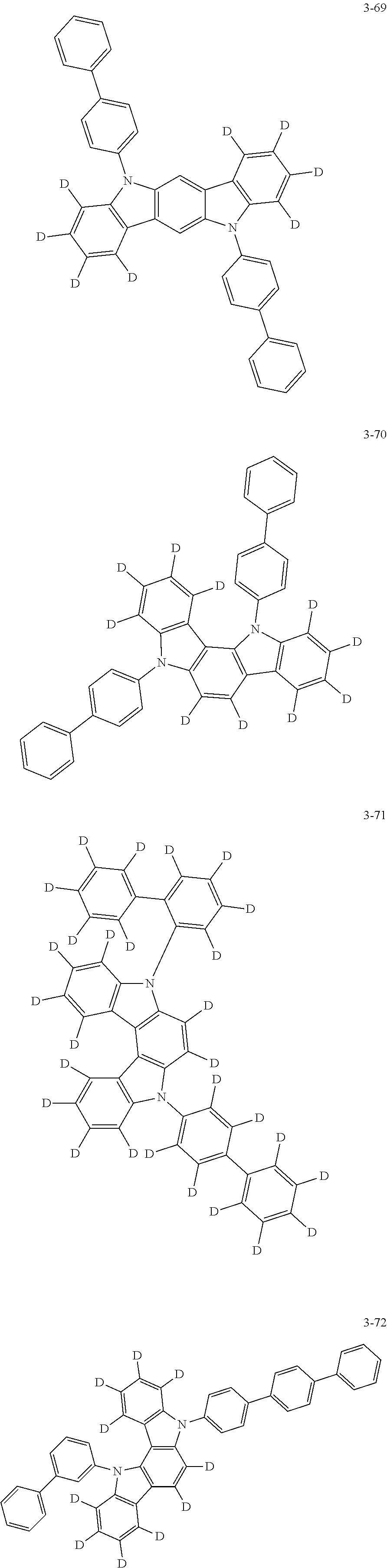

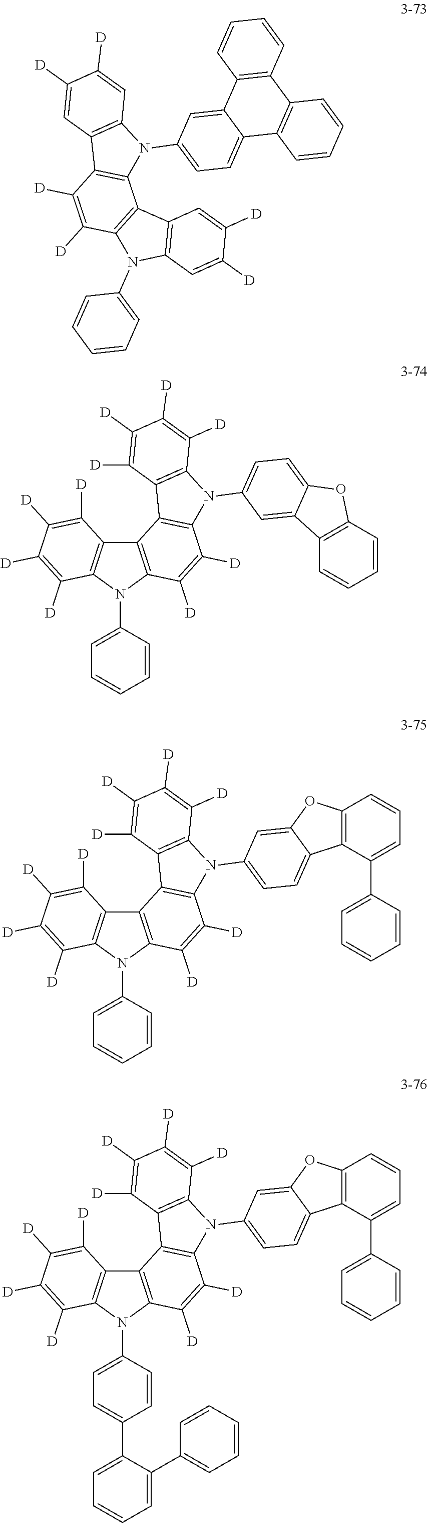

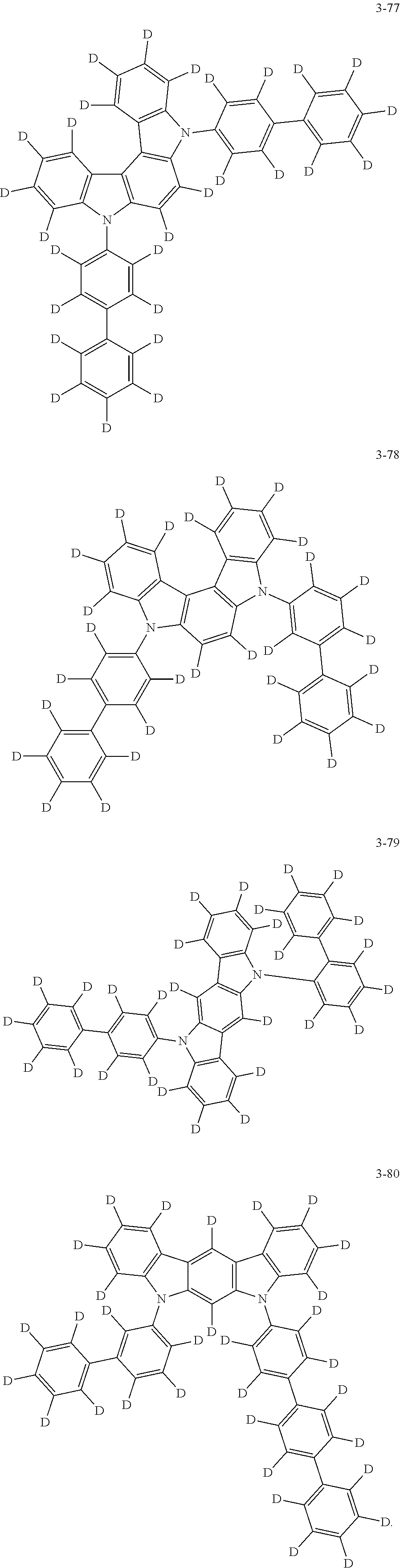

When the organic layer 300 includes the compound represented by Formula 4 or Formula 5 together with the heterocyclic compound, the electron and hole transport characteristics of the light-emitting device may be further enhanced, and the light-emitting efficiency and device lifetime may be further improved.

In one embodiment, the compound represented by Formula 4 or the compound represented by Formula 5 may be included in the same organic layer as the heterocyclic compound, and for example, may be included in the light-emitting layer 303. For example, the heterocyclic compound represented by Formula 1 may be a n-type host material, and the compound represented by Formula 4 or the compound represented by Formula 5 may be an p-type host material.

For example, the heterocyclic compound represented by Formula 1 may have high hole transport ability, thereby acting as a donor, and the compound represented by Formula 4 or the compound represented by Formula 5 may have a high electron transport ability, thereby acting as an acceptor. When these compounds are used together, an exciplex phenomenon may occur, thereby improving charge balance within the device.

In one embodiment, the weight ratio of the heterocyclic compound to the compound represented by Formula 4 or the compound represented by Formula 5 in the organic layer 300 or the light-emitting layer 303 may be 1:10 to 10:1, 1:8 to 8:1, 1:5 to 5:1, or 1:2 to 2:1. Within this range, both electron transport characteristics and hole transport characteristics in the organic layer 300 may be improved. Accordingly, the operating voltage of the light-emitting device may be lowered, and the light-emitting efficiency and lifetime characteristics may be further improved.

As the material of the hole injection layer 301, known hole injection layer materials may be used. For example, phthalocyanine compounds such as copper phthalocyanine disclosed in U.S. Pat. No. 4,356,429, or a starburst amine derivative described in the literature [Advanced Materials, 6, p. 677 (1994)], such as tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), 4,4′,4″-tris(N,N-(2-naphthyl)-phenylamino)triphenylamine (2-TNATA), or a soluble conductive polymer such as polyaniline/dodecylbenzenesulfonic acid, poly(3,4-ethylenedioxythiophene)/poly(4-styrenesulfonate), polyaniline/camphorsulfonic acid, or polyaniline/poly(4-styrenesulfonate) may be used.

As the materials of the hole transport layer 302, pyrazoline derivatives, arylamine derivatives, stilbene derivatives, or triphenyldiamine derivatives may be used, and low-molecular-weight or high-molecular-weight materials may also be used. As the materials of the electron transport layer, oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, or 8-hydroxyquinoline and metal complexes thereof may be used. Not only low-molecular-weight materials but also high-molecular-weight materials may be used.

For example, LiF is typically used in the art as the material of the electron injection layer 305, but the present application is not limited thereto.

As the materials of the electron transport layer 304, anthracene compounds, Alq₃ (tris(8-hydroxyquinolinato)aluminum), 1,3,5-tri[(3-pyridyl)-phen-3-yl]benzene, 2,4,6-tris(3′-(pyridin-3-yl) biphenyl-3-yl)-1,3,5-triazine, 2-(4-(N-phenylbenzoimidazol-1-yl)phenyl)-9,10-dinaphthylanthracene, TPBi (1,3,5-tri(1-phenyl-1H-benzo[d]imidazol-2-yl)benzene), BCP(2,9-dimethyl-4,7-diphenyl-1,10-phenanthroline), Bphen (4,7-diphenyl-1,10-phenanthroline), TAZ (3-(4-biphenylyl)-4-phenyl-5-tert-butylphenyl-1,2,4-triazole), NTAZ (4-(naphthalen-1-yl)-3,5-diphenyl-4H-1,2,4-triazole), tBu-PBD (2-(4-biphenylyl)-5-(4-tert-butylphenyl)-1,3,4-oxadiazole), BAlq (bis(2-methyl-8-quinolinolato-N1,08)-(1,1′-biphenyl-4-olato)aluminum), Bebq2 (beryllium bis(benzoquinolin-10-olate)), ADN (9,10-di(naphthalene-2-yl) anthracene), BmPyPhB (1,3-bis[3,5-di(pyridin-3-yl)phenyl]benzene), or a combination thereof may be used.

As used herein, the charge generation layer may be an N-type charge generation layer, and may further include a dopant known in the art.

According to exemplary embodiments, a method for manufacturing the light-emitting device may be provided.

According to exemplary embodiments, a substrate may be prepared. A first electrode may be formed on the substrate. One or more organic layers may be formed on the first electrode. A second electrode may be formed on the organic layer to manufacture a light-emitting device.

The light-emitting device may be manufactured using conventional methods and materials for manufacturing a light-emitting device, except that one or more organic layers are formed using the above-described heterocyclic compound.

When manufacturing an organic light-emitting device, an organic layer including the heterocyclic compound may be formed using a vacuum deposition method. Alternatively, the organic layer may be formed by applying a composition for an organic layer including the heterocyclic compound using a solution application method. Here, the solution application method may include spin coating, dip coating, inkjet printing, screen printing, spray coating, or roll coating, but is not limited thereto.

The organic layer may be formed using a composition for an organic layer including the heterocyclic compound represented by Formula 1.

The composition for an organic layer of an organic light-emitting device according to the present disclosure may include a heterocyclic compound represented by Formula 1 and a compound represented by Formula 4 or a compound represented by Formula 5. The two compounds may function as a dual host.

The organic layer may be formed by pre-mixing the heterocyclic compound represented by Formula 1 and the compound represented by Formula 4 or the compound represented by Formula 5 using a thermal vacuum deposition method.

The ratio of the weight of the compound represented by Formula 4 or the compound represented by Formula 5 to the weight of the heterocyclic compound represented by Formula 1, based on the total weight of the composition for an organic layer, may be 0.1 to 10. In some embodiments, the ratio of the weight of the compound represented by Formula 4 or the compound represented by Formula 5 to the weight of the heterocyclic compound represented by Formula 1, based on the total weight of the composition for an organic layer, may be 0.2 to 5, or 0.5 to 2.

The composition may be used in forming the organic layer of an organic light-emitting device, and in particular, preferably used as a host material for the light-emitting layer.

The composition is in a form in which two or more compounds are simply mixed, and may be prepared by mixing powdered materials prior to forming the organic layer of an organic light-emitting device, or by mixing compounds that are liquid at a temperature above a predetermined temperature. The composition is in a solid state at or below the melting point of each material and may be maintained in a liquid state under high-temperature conditions.

The composition may further include materials known in the art, such as solvents and additives.

Hereinafter, embodiments of the present invention will be further described with reference to specific experimental examples. The examples and comparative examples included in the experimental examples are merely illustrative of the present disclosure and do not limit the scope of the appended claims. It will be apparent to those skilled in the art that various changes and modifications to the examples can be made within the scope and technical spirit of the present disclosure, and it is also understood that such changes and modifications fall within the scope of the appended claims.

### <Preparative Example 1> Preparation of Compound 1

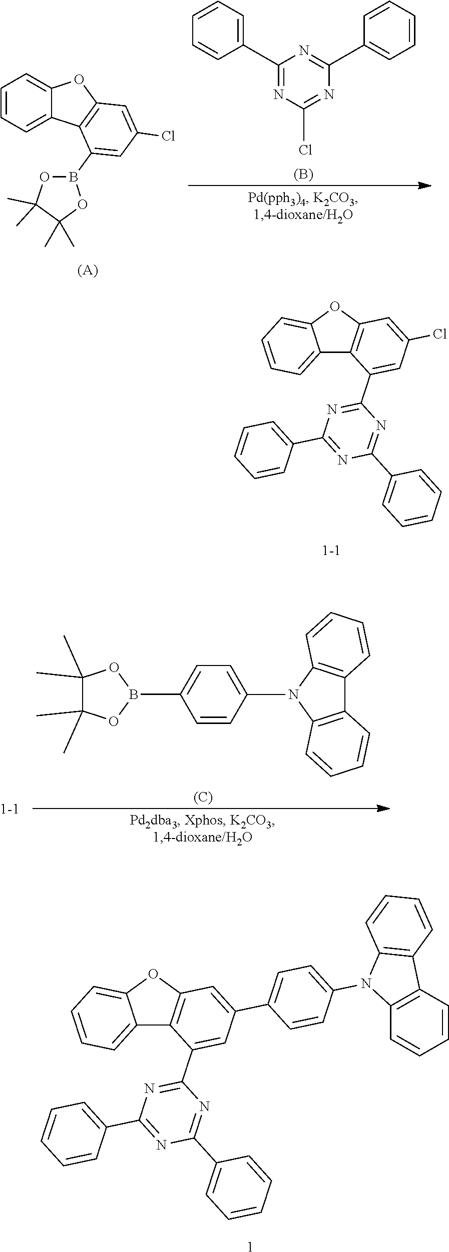

### 1) Preparation of Compound 1-1

20.0 g (60.9 mmol) of 2-(3-chlorodibenzo[b, d]furan-1-yl)-4,4,5,5-tetramethyl-1,3,2-dioxaborolane, 16.3 g (60.9 mmol) of 2-chloro-4,6-diphenyl-1,3,5-triazine, 3.5 g (3.0 mmol) of Pd(pph₃)₄, and 16.8 g (121.7 mmol) of K₂CO₃ were added to a 1 L two-neck flask, dissolved in 1,4-dioxane/H₂O(200 ml/40 mL), and refluxed for 2 hours. After completion of the reaction, the precipitated compound was filtered and recrystallized from toluene methanol to obtain 25.0 g of Compound 1-1 (94.7% yield).

### 2) Preparation of Compound 1

25 g (57.6 mmol) of Compound 1-1, 21.3 g (57.6 mmol) of 9-(4-(4,4,5,5-tetramethyl-1,3,2-dioxaborolan-2-yl)phenyl)-9H-carbazole, 2.6 g (2.9 mmol) of Pd₂dba₃, 2.8 g (5.8 mmol) of XPhos, 15.9 g (115.2 mmol) of K₂CO₃ were added to a 1 L two-neck flask, dissolved in 1,4-dioxane/H₂O(250 mL/50 mL), and refluxed for 6 hours. After completion of the reaction, the precipitated compound was filtered, dissolved in methylene chloride (MC), and purified by silica gel column chromatography. The product was recrystallized from toluene to obtain 30 g of Compound 1 (81% yield).

The following target compound was synthesized in the same manner as in Preparative Example 1, except that Intermediate A in Table 1 was used instead of 2-(3-chlorodibenzo[b,d]furan-1-yl)-4,4,5,5-tetramethyl-1,3,2-dioxaborolane (A), Intermediate B in Table 1 was used instead of 2-chloro-4,6-diphenyl-1,3,5-triazine (B), and Intermediate C in Table 1 was used instead of 9-(4-(4,4,5,5-tetramethyl-1,3,2-dioxaborolan-2-yl)phenyl)-9H-carbazole (C).

TABLE 1

| Com- | | | | |
|---|---|---|---|---|
| pound | (A) | (B) | (C) | Yield |
| 1 | 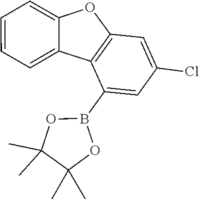 | 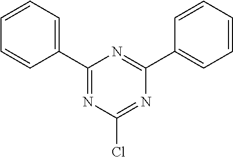 | 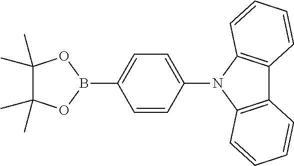 | 81% |
| 3 | 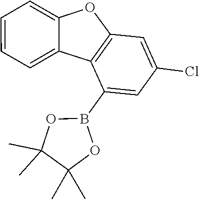 | 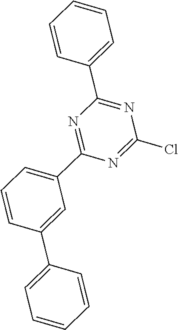 | 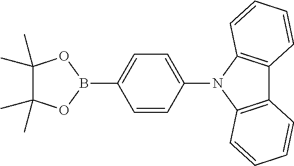 | 88% |
| 14 | 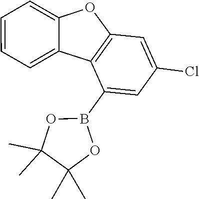 | 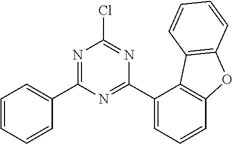 | 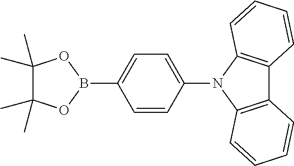 | 91% |
| 22 | 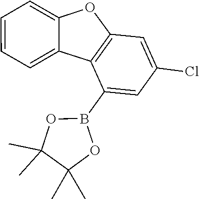 | 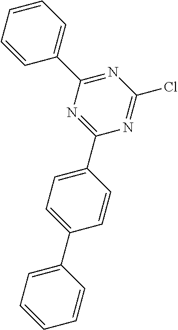 | 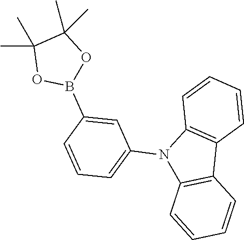 | 82% |
| 35 | 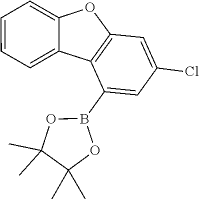 | 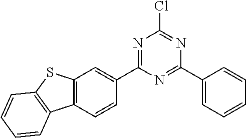 | 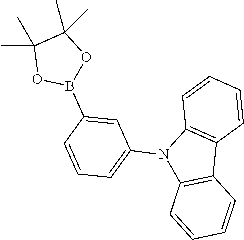 | 80% |
| 39 | 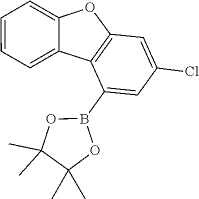 | 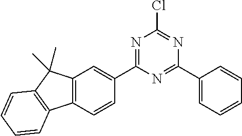 | 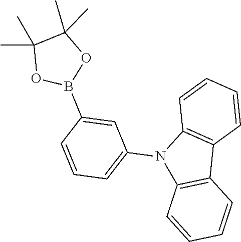 | 73% |
| 44 | 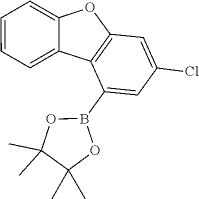 | 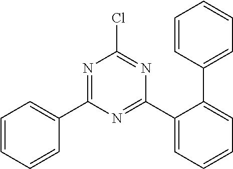 | 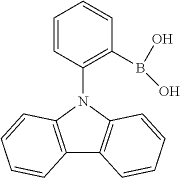 | 84% |
| 45 | 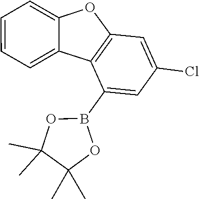 | 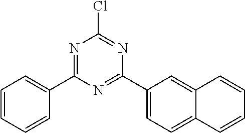 | 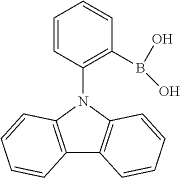 | 92% |
| 54 | 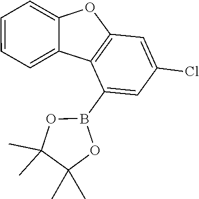 | 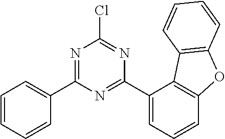 | 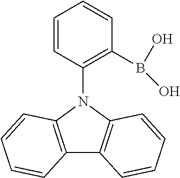 | 90% |
| 61 | 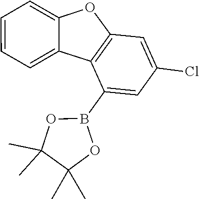 | 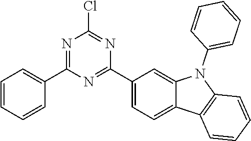 | 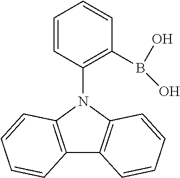 | 79% |
| 65 | 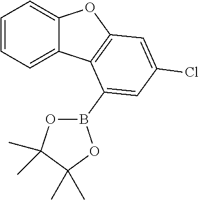 | 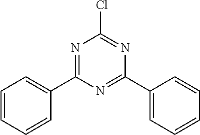 | 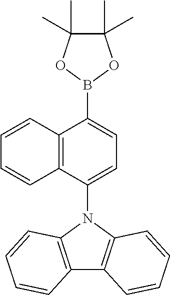 | 81% |
| 71 | 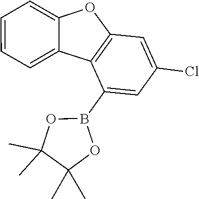 | 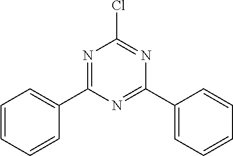 | 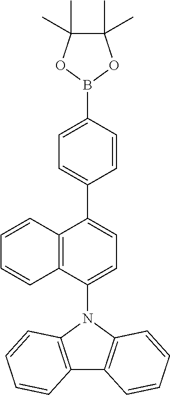 | 72% |
| 78 | 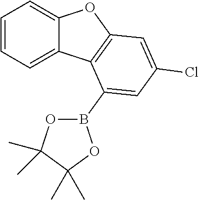 | 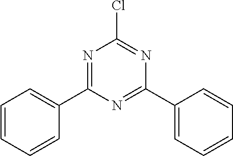 | 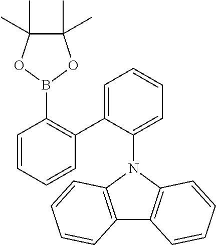 | 77% |
| 85 | 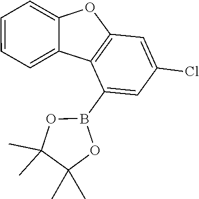 | 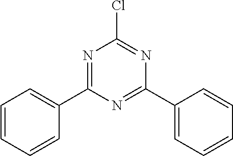 | 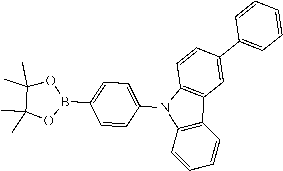 | 86% |
| 88 | 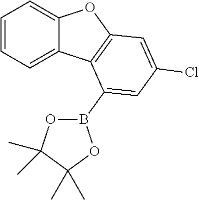 | 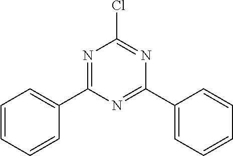 | 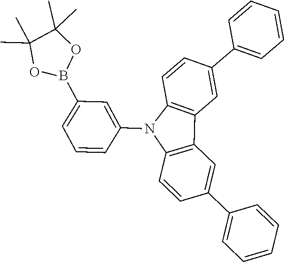 | 81% |
| 97 | 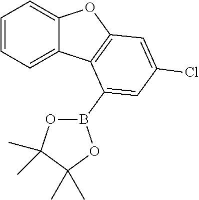 | 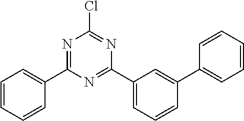 | 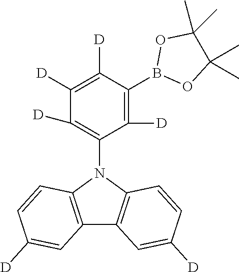 | 77% |
| 100 | 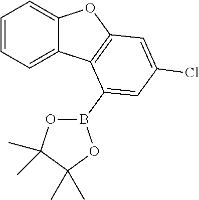 | 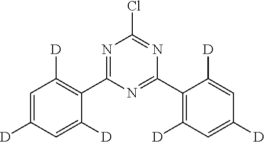 | 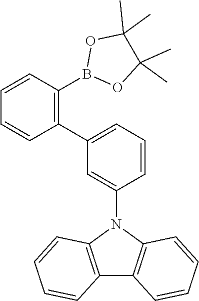 | 79% |
| 128 | 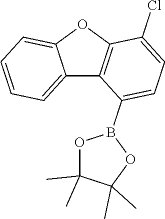 | 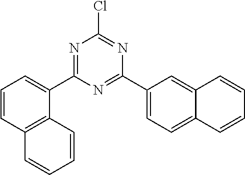 | 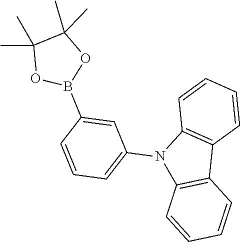 | 86% |
| 163 | 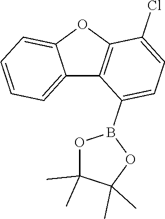 | 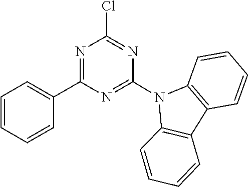 | 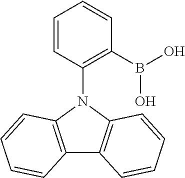 | 64% |
| 166 | 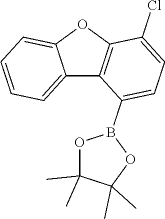 | 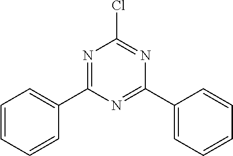 | 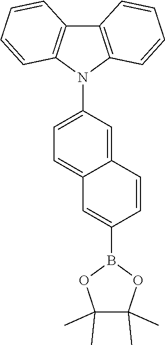 | 72% |
| 179 | 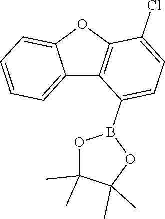 | 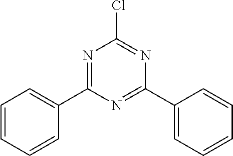 | 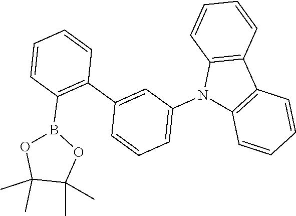 | 73% |
| 181 | 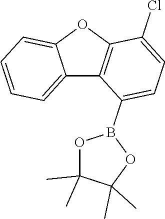 | 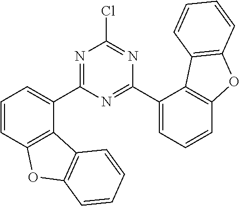 | 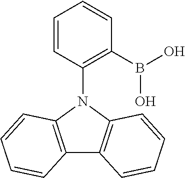 | 83% |
| 206 | 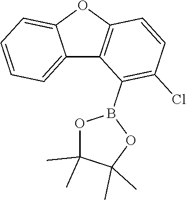 | 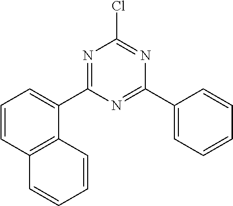 | 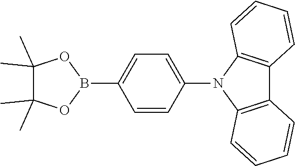 | 75% |
| 231 | 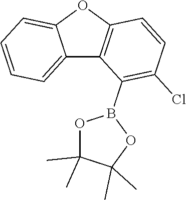 | 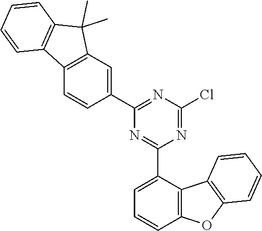 | 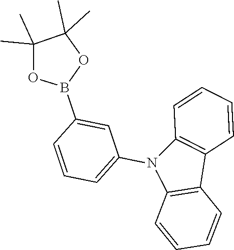 | 77% |
| 250 | 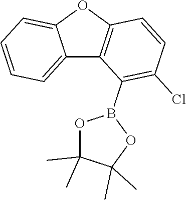 | 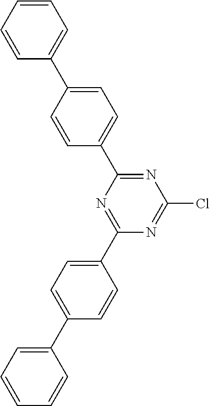 | 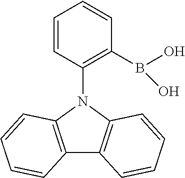 | 73% |
| 267 | 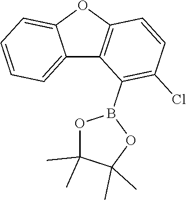 | 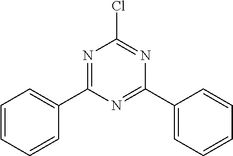 | 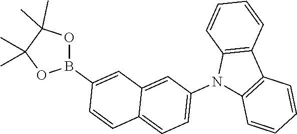 | 80% |
| 271 | 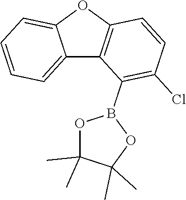 | 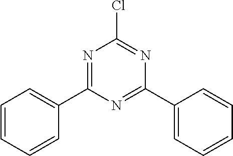 | 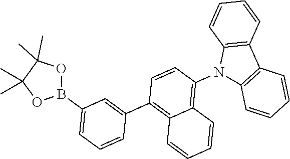 | 76% |
| 282 | 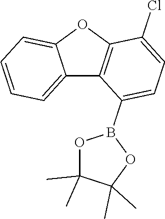 | 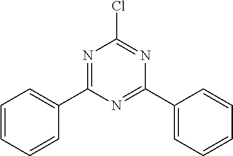 | 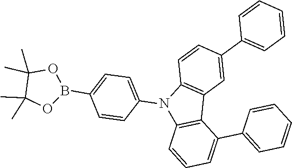 | 54% |
| 283 | 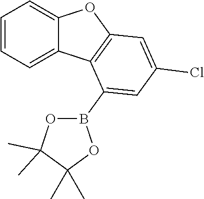 | 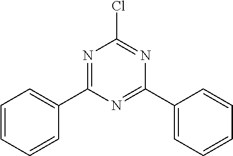 | 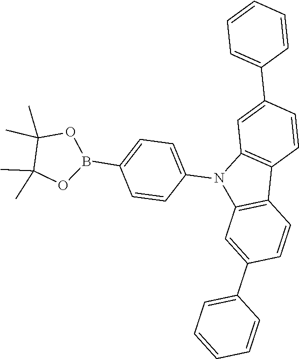 | 68% |
| 284 | 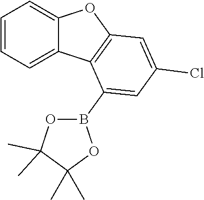 | 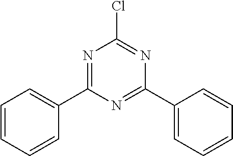 | 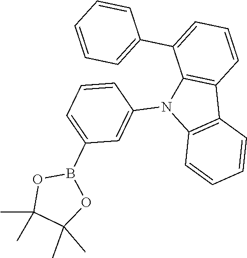 | 57% |
| 285 | 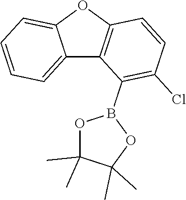 | 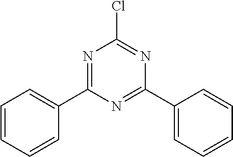 | 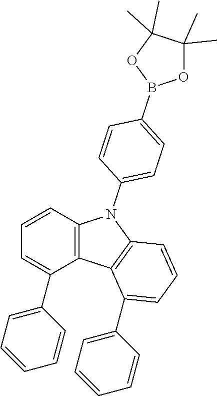 | 71% |
| 286 | 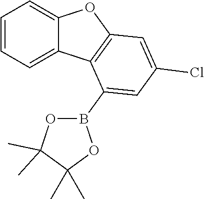 | 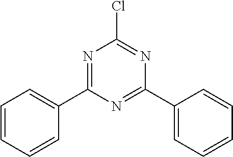 | 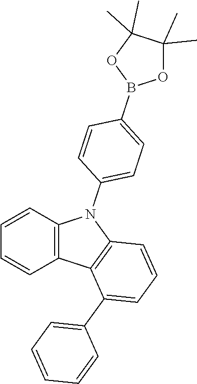 | 85% |
| 291 | 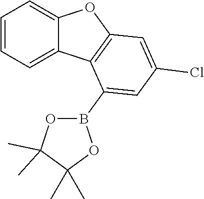 | 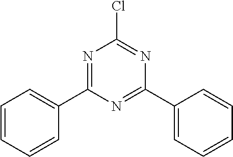 | 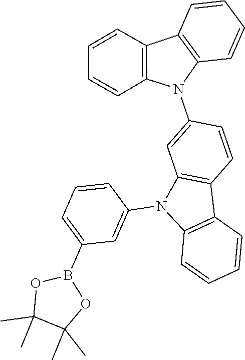 | 74% |
| 294 | 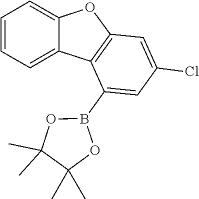 | 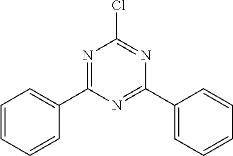 | 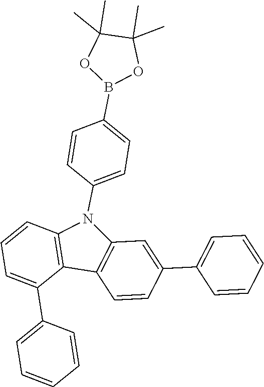 | 68% |

### <Preparative Example 2> Preparation of Compound 2-1

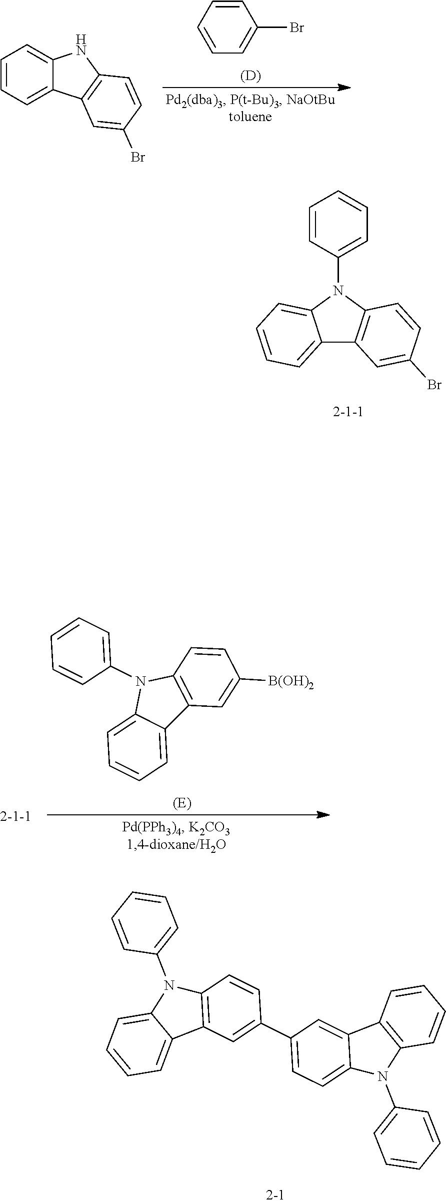

### 1) Preparation of Compound 2-1-1

10 g (49.6 mmol) of 3-bromo-9H-carbazole, 11.7 g (74.4 mmol) of 1-bromobenzene, 2.27 g (2.5 mmol) of Pd₂(dba)₃, 2.42 mL (9.9 mmol) of P(t-Bu)₃, and 9.53 g (99.2 mmol) of NaOtBu were added to a reaction flask. Then, 100 mL of toluene was added, and the mixture was refluxed for 12 hours. After completion of the reaction, the reaction mixture was extracted with MC and distilled water, and then purified by column chromatography to obtain 14 g of Compound 2-1-1 (98% yield).

### 2) Preparation of Compound 2-1

14 g (43.4 mmol) of Compound 2-1-1, 14.9 g (52 mmol) of (9-phenyl-9H-carbazol-3-yl) boronic acid, 2.5 g (2.17 mmol) of Pd(PPh₃)₄, and 17.9 g (130 mmol) of K₂CO₃ were added to a reaction flask, dissolved in 1,4-dioxane/H₂O(140 mL/30 mL), and stirred at 120° C. for 4 hours. The reaction mixture was 5 then cooled to room temperature, and the resulting solid was washed with distilled water and methanol to obtain 17 g of Compound 2-1 (80% yield).

The following target compound was synthesized in the same manner as in Preparative Example 2, except that Intermediate D in Table 2 below was used instead of 1-bromobenzene (D), and Intermediate E in Table 2 below was used instead of (9-phenyl-9H-carbazol-3-yl) boronic acid (E).

TABLE 2

| Com- | | | Target compound |
|---|---|---|---|
| pound | (D) | (E) | (Yield %) |
| 2-1 | 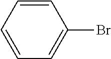 | 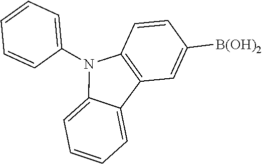 | 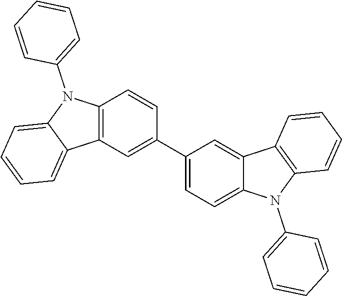 [SMILES: c1ccc(-n2c3ccccc3c3cc(-c4ccc5c(c4)c4ccccc4n5-c4ccccc4)ccc32)cc1] |
| | | | 80% |
| 2-2 | 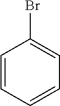 | 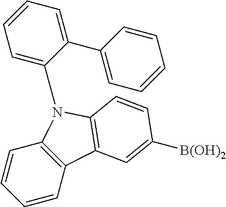 | 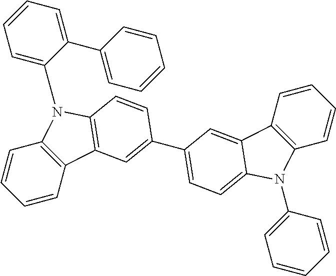 [SMILES: c1ccc(-c2ccccc2-n2c3ccccc3c3cc(-c4ccc5c(c4)c4ccccc4n5-c4ccccc4)ccc32)cc1] |
| | | | 82% |
| 2-3 | 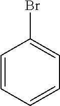 | 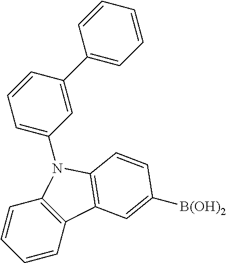 | 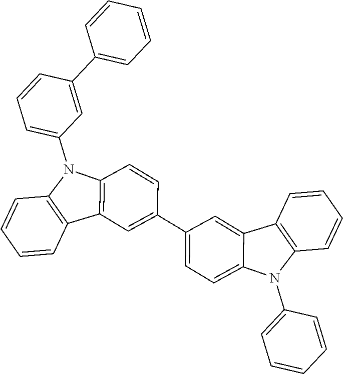 [SMILES: c1ccc(-c2cccc(-n3c4ccccc4c4cc(-c5ccc6c(c5)c5ccccc5n6-c5ccccc5)ccc43)c2)cc1] |
| | | | 80% |
| 2-27 | 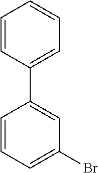 | 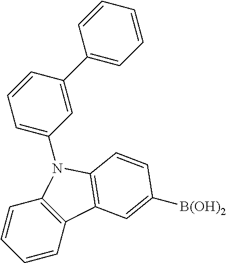 | 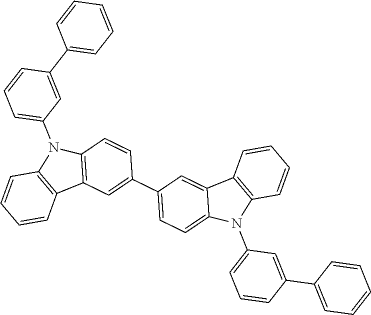 [SMILES: c1ccc(-c2cccc(-n3c4ccccc4c4cc(-c5ccc6c(c5)c5ccccc5n6-c5cccc(-c6ccccc6)c5)ccc43)c2)cc1] |
| | | | 85% |
| 2-29 | 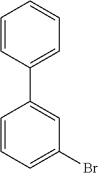 | 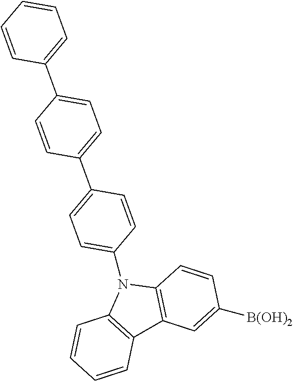 | 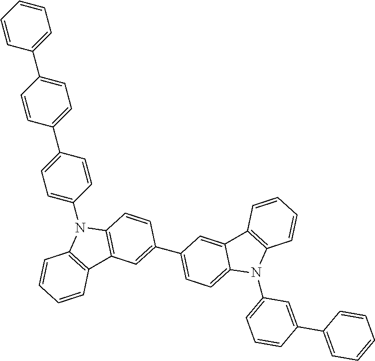 [SMILES: c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1] |
| | | | 80% |
| 2-30 | 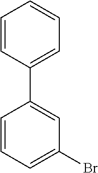 | 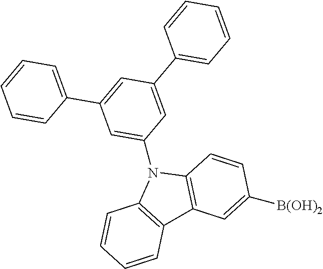 | 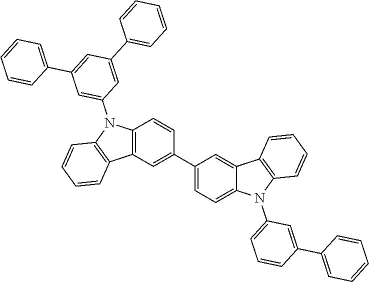 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1] |
| | | | 79% |

### <Preparative Example 3> Preparation of Compound 3-1

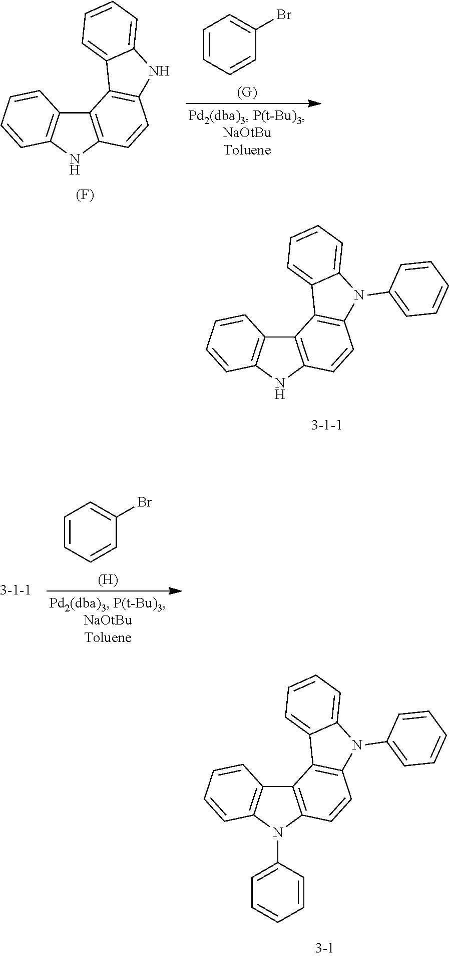

### 1) Preparation of Compound 3-1-1

10 g (39.0 mmol) of 5,8-dihydroindolo[2,3-c]carbazole, 6.12 g (39.0 mmol) of 1-bromobenzene, 1.79 g (1.95 mmol) of Pd₂(dba)₃, 0.92 mL (3.9 mmol) of P(t-Bu)₃, 7.50 g (78.0 mmol) of NaOtBu were added to a reaction flask. Then, 100 mL of toluene was added, and the mixture was refluxed at 135° C. for 2 hours. After completion of the reaction, the reaction mixture was extracted with MC and distilled water, and then purified by column chromatography to obtain 7.3 g of Compound 3-1-1 (56% yield).

### 2) Preparation of Compound 3-1

7.3 g (22.0 mmol) of Compound 3-1-1, 3.8 g (24.2 mmol) of 1-bromobenzene, 1.01 g (1.1 mmol) of Pd₂(dba)₃, 0.52 mL (3.9 mmol) of P(t-Bu)₃, and 4.23 g (44.0 mmol) of NaOtBu were added to a reaction flask. Then, 80 mL of toluene was added, and the mixture was refluxed at 135° C. for 8 hours. After completion of the reaction, the reaction mixture was extracted with MC and distilled water, and purified by column chromatography to obtain 8.3 g of Compound 3-1 (93% yield).

The target compound was synthesized in the same manner as in Preparative Example 3, except that Intermediate F in Table 3 was used instead of 5,8-dihydroindolo[2,3-c]carbazole (F), Intermediate Gin Table 3 was used instead of 1-bromobenzene (G), and Intermediate H in Table 3 was used instead of 1-bromobenzene (H).

TABLE 3

| Compound | (F) | (G) | (H) | Yield |
|---|---|---|---|---|
| 3-1 | 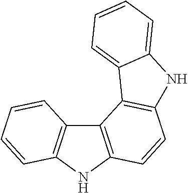 | 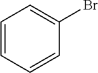 | 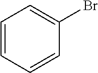 | 93% |
| 3-4 | 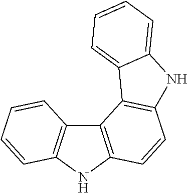 | 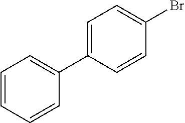 | 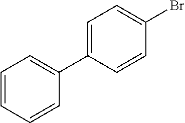 | 93% |
| 3-5 | 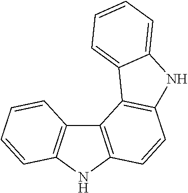 | 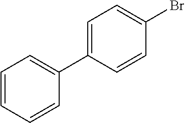 | 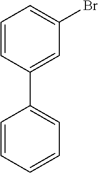 | 74% |
| 3-22 | 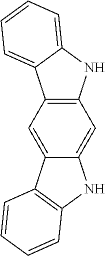 | 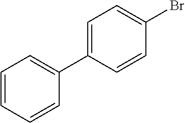 | 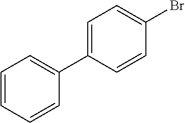 | 81% |
| 3-35 | 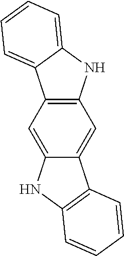 | 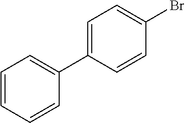 | 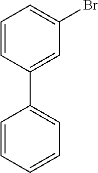 | 88% |
| 3-41 | 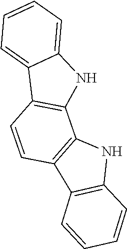 | 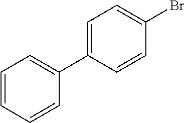 | 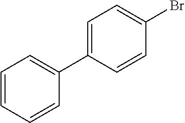 | 91% |

### <Preparative Example 4> Preparation of Compound 95

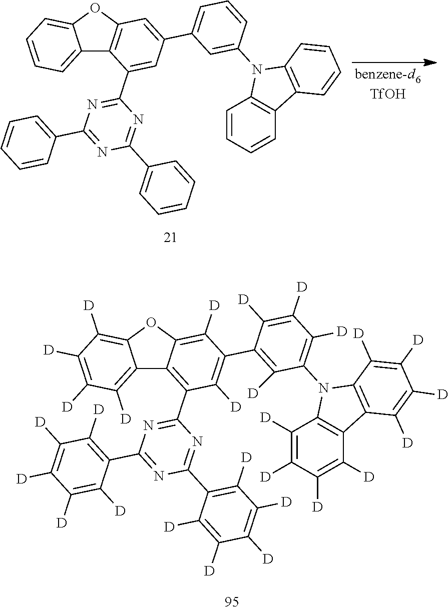

### 1) Preparation of Compound 95

9.8 g (15.3 mmol) of Compound 21, synthesized using the methods of Preparative Examples 1 to 3, was dissolved in 100 mL of benzene-do. After the temperature was adjusted to 0° C. using an ice bath, 9.6 mL (107.4 mmol) of trifluoromethanesulfonic acid was slowly added dropwise. The mixture was then stirred at room temperature for 5 hours. An ice bath was installed again, and the mixture was subsequently neutralized with an aqueous K₃PO₄ solution. The reaction mixture was extracted with MC and distilled water. The organic layer was treated with MgSO₄ concentrated. The residue was dissolved in MC, purified by silica gel chromatography, and the solid was precipitated with methanol and filtered to obtain 9 g of Compound 97 (88% yield).

The target compound was synthesized in the same manner as in Preparative Example 4, except that Intermediate I (Table 4) was used instead of Compound 21.

TABLE 4

| Compound | (I) | |
|---|---|---|
| 95 | 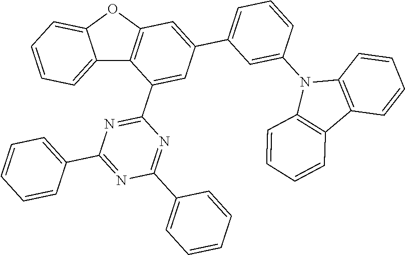 | |
| 194 | 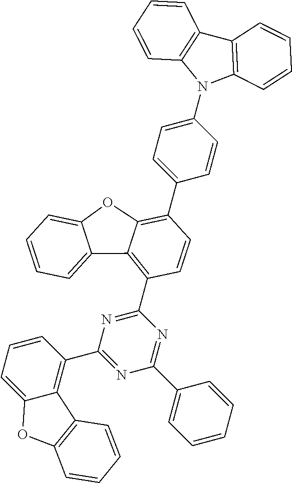 | |
| 277 | 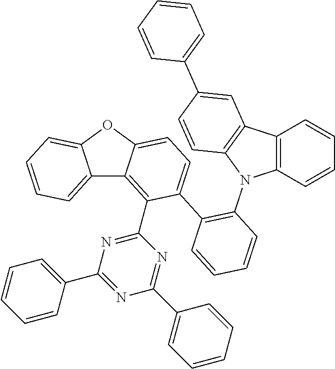 | |
| 2-54 | 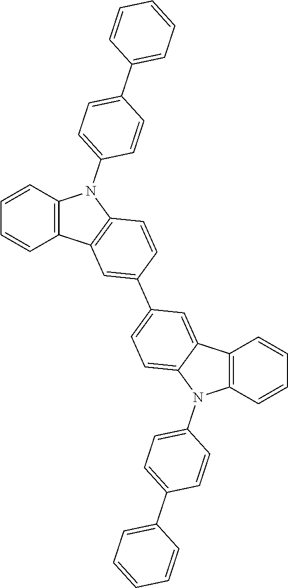 | |
| 3-64 | 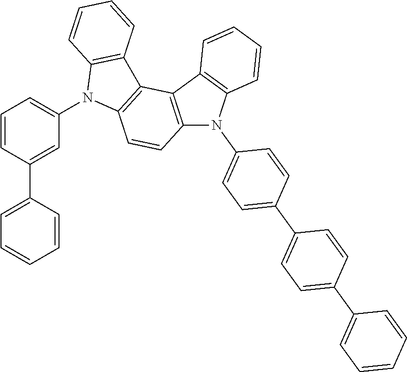 | |
| Compound | Target compound | Yield |
| 95 | 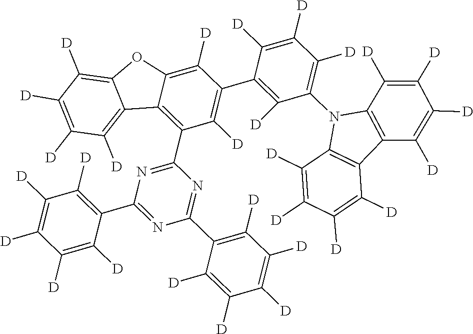 | 88% |
| 194 | 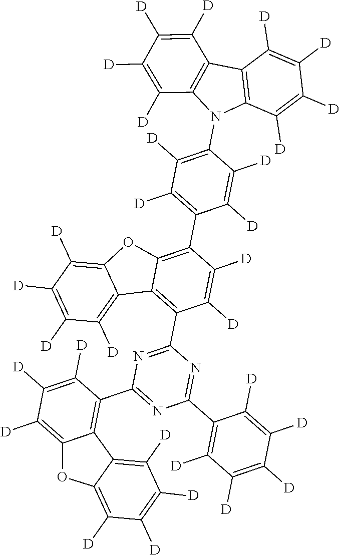 | 86% |
| 277 | 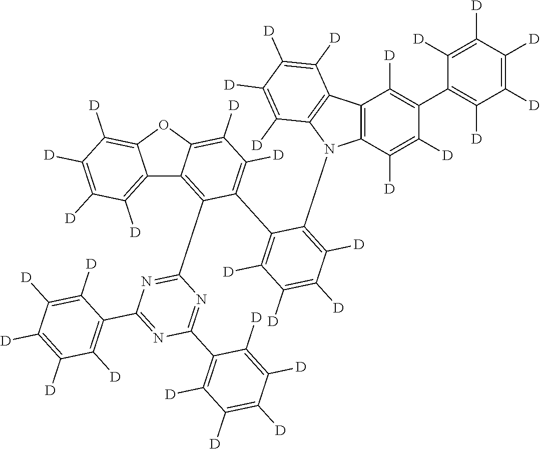 | 84% |
| 2-54 | 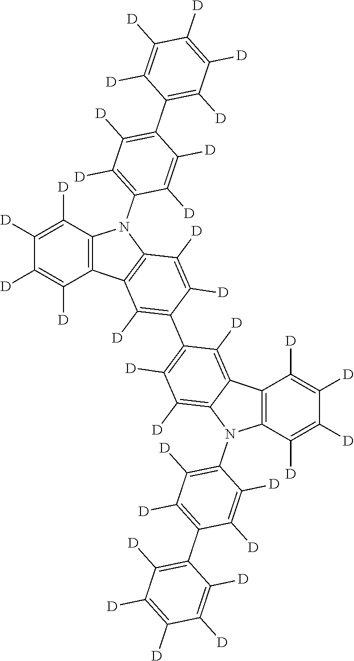 | 90% |
| 3-64 | 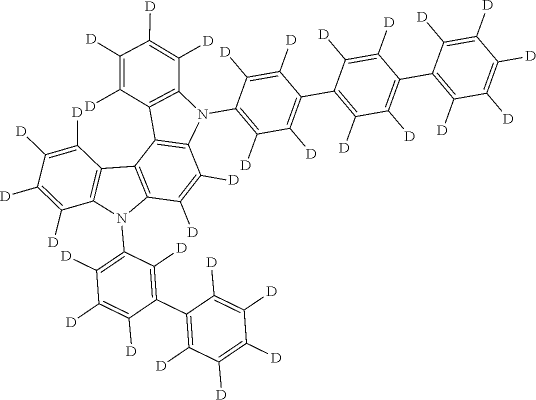 | 92% |

Tables 5 and 6 below present the ¹H NMR and FD-MS data of the synthesized compounds. These data confirm that the desired compounds were successfully synthesized.

For Compounds 95, 194, 277, 2-54, and 3-64, the deuterium substitution level was 100%, and therefore no ¹H NMR analysis results were obtained.

TABLE 5

| Compound | ¹H NMR (CDCl₃, 400 Mz) |
|---|---|
| 1 | δ = 8.55(1H, s), 8.36(4H, d), 8.19(1H, d), 7.98(1H, d), 7.94(1H, d), 7.92(2H, d), 7.91(2H, |
| | d), 7.86(1H, d), 7.81(1H, d), 7.58(1H, dd), 7.54(1H, d), 7.5(7H, d), 7.39(1H, dd), 7.35(1H, |
| | dd), 7.31(1H, s), 7.2 (1H, t), 7.16 (1H, t) |
| 3 | δ = 8.55(1H, s), 8.38(1H, d), 8.36(2H, d), 8.19(1H, d), 7.98(1H, d), 7.94(2H, d), 7.92(2H, |
| | d), 7.91(2H, d), 7.86(1H, d), 7.81(1H, dd), 7.75(2H, d), 7.73(1H, d), 7.61(1H, dd), 7.58(1H, |
| | dd), 7.54(1H, s), 7.5 (4H, t), 7.49 (2H, t), 7.39 (1H, dd), 7.35 (1H, dd), 7.31 (1H, t), 7.2 |
| | (1H, d), 7.16 (1H, d) |
| 14 | δ = 8.55(1H, s), 8.36(2H, d), 8.19(1H, d), 7.98(2H, d), 7.94(1H, d), 7.92(2H, d), 7.91(2H, d), |
| | 7.86(1H, d), 7.82(1H, d), 7.81(1H, dd), 7.69(1H, d), 7.58(1H, d), 7.57(1H, dd), 7.54(2H, |
| | dd), 7.5(6H, s), 7.39 (2H, t), 7.35 (1H, t), 7.31 (2H, dd), 7.2 (1H, dd), 7.16 (1H, t) |
| 22 | δ = 8.55(1H, s), 8.36(2H, d), 8.21(1H, d), 8.19(1H, d), 7.98(1H, d), 7.96(2H, d), 7.94(1H, d), |
| | 7.86(1H, d), 7.81(1H, d), 7.75(2H, dd), 7.68(1H, d), 7.6(1H, d), 7.58(1H, dd), 7.54(1H, dd), |
| | 7.5(4H, s), 7.49 (2H, t), 7.47 (1H, t), 7.39 (1H, dd), 7.35 (1H, dd), 7.31 (1H, t), 7.25 (2H, d), |
| | 7.2 (1H, d), 7.16 (1H, d) |
| 35 | δ = 8.55(1H, s), 8.45(1H, d), 8.36(2H, d), 8.24(1H, d), 8.21(1H, d), 8.2(1H, d), 8.19(1H, d), |
| | 7.98(1H, d), 7.94(2H, d), 7.93(1H, dd), 7.86(1H, d), 7.81(1H, d), 7.68(1H, dd), 7.6(1H, dd), |
| | 7.58(1H, s), 7.56 (1H, t), 7.54 (1H, t), 7.5 (4H, dd), 7.49 (1H, dd), 7.47 (1H, t), 7.39 (1H, d), |
| | 7.35 (1H, d), 7.31 (1H, d), 7.20 (1H, t), 7.16 (1H, t) |
| 39 | δ = 8.55(1H, s), 8.36(2H, d), 8.21(1H, d), 8.19(1H, d), 8.09(1H, d), 7.98(1H, d), 7.94(1H, d), |
| | 7.9(1H, d), 7.89(1H, d), 7.86(1H, dd), 7.81(1H, d), 7.78(1H, d), 7.68(1H, dd), 7.6(1H, dd), |
| | 7.58(1H, s), 7.55 (1H, t), 7.54 (1H, t), 7.5 (4H, dd), 7.47 (1H, dd), 7.39 (1H, t), 7.38 (1H, d), |
| | 7.35 (1H, d), 7.31 (1H, d), 1.69(6H, s) |
| 44 | δ = 8.55(1H, s), 8.36(2H, d), 8.19(1H, d), 7.98(1H, d), 7.96(2H, d), 7.94(1H, d), 7.92(1H, d), |
| | 7.91(1H, d), 7.86(1H, d), 7.81(1H, dd), 7.8(1H, d), 7.79(2H, d), 7.6(2H, dd), 7.58(1H, dd), |
| | 7.54(1H, s), 7.5 (4H, t), 7.46 (3H, t), 7.39 (1H, dd), 7.35 (1H, dd), 7.31 (1H, t), 7.2 (1H, d), |
| | 7.16 (1H, d) |
| 45 | δ = 9.09(1H, s), 8.55(1H, d), 8.49(1H, d), 8.36(2H, d), 8.19(1H, d), 8.16(1H, d), 8.08(1H, d), |
| | 8(1H, d), 7.98(1H, d), 7.94(1H, dd), 7.92(1H, d), 7.91(1H, d), 7.86(1H, dd), 7.81(1H, dd), |
| | 7.8(1H, s), 7.61 (1H, t), 7.59 (1H, t), 7.58 (1H, dd), 7.54 (1H, dd), 7.5 (5H, t), 7.46 (1H, d), |
| | 7.39 (1H, d), 7.35 (1H, d) |
| 54 | δ = 8.55(1H, s), 8.36(2H, d), 8.19(1H, d), 7.98(2H, d), 7.94(1H, d), 7.92(1H, d), 7.91(1H, d), |
| | 7.86(1H, d), 7.82(1H, d), 7.81(1H, dd), 7.8(1H, d), 7.69(1H, d), 7.58(1H, dd), 7.57(1H, dd), |
| | 7.54(2H, s), 7.5 (5H, t), 7.46 (1H, t), 7.39 (2H, dd), 7.35 (1H, dd), 7.31 (2H, t), 7.2 (1H, d), |
| | 7.16 (1H, d) |
| 61 | δ = 8.62(1H, s), 8.55(1H, d), 8.36(2H, d), 8.22(1H, d), 8.19(2H, d), 7.98(1H, d), 7.94(1H, |
| | d), 7.92(1H, d), 7.91(1H, d), 7.86(1H, dd), 7.81(1H, d), 7.8(1H, d), 7.74(1H, dd), 7.62(2H, |
| | dd), 7.58(3H, s), 7.54 (1H, t), 7.5 (7H, t), 7.39 (1H, dd), 7.35 (1H, dd), 7.31 (1H, t), 7.2 (2H, |
| | d), 7.16 (1H, d) |
| 65 | δ = 8.95(1H, s), 8.55(1H, d), 8.36(4H, d), 8.19(1H, d), 8.17(1H, d), 7.98(1H, d), 7.94(1H, |
| | d), 7.86(1H, d), 7.81(1H, d), 7.79(1H, dd), 7.58(2H, d), 7.56(1H, d), 7.54(1H, dd), 7.52(1H, dd), |
| | 7.5(9H, s), 7.46 (2H, t), 7.39 (1H, t), 7.35 (1H, dd), 7.31 (1H, dd), 7.2 (1H, t), 7.16 (1H, d) |
| 71 | δ = 8.95(1H, s), 8.55(1H, d), 8.36(4H, d), 8.19(1H, d), 8.17(1H, d), 7.98(1H, d), 7.94(1H, |
| | d), 7.86(1H, d), 7.81(1H, d), 7.79(1H, dd), 7.58(1H, d), 7.56(1H, d), 7.54(1H, dd), 7.52(1H, |
| | dd), 7.5(7H, s), 7.46 (1H, t), 7.39 (1H, t), 7.35 (1H, dd), 7.31 (1H, dd), 7.25 (4H, t), 7.2 (1H, |
| | d), 7.16 (1H, d) |
| 78 | δ = 8.55(1H, s), 8.36(4H, d), 8.19(1H, d), 7.98(1H, d), 7.96(2H, d), 7.94(1H, d), 7.92(1H, |
| | d), 7.91(1H, d), 7.86(1H, d), 7.81(1H, dd), 7.8(1H, d), 7.6(2H, d), 7.58(2H, dd), 7.54(1H, dd), |
| | 7.5(9H, s), 7.46 (1H, t), 7.39 (1H, t), 7.35 (1H, dd), 7.31 (1H, dd), 7.2 (1H, t), 7.16 (1H, d) |
| 85 | δ = 8.55(1H, s), 8.36(4H, d), 7.99(1H, d), 7.98(1H, d), 7.94(1H, d), 7.92(2H, d), 7.91(2H, |
| | d), 7.89(1H, d), 7.86(1H, d), 7.81(1H, dd), 7.77(1H, d), 7.75(2H, d), 7.54(1H, dd), 7.5(8H, |
| | dd), 7.49(2H, s), 7.39 (1H, t), 7.35 (1H, t), 7.31 (1H, dd), 7.16 (1H, dd) |
| 88 | δ = 8.36(4H, s), 8.3(1H, d), 8.21(1H, d), 8.13(1H, d), 7.99(1H, d), 7.98(1H, d), 7.89(2H, d), |
| | 7.86(1H, d), 7.81(1H, d), 7.77(1H, dd), 7.75(4H, d), 7.68(1H, d), 7.6(1H, dd), 7.54(1H, dd), |
| | 7.5(6H, s), 7.49 (4H, t), 7.47 (1H, t), 7.39 (1H, dd), 7.31 (1H, dd) |
| 97 | δ = 8.38(1H, s), 8.36(2H, d), 7.98(1H, d), 7.97(1H, d), 7.96(1H, d), 7.94(2H, d), 7.86(1H, |
| | d), 7.81(1H, d), 7.75(2H, d), 7.73(1H, dd), 7.61(1H, d), 7.58(1H, d), 7.54(1H, dd), 7.5(4H, |
| | dd), 7.49(3H, s), 7.41 (1H, t), 7.39 (1H, t), 7.31 (1H, dd) |
| 100 | δ = 8.55(1H, s), 8.21(1H, d), 8.19(1H, d), 7.98(1H, d), 7.96(2H, d), 7.94(1H, d), 7.86(1H, |
| | d), 7.81(1H, d), 7.68(1H, d), 7.6(3H, dd), 7.58(1H, d), 7.54(1H, d), 7.52(4H, dd), 7.5(1H, |
| | dd), 7.47(1H, s), 7.39 (1H, t), 7.35 (1H, t), 7.31 (1H, dd), 7.2 (1H, dd), 7.16 (1H, t) |
| 128 | δ = 9.09(1H, s), 8.97(1H, d), 8.55(1H, d), 8.49(1H, d), 8.25(1H, d), 8.21(1H, d), 8.19(1H, |
| | d), 8.16(1H, d), 8.15(1H, d), 8.1(1H, dd), 8.08(1H, d), 8.07(1H, d), 8.01(1H, dd), 8(2H, dd), |
| | 7.98(1H, s), 7.94 (1H, t), 7.68 (1H, t), 7.61 (1H, dd), 7.6 (1H, dd), 7.59 (2H, t), 7.58 (2H, d), |
| | 7.54 (1H, d), 7.5 (3H, d) |
| 163 | δ = 8.55(2H, s), 8.36(2H, d), 8.19(2H, d), 8.07(1H, d), 8.01(1H, d), 7.98(1H, d), 7.94(2H, |
| | d), 7.92(1H, d), 7.91(1H, d), 7.8(1H, dd), 7.58(3H, d), 7.54(1H, d), 7.5(7H, dd), 7.46(1H, |
| | dd), 7.39(1H, s), 7.35 (2H, t), 7.31 (1H, t), 7.2 (2H, dd), 7.16 (2H, dd) |
| 166 | δ = 8.55(1H, s), 8.36(4H, d), 8.19(1H, d), 8.07(1H, d), 8.01(2H, d), 7.99(1H, d), 7.98(1H, |
| | d), 7.94(1H, d), 7.89(1H, d), 7.58(1H, dd), 7.55(1H, d), 7.54(1H, d), 7.5(11H, dd), 7.39(1H, |
| | dd), 7.38(2H, s), 7.35 (1H, t), 7.31 (1H, t), 7.2 (1H, dd), 7.16 (1H, dd) |
| 179 | δ = 8.55(1H, s), 8.36(4H, d), 8.21(1H, d), 8.19(1H, d), 8.07(1H, d), 8.01(1H, d), 7.98(1H, |
| | d), 7.96(2H, d), 7.94(1H, d), 7.68(1H, dd), 7.6(3H, d), 7.58(1H, d), 7.54(1H, dd), 7.5(9H, |
| | dd), 7.47(1H, s), 7.39 (1H, t), 7.35 (1H, t), 7.31 (1H, dd), 7.2 (1H, dd), 7.16 (1H, t) |
| 181 | δ = 8.55(1H, s), 8.19(1H, d), 8.07(1H, d), 8.01(1H, d), 7.98(3H, d), 7.94(1H, d), 7.92(1H, |
| | d), 7.91(1H, d), 7.82(2H, d), 7.69(2H, dd), 7.58(2H, d), 7.57(2H, d), 7.54(3H, dd), 7.5(3H, |
| | dd), 7.39(3H, s), 7.35 (1H, t), 7.31 (3H, t), 7.2 (1H, dd), 7.16 (1H, dd) |
| 206 | δ = 8.97(1H, s), 8.55(1H, d), 8.36(2H, d), 8.25(1H, d), 8.19(1H, d), 8.15(1H, d), 8.1(1H, d), |
| | 8.07(1H, d), 8(1H, d), 7.98(1H, dd), 7.94(1H, d), 7.92(2H, d), 7.91(2H, dd), 7.89(1H, dd), |
| | 7.59(1H, s), 7.58 (1H, t), 7.54 (1H, t), 7.52 (1H, dd), 7.5 (4H, dd), 7.39 (1H, t), 7.35 (1H, d), |
| | 7.31 (1H, d), 7.2 (1H, d) |
| 231 | δ = 8.55(1H, s), 8.21(1H, d), 8.19(1H, d), 8.09(1H, d), 8.07(1H, d), 7.98(2H, d), 7.94(1H, |
| | d), 7.9(1H, d), 7.89(2H, d), 7.82(1H, dd), 7.78(1H, d), 7.69(1H, d), 7.6(1H, dd), 7.58(1H, |
| | dd), 7.57(1H, s), 7.55 (1H, t), 7.54 (2H, t), 7.5 (1H, dd), 7.47 (1H, dd), 7.39 (2H, t), 7.38 (1H, |
| | d), 7.35 (1H, d), 7.31 (2H, d) |
| 250 | δ = 8.55(1H, s), 8.19(1H, d), 8.07(1H, d), 7.98(1H, d), 7.96(4H, d), 7.94(1H, d), 7.92(1H, |
| | d), 7.91(1H, d), 7.89(1H, d), 7.75(4H, dd), 7.58(1H, d), 7.54(1H, d), 7.5(1H, dd), 7.49(4H, |
| | dd), 7.39(1H, s), 7.35 (1H, t), 7.31 (1H, t), 7.25 (4H, dd), 7.2 (1H, dd), 7.16 (1H, t) |
| 267 | δ = 8.55(1H, s), 8.36(4H, d), 8.19(1H, d), 8.09(1H, d), 8.07(1H, d), 7.99(1H, d), 7.98(1H, |
| | d), 7.94(1H, d), 7.89(1H, d), 7.81(1H, dd), 7.58(1H, d), 7.55(1H, d), 7.54(1H, dd), 7.5(11H, dd), |
| | 7.39(1H, s), 7.38 (1H, t), 7.37 (1H, t), 7.35 (1H, dd), 7.31 (1H, dd), 7.2 (1H, t), 7.16 (1H, d) |
| 271 | δ = 8.95(1H, s), 8.55(1H, d), 8.36(4H, d), 8.19(1H, d), 8.17(1H, d), 8.07(1H, d), 7.98(1H, |
| | d), 7.94(2H, d), 7.89(1H, d), 7.79(1H, dd), 7.73(1H, d), 7.61(2H, d), 7.58(1H, dd), 7.56(1H, |
| | dd), 7.54(1H, s), 7.52 (1H, t), 7.5 (7H, t), 7.46 (1H, dd), 7.39 (1H, dd), 7.35 (1H, t), 7.31 (1H, |
| | d), 7.2 (1H, d), 7.16 (1H, d) |
| 282 | δ = 8.36(4H, s), 8.3(1H, d), 8.21(1H, d), 8.13(1H, d), 8.07(1H, d), 8.01(1H, d), 7.98(1H, d), |
| | 7.91(1H, d), 7.89(1H, d), 7.79(2H, dd), 7.75(2H, d), 7.68(2H, d), 7.6(1H, dd), 7.56(1H, dd), |
| | 7.54(1H, s), 7.5 (6H, t), 7.49 (2H, t), 7.47 (1H, dd), 7.46 (2H, dd), 7.39 (1H, t), 7.31 (1H, d) |
| 283 | δ = 8.62(1H, s), 8.36(4H, d), 8.31(1H, d), 8.22(1H, d), 7.98(1H, d), 7.92(2H, d), 7.91(3H, |
| | d), 7.86(1H, d), 7.81(1H, d), 7.75(4H, dd), 7.74(2H, d), 7.54(1H, d), 7.5(6H, dd), 7.49(4H, |
| | dd), 7.39(1H, s), 7.31 (1H, t) |
| 284 | δ = 8.55(1H, s), 8.36(4H, d), 8.29(1H, d), 8.21(1H, d), 8.06(1H, d), 7.98(1H, d), 7.94(1H, |
| | d), 7.86(1H, d), 7.81(1H, d), 7.68(1H, dd), 7.6(1H, d), 7.54(1H, d), 7.5(8H, dd), 7.48(1H, |
| | dd), 7.47(1H, s), 7.39 (1H, t), 7.35 (1H, t), 7.31 (1H, dd), 7.19 (4H, dd), 7.16 (1H, t) |
| 285 | δ = 8.36(4H, s), 8.22(1H, d), 8.07(1H, d), 8.04(1H, d), 7.98(1H, d), 7.92(2H, d), 7.91(3H, |
| | d), 7.89(1H, d), 7.79(4H, d), 7.68(1H, dd), 7.56(1H, d), 7.54(1H, d), 7.52(1H, dd), 7.5(6H, |
| | dd), 7.46(4H, s), 7.39 (1H, t), 7.31 (1H, t) |
| 286 | δ = 8.55(1H, s), 8.36(4H, d), 7.98(1H, d), 7.94(1H, d), 7.92(2H, d), 7.91(3H, d), 7.86(1H, |
| | d), 7.81(1H, d), 7.79(2H, d), 7.68(1H, dd), 7.56(1H, d), 7.54(1H, d), 7.5(8H, dd), 7.46(2H, |
| | dd), 7.39(1H, s), 7.35 (1H, t), 7.31 (1H, t), 7.16 (1H, dd) |
| 291 | δ = 8.55(2H, s), 8.36(4H, d), 8.26(1H, d), 8.21(1H, d), 8.19(1H, d), 7.98(1H, d), 7.94(2H, |
| | d), 7.86(1H, d), 7.81(1H, d), 7.68(1H, dd), 7.6(1H, d), 7.58(1H, d), 7.54(1H, dd), 7.52(1H, |
| | dd), 7.5(7H, s), 7.47 (1H, t), 7.39 (1H, t), 7.35 (2H, dd), 7.31 (1H, dd), 7.3 (1H, t), 7.2 (1H, |
| | d), 7.16 (2H, d) |
| 294 | δ = 8.62(1H, s), 8.36(4H, d), 8.22(1H, d), 7.98(1H, d), 7.92(2H, d), 7.91(3H, d), 7.86(1H, |
| | d), 7.81(1H, d), 7.79(2H, d), 7.75(2H, dd), 7.74(1H, d), 7.68(1H, d), 7.56(1H, dd), 7.54(1H, |
| | dd), 7.5(6H, s), 7.49 (2H, t), 7.46 (2H, t), 7.39 (1H, dd), 7.31 (1H, dd) |
| 2-1 | δ = 8.55(d, 1H), 8.30(d, 1H), 8.19-8.13(m, 2H), 7.99-7.89(m, 4H), 7.77(d, 1H), 7.62- |
| | 7.50(m, 12H), 7.35(t, 1H), 7.20-7.16(m, 2H) |
| 2-2 | δ = 8.55(d, 1H), 8.30(d, 1H), 8.19-8.13(m, 2H), 7.99-7.89(m, 6H), 7.80-7.77(m, 2H), 7.62- |
| | 7.35(m, 10H), 7.20-7.16(m, 6H) |
| 2-3 | δ = 8.55(d, 1H), 8.18-8.09(m, 3H), 8.00-7.87(m, 3H), 7.77(s, 2H), 7.58-7.25(m, 18H) |
| 2-27 | δ = 8.55(m, 1H), 8.30(d, 1H), 8.21-8.13(m, 4h), 7.99-7.89(m, 4H), 7.77-7.35(m, 20H), |
| | 7.20-7.16(2H) |
| 2-29 | δ = 8.55(m, 1H), 8.18-8.09(m, 3H), 8.00-7.94(m, 2H0, 7.87(m, 1H), 7.87(m, 1H), 7.79- |
| | 7.77(m, 4H), 7.69-7.63(m, 4H), 7.52-7.25(m, 21H) |
| 2-30 | δ = 8.55(m, 1H), 8.31-8.30(m, 3H), 8.21-8.13(m, 3h), 7.99-7.89(m, 3H), 7.75-7.35(m, |
| | 22H), 7.20-7.16(m, 2H) |
| 3-1 | δ = 8.55(2H, d), 7.94(2H, d), 7.62-7.35(14H, m), 7.16(2H, d) |
| 3-4 | δ = 8.55(2H, d), 7.94-7.91(10H, m), 7.75(4H, d), 7.49-7.35(10H, m), 7.16(2H, t) |
| 3-5 | δ = 8.55(2H, d), 8.21(1H, s), 7.94-7.91(6H, m), 7.75-7.35(16H, m), 7.26(1H, d), 7.16(2H, t) |
| 3-22 | δ = 8.55(1H, d), 8.19(1H, d), 7.94-7.91(9H, m), 7.75(4H, d), 7.58-7.35(11H, m), 7.20-7.16(2H, m) |
| 3-35 | δ = 8.55(1H, d), 8.21-8.19(2H, m), 7.94-7.91(5H, m), 7.68-7.35(18H, m), 7.20-7.16(2H, m) |
| 3-41 | δ = 8.55(2H, d), 7.94-7.91(10H, m), 7.84(2H, d), 7.75(4H, d), 7.49-7.35(8H, m), 7.16(2H, t) |

TABLE 6

| Compound | FD-MS |
|---|---|
| 1 | m/z = 640.75(C45H28N4O = 640.23) |
| 3 | m/z = 716.84(C51H32N4O = 716.26) |
| 14 | m/z = 730.83(C51H30N4O2 = 730.24) |
| 22 | m/z = 716.84(C51H32N4O = 716.26) |
| 35 | m/z = 746.89(C51H30N4OS = 746.21) |
| 39 | m/z = 756.91(C54H36N4O = 756.29) |
| 44 | m/z = 716.84(C51H32N4O = 716.26) |
| 45 | m/z = 690.81(C49H30N4O = 690.24) |
| 54 | m/z = 730.83(C51H30N4O2 = 730.24) |
| 61 | m/z = 805.94(C57H35N5O = 805.28) |
| 65 | m/z = 690.81(C49H30N4O = 690.24) |
| 71 | m/z = 766.90(C55H34N4O = 766.27) |
| 78 | m/z = 716.84(C51H32N4O = 716.26) |
| 85 | m/z = 716.84(C51H32N4O = 716.26) |
| 88 | m/z = 792.94(C57H36N4O = 792.29) |
| 95 | m/z = 668.92(C45D28N4O = 668.40) |
| 97 | m/z = 722.88(C51H26D6N4O = 722.30) |
| 100 | m/z = 722.88(C51H26D6N4O = 722.30) |
| 128 | m/z = 740.87(C53H32N4O = 740.26) |
| 163 | m/z = 729.84(C51H31N5O = 729.25) |
| 166 | m/z = 690.81(C49H30N4O = 690.24) |
| 179 | m/z = 716.84(C51H32N4O = 716.26) |
| 181 | m/z = 820.91(C57H32N4O3 = 820.25) |
| 194 | m/z = 761.43(C51D30N4O2 = 761.01) |
| 206 | m/z = 690.81(C49H30N4O = 690.24) |
| 231 | m/z = 846.99(C60H38N4O2 = 846.30) |
| 250 | m/z = 792.94(C57H36N4O = 792.29) |
| 267 | m/z = 690.81(C49H30N4O = 690.24) |
| 271 | m/z = 766.90(C55H34N4O = 766.27) |
| 277 | m/z = 749.04(C51D32N4O = 748.46) |
| 282 | m/z = 792.94(C57H36N4O = 792.29) |
| 283 | m/z = 792.94(C57H36N4O = 792.29) |
| 284 | m/z = 716.84(C51H32N4O = 716.26) |
| 285 | m/z = 792.94(C57H36N4O = 792.29) |
| 286 | m/z = 716.84(C51H32N4O = 716.26) |
| 291 | m/z = 805.94(C57H35N5O = 805.28) |
| 294 | m/z = 792.94(C57H36N4O = 792.29) |
| 2-1 | m/z = 484.59(C₃₆H₂₄N₂ = 484.19) |
| 2-2 | m/z = 560.69(C₄₂H₂₈N₂ = 560.23) |
| 2-3 | m/z = 560.69(C₄₂H₂₈N₂ = 560.23) |
| 2-27 | m/z = 712.88(C₅₄H₃₆N₂ = 712.29) |
| 2-29 | m/z = 712.88(C₅₄H₃₆N₂ = 712.29) |
| 2-30 | m/z = 712.88(C₅₄H₃₆N₂ = 712.29) |
| 2-54 | m/z = 668.99(C48D32N2 = 668.46) |
| 3-1 | m/z = 408.16(C₃₀H₂₀N₂ = 408.50) |
| 3-4 | m/z = 560.23(C₄₂H₂₈N₂ = 560.70) |
| 3-5 | m/z = 560.23(C₄₂H₂₈N₂ = 560.70) |
| 3-22 | m/z = 560.23(C₄₂H₂₈N₂ = 560.70) |
| 3-35 | m/z = 560.23(C₄₂H₂₈N₂ = 560.70) |
| 3-41 | m/z = 560.23(C₄₂H₂₈N₂ = 560.70) |
| 3-64 | m/z = 668.99(C48D32N2 = 668.46) |

### Experimental Example 1

### 1) Manufacture of Organic Light-Emitting Device

A glass substrate coated with an ITO film having a thickness of 1,500 Å was ultrasonically washed in distilled water. After the washing with distilled water, the substrate was ultrasonically cleaned with solvents such as acetone, methanol, and isopropyl alcohol, dried, and then subjected to UVO treatment for 5 minutes in a UV cleaner. The substrate was subsequently transferred to a plasma cleaner (PT) and plasma-treated under vacuum to adjust the ITO work function and remove residual films. The substrate was then transferred to a thermal evaporation apparatus for organic deposition.

A common layer, a hole injection layer (2-TNATA) (4,4′,4″-tris[2-naphthyl(phenyl)amino]triphenylamine) and a hole transport layer (NPB) (N,N′-di(1-naphthyl)-N,N′-diphenyl-(1,1′-biphenyl)-4,4′-diamine) were formed on the ITO transparent electrode (anode).

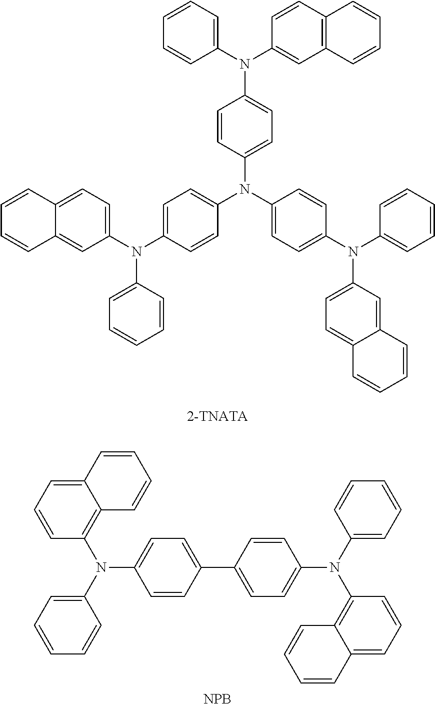 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

A light-emitting layer was thermally vacuum-deposited on the hole transport layer as follows. The light-emitting layer used a heterocyclic compound of Formula 1 as a host material and Ir(ppy)₃ (tris(2-phenylpyridine) iridium) as a green phosphorescent dopant, and 7 wt % of Ir(ppy)₃ was doped into the host and deposited to a thickness of 360 Å. Thereafter, BCP was deposited to a thickness of 60 Å as a hole blocking layer, and Alq₃ was deposited to a thickness of 200 Å thereon as an electron transport layer. Finally, lithium fluoride (LiF) was deposited to a thickness of 10 Å on the electron transport layer to form an electron injection layer, and an aluminum (Al) cathode was then deposited to a thickness of 1200 Å on the electron injection layer to form a cathode, thereby manufacturing an organic electroluminescent device.

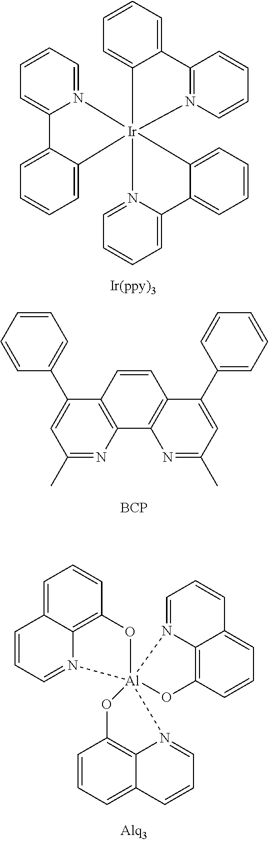

Meanwhile, all organic compounds required for manufacture of the OLED device were purified by vacuum sublimation at pressures of 108 to 106 torr for each material and were then used in OLED manufacture.

### 2) Operating Voltage and Light-Emitting Efficiency of the Organic Electroluminescent Device

The electroluminescence (EL) characteristics of the organic electroluminescent devices manufactured as described above were measured using an I-V-L meter (Current-Voltage-Luminance: M7000, MacScience, Korea). Based on these measurements, Too was determined using a lifetime measurement system (M6000, MacScience, Korea) at a reference luminance of 6,000 cd/m². The results of measuring the operating voltage, light-emitting efficiency, color coordinates (CIE), and lifetime of the organic light-emitting devices manufactured according to the present invention are shown in Table 7 below. Here, To refers to the lifetime (unit: h, hours), which is the time required for the initial luminance to decrease to 90%.

TABLE 7

| | | | Light- | | |
|---|---|---|---|---|---|
| | | Operating | emitting | Color | Life- |
| | Com- | voltage | efficiency | coordinates | time |
| | pound | (V) | (cd/A) | (x, y) | (T₉₀) |
| Example 1 | 1 | 4.08 | 63.4 | (0.234, 0.704) | 91 |
| Example 2 | 3 | 4.01 | 63.8 | (0.233, 0.715) | 93 |
| Example 3 | 14 | 4.11 | 61.2 | (0.242, 0.692) | 89 |
| Example 4 | 22 | 4.06 | 64.9 | (0.250, 0.724) | 91 |
| Example 5 | 35 | 4.11 | 63.5 | (0.242, 0.713) | 87 |
| Example 6 | 39 | 3.98 | 63.9 | (0.243, 0.712) | 82 |
| Example 7 | 44 | 4.07 | 64.3 | (0.241, 0.711) | 92 |
| Example 8 | 45 | 4.02 | 65.7 | (0.243, 0.717) | 85 |
| Example 9 | 54 | 4.07 | 62.2 | (0.246, 0.717) | 93 |
| Example 10 | 61 | 4.13 | 64.4 | (0.233, 0.701) | 95 |
| Example 11 | 65 | 4.20 | 62.5 | (0.251, 0.713) | 87 |
| Example 12 | 71 | 4.15 | 61.8 | (0.246, 0.717) | 83 |
| Example 13 | 78 | 4.21 | 63.0 | (0.254, 0.724) | 91 |
| Example 14 | 85 | 4.01 | 62.6 | (0.233, 0.703) | 93 |
| Example 15 | 88 | 4.02 | 62.2 | (0.234, 0.706) | 95 |
| Example 16 | 95 | 4.06 | 63.8 | (0.250, 0.724) | 122 |
| Example 17 | 97 | 4.08 | 62.9 | (0.246, 0.717) | 108 |
| Example 18 | 100 | 4.08 | 63.4 | (0.231, 0.711) | 99 |
| Example 19 | 128 | 4.11 | 63.8 | (0.251, 0.713) | 87 |
| Example 20 | 163 | 4.18 | 64.3 | (0.245, 0.716) | 92 |
| Example 21 | 166 | 4.13 | 62.8 | (0.242, 0.713) | 85 |
| Example 22 | 179 | 4.15 | 63.1 | (0.250, 0.724) | 97 |
| Example 23 | 181 | 4.13 | 62.2 | (0.242, 0.713) | 94 |
| Example 24 | 194 | 4.13 | 62.4 | (0.672, 0.320) | 117 |
| Example 25 | 206 | 4.11 | 63.3 | (0.243, 0.693) | 92 |
| Example 26 | 231 | 4.07 | 64.1 | (0.231, 0.711) | 86 |
| Example 27 | 250 | 4.06 | 62.8 | (0.242, 0.713) | 83 |
| Example 28 | 267 | 4.07 | 63.7 | (0.233, 0.703) | 84 |
| Example 29 | 271 | 4.10 | 61.4 | (0.233, 0.701) | 88 |
| Example 30 | 277 | 4.07 | 63.1 | (0.248, 0.715) | 106 |
| Example 31 | 282 | 4.03 | 62.1 | (0.234, 0.706) | 91 |
| Example 32 | 283 | 4.00 | 61.8 | (0.233, 0.701) | 86 |
| Example 33 | 284 | 4.02 | 62.4 | (0.251, 0.713) | 97 |
| Example 34 | 285 | 4.10 | 61.5 | (0.254, 0.724) | 95 |
| Example 35 | 286 | 4.04 | 62.6 | (0.233, 0.703) | 91 |
| Example 36 | 291 | 4.01 | 61.0 | (0.234, 0.714) | 99 |
| Example 37 | 294 | 4.15 | 60.8 | (0.248, 0.715) | 87 |
| Comparative | GH-1 | 4.84 | 46.3 | (0.254, 0.724) | 34 |
| Example 1 | | | | | |
| Comparative | GH-2 | 4.76 | 52.9 | (0.233, 0.703) | 49 |
| Example 2 | | | | | |
| Comparative | GH-3 | 4.82 | 49.2 | (0.234, 0.714) | 42 |
| Example 3 | | | | | |
| Comparative | GH-4 | 4.91 | 45.6 | (0.248, 0.715) | 33 |
| Example 4 | | | | | |
| Comparative | GH-5 | 4.95 | 45.4 | (0.251, 0.714) | 27 |
| Example 5 | | | | | |
| Comparative | GH-6 | 4.83 | 48.7 | (0.243, 0.712) | 32 |
| Example 6 | | | | | |
| Comparative | GH-7 | 4.94 | 46.2 | (0.242, 0.716) | 30 |
| Example 7 | | | | | |

The compounds used in the comparative examples in Table 7 are as follows:

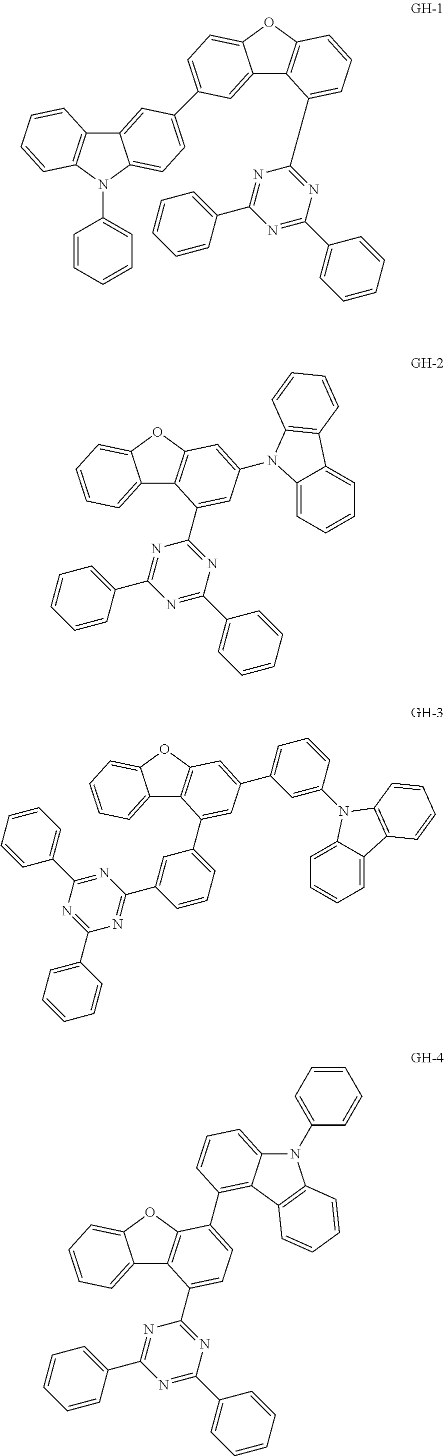 [SMILES: c1ccc(-c2nc(-c3ccccc3)nc(-c3cccc4oc5ccc(-c6ccc7c(c6)c6ccccc6n7-c6ccccc6)cc5c34)n2)cc1 ; c1ccc(-c2nc(-c3ccccc3)nc(-c3cc(-n4c5ccccc5c5ccccc54)cc4oc5ccccc5c34)n2)cc1 ; c1ccc(-c2nc(-c3ccccc3)nc(-c3cccc(-c4cc(-c5cccc(-n6c7ccccc7c7ccccc76)c5)cc5oc6ccccc6c45)c3)n2)cc1 ; c1ccc(-c2nc(-c3ccccc3)nc(-c3ccc(-c4cccc5c4c4ccccc4n5-c4ccccc4)c4oc5ccccc5c34)n2)cc1]

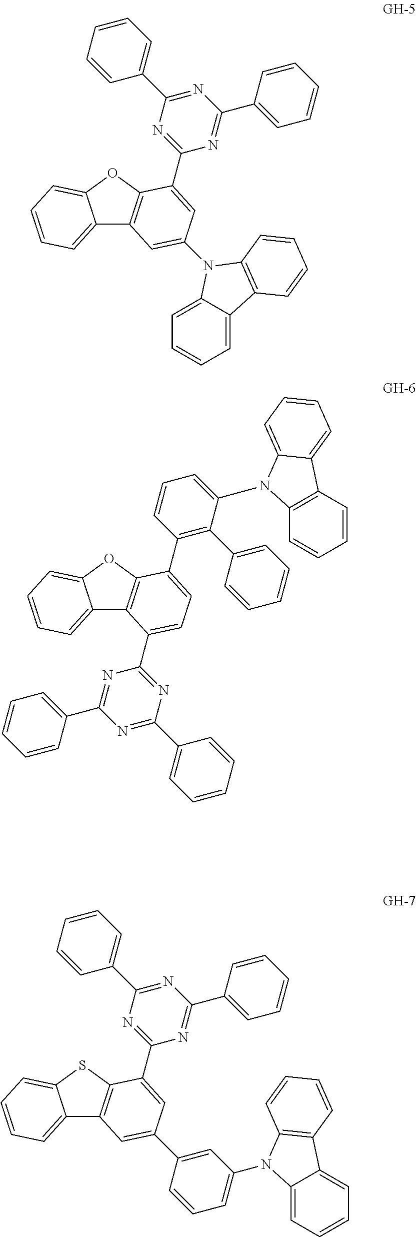 [SMILES: c1ccc(-c2nc(-c3ccccc3)nc(-c3cc(-n4c5ccccc5c5ccccc54)cc4c3oc3ccccc34)n2)cc1 ; c1ccc(-c2nc(-c3ccccc3)nc(-c3ccc(-c4cccc(-n5c6ccccc6c6ccccc65)c4-c4ccccc4)c4oc5ccccc5c34)n2)cc1 ; c1ccc(-c2nc(-c3ccccc3)nc(-c3cc(-c4cccc(-n5c6ccccc6c6ccccc65)c4)cc4c3sc3ccccc34)n2)cc1]

The heterocyclic compound of Formula 1 of the present invention has an appropriate molecular weight and band gap. Such an appropriate band gap in the light-emitting layer prevents the loss of electrons and holes, and thereby facilitates the formation of an effective recombination zone. As shown in Table 7, the organic light-emitting device employing the compound of Formula 1 exhibited improved performance compared to the devices employing the compounds of Comparative Examples 1 to 7. This improvement is attributed to the fact that substitution of the triazine at the 1-position of the dibenzofuran provides more suitable electron mobility than substitution at other positions, as confirmed from the operating characteristics of Comparative Examples 5 and 7. In Comparative Example 3, the triazine is not directly bonded but is substituted through a phenyl linker, which slows electron mobility and results in inferior performance relative to the examples. In Comparative Examples 1 and 4, the carbazole moiety is bonded through the carbazole ring rather than toward the nitrogen direction, and in such cases, the hole mobility becomes slower than when substitution occurs toward the nitrogen, resulting in lower performance. In Comparative Example 2, the carbazole is directly bonded, causing excessively fast hole mobility. In Comparative

Example 6, the phenyl group additionally substituted on the carbazole-substituted phenyl ring slows hole mobility, again resulting in lower performance relative to the examples.

Furthermore, in Examples 16, 17, 18, 24, and 30, replacing all or at least a portion of the hydrogen atoms with deuterium was found to increase device lifetime characteristics to varying degrees. This is believed to result from the fact that deuterium has twice the mass of hydrogen, leading to a lower vibrational energy and consequently a lower molecular energy, thus providing greater molecular stability. Since the bond dissociation energy of a carbon-deuterium bond is higher than that of a carbon-hydrogen bond, the molecule becomes further stabilized, which is thought to contribute to improved lifetime.

Referring to Table 7, the organic light-emitting devices incorporating the heterocyclic compound of the present disclosure exhibited lower operating voltages, higher light-emitting efficiencies, and improved lifetimes compared to the organic light-emitting devices of the comparative examples.

The organic light-emitting devices of the examples exhibited a Too lifetime of 82 or more, an operating voltage of 4.21 V or less, and a light-emitting efficiency of 60.8 cd/A or more, thereby demonstrating improved driving efficiency. In contrast, the organic light-emitting devices of the comparative examples exhibited a T90 lifetime of 49 or less, an operating voltage of 4.76 V or more, and a light-emitting efficiency of 52.9 cd/A or less, resulting in inferior efficiency compared to the organic light-emitting devices of the examples.

### Experimental Example 2

### 1) Manufacture of Organic Light-Emitting Device

A glass substrate coated with an ITO film having a thickness of 1,500 Å was ultrasonically washed in distilled water. After the washing with distilled water, the substrate was ultrasonically cleaned with solvents such as acetone, methanol, and isopropyl alcohol, dried, and then subjected to UVO treatment for 5 minutes in a UV cleaner. The substrate was subsequently transferred to a plasma cleaner (PT) and plasma-treated under vacuum to adjust the ITO work function and remove residual films. The substrate was then transferred to a thermal evaporation apparatus for organic deposition.

A common layer, a hole injection layer (2-TNATA) (4,4′,4″-tris[2-naphthyl(phenyl)amino]triphenylamine) and a hole transport layer (NPB) (N,N′-di(1-naphthyl)-N,N′-diphenyl-(1,1′-biphenyl)-4,4′-diamine) were formed on the ITO transparent electrode (anode).

A light-emitting layer was thermally vacuum-deposited on the hole transport layer as follows. The light-emitting layer was prepared by premixing one heterocyclic compound of Formula 1 as a host with one compound of each of Formulae 4 and 5, and the mixture was co-deposited from a single source to a thickness of 360 Å. The green phosphorescent dopant Ir(ppy)₃ was deposited by doping it at 7% of the deposition thickness of the light-emitting layer. Subsequently, BCP was deposited to a thickness of 60 Å as the hole blocking layer, and Alq₃ was deposited to a thickness of 200 Å as the electron transport layer.

Finally, lithium fluoride (LiF) was deposited to a thickness of 10 Å on the electron transport layer to form an electron injection layer, and an aluminum (Al) cathode was deposited to a thickness of 1,200 Å on the electron injection layer to form the cathode, thereby manufacturing an organic electroluminescent device.

Meanwhile, all organic compounds required for manufacture of the OLED device were purified by vacuum sublimation at pressures of 10-8 to 106 torr for each material and were then used in OLED manufacture.

2) Operating Voltage and Light-Emitting Efficiency of the Organic Electroluminescent Device

The electroluminescence (EL) characteristics of the organic electroluminescent devices manufactured as described above were measured using an I-V-L meter (Current-Voltage-Luminance: M7000, MacScience, Korea). Based on these measurements, T90 was determined using a lifetime measurement system (M6000, MacScience, Korea) at a reference luminance of 6,000 cd/m².

The results of measuring the operating voltage, light-emitting efficiency, color coordinates (CIE), and lifetime of the organic light-emitting devices manufactured according to the present invention are shown in Table 8 below.

TABLE 8

| | | | | Light- | | |
|---|---|---|---|---|---|---|
| | Light-emitting | | Operating | emitting | | |
| | layer | | voltage | efficiency | Color | Lifetime |
| | Compound | Ratio | (V) | (cd/A) | coordinates (x, y) | (T₉₀) |
| Example 38 | 1:2-1 | 1:1 | 3.77 | 68.5 | (0.235, 0.655) | 128 |
| Example 39 | | 1:2 | 3.80 | 68.1 | (0.236, 0.624) | 136 |
| Example 40 | | 1:3 | 3.83 | 67.9 | (0.255, 0.692) | 139 |
| Example 41 | 3:2-2 | 1:1 | 3.71 | 70.1 | (0.235, 0.655) | 131 |
| Example 42 | | 1:2 | 3.75 | 68.3 | (0.236, 0.624) | 138 |
| Example 43 | | 1:3 | 3.80 | 67.1 | (0.255, 0.692) | 142 |
| Example 44 | 14:2-3 | 1:1 | 3.82 | 66.2 | (0.253, 0.724) | 128 |
| Example 45 | | 1:2 | 3.88 | 65.1 | (0.242, 0.625) | 131 |
| Example 46 | | 1:3 | 3.91 | 64.8 | (0.261, 0.623) | 139 |
| Example 47 | 22:3-1 | 1:1 | 3.75 | 70.7 | (0.253, 0.724) | 125 |
| Example 48 | | 1:2 | 3.81 | 69.1 | (0.242, 0.625) | 127 |
| Example 49 | | 1:3 | 3.83 | 68.4 | (0.261, 0.623) | 130 |
| Example 50 | 35:2-27 | 1:1 | 3.81 | 68.5 | (0.253, 0.614) | 123 |
| Example 51 | | 1:2 | 3.83 | 66.3 | (0.254, 0.659) | 129 |
| Example 52 | | 1:3 | 3.87 | 65.9 | (0.255, 0.635) | 131 |
| Example 53 | 39:2-29 | 1:1 | 3.69 | 70.3 | (0.253, 0.614) | 116 |
| Example 54 | | 1:2 | 3.71 | 68.2 | (0.254, 0.659) | 128 |
| Example 55 | | 1:3 | 3.74 | 67.9 | (0.251, 0.693) | 134 |
| Example 56 | 44:3-4 | 1:1 | 3.76 | 69.2 | (0.245, 0.677) | 129 |
| Example 57 | | 1:2 | 3.79 | 68.3 | (0.258, 0.647) | 134 |
| Example 58 | | 1:3 | 3.83 | 67.7 | (0.266, 0.645) | 137 |
| Example 59 | 45:2-30 | 1:1 | 3.72 | 71.6 | (0.245, 0.677) | 119 |
| Example 60 | | 1:2 | 3.74 | 69.1 | (0.258, 0.647) | 124 |
| Example 61 | | 1:3 | 3.76 | 67.9 | (0.266, 0.645) | 129 |
| Example 62 | 54:3-5 | 1:1 | 3.78 | 67.2 | (0.256, 0.673) | 131 |
| Example 63 | | 1:2 | 3.81 | 66.5 | (0.237, 0.644) | 135 |
| Example 64 | | 1:3 | 3.84 | 65.9 | (0.237, 0.624) | 137 |
| Example 65 | 61:2-54 | 1:1 | 3.82 | 70.8 | (0.256, 0.673) | 176 |
| Example 66 | | 1:2 | 3.84 | 69.7 | (0.237, 0.644) | 179 |
| Example 67 | | 1:3 | 3.85 | 68.1 | (0.237, 0.624) | 182 |
| Example 68 | 65:3-22 | 1:1 | 3.92 | 68.1 | (0.245, 0.617) | 128 |
| Example 69 | | 1:2 | 3.95 | 67.6 | (0.257, 0.624) | 131 |
| Example 70 | | 1:3 | 3.96 | 67.1 | (0.259, 0.712) | 133 |
| Example 71 | 71 :2-30 | 1:1 | 3.84 | 67.3 | (0.245, 0.677) | 116 |
| Example 72 | | 1:2 | 3.86 | 65.0 | (0.258, 0.647) | 121 |
| Example 73 | | 1:3 | 3.88 | 63.9 | (0.266, 0.645) | 126 |
| Example 74 | 85:3-35 | 1:1 | 3.70 | 67.6 | (0.245, 0.617) | 131 |
| Example 75 | | 1:2 | 3.72 | 67.1 | (0.257, 0.624) | 138 |
| Example 76 | | 1:3 | 3.75 | 66.3 | (0.259, 0.712) | 140 |
| Example 77 | 88:3-35 | 1:1 | 3.71 | 67.2 | (0.258, 0.628) | 134 |
| Example 78 | | 1:2 | 3.73 | 66.7 | (0.254, 0.653) | 141 |
| Example 79 | | 1:3 | 3.76 | 65.9 | (0.275, 0.657) | 143 |
| Example 80 | 95 :3-41 | 1:1 | 3.76 | 70.0 | (0.243, 0.643) | 172 |
| Example 81 | | 1:2 | 3.79 | 69.2 | (0.261, 0.764) | 174 |
| Example 82 | | 1:3 | 3.81 | 68.4 | (0.258, 0.628) | 181 |
| Example 83 | 97:3-64 | 1:1 | 3.79 | 67.9 | (0.243, 0.643) | 192 |
| Example 84 | | 1:2 | 3.81 | 67.0 | (0.261, 0.764) | 195 |
| Example 85 | | 1:3 | 3.84 | 66.4 | (0.258, 0.628) | 200 |
| Example 86 | 100:3-4 | 1:1 | 3.77 | 69.1 | (0.254, 0.653) | 139 |
| Example 87 | | 1:2 | 3.79 | 68.4 | (0.275, 0.657) | 143 |
| Example 88 | | 1:3 | 3.80 | 66.9 | (0.264, 0.642) | 148 |
| Example 89 | 128:2-27 | 1:1 | 3.81 | 68.9 | (0.254, 0.653) | 123 |
| Example 90 | | 1:2 | 3.84 | 67.6 | (0.275, 0.657) | 130 |
| Example 91 | | 1:3 | 3.87 | 66.2 | (0.264, 0.642) | 141 |
| Example 92 | 163:2-54 | 1:1 | 3.89 | 70.6 | (0.256, 0.638) | 165 |
| Example 93 | | 1:2 | 3.91 | 68.2 | (0.251, 0.632) | 171 |
| Example 94 | | 1:3 | 3.94 | 67.9 | (0.253, 0.684) | 178 |
| Example 95 | 166:2-30 | 1:1 | 3.82 | 68.4 | (0.253, 0.724) | 119 |
| Example 96 | | 1:2 | 3.84 | 66.0 | (0.242, 0.625) | 124 |
| Example 97 | | 1:3 | 3.86 | 64.9 | (0.261, 0.623) | 129 |
| Example 98 | 179:3-64 | 1:1 | 3.85 | 68.1 | (0.256, 0.638) | 172 |
| Example 99 | | 1:2 | 3.88 | 67.2 | (0.251, 0.632) | 175 |
| Example 100 | | 1:3 | 3.90 | 66.6 | (0.253, 0.724) | 180 |
| Example 101 | 181:3-35 | 1:1 | 3.82 | 67.6 | (0.256, 0.638) | 132 |
| Example 102 | | 1:2 | 3.84 | 66.2 | (0.251, 0.632) | 137 |
| Example 103 | | 1:3 | 3.85 | 65.9 | (0.253, 0.684) | 140 |
| Example 104 | 194:3-4 | 1:1 | 3.83 | 68.0 | (0.235, 0.655) | 164 |
| Example 105 | | 1:2 | 3.86 | 66.9 | (0.236, 0.624) | 168 |
| Example 106 | | 1:3 | 3.90 | 66.1 | (0.255, 0.692) | 179 |
| Example 107 | 206:3-4 | 1:1 | 3.82 | 68.3 | (0.235, 0.655) | 128 |
| Example 108 | | 1:2 | 3.85 | 67.2 | (0.236, 0.624) | 133 |
| Example 109 | | 1:3 | 3.87 | 66.7 | (0.255, 0.692) | 139 |
| Example 110 | 231:2-30 | 1:1 | 3.76 | 70.5 | (0.253, 0.724) | 122 |
| Example 111 | | 1:2 | 3.79 | 68.7 | (0.242, 0.625) | 129 |
| Example 112 | | 1:3 | 3.82 | 68.1 | (0.261, 0.623) | 131 |
| Example 113 | 250:3-4 | 1:1 | 3.76 | 67.8 | (0.253, 0.724) | 117 |
| Example 114 | | 1:2 | 3.78 | 66.4 | (0.242, 0.625) | 120 |
| Example 115 | | 1:3 | 3.82 | 65.4 | (0.261, 0.623) | 123 |
| Example 116 | 267:3-64 | 1:1 | 3.78 | 68.8 | (0.253, 0.614) | 149 |
| Example 117 | | 1:2 | 3.80 | 67.9 | (0.254, 0.659) | 152 |
| Example 118 | | 1:3 | 3.83 | 67.2 | (0.255, 0.635) | 156 |
| Example 119 | 271:2-30 | 1:1 | 3.79 | 66.9 | (0.235, 0.655) | 123 |
| Example 120 | | 1:2 | 3.81 | 64.6 | (0.236, 0.624) | 128 |
| Example 121 | | 1:3 | 3.83 | 63.5 | (0.235, 0.655) | 134 |
| Example 122 | 277:3-4 | 1:1 | 3.78 | 68.8 | (0.253, 0.614) | 149 |
| Example 123 | | 1:2 | 3.80 | 67.6 | (0.254, 0.659) | 152 |
| Example 124 | | 1:3 | 3.83 | 66.9 | (0.255, 0.635) | 153 |
| Example 125 | 282:3-35 | 1:1 | 3.72 | 67.1 | (0.243, 0.643) | 128 |
| Example 126 | | 1:2 | 3.74 | 66.6 | (0.261, 0.764) | 135 |
| Example 127 | | 1:3 | 3.77 | 65.8 | (0.258, 0.628) | 137 |
| Example 128 | 283:2-27 | 1:1 | 3.71 | 66.7 | (0.243, 0.643) | 122 |
| Example 129 | | 1:2 | 3.74 | 65.5 | (0.261, 0.764) | 129 |
| Example 130 | | 1:3 | 3.77 | 64.1 | (0.258, 0.628) | 135 |
| Example 131 | 284:3-35 | 1:1 | 3.71 | 67.4 | (0.236, 0.624) | 137 |
| Example 132 | | 1:2 | 3.73 | 66.9 | (0.255, 0.692) | 144 |
| Example 133 | | 1:3 | 3.76 | 66.1 | (0.253, 0.724) | 146 |
| Example 134 | 285:3-35 | 1:1 | 3.78 | 66.4 | (0.242, 0.625) | 134 |
| Example 135 | | 1:2 | 3.80 | 65.9 | (0.235, 0.655) | 141 |
| Example 136 | | 1:3 | 3.83 | 65.1 | (0.236, 0.624) | 143 |
| Example 137 | 286:2-27 | 1:1 | 3.75 | 67.6 | (0.255, 0.692) | 129 |
| Example 138 | | 1:2 | 3.77 | 66.3 | (0.235, 0.655) | 134 |
| Example 139 | | 1:3 | 3.80 | 65.0 | (0.236, 0.624) | 144 |
| Example 140 | 291:2-27 | 1:1 | 3.72 | 65.9 | (0.243, 0.643) | 140 |
| Example 141 | | 1:2 | 3.75 | 64.6 | (0.261, 0.764) | 148 |
| Example 142 | | 1:3 | 3.78 | 63.3 | (0.258, 0.628) | 151 |
| Example 143 | 294:3-35 | 1:1 | 3.83 | 65.7 | (0.242, 0.625) | 123 |
| Example 144 | | 1:2 | 3.85 | 65.2 | (0.261, 0.623) | 129 |
| Example 145 | | 1:3 | 3.88 | 64.4 | (0.253, 0.614) | 131 |
| Comparative | GH-1:3-4 | 1:1 | 4.53 | 50.0 | (0.263, 0.621) | 48 |
| Example 8 | | | | | | |
| Comparative | | 1:2 | 4.55 | 48.9 | (0.256, 0.670) | 55 |
| Example 9 | | | | | | |
| Comparative | | 1:3 | 4.57 | 48.3 | (0.245, 0.637) | 60 |
| Example 10 | | | | | | |
| Comparative | GH-2:2-1 | 1:1 | 4.46 | 58.1 | (0.263, 0.621) | 69 |
| Example 11 | | | | | | |
| Comparative | | 1:2 | 4.50 | 57.2 | (0.256, 0.670) | 74 |
| Example 12 | | | | | | |
| Comparative | | 1:3 | 4.58 | 56.7 | (0.245, 0.637) | 75 |
| Example 13 | | | | | | |
| Comparative | GH-3:2-27 | 1:1 | 4.53 | 53.1 | (0.257, 0.714) | 59 |
| Example 14 | | | | | | |
| Comparative | | 1:2 | 4.56 | 50.0 | (0.249, 0.666) | 61 |
| Example 15 | | | | | | |
| Comparative | | 1:3 | 4.58 | 49.7 | (0.253, 0.635) | 63 |
| Example 16 | | | | | | |
| Comparative | GH-4:3-5 | 1:1 | 4.61 | 49.3 | (0.257,0.714) | 46 |
| Example 17 | | | | | | |
| Comparative | | 1:2 | 4.64 | 48.0 | (0.249, 0.666) | 50 |
| Example 18 | | | | | | |
| Comparative | | 1:3 | 4.69 | 47.7 | (0.253, 0.635) | 51 |
| Example 19 | | | | | | |
| Comparative | GH-5:3-1 | 1:1 | 4.65 | 49.1 | (0.687, 0.643) | 38 |
| Example 20 | | | | | | |
| Comparative | | 1:2 | 4.70 | 48.7 | (0.267, 0.628) | 42 |
| Example 21 | | | | | | |
| Comparative | | 1:3 | 4.78 | 47.3 | (0.265, 0.624) | 48 |
| Example 22 | | | | | | |
| Comparative | GH-6:2-3 | 1:1 | 4.54 | 53.5 | (0.276, 0.613) | 45 |
| Example 23 | | | | | | |
| Comparative | | 1:2 | 4.59 | 52.1 | (0.259, 0.628) | 49 |
| Example 24 | | | | | | |
| Comparative | | 1:3 | 4.62 | 51.7 | (0.244, 0.628) | 50 |
| Example 25 | | | | | | |
| Comparative | GH-7:2-30 | 1:1 | 4.63 | 49.9 | (0.256, 0.723) | 42 |
| Example 26 | | | | | | |
| Comparative | | 1:2 | 4.68 | 49.1 | (0.243, 0.629) | 47 |
| Example 27 | | | | | | |
| Comparative | | 1:3 | 4.70 | 48.4 | (0.268, 0.734) | 48 |
| Example 28 | | | | | | |

The comparative compounds used in Table 8 are identical to those in Table 7.

Comparing the results in Table 8 with those in Table 7, it was confirmed that when the heterocyclic compound of Formula 1 and the compounds of Formulae 4 and 5 were simultaneously used as hosts in the light-emitting layer, the operating voltage, light-emitting efficiency, and lifetime were all improved.

This result suggests that an exciplex phenomenon may occur when the two compounds are used together. The exciplex phenomenon refers to the release of energy that corresponds to the HOMO level of the donor (p-host) and the LUMO level of the acceptor (n-host) through electron exchange between two molecules. When an exciplex occurs between two molecules, reverse intersystem crossing (RISC) can occur, which may increase the internal quantum efficiency of fluorescence up to 100%. When a donor (p-host) with good hole-transport capability and an acceptor (n-host) with good electron-transport capability are used together as hosts in the light-emitting layer, holes are injected into the p-host and electrons into the n-host, thereby lowering the operating voltage and contributing to lifetime enhancement. In the present invention, it was confirmed that excellent device characteristics were achieved when the compounds of Formulae 4 and 5 served as the donor and the compound of Formula 1 served as the acceptor in the light-emitting layer. In contrast, when compounds outside the scope of the present invention (Comparative Examples 8 to 28) were used in combination with the compounds of Formulae 4 and 5, the operating voltage, light-emitting efficiency, and lifetime were also improved, but remained inferior to those of the present invention.

It could be confirmed that when some of the hydrogens were replaced with deuterium, the lifetime characteristics were improved, consistent with the results described in Table 7. In particular, as observed in Examples 83 to 85, even better lifetime characteristics were obtained when both the n-host and the p-host were deuterated.

Referring to Table 8, the organic light-emitting devices incorporating the heterocyclic compound of the present disclosure exhibited lower operating voltages, higher light-emitting efficiencies, and improved lifetimes compared to the organic light-emitting devices of the comparative examples.

The organic light-emitting devices of the examples exhibited a T90 lifetime of 116 or more, an operating voltage of 3.96 V or less, and a light-emitting efficiency of 63.3 cd/A or more, demonstrating improved operating efficiency. In contrast, the organic light-emitting devices of the comparative examples exhibited a T90 lifetime of 75 or less, an operating voltage of 4.50 V or more, and a light-emitting efficiency of 58.1 cd/A or less, showing degraded efficiency compared to the organic light-emitting devices of the examples.

The contents described above are merely examples of applying the principles of the present disclosure, and other configurations may be further included without departing from the scope of the present disclosure.

What is claimed is:

1. A heterocyclic compound represented by Formula 1: 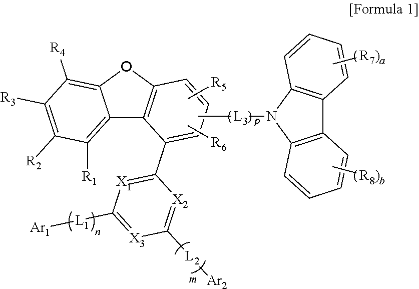
wherein X₁ to X₃ are each independently N or CR₉, and
R₁ to R₉ are each independently selected from the group consisting of hydrogen, deuterium, a halogen, a substituted or unsubstituted alkyl group having 1 to 60 carbon atoms, a substituted or unsubstituted alkenyl group having 2 to 60 carbon atoms, a substituted or unsubstituted alkynyl group having 2 to 60 carbon atoms, a substituted or unsubstituted alkoxy group having 1 to 60 carbon atoms, a substituted or unsubstituted cycloalkyl group having 3 to 60 carbon atoms, a substituted or unsubstituted heterocycloalkyl group having 2 to 60 carbon atoms, a substituted or unsubstituted aryl group having 6 to 60 carbon atoms, a substituted or unsubstituted heteroaryl group having 2 to 60 carbon atoms, —SiRR′R″, and —P(═O)RR′, wherein adjacent groups are linked to form a substituted or unsubstituted aryl group having 6 to 60 carbon atoms, a substituted or unsubstituted heterocycloalkyl group having 2 to 60 carbon atoms, or a substituted or unsubstituted heteroaryl group having 2 to 60 carbon atoms,
R, R′ and R″ are each independently hydrogen, deuterium, a cyano group (—CN), a substituted or unsubstituted alkyl group having 1 to 60 carbon atoms, a substituted or unsubstituted cycloalkyl group having 3 to 60 carbon atoms, a substituted or unsubstituted aryl group having 6 to 60 carbon atoms, or a substituted or unsubstituted heteroaryl group having 2 to 60 carbon atoms,
Ar₁ and Ar₂ are each independently a substituted or unsubstituted aryl group having 6 to 60 carbon atoms, or a substituted or unsubstituted heteroaryl group having 2 to 60 carbon atoms,
L₁ and L₂ are each independently a direct linkage, a substituted or unsubstituted arylene group having 6 to 60 carbon atoms, or a substituted or unsubstituted heteroarylene group having 2 to 60 carbon atoms,
L₃ is a substituted or unsubstituted arylene group having 6 to 60 carbon atoms, or a substituted or unsubstituted heteroarylene group having 2 to 60 carbon atoms, and
with a proviso that L₃ is not a group represented by Formula 2 below, 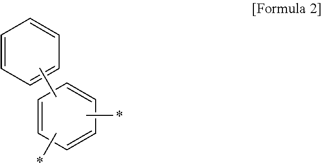
wherein n and m are each independently 0 or an integer of 1 to 5,
p is an integer of 1 to 5,
when n, m, and p are each independently 2 or more, a plurality of L₁ groups, L₂ groups, and L₃ groups are each independently the same or different,
a and b are each independently 0 or an integer of 1 to 4,
when a and b are each independently 2 or more, a plurality of R₇ groups and R₈ groups are each independently the same or different, and
* represents a bonding site.

2. The heterocyclic compound according to claim 1, wherein the heterocyclic compound is represented by any one of Formulae 1-1 to 1-3:

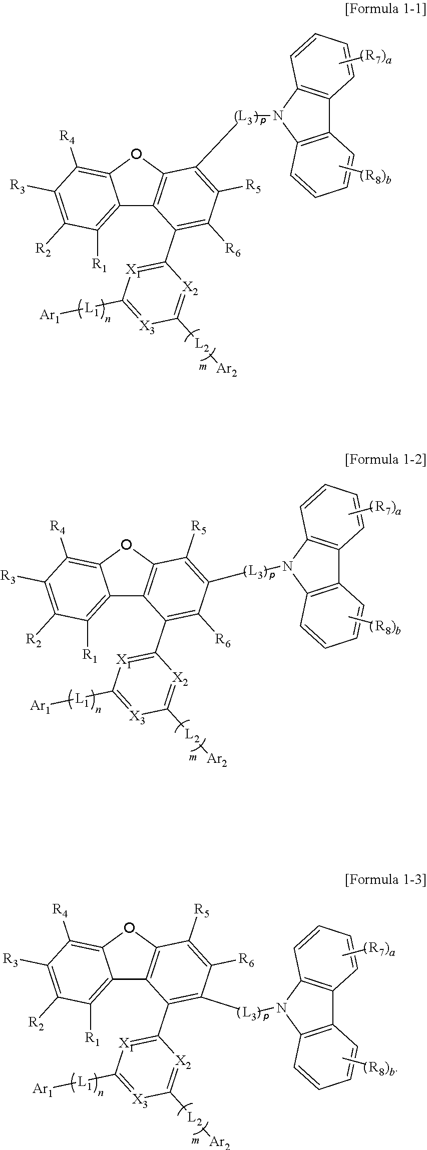

3. The heterocyclic compound according to claim 1, wherein the heterocyclic compound is represented by any one of Formulae 1-4 to 1-5: 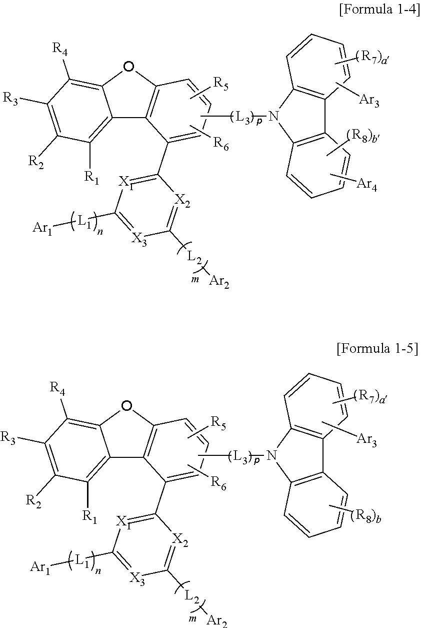
wherein, in Formulae 1-4 to 1-5, Ar₃ and Ar₄ are each independently a substituted or unsubstituted aryl group having 6 to 60 carbon atoms, or a substituted or unsubstituted heteroaryl group having 2 to 60 carbon atoms,
a′ and b′ are each independently 0 or an integer of 1 to 3, and
when a′ and b′ are each independently 2 or more, a plurality of R₇ groups and R₈ groups are each independently the same or different.

4. The heterocyclic compound according to claim 1, wherein in Formula 1, L₃ is represented by one of Formulae 3-1 to 3-8: 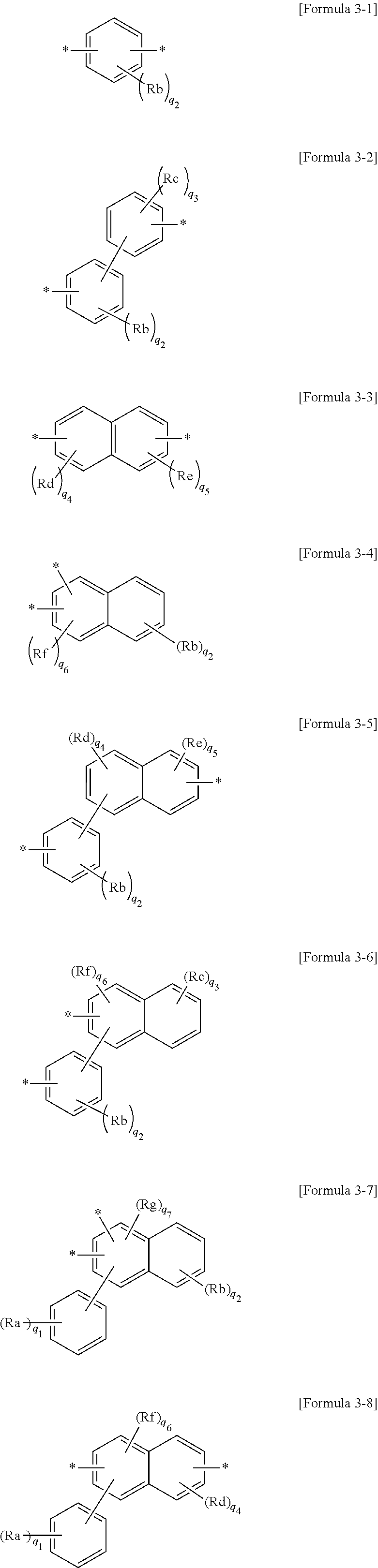
wherein, in Formulae 3-1 to 3-8, Rb is hydrogen or deuterium,
Ra, Rc, Rd, Re, Rf, and Rg are each independently selected from the group consisting of hydrogen, deuterium, a halogen, a substituted or unsubstituted alkyl group having 1 to 60 carbon atoms, a substituted or unsubstituted alkenyl group having 2 to 60 carbon atoms, a substituted or unsubstituted alkynyl group having 2 to 60 carbon atoms, a substituted or unsubstituted alkoxy group having 1 to 60 carbon atoms, a substituted or unsubstituted cycloalkyl group having 3 to 60 carbon atoms, a substituted or unsubstituted heterocycloalkyl group having 2 to 60 carbon atoms, a substituted or unsubstituted aryl group having 6 to 60 carbon atoms, a substituted or unsubstituted heteroaryl group having 2 to 60 carbon atoms, —SiRR′R″, and —P(═O)RR′, wherein adjacent groups are linked to form a substituted or unsubstituted aryl group having 6 to 60 carbon atoms, a substituted or unsubstituted heterocycloalkyl group having 2 to 60 carbon atoms, or a substituted or unsubstituted heteroaryl group having 2 to 60 carbon atoms,
q₁ is 0 or an integer of 1 to 5,
q₂ and q₃ are 0 or integers from 1 to 4,
q₄ and q₅ are each independently 0 or an integer of 1 to 3,
q₆ is 0, 1, or 2,
q₇ is 0 or 1, and
when q₁, q₂, q₃, 94, 95, and q₆ are each independently 2 or more, a plurality of Ra groups, Rb groups, Rc groups, Rd groups, Re groups, and Rf groups are each independently the same or different, and
* represents a bonding site.

5. The heterocyclic compound according to claim 1, wherein in Formula 1, Ar₁ and Ar₂ are each independently a substituted or unsubstituted aryl group having 6 to 40 carbon atoms, or a substituted or unsubstituted heteroaryl group having 2 to 40 carbon atoms.

6. The heterocyclic compound according to claim 1, wherein in Formula 1, Ar₁ and Ar₂ are the same or different.

7. The heterocyclic compound according to claim 1, wherein the deuterium content is 0, or greater than 0 and not more than 100%.

8. The heterocyclic compound according to claim 1, wherein the heterocyclic compound is represented by any one of formulae below:

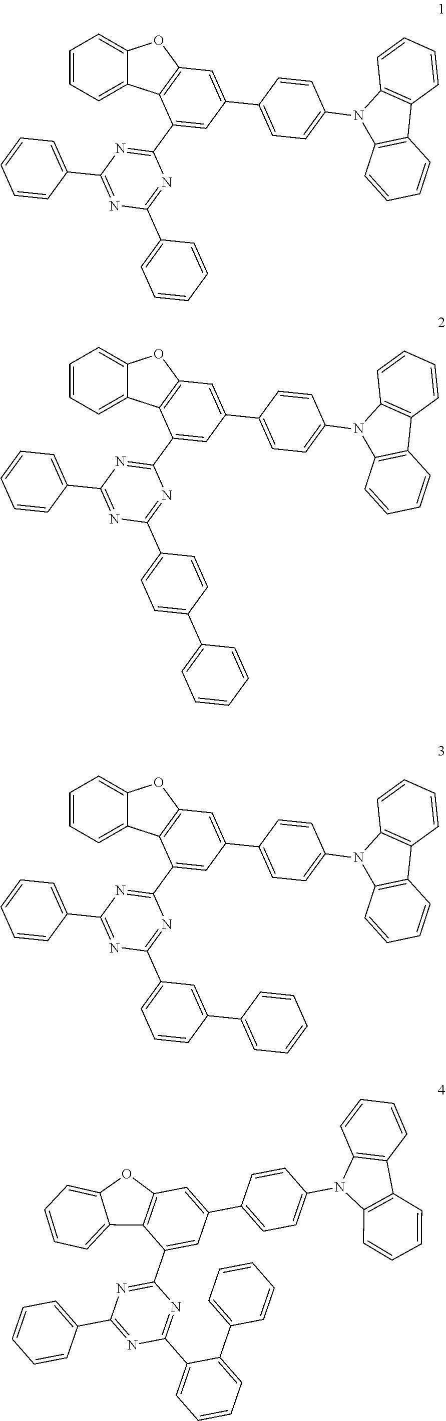 [SMILES: c1ccc(-c2nc(-c3ccccc3)nc(-c3cc(-c4ccc(-n5c6ccccc6c6ccccc65)cc4)cc4oc5ccccc5c34)n2)cc1 ; c1ccc(-c2ccc(-c3nc(-c4ccccc4)nc(-c4cc(-c5ccc(-n6c7ccccc7c7ccccc76)cc5)cc5oc6ccccc6c45)n3)cc2)cc1 ; c1ccc(-c2cccc(-c3nc(-c4ccccc4)nc(-c4cc(-c5ccc(-n6c7ccccc7c7ccccc76)cc5)cc5oc6ccccc6c45)n3)c2)cc1 ; c1ccc(-c2nc(-c3ccccc3-c3ccccc3)nc(-c3cc(-c4ccc(-n5c6ccccc6c6ccccc65)cc4)cc4oc5ccccc5c34)n2)cc1]

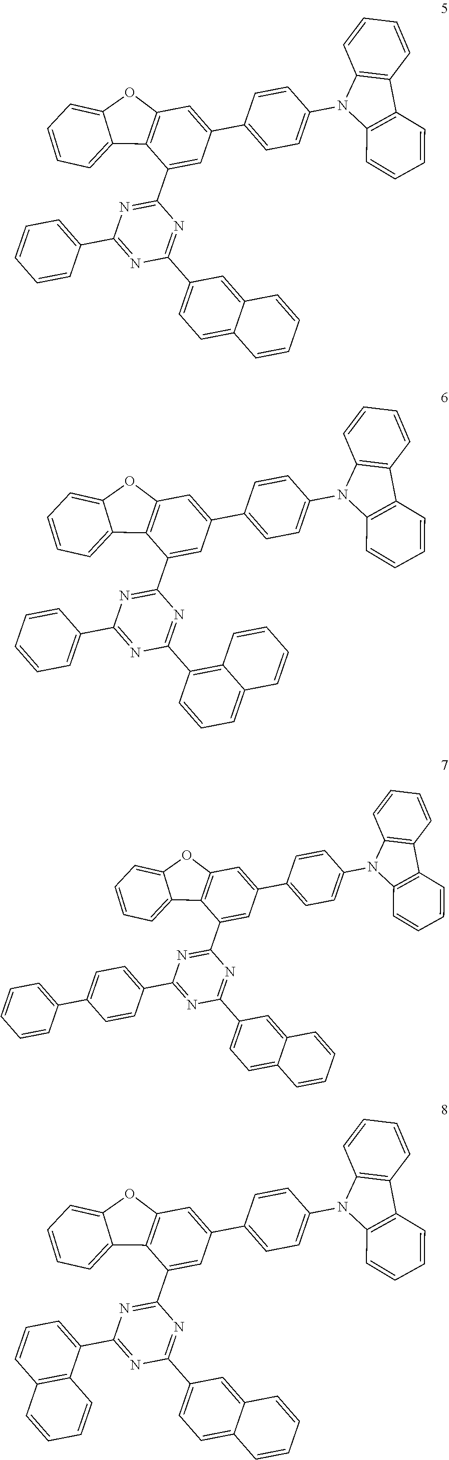 [SMILES: c1ccc(-c2nc(-c3ccc4ccccc4c3)nc(-c3cc(-c4ccc(-n5c6ccccc6c6ccccc65)cc4)cc4oc5ccccc5c34)n2)cc1 ; c1ccc(-c2nc(-c3cccc4ccccc34)nc(-c3cc(-c4ccc(-n5c6ccccc6c6ccccc65)cc4)cc4oc5ccccc5c34)n2)cc1 ; c1ccc(-c2ccc(-c3nc(-c4ccc5ccccc5c4)nc(-c4cc(-c5ccc(-n6c7ccccc7c7ccccc76)cc5)cc5oc6ccccc6c45)n3)cc2)cc1 ; c1ccc2cc(-c3nc(-c4cccc5ccccc45)nc(-c4cc(-c5ccc(-n6c7ccccc7c7ccccc76)cc5)cc5oc6ccccc6c45)n3)ccc2c1]

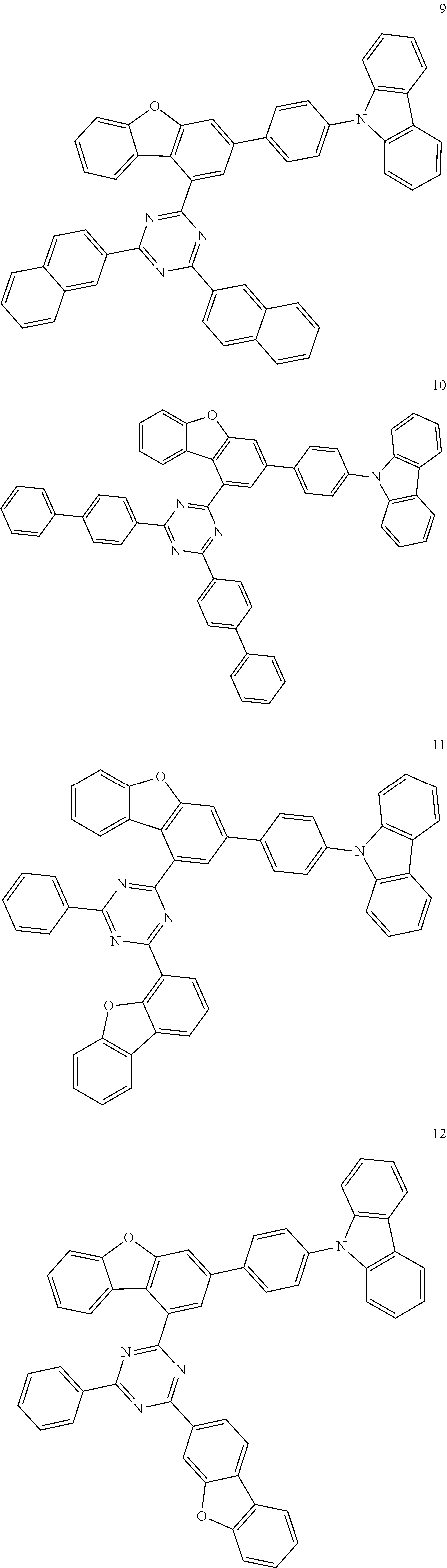 [SMILES: c1ccc2cc(-c3nc(-c4ccc5ccccc5c4)nc(-c4cc(-c5ccc(-n6c7ccccc7c7ccccc76)cc5)cc5oc6ccccc6c45)n3)ccc2c1 ; c1ccc(-c2ccc(-c3nc(-c4ccc(-c5ccccc5)cc4)nc(-c4cc(-c5ccc(-n6c7ccccc7c7ccccc76)cc5)cc5oc6ccccc6c45)n3)cc2)cc1 ; c1ccc(-c2nc(-c3cccc4c3oc3ccccc34)nc(-c3cc(-c4ccc(-n5c6ccccc6c6ccccc65)cc4)cc4oc5ccccc5c34)n2)cc1 ; c1ccc(-c2nc(-c3ccc4c(c3)oc3ccccc34)nc(-c3cc(-c4ccc(-n5c6ccccc6c6ccccc65)cc4)cc4oc5ccccc5c34)n2)cc1]

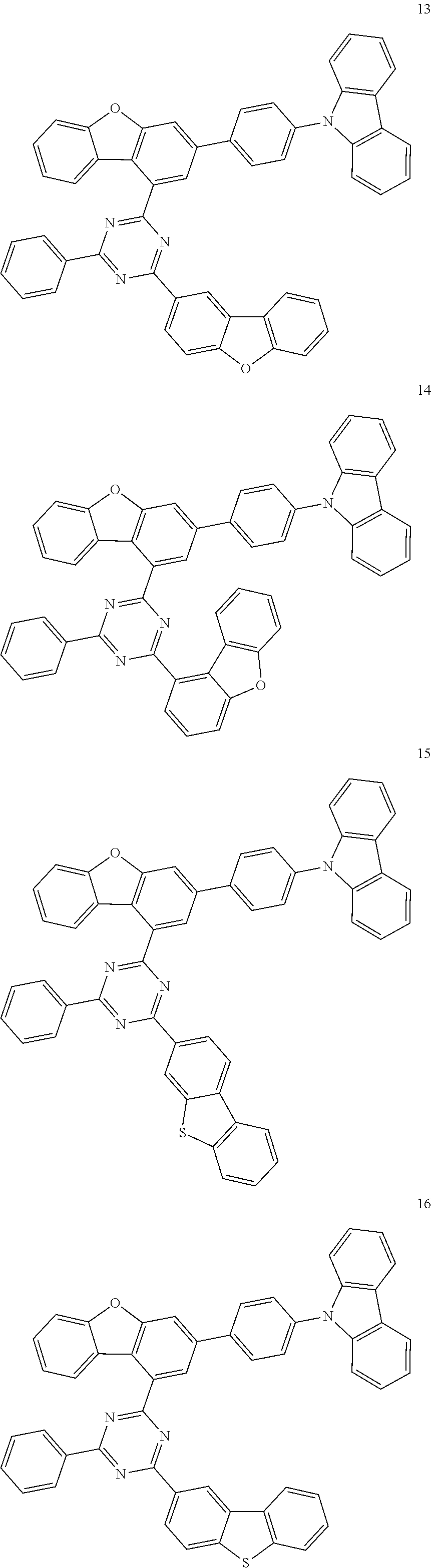 [SMILES: c1ccc(-c2nc(-c3ccc4oc5ccccc5c4c3)nc(-c3cc(-c4ccc(-n5c6ccccc6c6ccccc65)cc4)cc4oc5ccccc5c34)n2)cc1 ; c1ccc(-c2nc(-c3cccc4oc5ccccc5c34)nc(-c3cc(-c4ccc(-n5c6ccccc6c6ccccc65)cc4)cc4oc5ccccc5c34)n2)cc1 ; c1ccc(-c2nc(-c3ccc4c(c3)sc3ccccc34)nc(-c3cc(-c4ccc(-n5c6ccccc6c6ccccc65)cc4)cc4oc5ccccc5c34)n2)cc1 ; c1ccc(-c2nc(-c3ccc4sc5ccccc5c4c3)nc(-c3cc(-c4ccc(-n5c6ccccc6c6ccccc65)cc4)cc4oc5ccccc5c34)n2)cc1]

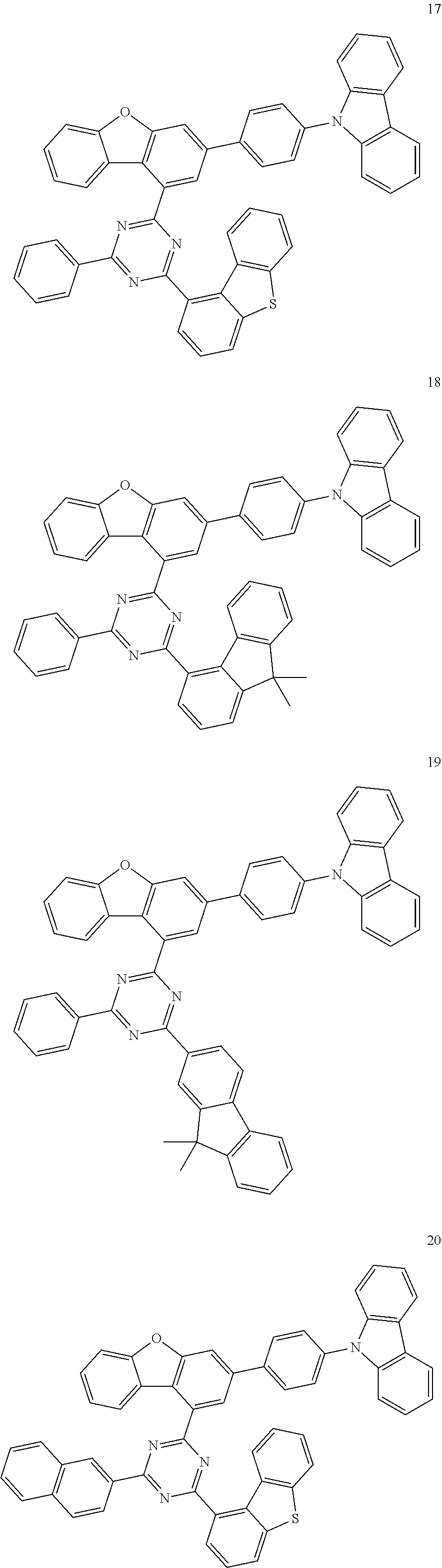 [SMILES: c1ccc(-c2nc(-c3cc(-c4ccc(-n5c6ccccc6c6ccccc65)cc4)cc4oc5ccccc5c34)nc(-c3cccc4sc5ccccc5c34)n2)cc1 ; CC1(C)c2ccccc2-c2c(-c3nc(-c4ccccc4)nc(-c4cc(-c5ccc(-n6c7ccccc7c7ccccc76)cc5)cc5oc6ccccc6c45)n3)cccc21 ; CC1(C)c2ccccc2-c2ccc(-c3nc(-c4ccccc4)nc(-c4cc(-c5ccc(-n6c7ccccc7c7ccccc76)cc5)cc5oc6ccccc6c45)n3)cc21 ; c1ccc2cc(-c3nc(-c4cc(-c5ccc(-n6c7ccccc7c7ccccc76)cc5)cc5oc6ccccc6c45)nc(-c4cccc5sc6ccccc6c45)n3)ccc2c1]

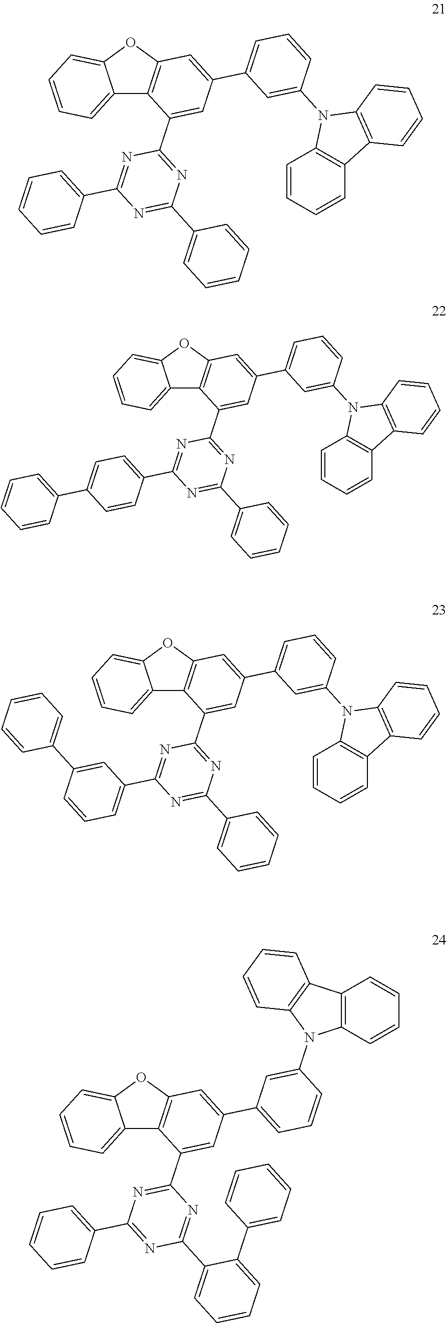 [SMILES: c1ccc(-c2nc(-c3ccccc3)nc(-c3cc(-c4cccc(-n5c6ccccc6c6ccccc65)c4)cc4oc5ccccc5c34)n2)cc1 ; c1ccc(-c2ccc(-c3nc(-c4ccccc4)nc(-c4cc(-c5cccc(-n6c7ccccc7c7ccccc76)c5)cc5oc6ccccc6c45)n3)cc2)cc1 ; c1ccc(-c2cccc(-c3nc(-c4ccccc4)nc(-c4cc(-c5cccc(-n6c7ccccc7c7ccccc76)c5)cc5oc6ccccc6c45)n3)c2)cc1 ; c1ccc(-c2nc(-c3ccccc3-c3ccccc3)nc(-c3cc(-c4cccc(-n5c6ccccc6c6ccccc65)c4)cc4oc5ccccc5c34)n2)cc1]

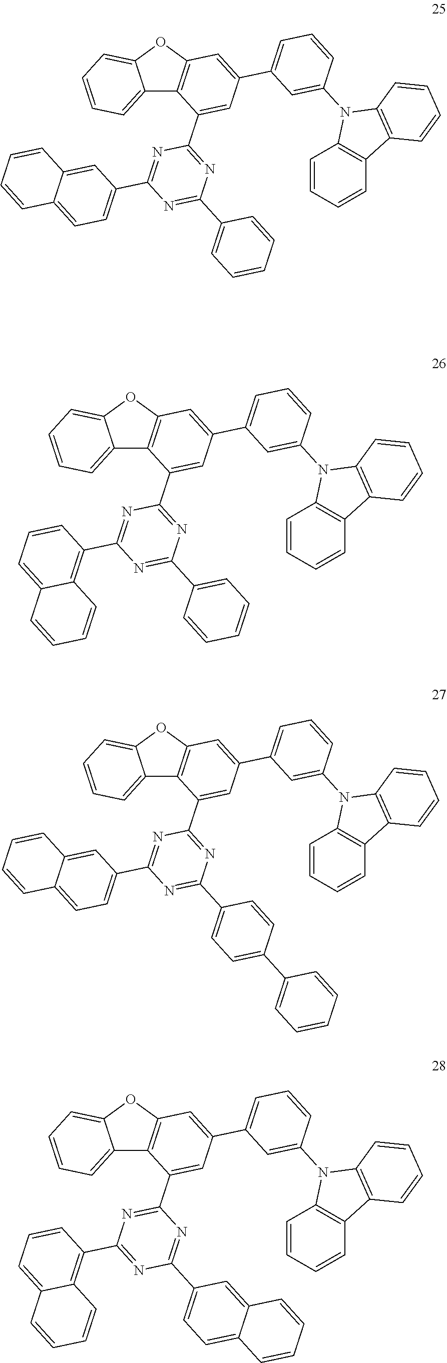 [SMILES: c1ccc(-c2nc(-c3ccc4ccccc4c3)nc(-c3cc(-c4cccc(-n5c6ccccc6c6ccccc65)c4)cc4oc5ccccc5c34)n2)cc1 ; c1ccc(-c2nc(-c3cccc4ccccc34)nc(-c3cc(-c4cccc(-n5c6ccccc6c6ccccc65)c4)cc4oc5ccccc5c34)n2)cc1 ; c1ccc(-c2ccc(-c3nc(-c4ccc5ccccc5c4)nc(-c4cc(-c5cccc(-n6c7ccccc7c7ccccc76)c5)cc5oc6ccccc6c45)n3)cc2)cc1 ; c1cc(-c2cc(-c3nc(-c4ccc5ccccc5c4)nc(-c4cccc5ccccc45)n3)c3c(c2)oc2ccccc23)cc(-n2c3ccccc3c3ccccc32)c1]

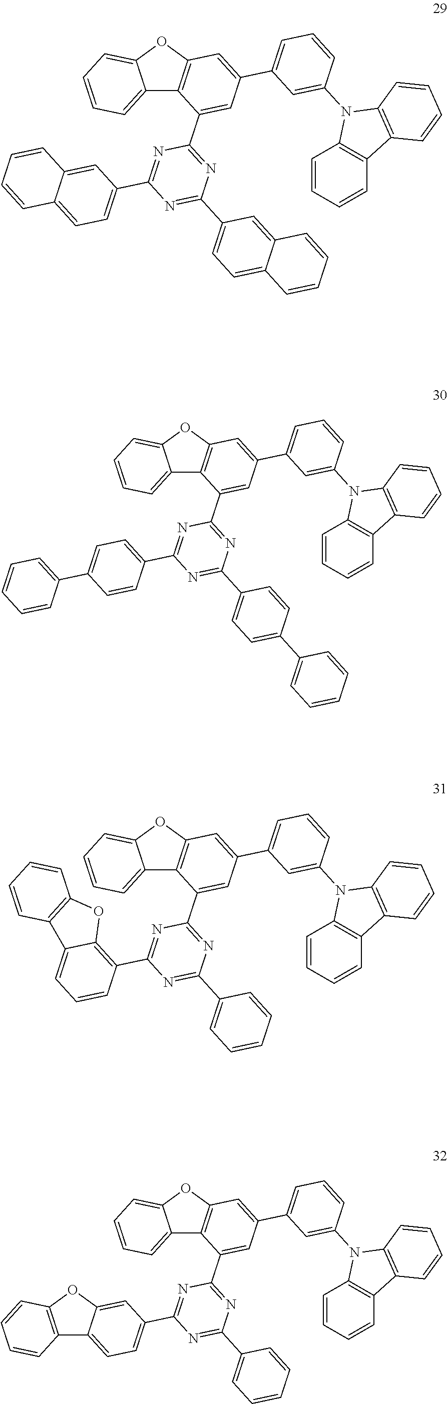 [SMILES: c1cc(-c2cc(-c3nc(-c4ccc5ccccc5c4)nc(-c4ccc5ccccc5c4)n3)c3c(c2)oc2ccccc23)cc(-n2c3ccccc3c3ccccc32)c1 ; c1ccc(-c2ccc(-c3nc(-c4ccc(-c5ccccc5)cc4)nc(-c4cc(-c5cccc(-n6c7ccccc7c7ccccc76)c5)cc5oc6ccccc6c45)n3)cc2)cc1 ; c1ccc(-c2nc(-c3cccc4c3oc3ccccc34)nc(-c3cc(-c4cccc(-n5c6ccccc6c6ccccc65)c4)cc4oc5ccccc5c34)n2)cc1 ; c1ccc(-c2nc(-c3ccc4c(c3)oc3ccccc34)nc(-c3cc(-c4cccc(-n5c6ccccc6c6ccccc65)c4)cc4oc5ccccc5c34)n2)cc1]

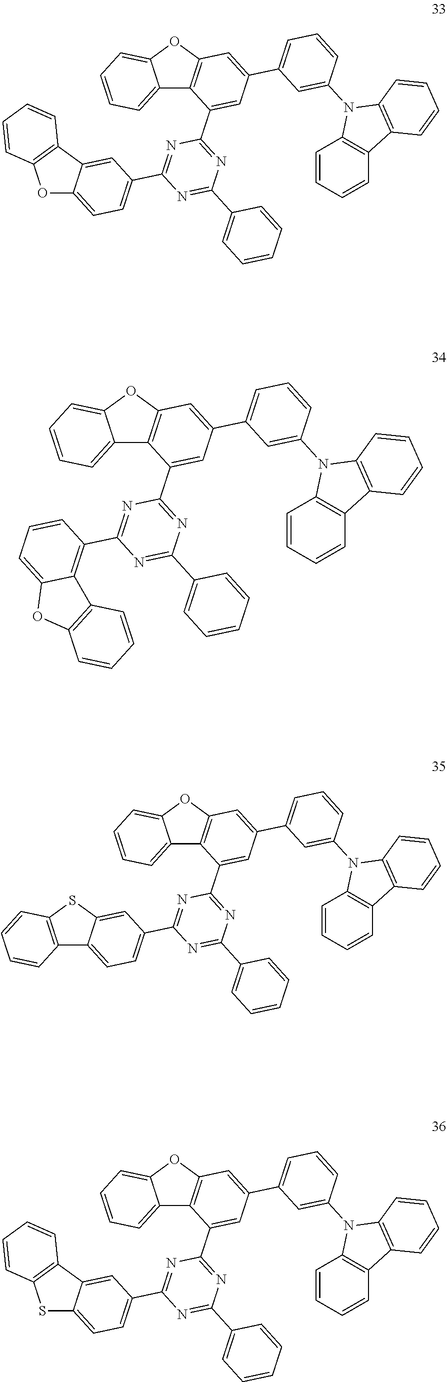 [SMILES: c1ccc(-c2nc(-c3ccc4oc5ccccc5c4c3)nc(-c3cc(-c4cccc(-n5c6ccccc6c6ccccc65)c4)cc4oc5ccccc5c34)n2)cc1 ; c1ccc(-c2nc(-c3cccc4oc5ccccc5c34)nc(-c3cc(-c4cccc(-n5c6ccccc6c6ccccc65)c4)cc4oc5ccccc5c34)n2)cc1 ; c1ccc(-c2nc(-c3ccc4c(c3)sc3ccccc34)nc(-c3cc(-c4cccc(-n5c6ccccc6c6ccccc65)c4)cc4oc5ccccc5c34)n2)cc1 ; c1ccc(-c2nc(-c3ccc4sc5ccccc5c4c3)nc(-c3cc(-c4cccc(-n5c6ccccc6c6ccccc65)c4)cc4oc5ccccc5c34)n2)cc1]

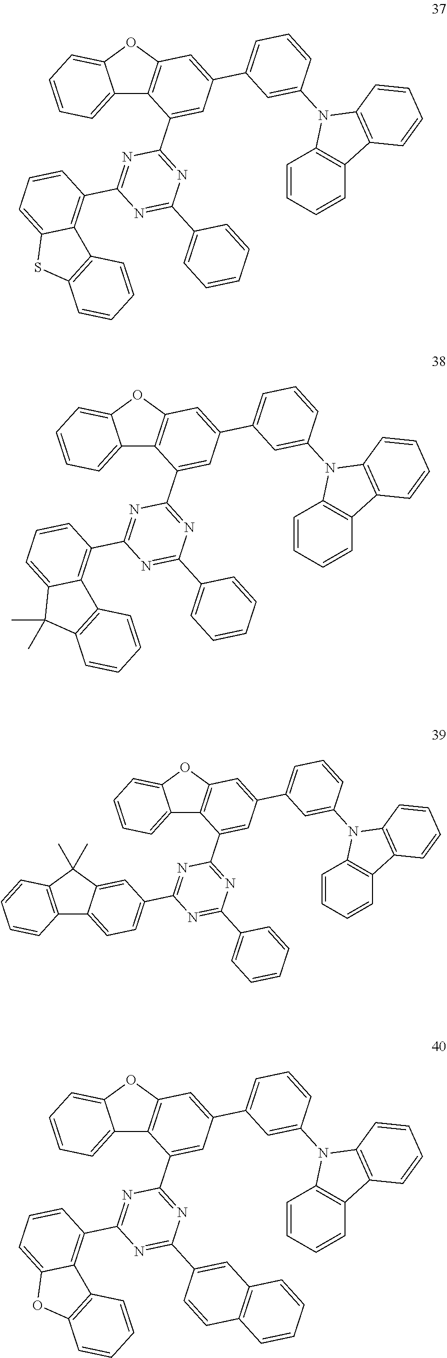 [SMILES: c1ccc(-c2nc(-c3cc(-c4cccc(-n5c6ccccc6c6ccccc65)c4)cc4oc5ccccc5c34)nc(-c3cccc4sc5ccccc5c34)n2)cc1 ; CC1(C)c2ccccc2-c2c(-c3nc(-c4ccccc4)nc(-c4cc(-c5cccc(-n6c7ccccc7c7ccccc76)c5)cc5oc6ccccc6c45)n3)cccc21 ; CC1(C)c2ccccc2-c2ccc(-c3nc(-c4ccccc4)nc(-c4cc(-c5cccc(-n6c7ccccc7c7ccccc76)c5)cc5oc6ccccc6c45)n3)cc21 ; c1cc(-c2cc(-c3nc(-c4ccc5ccccc5c4)nc(-c4cccc5oc6ccccc6c45)n3)c3c(c2)oc2ccccc23)cc(-n2c3ccccc3c3ccccc32)c1]

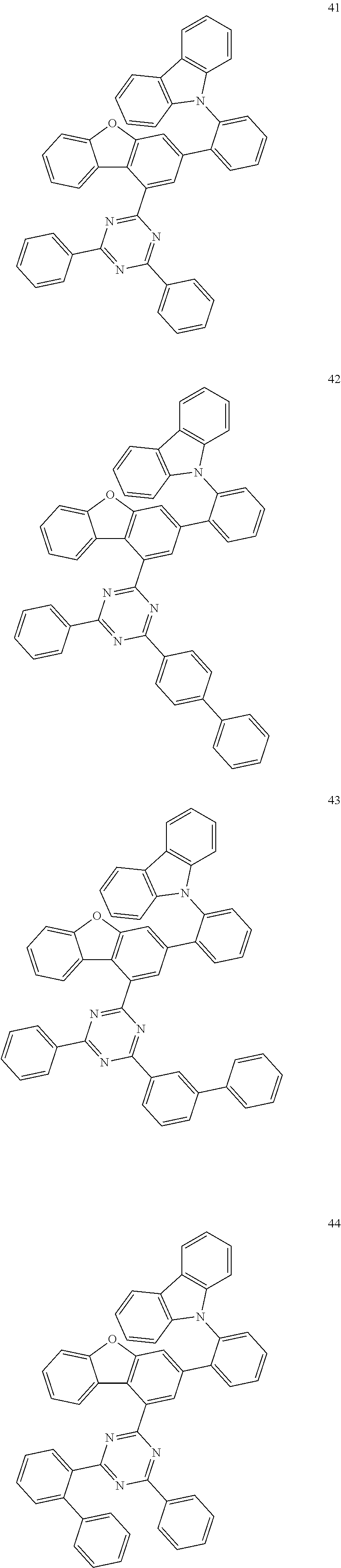 [SMILES: c1ccc(-c2nc(-c3ccccc3)nc(-c3cc(-c4ccccc4-n4c5ccccc5c5ccccc54)cc4oc5ccccc5c34)n2)cc1 ; c1ccc(-c2ccc(-c3nc(-c4ccccc4)nc(-c4cc(-c5ccccc5-n5c6ccccc6c6ccccc65)cc5oc6ccccc6c45)n3)cc2)cc1 ; c1ccc(-c2cccc(-c3nc(-c4ccccc4)nc(-c4cc(-c5ccccc5-n5c6ccccc6c6ccccc65)cc5oc6ccccc6c45)n3)c2)cc1 ; c1ccc(-c2nc(-c3ccccc3-c3ccccc3)nc(-c3cc(-c4ccccc4-n4c5ccccc5c5ccccc54)cc4oc5ccccc5c34)n2)cc1]

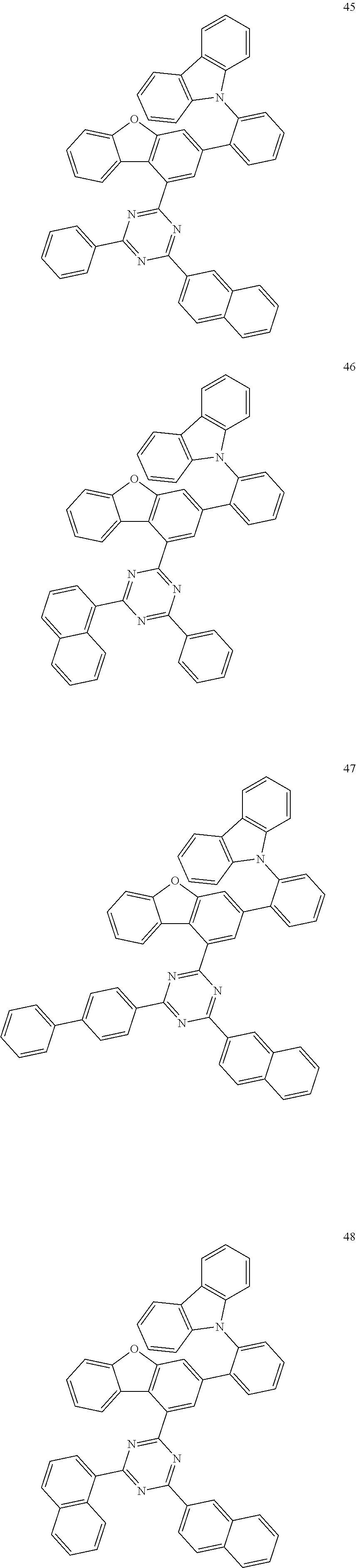 [SMILES: c1ccc(-c2nc(-c3ccc4ccccc4c3)nc(-c3cc(-c4ccccc4-n4c5ccccc5c5ccccc54)cc4oc5ccccc5c34)n2)cc1 ; c1ccc(-c2nc(-c3cccc4ccccc34)nc(-c3cc(-c4ccccc4-n4c5ccccc5c5ccccc54)cc4oc5ccccc5c34)n2)cc1 ; c1ccc(-c2ccc(-c3nc(-c4ccc5ccccc5c4)nc(-c4cc(-c5ccccc5-n5c6ccccc6c6ccccc65)cc5oc6ccccc6c45)n3)cc2)cc1 ; c1ccc(-n2c3ccccc3c3ccccc32)c(-c2cc(-c3nc(-c4ccc5ccccc5c4)nc(-c4cccc5ccccc45)n3)c3c(c2)oc2ccccc23)c1]

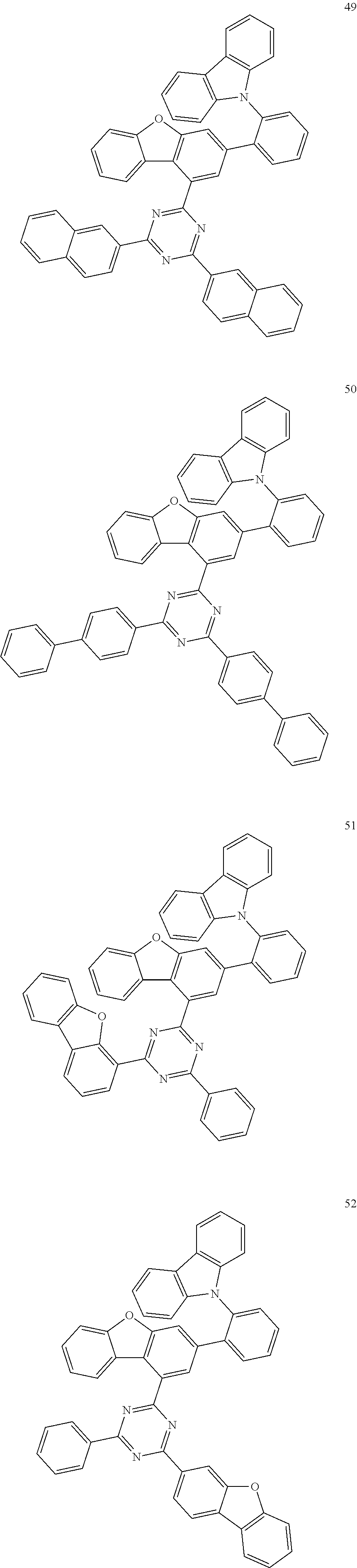 [SMILES: c1ccc(-n2c3ccccc3c3ccccc32)c(-c2cc(-c3nc(-c4ccc5ccccc5c4)nc(-c4ccc5ccccc5c4)n3)c3c(c2)oc2ccccc23)c1 ; c1ccc(-c2ccc(-c3nc(-c4ccc(-c5ccccc5)cc4)nc(-c4cc(-c5ccccc5-n5c6ccccc6c6ccccc65)cc5oc6ccccc6c45)n3)cc2)cc1 ; c1ccc(-c2nc(-c3cccc4c3oc3ccccc34)nc(-c3cc(-c4ccccc4-n4c5ccccc5c5ccccc54)cc4oc5ccccc5c34)n2)cc1 ; c1ccc(-c2nc(-c3ccc4c(c3)oc3ccccc34)nc(-c3cc(-c4ccccc4-n4c5ccccc5c5ccccc54)cc4oc5ccccc5c34)n2)cc1]

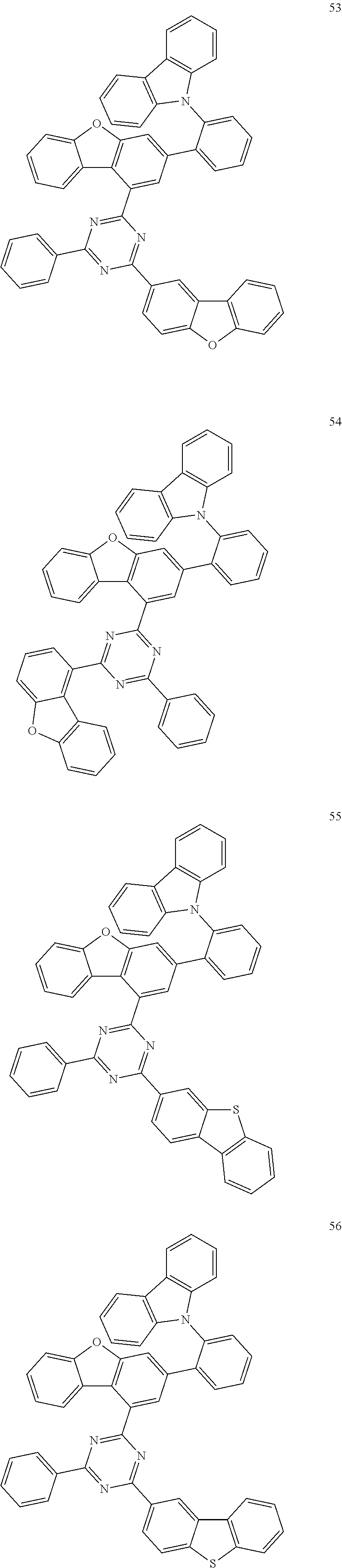 [SMILES: c1ccc(-c2nc(-c3ccc4oc5ccccc5c4c3)nc(-c3cc(-c4ccccc4-n4c5ccccc5c5ccccc54)cc4oc5ccccc5c34)n2)cc1 ; c1ccc(-c2nc(-c3cccc4oc5ccccc5c34)nc(-c3cc(-c4ccccc4-n4c5ccccc5c5ccccc54)cc4oc5ccccc5c34)n2)cc1 ; c1ccc(-c2nc(-c3ccc4c(c3)sc3ccccc34)nc(-c3cc(-c4ccccc4-n4c5ccccc5c5ccccc54)cc4oc5ccccc5c34)n2)cc1 ; c1ccc(-c2nc(-c3ccc4sc5ccccc5c4c3)nc(-c3cc(-c4ccccc4-n4c5ccccc5c5ccccc54)cc4oc5ccccc5c34)n2)cc1]

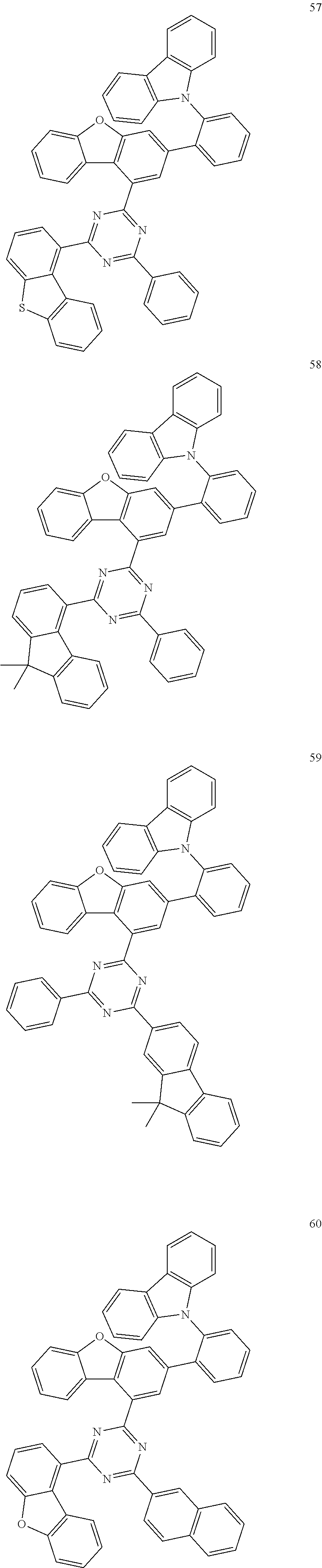 [SMILES: c1ccc(-c2nc(-c3cc(-c4ccccc4-n4c5ccccc5c5ccccc54)cc4oc5ccccc5c34)nc(-c3cccc4sc5ccccc5c34)n2)cc1 ; CC1(C)c2ccccc2-c2c(-c3nc(-c4ccccc4)nc(-c4cc(-c5ccccc5-n5c6ccccc6c6ccccc65)cc5oc6ccccc6c45)n3)cccc21 ; CC1(C)c2ccccc2-c2ccc(-c3nc(-c4ccccc4)nc(-c4cc(-c5ccccc5-n5c6ccccc6c6ccccc65)cc5oc6ccccc6c45)n3)cc21 ; c1ccc(-n2c3ccccc3c3ccccc32)c(-c2cc(-c3nc(-c4ccc5ccccc5c4)nc(-c4cccc5oc6ccccc6c45)n3)c3c(c2)oc2ccccc23)c1]

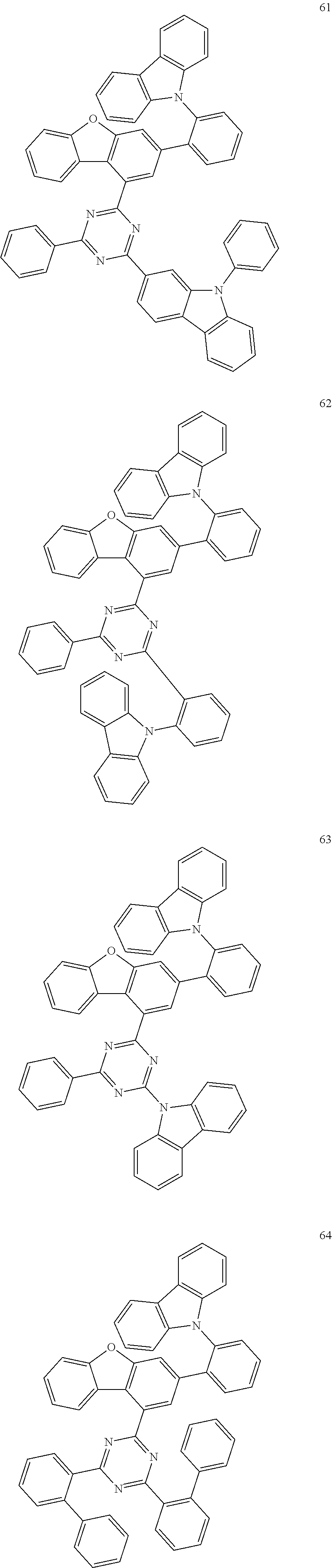 [SMILES: c1ccc(-c2nc(-c3ccc4c5ccccc5n(-c5ccccc5)c4c3)nc(-c3cc(-c4ccccc4-n4c5ccccc5c5ccccc54)cc4oc5ccccc5c34)n2)cc1 ; c1ccc(-c2nc(-c3ccccc3-n3c4ccccc4c4ccccc43)nc(-c3cc(-c4ccccc4-n4c5ccccc5c5ccccc54)cc4oc5ccccc5c34)n2)cc1 ; c1ccc(-c2nc(-c3cc(-c4ccccc4-n4c5ccccc5c5ccccc54)cc4oc5ccccc5c34)nc(-n3c4ccccc4c4ccccc43)n2)cc1 ; c1ccc(-c2ccccc2-c2nc(-c3ccccc3-c3ccccc3)nc(-c3cc(-c4ccccc4-n4c5ccccc5c5ccccc54)cc4oc5ccccc5c34)n2)cc1]

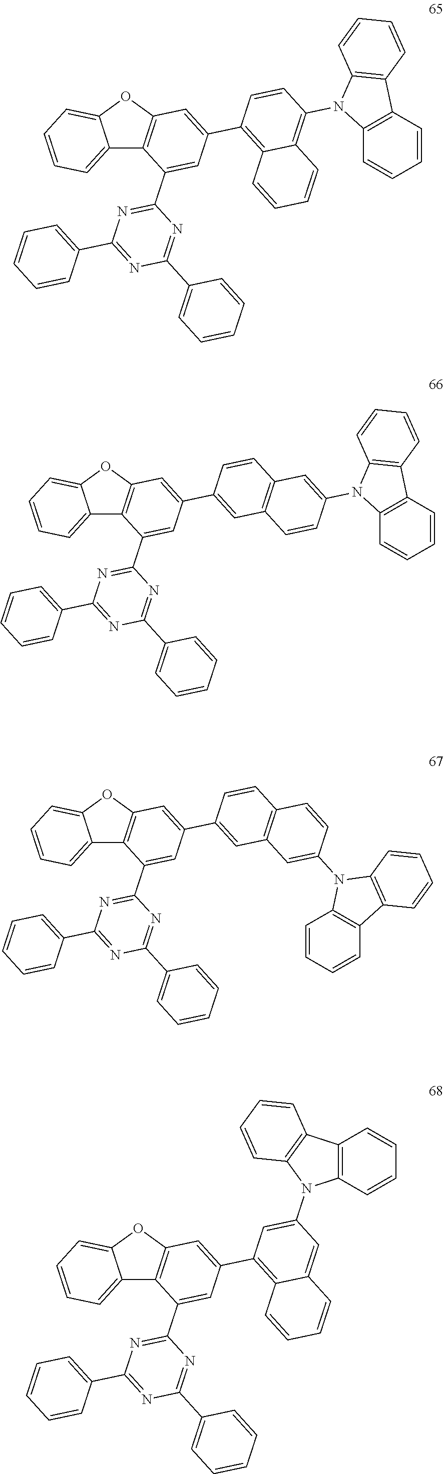 [SMILES: c1ccc(-c2nc(-c3ccccc3)nc(-c3cc(-c4ccc(-n5c6ccccc6c6ccccc65)c5ccccc45)cc4oc5ccccc5c34)n2)cc1 ; c1ccc(-c2nc(-c3ccccc3)nc(-c3cc(-c4ccc5cc(-n6c7ccccc7c7ccccc76)ccc5c4)cc4oc5ccccc5c34)n2)cc1 ; c1ccc(-c2nc(-c3ccccc3)nc(-c3cc(-c4ccc5ccc(-n6c7ccccc7c7ccccc76)cc5c4)cc4oc5ccccc5c34)n2)cc1 ; c1ccc(-c2nc(-c3ccccc3)nc(-c3cc(-c4cc(-n5c6ccccc6c6ccccc65)cc5ccccc45)cc4oc5ccccc5c34)n2)cc1]

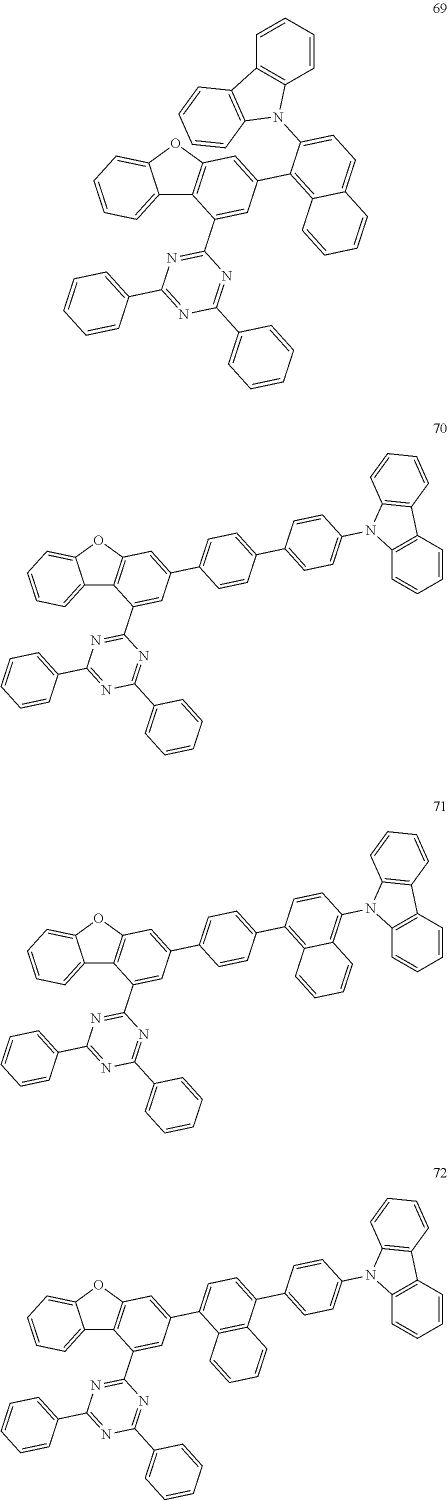 [SMILES: c1ccc(-c2nc(-c3ccccc3)nc(-c3cc(-c4c(-n5c6ccccc6c6ccccc65)ccc5ccccc45)cc4oc5ccccc5c34)n2)cc1 ; c1ccc(-c2nc(-c3ccccc3)nc(-c3cc(-c4ccc(-c5ccc(-n6c7ccccc7c7ccccc76)cc5)cc4)cc4oc5ccccc5c34)n2)cc1 ; c1ccc(-c2nc(-c3ccccc3)nc(-c3cc(-c4ccc(-c5ccc(-n6c7ccccc7c7ccccc76)c6ccccc56)cc4)cc4oc5ccccc5c34)n2)cc1 ; c1ccc(-c2nc(-c3ccccc3)nc(-c3cc(-c4ccc(-c5ccc(-n6c7ccccc7c7ccccc76)cc5)c5ccccc45)cc4oc5ccccc5c34)n2)cc1]

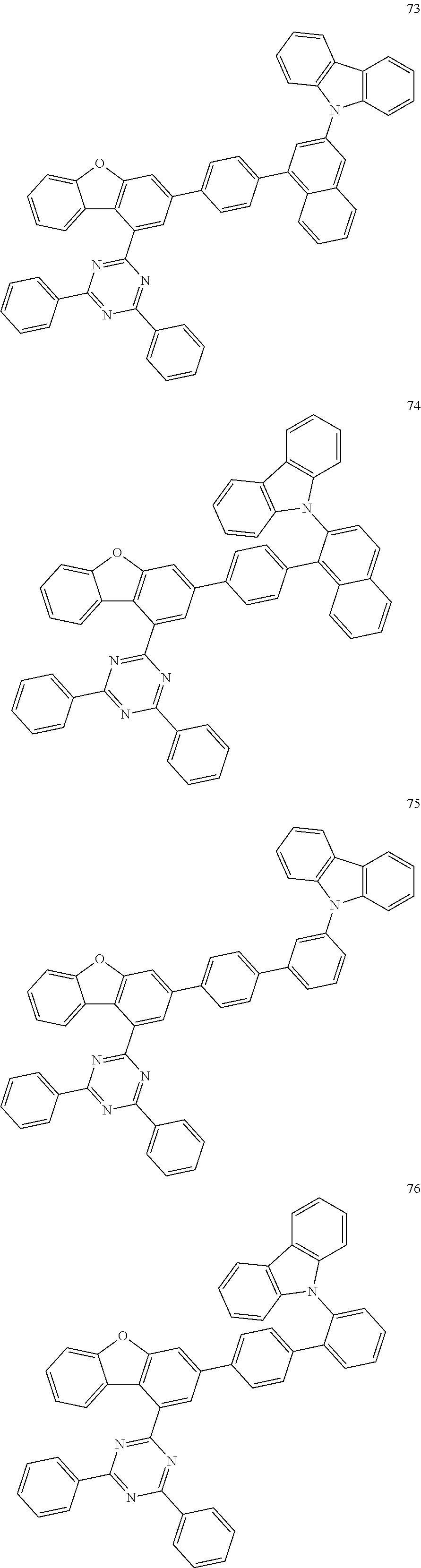 [SMILES: c1ccc(-c2nc(-c3ccccc3)nc(-c3cc(-c4ccc(-c5cc(-n6c7ccccc7c7ccccc76)cc6ccccc56)cc4)cc4oc5ccccc5c34)n2)cc1 ; c1ccc(-c2nc(-c3ccccc3)nc(-c3cc(-c4ccc(-c5c(-n6c7ccccc7c7ccccc76)ccc6ccccc56)cc4)cc4oc5ccccc5c34)n2)cc1 ; c1ccc(-c2nc(-c3ccccc3)nc(-c3cc(-c4ccc(-c5cccc(-n6c7ccccc7c7ccccc76)c5)cc4)cc4oc5ccccc5c34)n2)cc1 ; c1ccc(-c2nc(-c3ccccc3)nc(-c3cc(-c4ccc(-c5ccccc5-n5c6ccccc6c6ccccc65)cc4)cc4oc5ccccc5c34)n2)cc1]

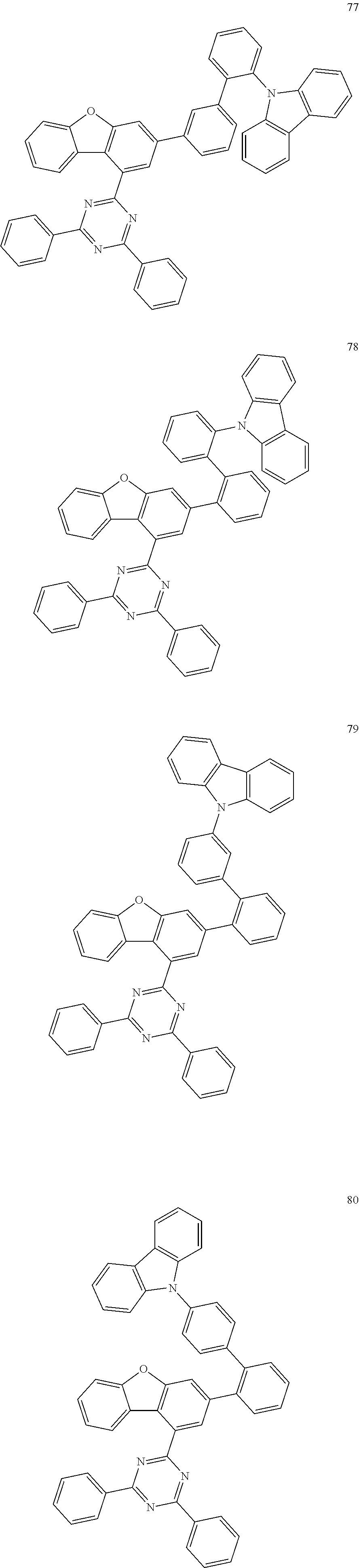 [SMILES: c1ccc(-c2nc(-c3ccccc3)nc(-c3cc(-c4cccc(-c5ccccc5-n5c6ccccc6c6ccccc65)c4)cc4oc5ccccc5c34)n2)cc1 ; c1ccc(-c2nc(-c3ccccc3)nc(-c3cc(-c4ccccc4-c4ccccc4-n4c5ccccc5c5ccccc54)cc4oc5ccccc5c34)n2)cc1 ; c1ccc(-c2nc(-c3ccccc3)nc(-c3cc(-c4ccccc4-c4cccc(-n5c6ccccc6c6ccccc65)c4)cc4oc5ccccc5c34)n2)cc1 ; c1ccc(-c2nc(-c3ccccc3)nc(-c3cc(-c4ccccc4-c4ccc(-n5c6ccccc6c6ccccc65)cc4)cc4oc5ccccc5c34)n2)cc1]

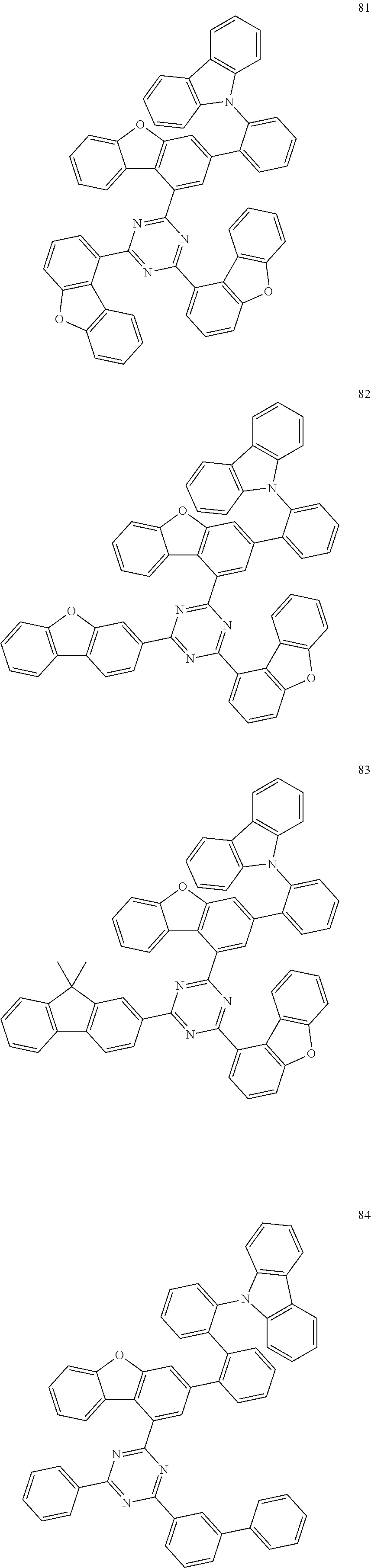 [SMILES: c1ccc(-n2c3ccccc3c3ccccc32)c(-c2cc(-c3nc(-c4cccc5oc6ccccc6c45)nc(-c4cccc5oc6ccccc6c45)n3)c3c(c2)oc2ccccc23)c1 ; c1ccc(-n2c3ccccc3c3ccccc32)c(-c2cc(-c3nc(-c4ccc5c(c4)oc4ccccc45)nc(-c4cccc5oc6ccccc6c45)n3)c3c(c2)oc2ccccc23)c1 ; CC1(C)c2ccccc2-c2ccc(-c3nc(-c4cccc5oc6ccccc6c45)nc(-c4cc(-c5ccccc5-n5c6ccccc6c6ccccc65)cc5oc6ccccc6c45)n3)cc21 ; c1ccc(-c2cccc(-c3nc(-c4ccccc4)nc(-c4cc(-c5ccccc5-c5ccccc5-n5c6ccccc6c6ccccc65)cc5oc6ccccc6c45)n3)c2)cc1]

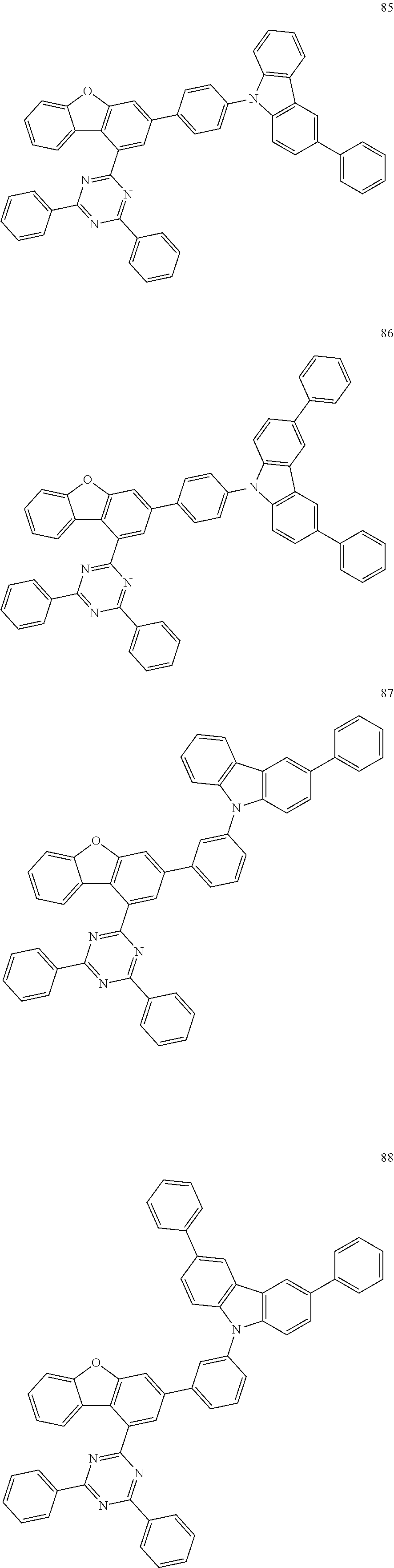 [SMILES: c1ccc(-c2ccc3c(c2)c2ccccc2n3-c2ccc(-c3cc(-c4nc(-c5ccccc5)nc(-c5ccccc5)n4)c4c(c3)oc3ccccc34)cc2)cc1 ; c1ccc(-c2ccc3c(c2)c2cc(-c4ccccc4)ccc2n3-c2ccc(-c3cc(-c4nc(-c5ccccc5)nc(-c5ccccc5)n4)c4c(c3)oc3ccccc34)cc2)cc1 ; c1ccc(-c2ccc3c(c2)c2ccccc2n3-c2cccc(-c3cc(-c4nc(-c5ccccc5)nc(-c5ccccc5)n4)c4c(c3)oc3ccccc34)c2)cc1 ; c1ccc(-c2ccc3c(c2)c2cc(-c4ccccc4)ccc2n3-c2cccc(-c3cc(-c4nc(-c5ccccc5)nc(-c5ccccc5)n4)c4c(c3)oc3ccccc34)c2)cc1]

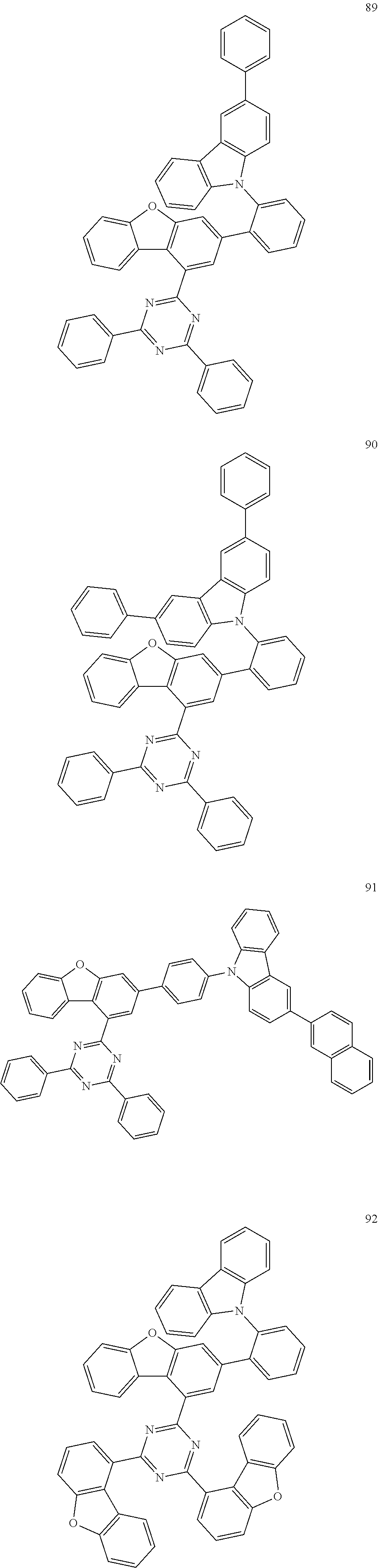 [SMILES: c1ccc(-c2ccc3c(c2)c2ccccc2n3-c2ccccc2-c2cc(-c3nc(-c4ccccc4)nc(-c4ccccc4)n3)c3c(c2)oc2ccccc23)cc1 ; c1ccc(-c2ccc3c(c2)c2cc(-c4ccccc4)ccc2n3-c2ccccc2-c2cc(-c3nc(-c4ccccc4)nc(-c4ccccc4)n3)c3c(c2)oc2ccccc23)cc1 ; c1ccc(-c2nc(-c3ccccc3)nc(-c3cc(-c4ccc(-n5c6ccccc6c6cc(-c7ccc8ccccc8c7)ccc65)cc4)cc4oc5ccccc5c34)n2)cc1 ; c1ccc(-n2c3ccccc3c3ccccc32)c(-c2cc(-c3nc(-c4cccc5oc6ccccc6c45)nc(-c4cccc5oc6ccccc6c45)n3)c3c(c2)oc2ccccc23)c1]

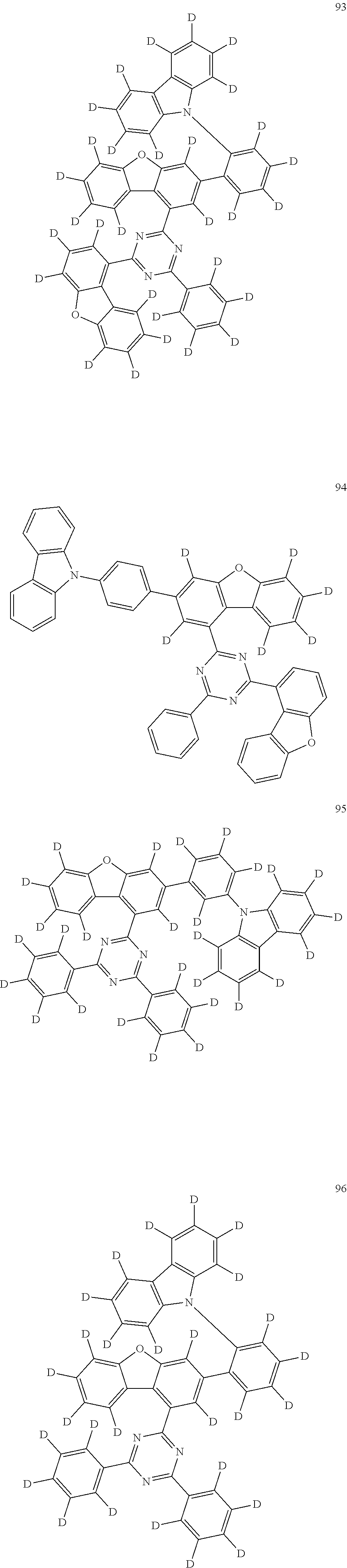

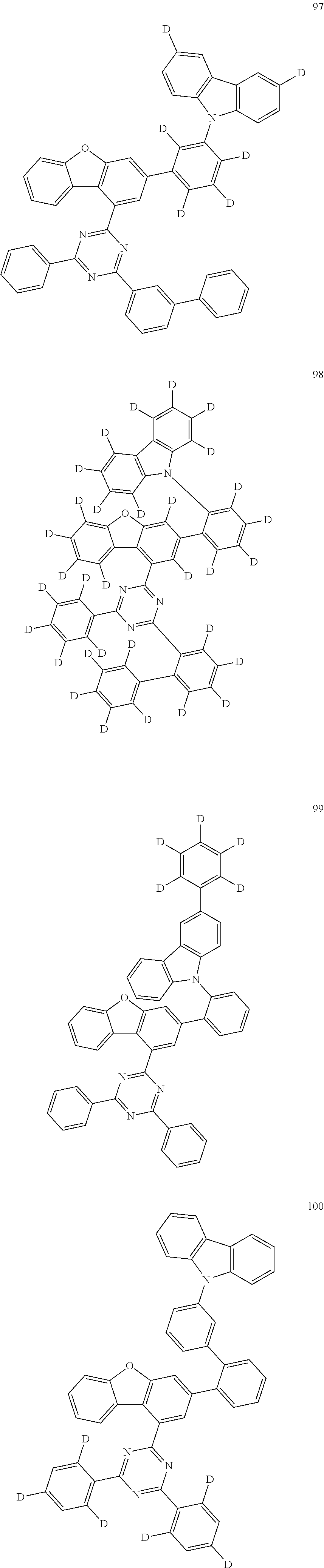

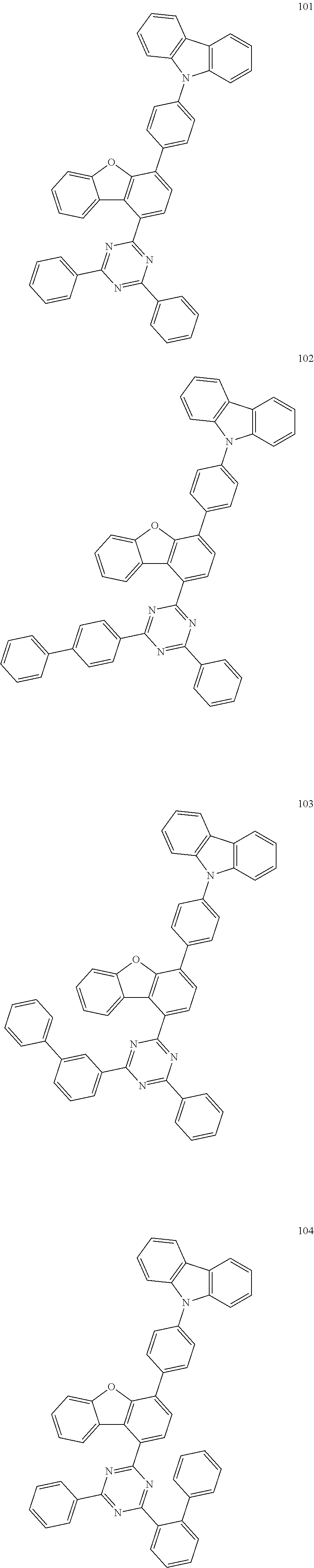 [SMILES: c1ccc(-c2nc(-c3ccccc3)nc(-c3ccc(-c4ccc(-n5c6ccccc6c6ccccc65)cc4)c4oc5ccccc5c34)n2)cc1 ; c1ccc(-c2ccc(-c3nc(-c4ccccc4)nc(-c4ccc(-c5ccc(-n6c7ccccc7c7ccccc76)cc5)c5oc6ccccc6c45)n3)cc2)cc1 ; c1ccc(-c2cccc(-c3nc(-c4ccccc4)nc(-c4ccc(-c5ccc(-n6c7ccccc7c7ccccc76)cc5)c5oc6ccccc6c45)n3)c2)cc1 ; c1ccc(-c2nc(-c3ccccc3-c3ccccc3)nc(-c3ccc(-c4ccc(-n5c6ccccc6c6ccccc65)cc4)c4oc5ccccc5c34)n2)cc1]

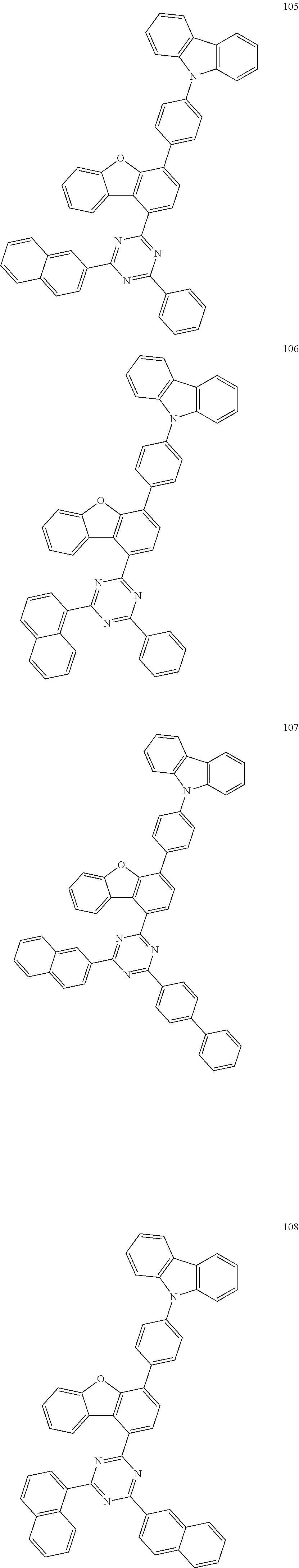

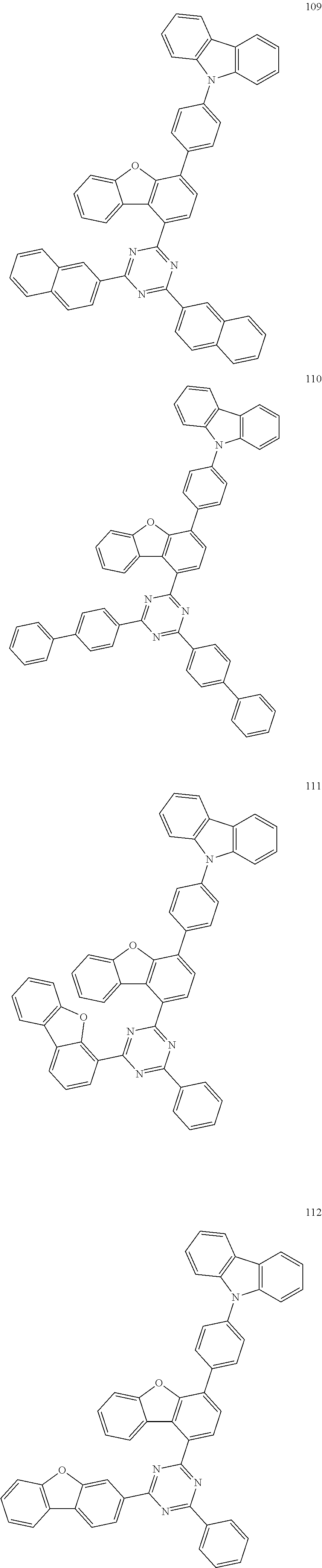

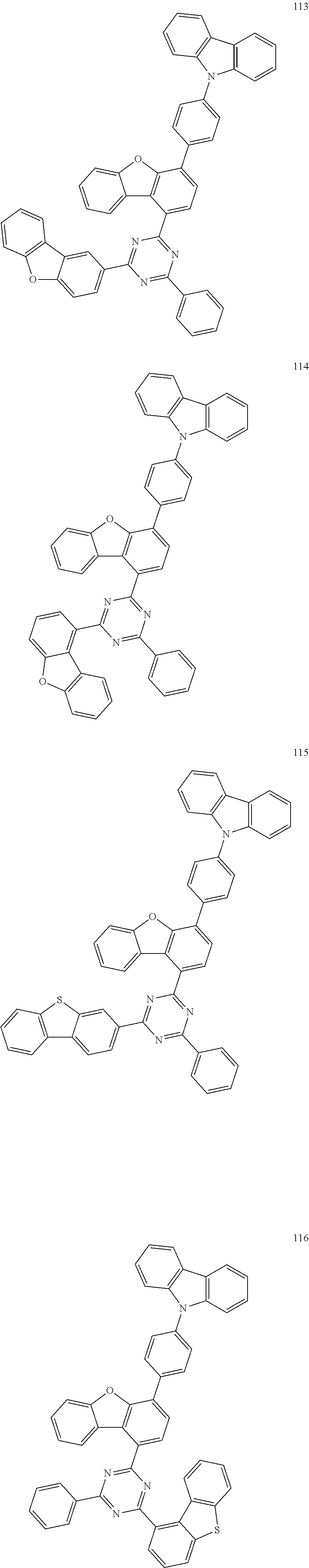 [SMILES: c1ccc(-c2nc(-c3ccc4oc5ccccc5c4c3)nc(-c3ccc(-c4ccc(-n5c6ccccc6c6ccccc65)cc4)c4oc5ccccc5c34)n2)cc1 ; c1ccc(-c2nc(-c3cccc4oc5ccccc5c34)nc(-c3ccc(-c4ccc(-n5c6ccccc6c6ccccc65)cc4)c4oc5ccccc5c34)n2)cc1 ; c1ccc(-c2nc(-c3ccc4c(c3)sc3ccccc34)nc(-c3ccc(-c4ccc(-n5c6ccccc6c6ccccc65)cc4)c4oc5ccccc5c34)n2)cc1 ; c1ccc(-c2nc(-c3cccc4sc5ccccc5c34)nc(-c3ccc(-c4ccc(-n5c6ccccc6c6ccccc65)cc4)c4oc5ccccc5c34)n2)cc1]

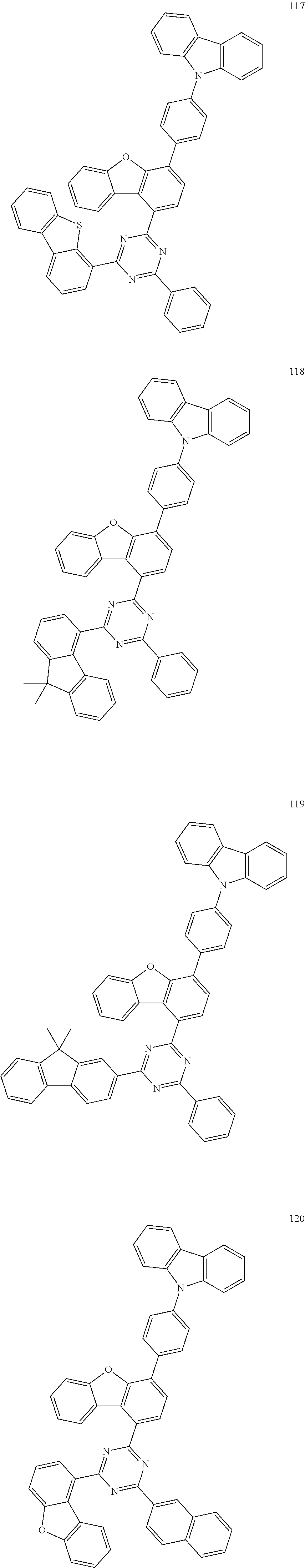 [SMILES: c1ccc(-c2nc(-c3cccc4c3sc3ccccc34)nc(-c3ccc(-c4ccc(-n5c6ccccc6c6ccccc65)cc4)c4oc5ccccc5c34)n2)cc1 ; CC1(C)c2ccccc2-c2c(-c3nc(-c4ccccc4)nc(-c4ccc(-c5ccc(-n6c7ccccc7c7ccccc76)cc5)c5oc6ccccc6c45)n3)cccc21 ; CC1(C)c2ccccc2-c2ccc(-c3nc(-c4ccccc4)nc(-c4ccc(-c5ccc(-n6c7ccccc7c7ccccc76)cc5)c5oc6ccccc6c45)n3)cc21 ; c1ccc2cc(-c3nc(-c4cccc5oc6ccccc6c45)nc(-c4ccc(-c5ccc(-n6c7ccccc7c7ccccc76)cc5)c5oc6ccccc6c45)n3)ccc2c1]

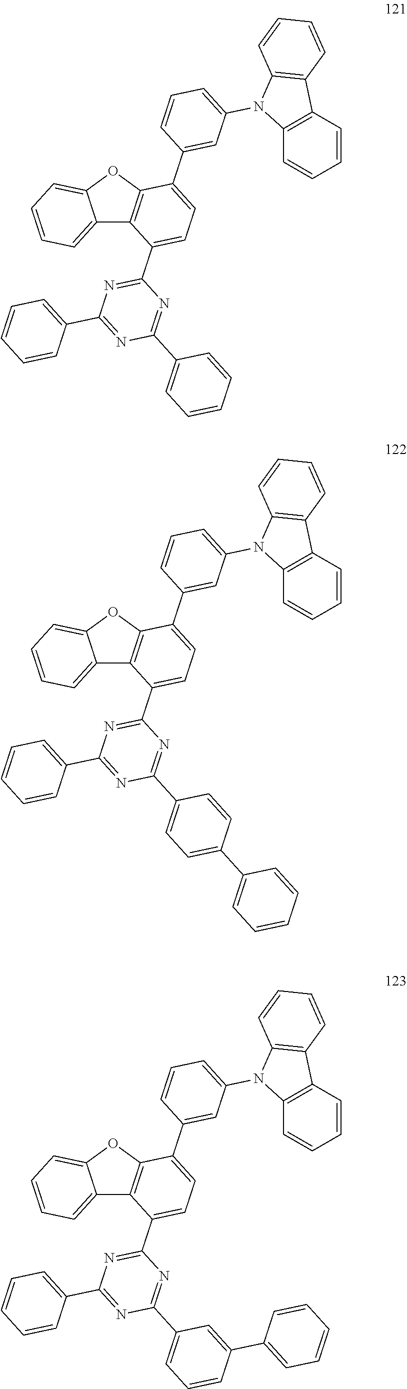 [SMILES: c1ccc(-c2nc(-c3ccccc3)nc(-c3ccc(-c4cccc(-n5c6ccccc6c6ccccc65)c4)c4oc5ccccc5c34)n2)cc1 ; c1ccc(-c2ccc(-c3nc(-c4ccccc4)nc(-c4ccc(-c5cccc(-n6c7ccccc7c7ccccc76)c5)c5oc6ccccc6c45)n3)cc2)cc1 ; c1ccc(-c2cccc(-c3nc(-c4ccccc4)nc(-c4ccc(-c5cccc(-n6c7ccccc7c7ccccc76)c5)c5oc6ccccc6c45)n3)c2)cc1]

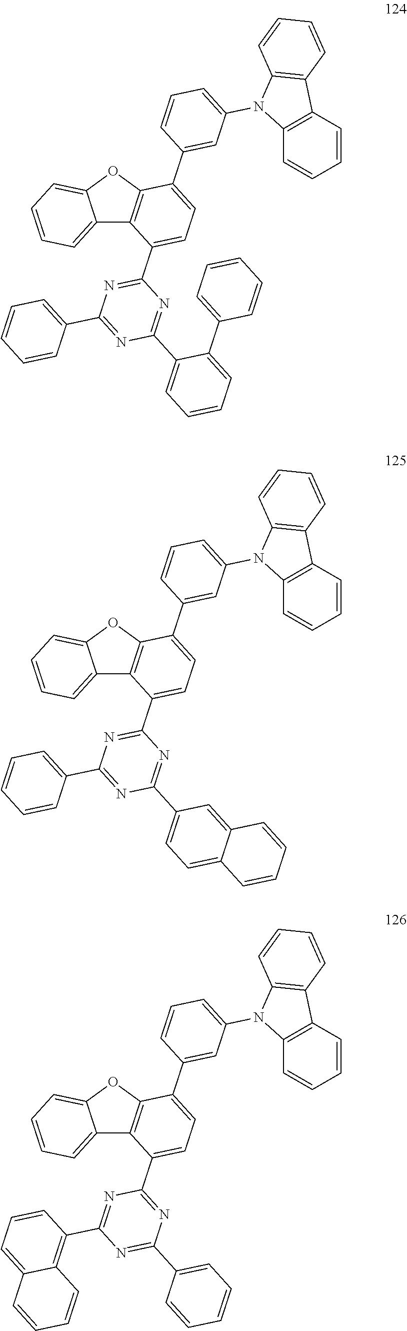 [SMILES: c1ccc(-c2nc(-c3ccccc3-c3ccccc3)nc(-c3ccc(-c4cccc(-n5c6ccccc6c6ccccc65)c4)c4oc5ccccc5c34)n2)cc1 ; c1ccc(-c2nc(-c3ccc4ccccc4c3)nc(-c3ccc(-c4cccc(-n5c6ccccc6c6ccccc65)c4)c4oc5ccccc5c34)n2)cc1 ; c1ccc(-c2nc(-c3cccc4ccccc34)nc(-c3ccc(-c4cccc(-n5c6ccccc6c6ccccc65)c4)c4oc5ccccc5c34)n2)cc1]

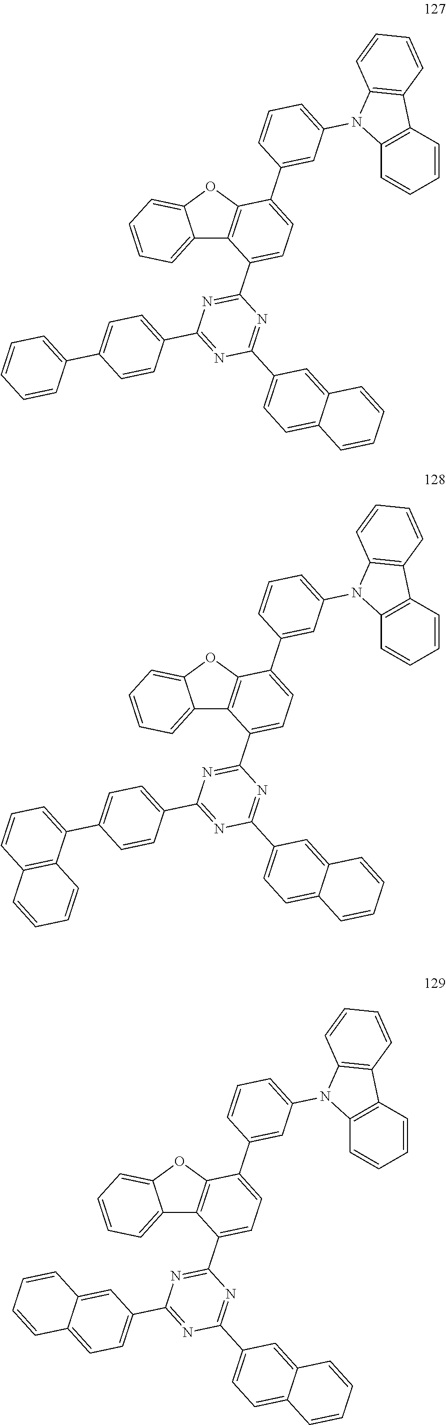 [SMILES: c1ccc(-c2ccc(-c3nc(-c4ccc5ccccc5c4)nc(-c4ccc(-c5cccc(-n6c7ccccc7c7ccccc76)c5)c5oc6ccccc6c45)n3)cc2)cc1 ; c1cc(-c2ccc(-c3nc(-c4ccc(-c5cccc6ccccc56)cc4)nc(-c4ccc5ccccc5c4)n3)c3c2oc2ccccc23)cc(-n2c3ccccc3c3ccccc32)c1 ; c1cc(-c2ccc(-c3nc(-c4ccc5ccccc5c4)nc(-c4ccc5ccccc5c4)n3)c3c2oc2ccccc23)cc(-n2c3ccccc3c3ccccc32)c1]

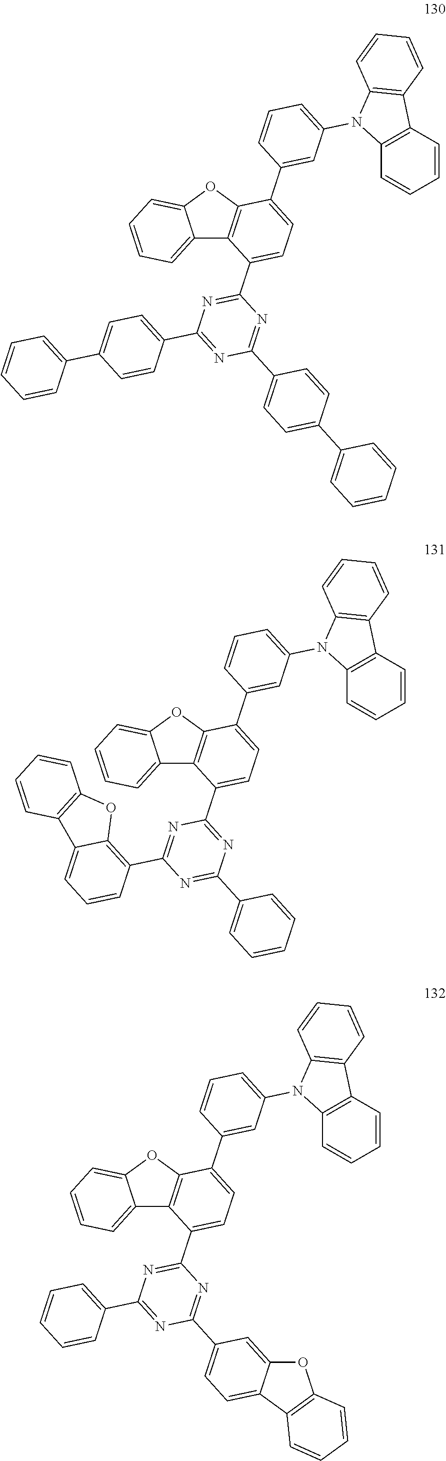 [SMILES: c1ccc(-c2ccc(-c3nc(-c4ccc(-c5ccccc5)cc4)nc(-c4ccc(-c5cccc(-n6c7ccccc7c7ccccc76)c5)c5oc6ccccc6c45)n3)cc2)cc1 ; c1ccc(-c2nc(-c3cccc4c3oc3ccccc34)nc(-c3ccc(-c4cccc(-n5c6ccccc6c6ccccc65)c4)c4oc5ccccc5c34)n2)cc1 ; c1ccc(-c2nc(-c3ccc4c(c3)oc3ccccc34)nc(-c3ccc(-c4cccc(-n5c6ccccc6c6ccccc65)c4)c4oc5ccccc5c34)n2)cc1]

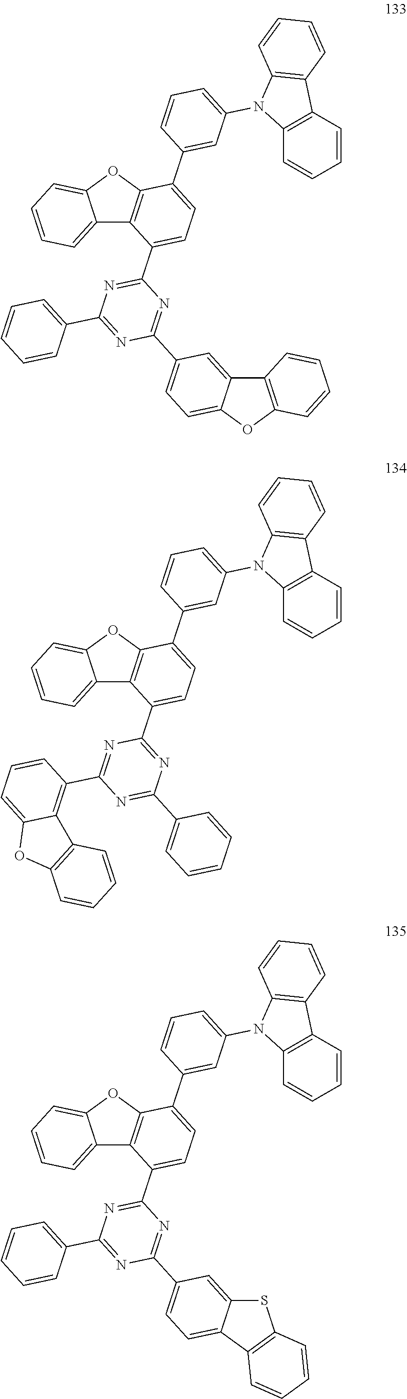 [SMILES: c1ccc(-c2nc(-c3ccc4oc5ccccc5c4c3)nc(-c3ccc(-c4cccc(-n5c6ccccc6c6ccccc65)c4)c4oc5ccccc5c34)n2)cc1 ; c1ccc(-c2nc(-c3cccc4oc5ccccc5c34)nc(-c3ccc(-c4cccc(-n5c6ccccc6c6ccccc65)c4)c4oc5ccccc5c34)n2)cc1 ; c1ccc(-c2nc(-c3ccc4c(c3)sc3ccccc34)nc(-c3ccc(-c4cccc(-n5c6ccccc6c6ccccc65)c4)c4oc5ccccc5c34)n2)cc1]

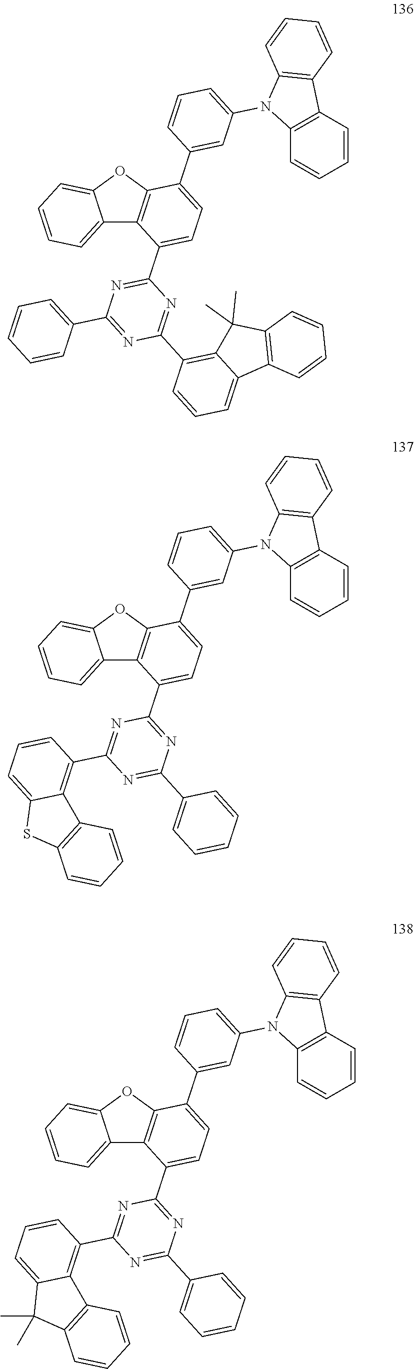 [SMILES: CC1(C)c2ccccc2-c2cccc(-c3nc(-c4ccccc4)nc(-c4ccc(-c5cccc(-n6c7ccccc7c7ccccc76)c5)c5oc6ccccc6c45)n3)c21 ; c1ccc(-c2nc(-c3cccc4sc5ccccc5c34)nc(-c3ccc(-c4cccc(-n5c6ccccc6c6ccccc65)c4)c4oc5ccccc5c34)n2)cc1 ; CC1(C)c2ccccc2-c2c(-c3nc(-c4ccccc4)nc(-c4ccc(-c5cccc(-n6c7ccccc7c7ccccc76)c5)c5oc6ccccc6c45)n3)cccc21]

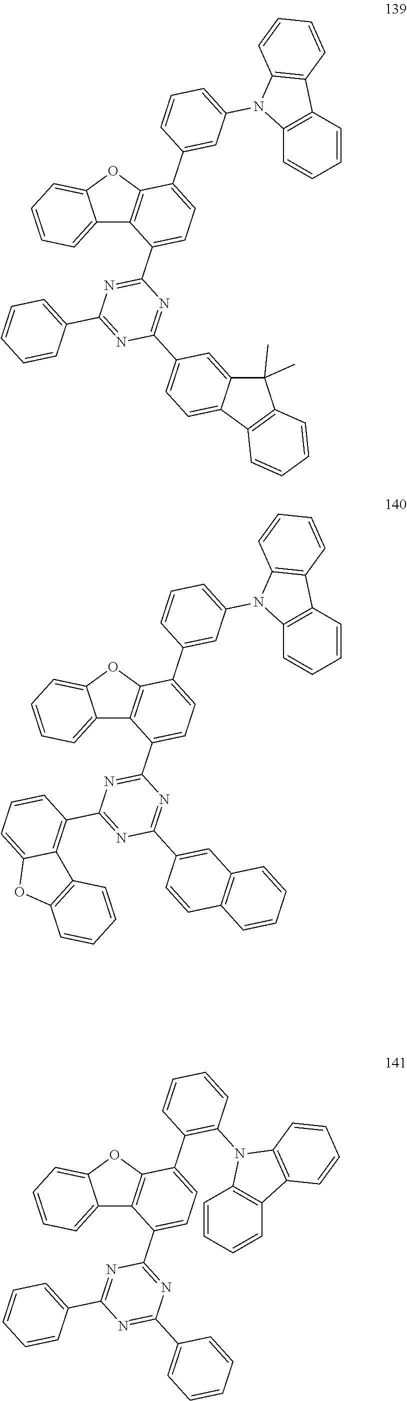

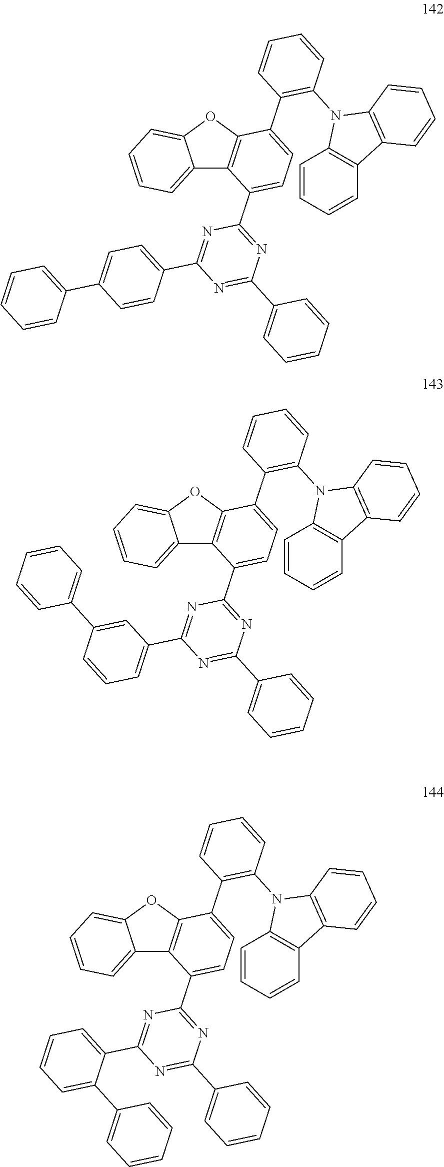 [SMILES: c1ccc(-c2ccc(-c3nc(-c4ccccc4)nc(-c4ccc(-c5ccccc5-n5c6ccccc6c6ccccc65)c5oc6ccccc6c45)n3)cc2)cc1 ; c1ccc(-c2cccc(-c3nc(-c4ccccc4)nc(-c4ccc(-c5ccccc5-n5c6ccccc6c6ccccc65)c5oc6ccccc6c45)n3)c2)cc1 ; c1ccc(-c2nc(-c3ccccc3-c3ccccc3)nc(-c3ccc(-c4ccccc4-n4c5ccccc5c5ccccc54)c4oc5ccccc5c34)n2)cc1]

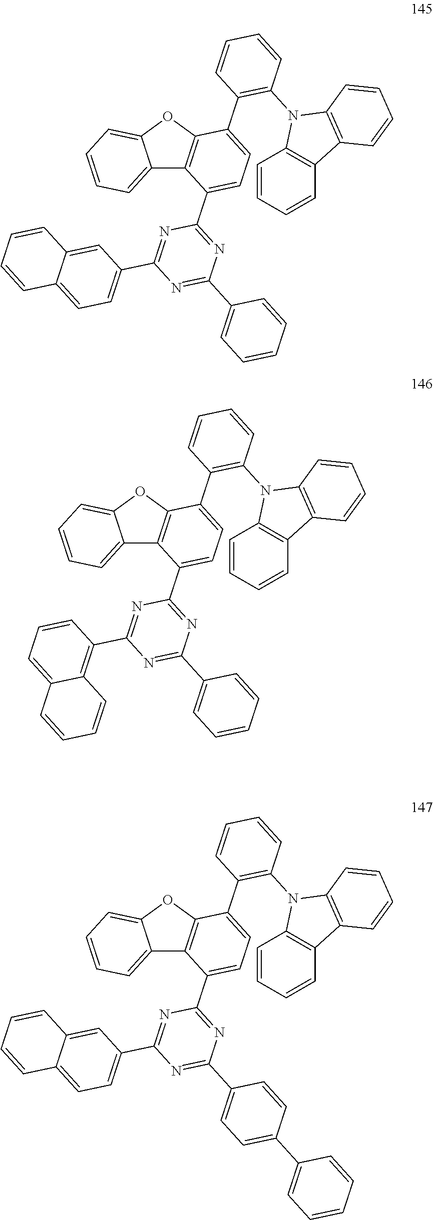 [SMILES: c1ccc(-c2nc(-c3ccc4ccccc4c3)nc(-c3ccc(-c4ccccc4-n4c5ccccc5c5ccccc54)c4oc5ccccc5c34)n2)cc1 ; c1ccc(-c2nc(-c3cccc4ccccc34)nc(-c3ccc(-c4ccccc4-n4c5ccccc5c5ccccc54)c4oc5ccccc5c34)n2)cc1 ; c1ccc(-c2ccc(-c3nc(-c4ccc5ccccc5c4)nc(-c4ccc(-c5ccccc5-n5c6ccccc6c6ccccc65)c5oc6ccccc6c45)n3)cc2)cc1]

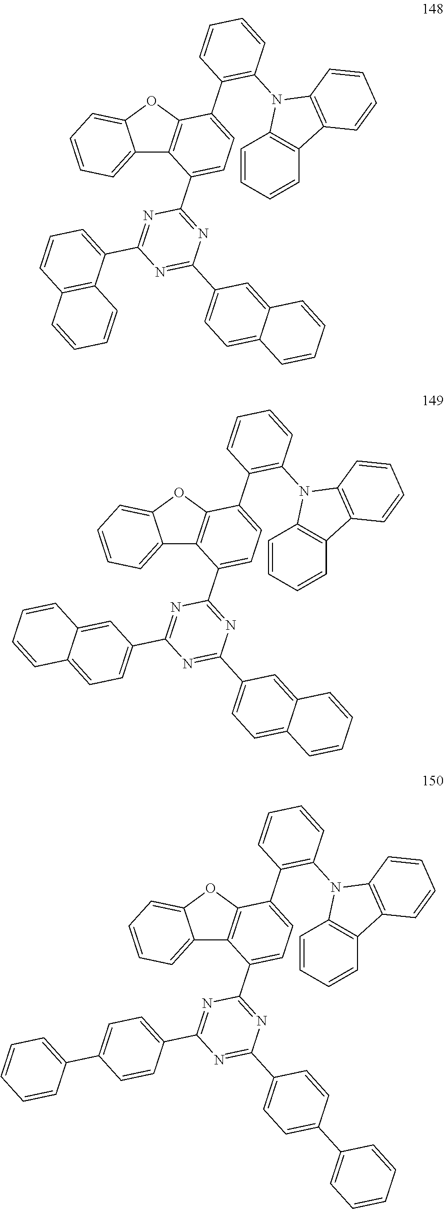 [SMILES: c1ccc(-n2c3ccccc3c3ccccc32)c(-c2ccc(-c3nc(-c4ccc5ccccc5c4)nc(-c4cccc5ccccc45)n3)c3c2oc2ccccc23)c1 ; c1ccc(-n2c3ccccc3c3ccccc32)c(-c2ccc(-c3nc(-c4ccc5ccccc5c4)nc(-c4ccc5ccccc5c4)n3)c3c2oc2ccccc23)c1 ; c1ccc(-c2ccc(-c3nc(-c4ccc(-c5ccccc5)cc4)nc(-c4ccc(-c5ccccc5-n5c6ccccc6c6ccccc65)c5oc6ccccc6c45)n3)cc2)cc1]

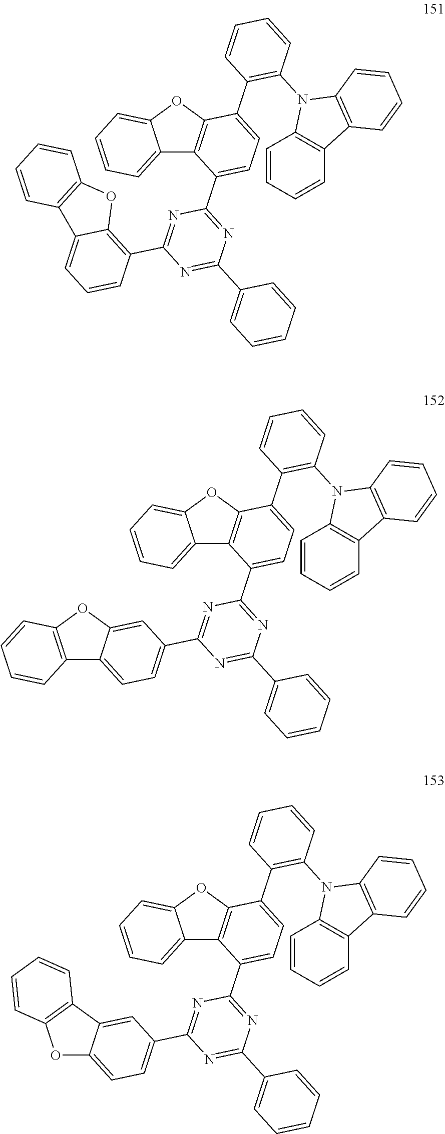 [SMILES: c1ccc(-c2nc(-c3cccc4c3oc3ccccc34)nc(-c3ccc(-c4ccccc4-n4c5ccccc5c5ccccc54)c4oc5ccccc5c34)n2)cc1 ; c1ccc(-c2nc(-c3ccc4c(c3)oc3ccccc34)nc(-c3ccc(-c4ccccc4-n4c5ccccc5c5ccccc54)c4oc5ccccc5c34)n2)cc1 ; c1ccc(-c2nc(-c3ccc4oc5ccccc5c4c3)nc(-c3ccc(-c4ccccc4-n4c5ccccc5c5ccccc54)c4oc5ccccc5c34)n2)cc1]

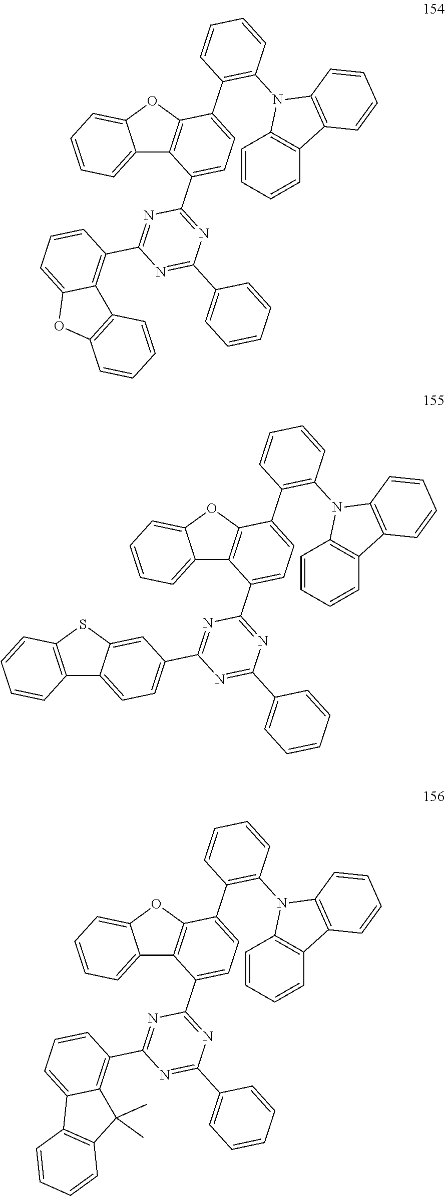 [SMILES: c1ccc(-c2nc(-c3cccc4oc5ccccc5c34)nc(-c3ccc(-c4ccccc4-n4c5ccccc5c5ccccc54)c4oc5ccccc5c34)n2)cc1 ; c1ccc(-c2nc(-c3ccc4c(c3)sc3ccccc34)nc(-c3ccc(-c4ccccc4-n4c5ccccc5c5ccccc54)c4oc5ccccc5c34)n2)cc1 ; CC1(C)c2ccccc2-c2cccc(-c3nc(-c4ccccc4)nc(-c4ccc(-c5ccccc5-n5c6ccccc6c6ccccc65)c5oc6ccccc6c45)n3)c21]

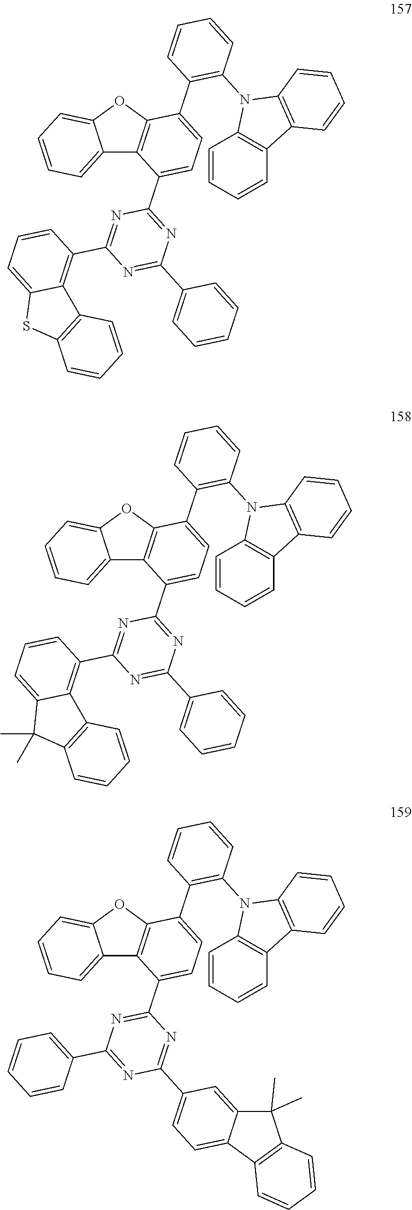 [SMILES: c1ccc(-c2nc(-c3cccc4sc5ccccc5c34)nc(-c3ccc(-c4ccccc4-n4c5ccccc5c5ccccc54)c4oc5ccccc5c34)n2)cc1 ; CC1(C)c2ccccc2-c2c(-c3nc(-c4ccccc4)nc(-c4ccc(-c5ccccc5-n5c6ccccc6c6ccccc65)c5oc6ccccc6c45)n3)cccc21 ; CC1(C)c2ccccc2-c2ccc(-c3nc(-c4ccccc4)nc(-c4ccc(-c5ccccc5-n5c6ccccc6c6ccccc65)c5oc6ccccc6c45)n3)cc21]

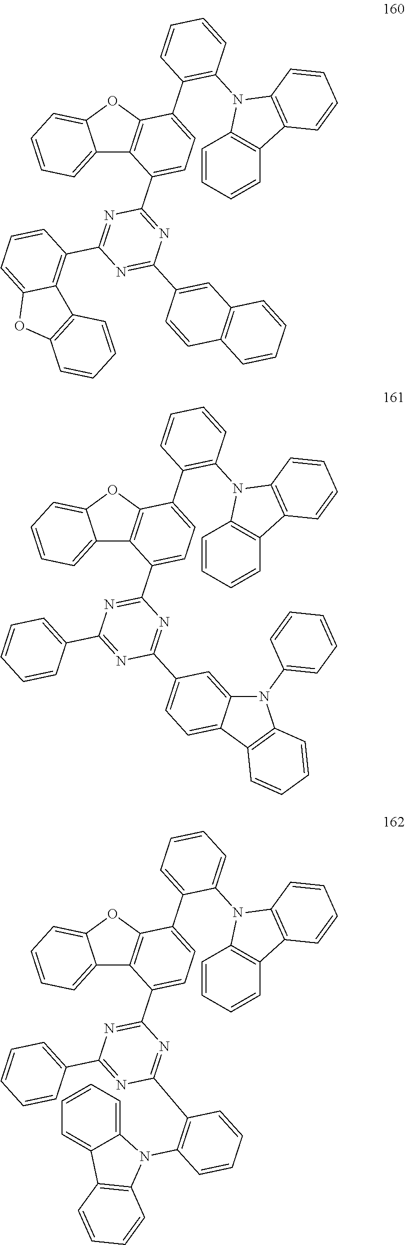 [SMILES: c1ccc(-n2c3ccccc3c3ccccc32)c(-c2ccc(-c3nc(-c4ccc5ccccc5c4)nc(-c4cccc5oc6ccccc6c45)n3)c3c2oc2ccccc23)c1 ; c1ccc(-c2nc(-c3ccc4c5ccccc5n(-c5ccccc5)c4c3)nc(-c3ccc(-c4ccccc4-n4c5ccccc5c5ccccc54)c4oc5ccccc5c34)n2)cc1 ; c1ccc(-c2nc(-c3ccccc3-n3c4ccccc4c4ccccc43)nc(-c3ccc(-c4ccccc4-n4c5ccccc5c5ccccc54)c4oc5ccccc5c34)n2)cc1]

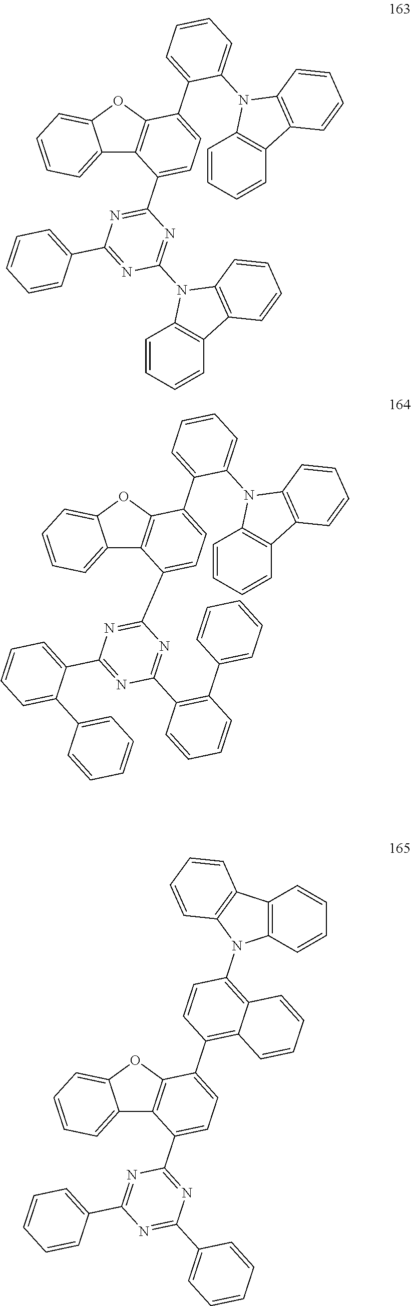

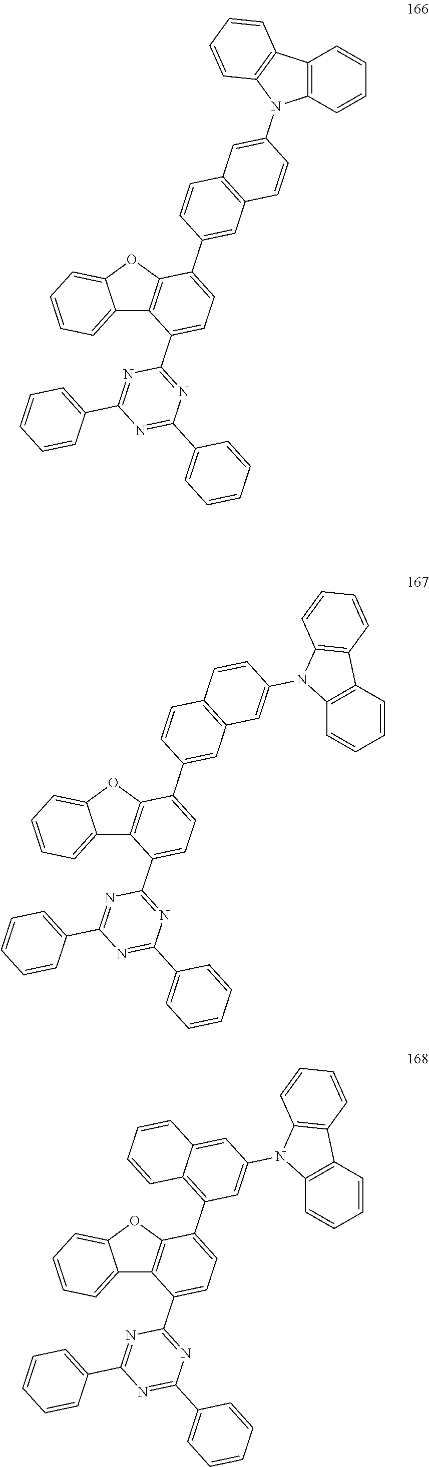 [SMILES: c1ccc(-c2nc(-c3ccccc3)nc(-c3ccc(-c4ccc5cc(-n6c7ccccc7c7ccccc76)ccc5c4)c4oc5ccccc5c34)n2)cc1 ; c1ccc(-c2nc(-c3ccccc3)nc(-c3ccc(-c4ccc5ccc(-n6c7ccccc7c7ccccc76)cc5c4)c4oc5ccccc5c34)n2)cc1 ; c1ccc(-c2nc(-c3ccccc3)nc(-c3ccc(-c4cc(-n5c6ccccc6c6ccccc65)cc5ccccc45)c4oc5ccccc5c34)n2)cc1]

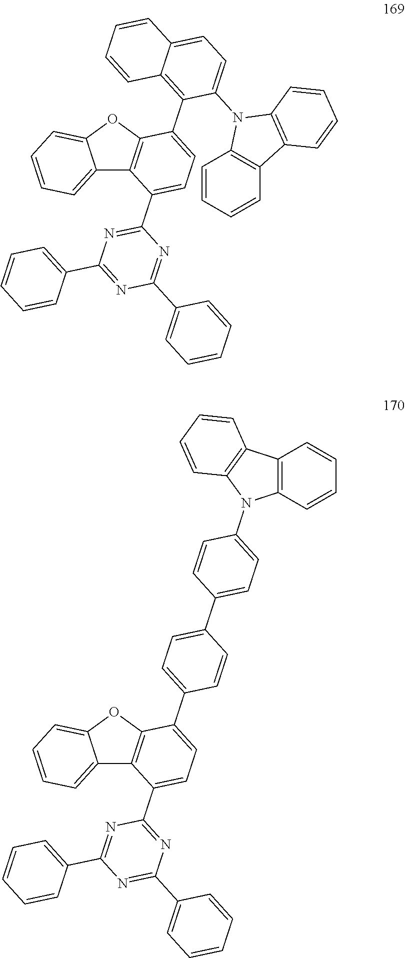 [SMILES: c1ccc(-c2nc(-c3ccccc3)nc(-c3ccc(-c4c(-n5c6ccccc6c6ccccc65)ccc5ccccc45)c4oc5ccccc5c34)n2)cc1 ; c1ccc(-c2nc(-c3ccccc3)nc(-c3ccc(-c4ccc(-c5ccc(-n6c7ccccc7c7ccccc76)cc5)cc4)c4oc5ccccc5c34)n2)cc1]

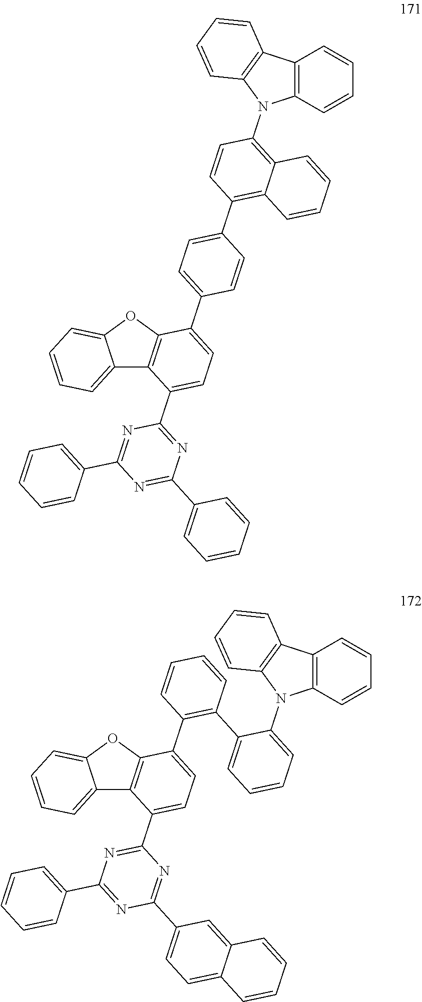 [SMILES: c1ccc(-c2nc(-c3ccccc3)nc(-c3ccc(-c4ccc(-c5ccc(-n6c7ccccc7c7ccccc76)c6ccccc56)cc4)c4oc5ccccc5c34)n2)cc1 ; c1ccc(-c2nc(-c3ccc4ccccc4c3)nc(-c3ccc(-c4ccccc4-c4ccccc4-n4c5ccccc5c5ccccc54)c4oc5ccccc5c34)n2)cc1]

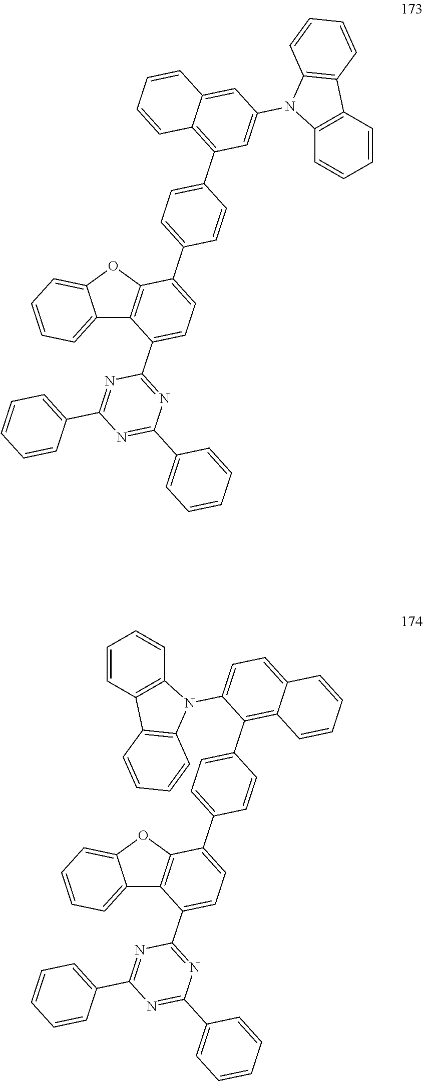 [SMILES: c1ccc(-c2nc(-c3ccccc3)nc(-c3ccc(-c4ccc(-c5cc(-n6c7ccccc7c7ccccc76)cc6ccccc56)cc4)c4oc5ccccc5c34)n2)cc1 ; c1ccc(-c2nc(-c3ccccc3)nc(-c3ccc(-c4ccc(-c5c(-n6c7ccccc7c7ccccc76)ccc6ccccc56)cc4)c4oc5ccccc5c34)n2)cc1]

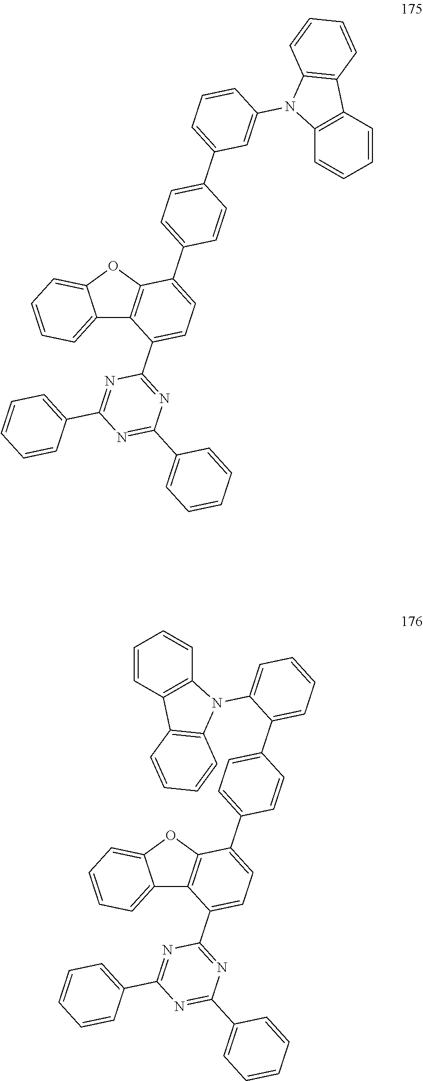 [SMILES: c1ccc(-c2nc(-c3ccccc3)nc(-c3ccc(-c4ccc(-c5cccc(-n6c7ccccc7c7ccccc76)c5)cc4)c4oc5ccccc5c34)n2)cc1 ; c1ccc(-c2nc(-c3ccccc3)nc(-c3ccc(-c4ccc(-c5ccccc5-n5c6ccccc6c6ccccc65)cc4)c4oc5ccccc5c34)n2)cc1]

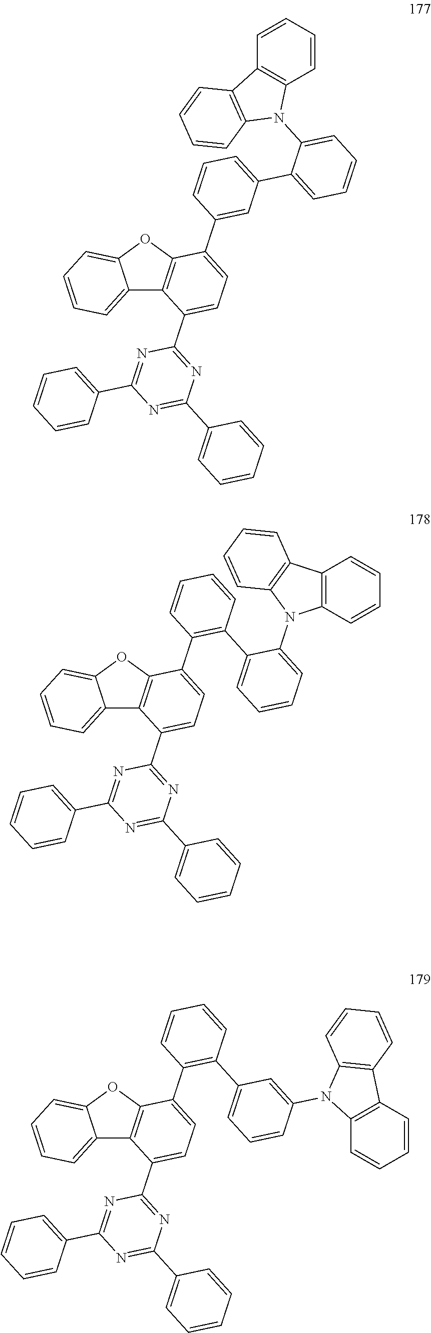 [SMILES: c1ccc(-c2nc(-c3ccccc3)nc(-c3ccc(-c4cccc(-c5ccccc5-n5c6ccccc6c6ccccc65)c4)c4oc5ccccc5c34)n2)cc1 ; c1ccc(-c2nc(-c3ccccc3)nc(-c3ccc(-c4ccccc4-c4ccccc4-n4c5ccccc5c5ccccc54)c4oc5ccccc5c34)n2)cc1 ; c1ccc(-c2nc(-c3ccccc3)nc(-c3ccc(-c4ccccc4-c4cccc(-n5c6ccccc6c6ccccc65)c4)c4oc5ccccc5c34)n2)cc1]

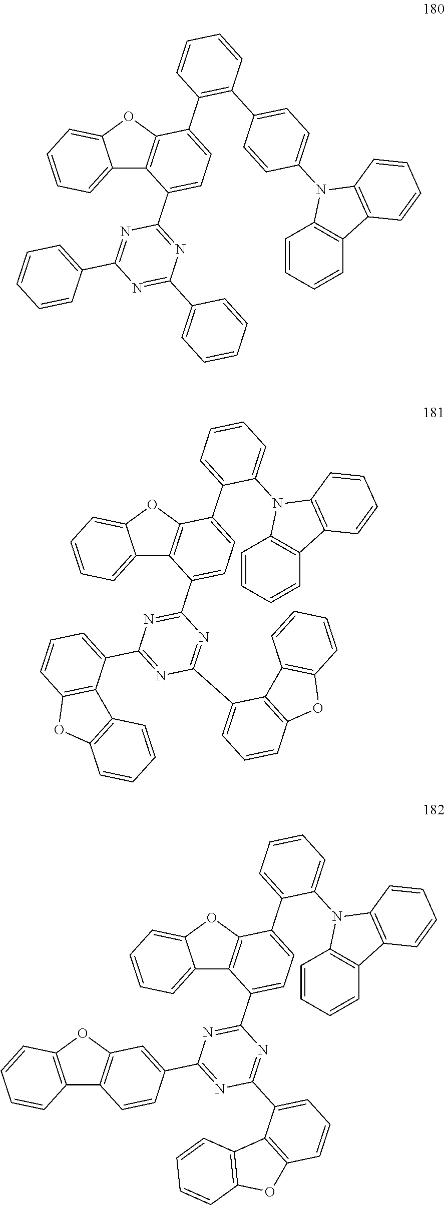 [SMILES: c1ccc(-c2nc(-c3ccccc3)nc(-c3ccc(-c4ccccc4-c4ccc(-n5c6ccccc6c6ccccc65)cc4)c4oc5ccccc5c34)n2)cc1 ; c1ccc(-n2c3ccccc3c3ccccc32)c(-c2ccc(-c3nc(-c4cccc5oc6ccccc6c45)nc(-c4cccc5oc6ccccc6c45)n3)c3c2oc2ccccc23)c1 ; c1ccc(-n2c3ccccc3c3ccccc32)c(-c2ccc(-c3nc(-c4ccc5c(c4)oc4ccccc45)nc(-c4cccc5oc6ccccc6c45)n3)c3c2oc2ccccc23)c1]

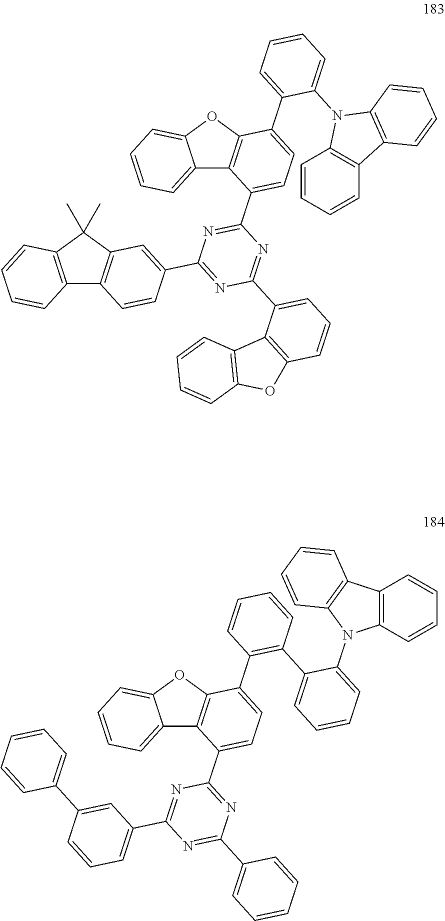 [SMILES: CC1(C)c2ccccc2-c2ccc(-c3nc(-c4cccc5oc6ccccc6c45)nc(-c4ccc(-c5ccccc5-n5c6ccccc6c6ccccc65)c5oc6ccccc6c45)n3)cc21 ; c1ccc(-c2cccc(-c3nc(-c4ccccc4)nc(-c4ccc(-c5ccccc5-c5ccccc5-n5c6ccccc6c6ccccc65)c5oc6ccccc6c45)n3)c2)cc1]

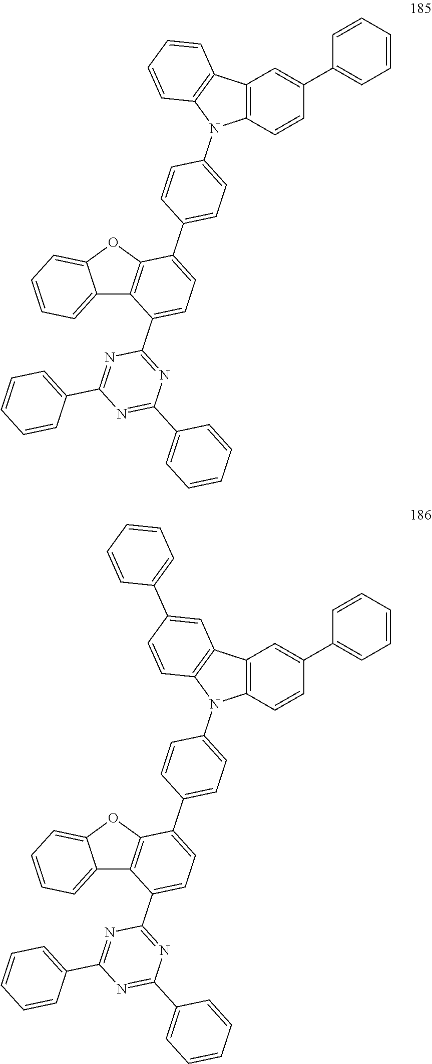 [SMILES: c1ccc(-c2ccc3c(c2)c2ccccc2n3-c2ccc(-c3ccc(-c4nc(-c5ccccc5)nc(-c5ccccc5)n4)c4c3oc3ccccc34)cc2)cc1 ; c1ccc(-c2ccc3c(c2)c2cc(-c4ccccc4)ccc2n3-c2ccc(-c3ccc(-c4nc(-c5ccccc5)nc(-c5ccccc5)n4)c4c3oc3ccccc34)cc2)cc1]

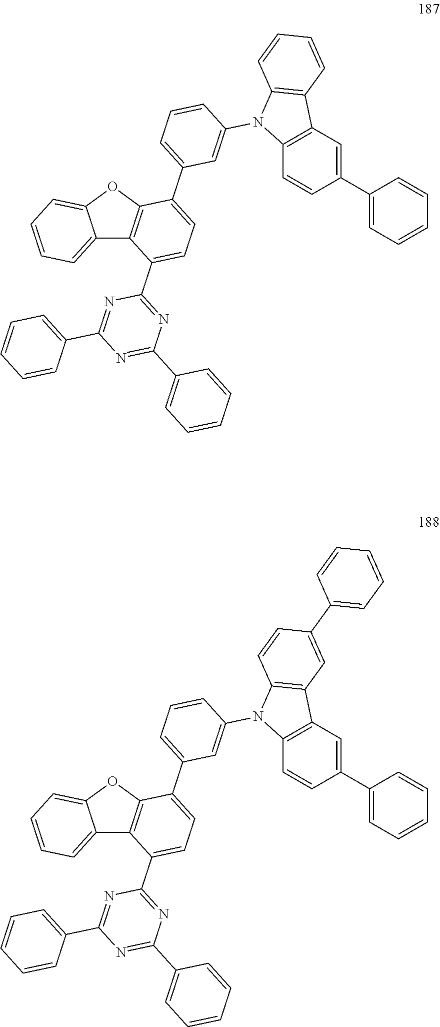 [SMILES: c1ccc(-c2ccc3c(c2)c2ccccc2n3-c2cccc(-c3ccc(-c4nc(-c5ccccc5)nc(-c5ccccc5)n4)c4c3oc3ccccc34)c2)cc1 ; c1ccc(-c2ccc3c(c2)c2cc(-c4ccccc4)ccc2n3-c2cccc(-c3ccc(-c4nc(-c5ccccc5)nc(-c5ccccc5)n4)c4c3oc3ccccc34)c2)cc1]

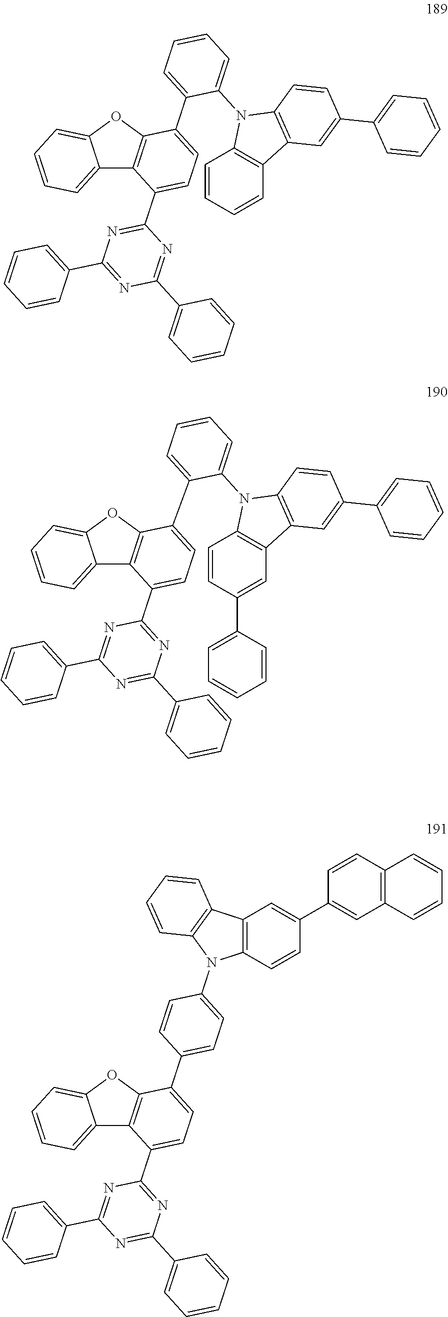 [SMILES: c1ccc(-c2ccc3c(c2)c2ccccc2n3-c2ccccc2-c2ccc(-c3nc(-c4ccccc4)nc(-c4ccccc4)n3)c3c2oc2ccccc23)cc1 ; c1ccc(-c2ccc3c(c2)c2cc(-c4ccccc4)ccc2n3-c2ccccc2-c2ccc(-c3nc(-c4ccccc4)nc(-c4ccccc4)n3)c3c2oc2ccccc23)cc1 ; c1ccc(-c2nc(-c3ccccc3)nc(-c3ccc(-c4ccc(-n5c6ccccc6c6cc(-c7ccc8ccccc8c7)ccc65)cc4)c4oc5ccccc5c34)n2)cc1]

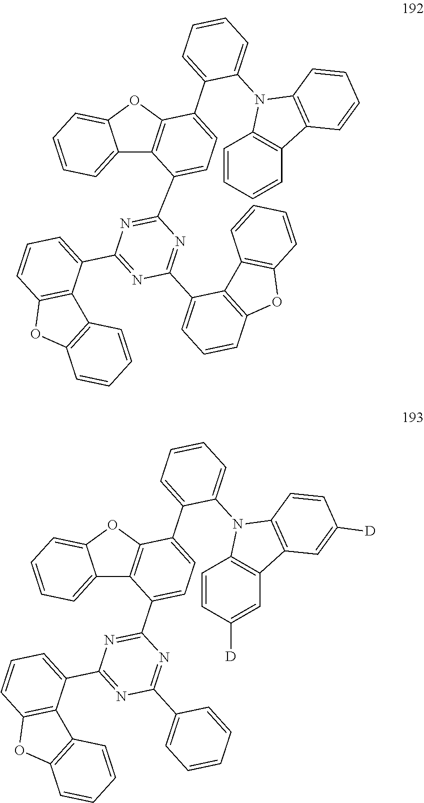 [SMILES: c1ccc(-n2c3ccccc3c3ccccc32)c(-c2ccc(-c3nc(-c4cccc5oc6ccccc6c45)nc(-c4cccc5oc6ccccc6c45)n3)c3c2oc2ccccc23)c1 ; [2H]c1ccc2c(c1)c1cc([2H])ccc1n2-c1ccccc1-c1ccc(-c2nc(-c3ccccc3)nc(-c3cccc4oc5ccccc5c34)n2)c2c1oc1ccccc12]

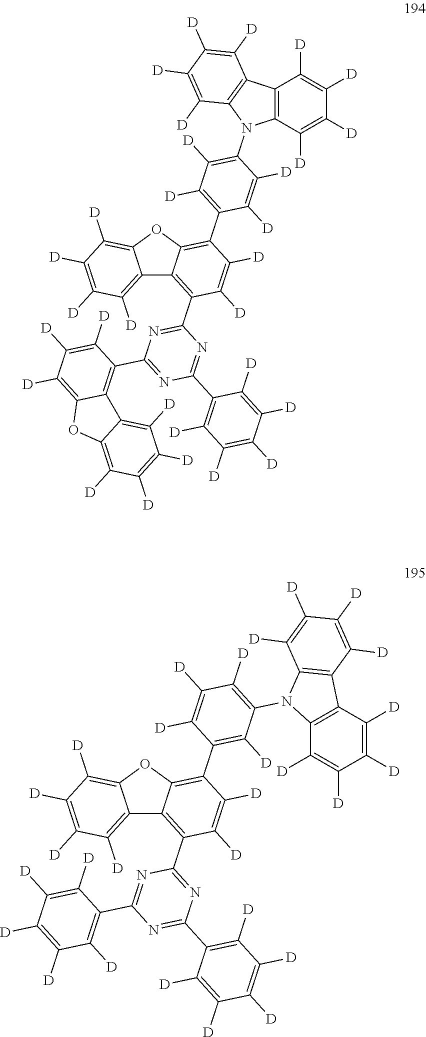

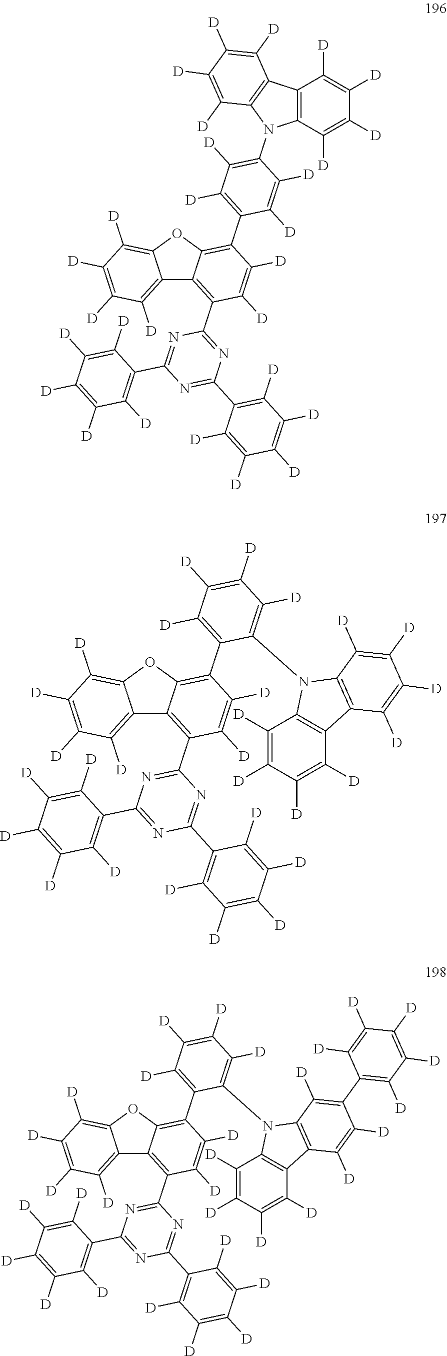

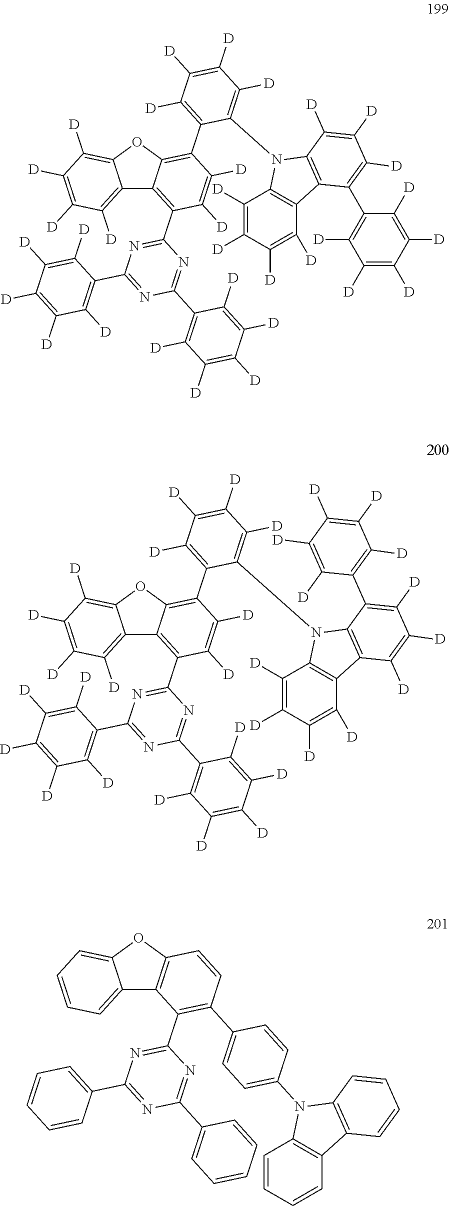

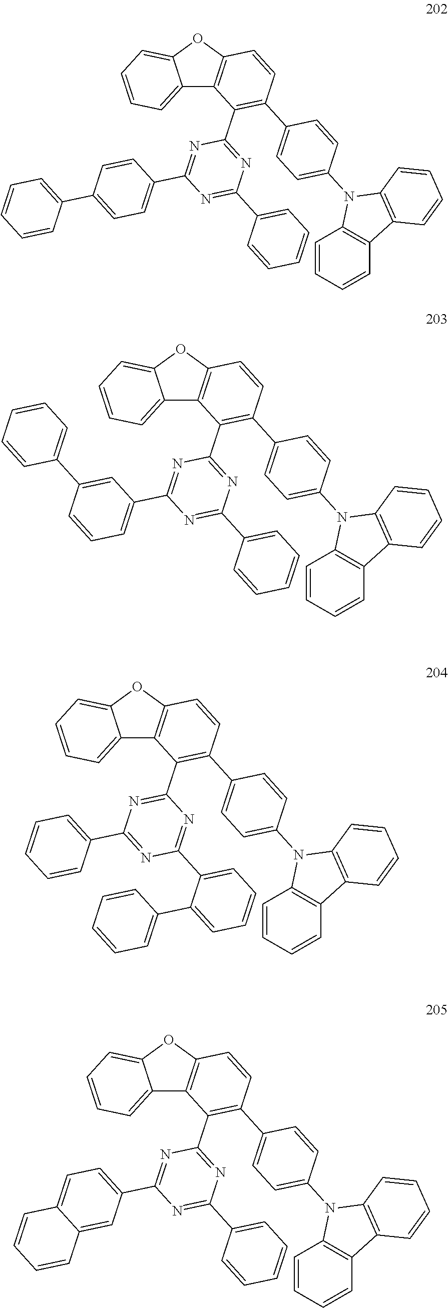 [SMILES: c1ccc(-c2ccc(-c3nc(-c4ccccc4)nc(-c4c(-c5ccc(-n6c7ccccc7c7ccccc76)cc5)ccc5oc6ccccc6c45)n3)cc2)cc1 ; c1ccc(-c2cccc(-c3nc(-c4ccccc4)nc(-c4c(-c5ccc(-n6c7ccccc7c7ccccc76)cc5)ccc5oc6ccccc6c45)n3)c2)cc1 ; c1ccc(-c2nc(-c3ccccc3-c3ccccc3)nc(-c3c(-c4ccc(-n5c6ccccc6c6ccccc65)cc4)ccc4oc5ccccc5c34)n2)cc1 ; c1ccc(-c2nc(-c3ccc4ccccc4c3)nc(-c3c(-c4ccc(-n5c6ccccc6c6ccccc65)cc4)ccc4oc5ccccc5c34)n2)cc1]

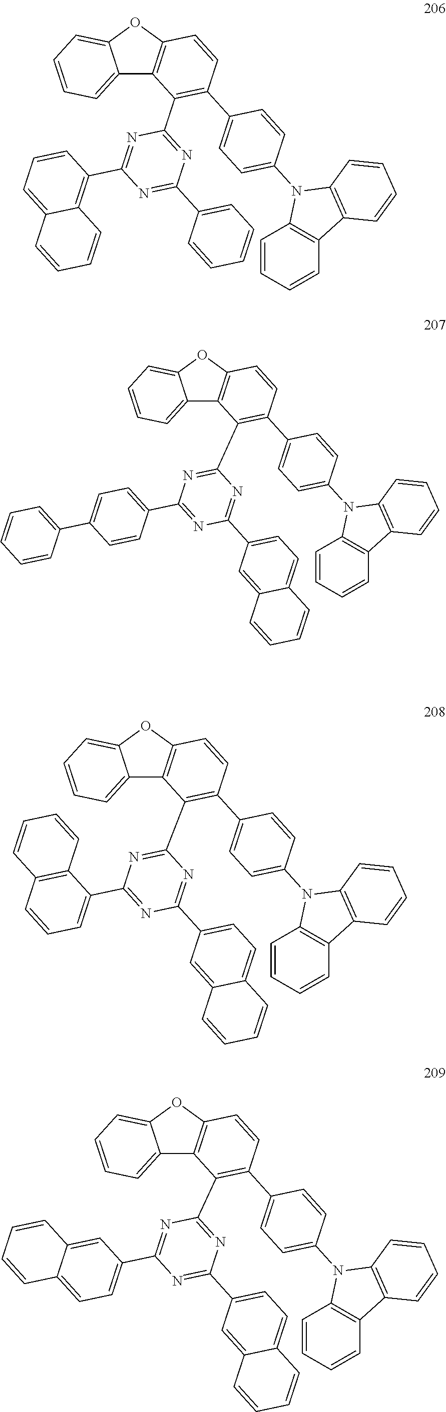 [SMILES: c1ccc(-c2nc(-c3cccc4ccccc34)nc(-c3c(-c4ccc(-n5c6ccccc6c6ccccc65)cc4)ccc4oc5ccccc5c34)n2)cc1 ; c1ccc(-c2ccc(-c3nc(-c4ccc5ccccc5c4)nc(-c4c(-c5ccc(-n6c7ccccc7c7ccccc76)cc5)ccc5oc6ccccc6c45)n3)cc2)cc1 ; c1ccc2cc(-c3nc(-c4cccc5ccccc45)nc(-c4c(-c5ccc(-n6c7ccccc7c7ccccc76)cc5)ccc5oc6ccccc6c45)n3)ccc2c1 ; c1ccc2cc(-c3nc(-c4ccc5ccccc5c4)nc(-c4c(-c5ccc(-n6c7ccccc7c7ccccc76)cc5)ccc5oc6ccccc6c45)n3)ccc2c1]

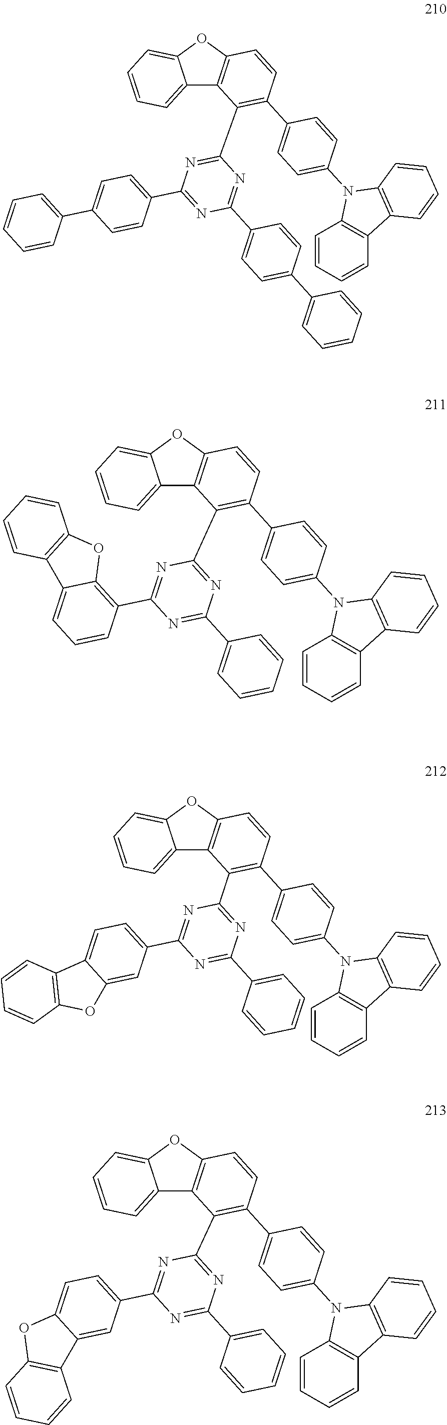 [SMILES: c1ccc(-c2ccc(-c3nc(-c4ccc(-c5ccccc5)cc4)nc(-c4c(-c5ccc(-n6c7ccccc7c7ccccc76)cc5)ccc5oc6ccccc6c45)n3)cc2)cc1 ; c1ccc(-c2nc(-c3cccc4c3oc3ccccc34)nc(-c3c(-c4ccc(-n5c6ccccc6c6ccccc65)cc4)ccc4oc5ccccc5c34)n2)cc1 ; c1ccc(-c2nc(-c3ccc4c(c3)oc3ccccc34)nc(-c3c(-c4ccc(-n5c6ccccc6c6ccccc65)cc4)ccc4oc5ccccc5c34)n2)cc1 ; c1ccc(-c2nc(-c3ccc4oc5ccccc5c4c3)nc(-c3c(-c4ccc(-n5c6ccccc6c6ccccc65)cc4)ccc4oc5ccccc5c34)n2)cc1]

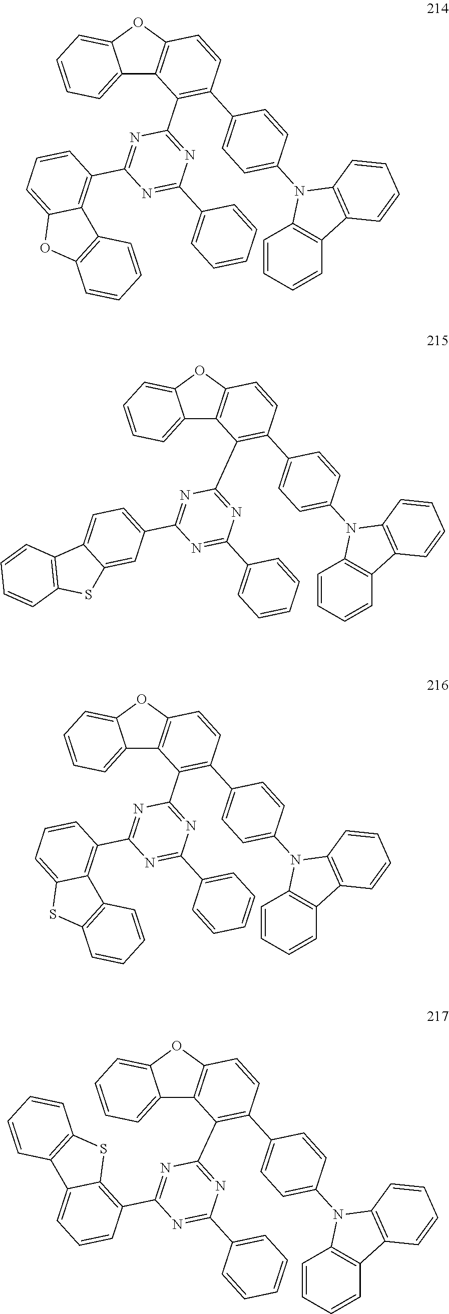 [SMILES: c1ccc(-c2nc(-c3cccc4oc5ccccc5c34)nc(-c3c(-c4ccc(-n5c6ccccc6c6ccccc65)cc4)ccc4oc5ccccc5c34)n2)cc1 ; c1ccc(-c2nc(-c3ccc4c(c3)sc3ccccc34)nc(-c3c(-c4ccc(-n5c6ccccc6c6ccccc65)cc4)ccc4oc5ccccc5c34)n2)cc1 ; c1ccc(-c2nc(-c3c(-c4ccc(-n5c6ccccc6c6ccccc65)cc4)ccc4oc5ccccc5c34)nc(-c3cccc4sc5ccccc5c34)n2)cc1 ; c1ccc(-c2nc(-c3cccc4c3sc3ccccc34)nc(-c3c(-c4ccc(-n5c6ccccc6c6ccccc65)cc4)ccc4oc5ccccc5c34)n2)cc1]

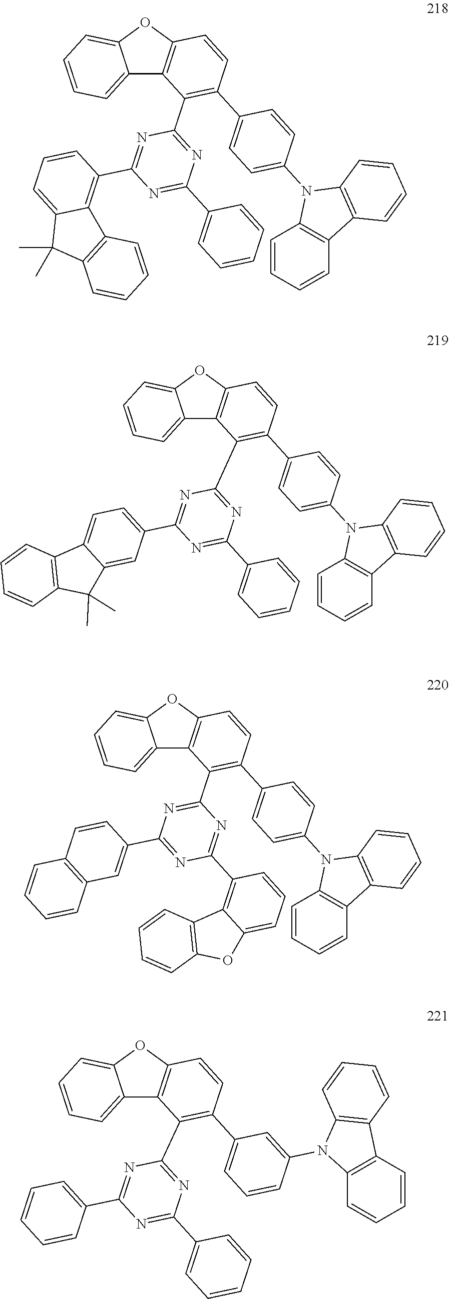 [SMILES: CC1(C)c2ccccc2-c2c(-c3nc(-c4ccccc4)nc(-c4c(-c5ccc(-n6c7ccccc7c7ccccc76)cc5)ccc5oc6ccccc6c45)n3)cccc21 ; CC1(C)c2ccccc2-c2ccc(-c3nc(-c4ccccc4)nc(-c4c(-c5ccc(-n6c7ccccc7c7ccccc76)cc5)ccc5oc6ccccc6c45)n3)cc21 ; c1ccc2cc(-c3nc(-c4cccc5oc6ccccc6c45)nc(-c4c(-c5ccc(-n6c7ccccc7c7ccccc76)cc5)ccc5oc6ccccc6c45)n3)ccc2c1 ; c1ccc(-c2nc(-c3ccccc3)nc(-c3c(-c4cccc(-n5c6ccccc6c6ccccc65)c4)ccc4oc5ccccc5c34)n2)cc1]

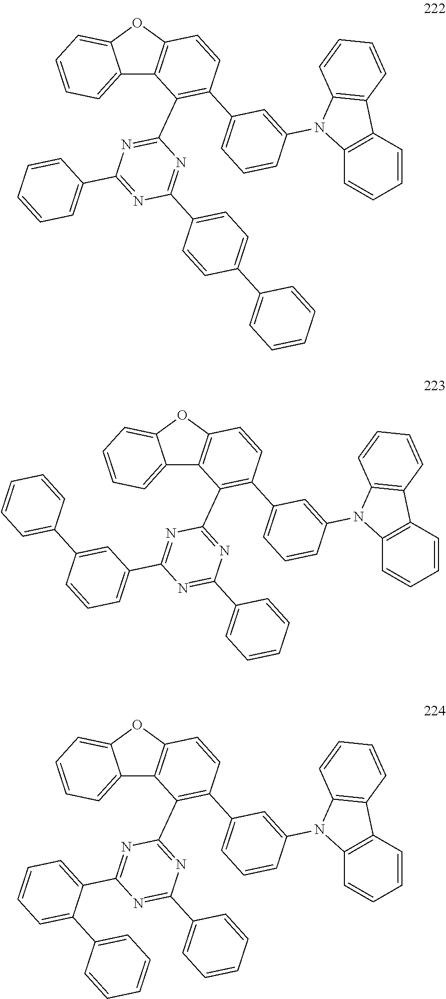 [SMILES: c1ccc(-c2ccc(-c3nc(-c4ccccc4)nc(-c4c(-c5cccc(-n6c7ccccc7c7ccccc76)c5)ccc5oc6ccccc6c45)n3)cc2)cc1 ; c1ccc(-c2cccc(-c3nc(-c4ccccc4)nc(-c4c(-c5cccc(-n6c7ccccc7c7ccccc76)c5)ccc5oc6ccccc6c45)n3)c2)cc1 ; c1ccc(-c2nc(-c3ccccc3-c3ccccc3)nc(-c3c(-c4cccc(-n5c6ccccc6c6ccccc65)c4)ccc4oc5ccccc5c34)n2)cc1]

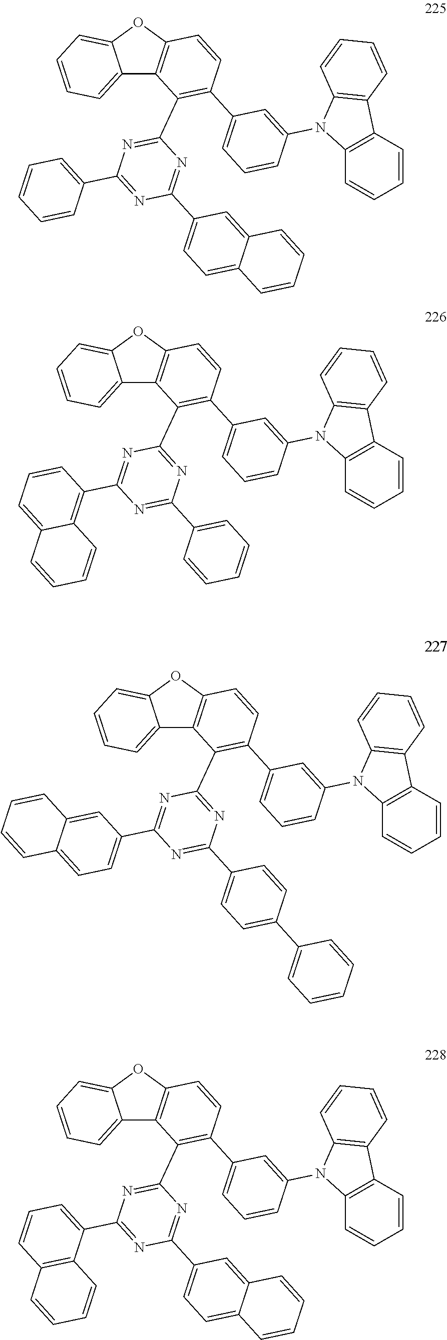 [SMILES: c1ccc(-c2nc(-c3ccc4ccccc4c3)nc(-c3c(-c4cccc(-n5c6ccccc6c6ccccc65)c4)ccc4oc5ccccc5c34)n2)cc1 ; c1ccc(-c2nc(-c3cccc4ccccc34)nc(-c3c(-c4cccc(-n5c6ccccc6c6ccccc65)c4)ccc4oc5ccccc5c34)n2)cc1 ; c1ccc(-c2ccc(-c3nc(-c4ccc5ccccc5c4)nc(-c4c(-c5cccc(-n6c7ccccc7c7ccccc76)c5)ccc5oc6ccccc6c45)n3)cc2)cc1 ; c1cc(-c2ccc3oc4ccccc4c3c2-c2nc(-c3ccc4ccccc4c3)nc(-c3cccc4ccccc34)n2)cc(-n2c3ccccc3c3ccccc32)c1]

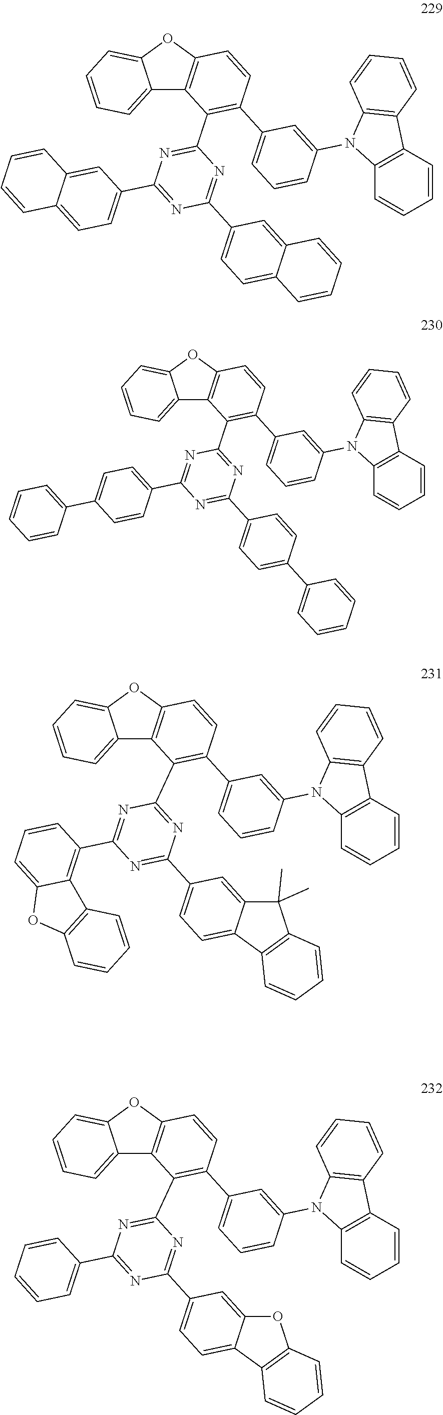 [SMILES: c1cc(-c2ccc3oc4ccccc4c3c2-c2nc(-c3ccc4ccccc4c3)nc(-c3ccc4ccccc4c3)n2)cc(-n2c3ccccc3c3ccccc32)c1 ; c1ccc(-c2ccc(-c3nc(-c4ccc(-c5ccccc5)cc4)nc(-c4c(-c5cccc(-n6c7ccccc7c7ccccc76)c5)ccc5oc6ccccc6c45)n3)cc2)cc1 ; CC1(C)c2ccccc2-c2ccc(-c3nc(-c4cccc5oc6ccccc6c45)nc(-c4c(-c5cccc(-n6c7ccccc7c7ccccc76)c5)ccc5oc6ccccc6c45)n3)cc21 ; c1ccc(-c2nc(-c3ccc4c(c3)oc3ccccc34)nc(-c3c(-c4cccc(-n5c6ccccc6c6ccccc65)c4)ccc4oc5ccccc5c34)n2)cc1]

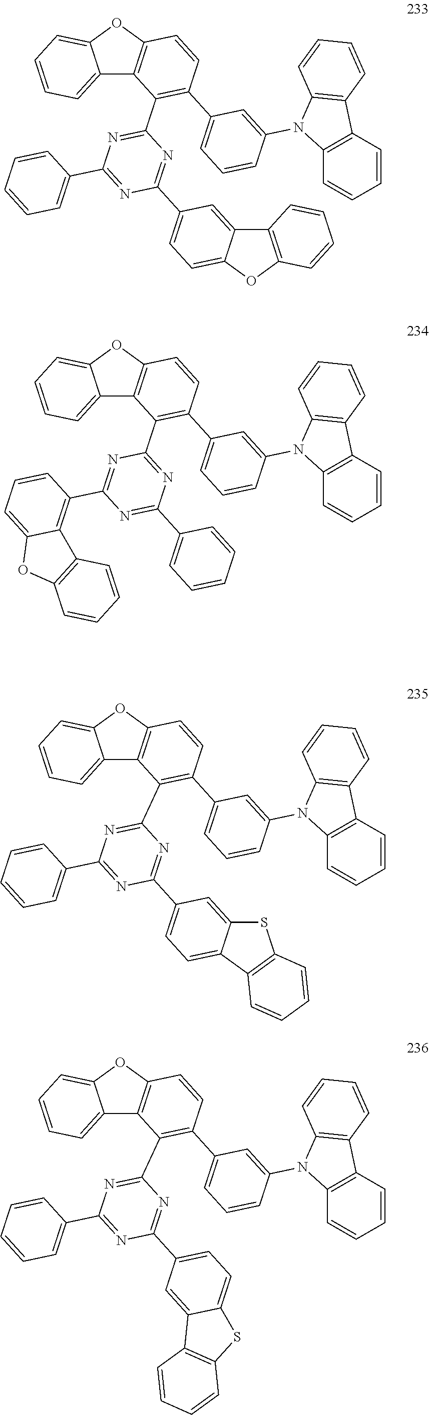 [SMILES: c1ccc(-c2nc(-c3ccc4oc5ccccc5c4c3)nc(-c3c(-c4cccc(-n5c6ccccc6c6ccccc65)c4)ccc4oc5ccccc5c34)n2)cc1 ; c1ccc(-c2nc(-c3cccc4oc5ccccc5c34)nc(-c3c(-c4cccc(-n5c6ccccc6c6ccccc65)c4)ccc4oc5ccccc5c34)n2)cc1 ; c1ccc(-c2nc(-c3ccc4c(c3)sc3ccccc34)nc(-c3c(-c4cccc(-n5c6ccccc6c6ccccc65)c4)ccc4oc5ccccc5c34)n2)cc1 ; c1ccc(-c2nc(-c3ccc4sc5ccccc5c4c3)nc(-c3c(-c4cccc(-n5c6ccccc6c6ccccc65)c4)ccc4oc5ccccc5c34)n2)cc1]

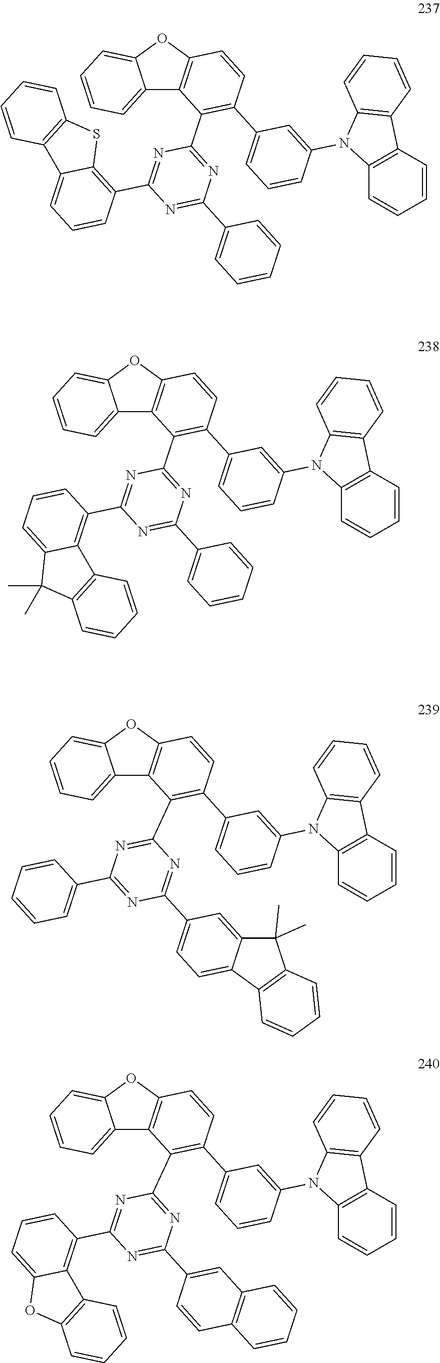 [SMILES: c1ccc(-c2nc(-c3cccc4c3sc3ccccc34)nc(-c3c(-c4cccc(-n5c6ccccc6c6ccccc65)c4)ccc4oc5ccccc5c34)n2)cc1 ; CC1(C)c2ccccc2-c2c(-c3nc(-c4ccccc4)nc(-c4c(-c5cccc(-n6c7ccccc7c7ccccc76)c5)ccc5oc6ccccc6c45)n3)cccc21 ; CC1(C)c2ccccc2-c2ccc(-c3nc(-c4ccccc4)nc(-c4c(-c5cccc(-n6c7ccccc7c7ccccc76)c5)ccc5oc6ccccc6c45)n3)cc21 ; c1cc(-c2ccc3oc4ccccc4c3c2-c2nc(-c3ccc4ccccc4c3)nc(-c3cccc4oc5ccccc5c34)n2)cc(-n2c3ccccc3c3ccccc32)c1]

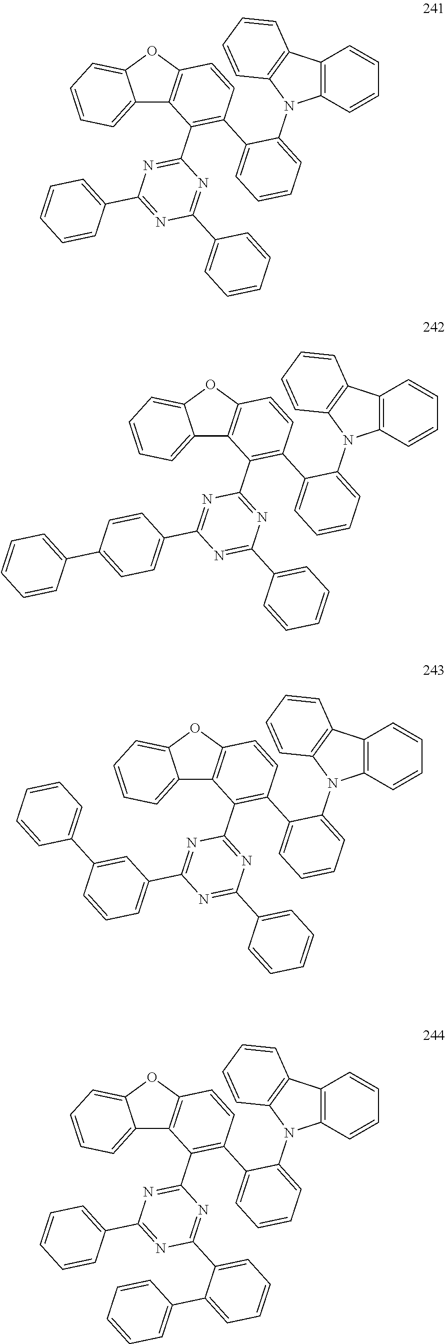 [SMILES: c1ccc(-c2nc(-c3ccccc3)nc(-c3c(-c4ccccc4-n4c5ccccc5c5ccccc54)ccc4oc5ccccc5c34)n2)cc1 ; c1ccc(-c2ccc(-c3nc(-c4ccccc4)nc(-c4c(-c5ccccc5-n5c6ccccc6c6ccccc65)ccc5oc6ccccc6c45)n3)cc2)cc1 ; c1ccc(-c2cccc(-c3nc(-c4ccccc4)nc(-c4c(-c5ccccc5-n5c6ccccc6c6ccccc65)ccc5oc6ccccc6c45)n3)c2)cc1 ; c1ccc(-c2nc(-c3ccccc3-c3ccccc3)nc(-c3c(-c4ccccc4-n4c5ccccc5c5ccccc54)ccc4oc5ccccc5c34)n2)cc1]

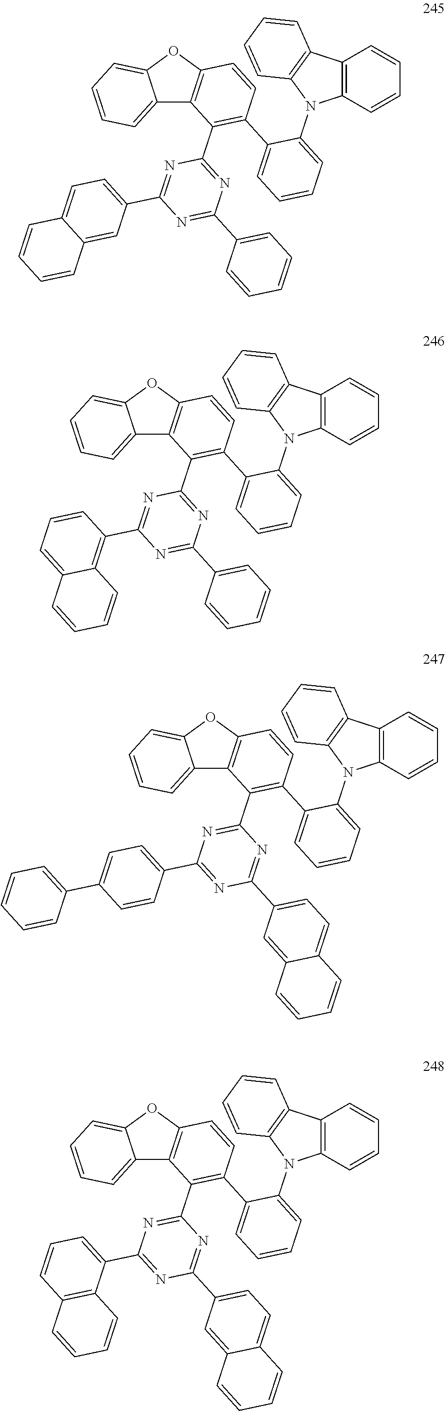 [SMILES: c1ccc(-c2nc(-c3ccc4ccccc4c3)nc(-c3c(-c4ccccc4-n4c5ccccc5c5ccccc54)ccc4oc5ccccc5c34)n2)cc1 ; c1ccc(-c2nc(-c3cccc4ccccc34)nc(-c3c(-c4ccccc4-n4c5ccccc5c5ccccc54)ccc4oc5ccccc5c34)n2)cc1 ; c1ccc(-c2ccc(-c3nc(-c4ccc5ccccc5c4)nc(-c4c(-c5ccccc5-n5c6ccccc6c6ccccc65)ccc5oc6ccccc6c45)n3)cc2)cc1 ; c1ccc(-n2c3ccccc3c3ccccc32)c(-c2ccc3oc4ccccc4c3c2-c2nc(-c3ccc4ccccc4c3)nc(-c3cccc4ccccc34)n2)c1]

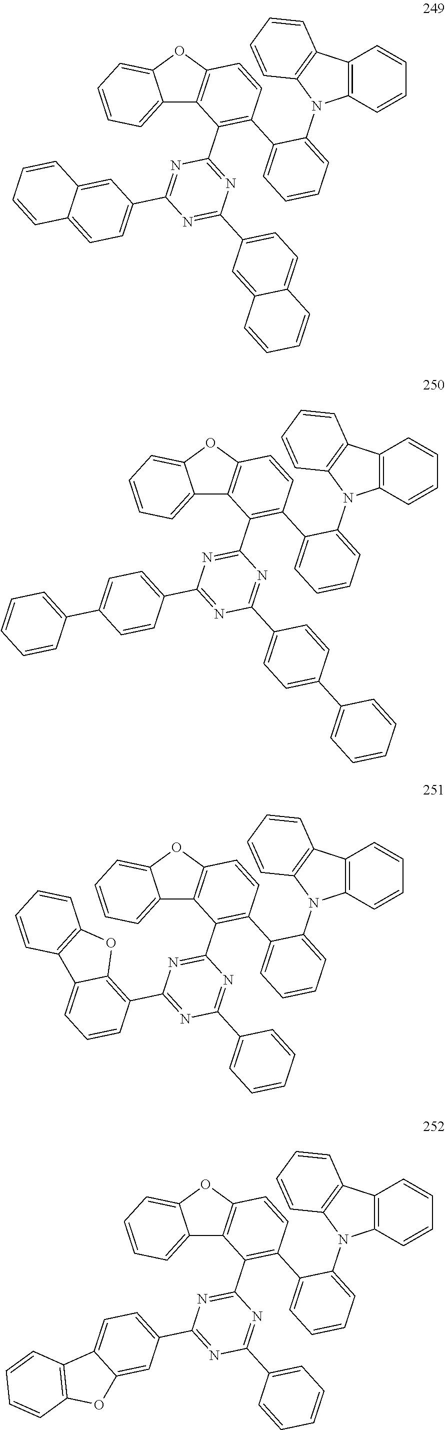 [SMILES: c1ccc(-n2c3ccccc3c3ccccc32)c(-c2ccc3oc4ccccc4c3c2-c2nc(-c3ccc4ccccc4c3)nc(-c3ccc4ccccc4c3)n2)c1 ; c1ccc(-c2ccc(-c3nc(-c4ccc(-c5ccccc5)cc4)nc(-c4c(-c5ccccc5-n5c6ccccc6c6ccccc65)ccc5oc6ccccc6c45)n3)cc2)cc1 ; c1ccc(-c2nc(-c3cccc4c3oc3ccccc34)nc(-c3c(-c4ccccc4-n4c5ccccc5c5ccccc54)ccc4oc5ccccc5c34)n2)cc1 ; c1ccc(-c2nc(-c3ccc4c(c3)oc3ccccc34)nc(-c3c(-c4ccccc4-n4c5ccccc5c5ccccc54)ccc4oc5ccccc5c34)n2)cc1]

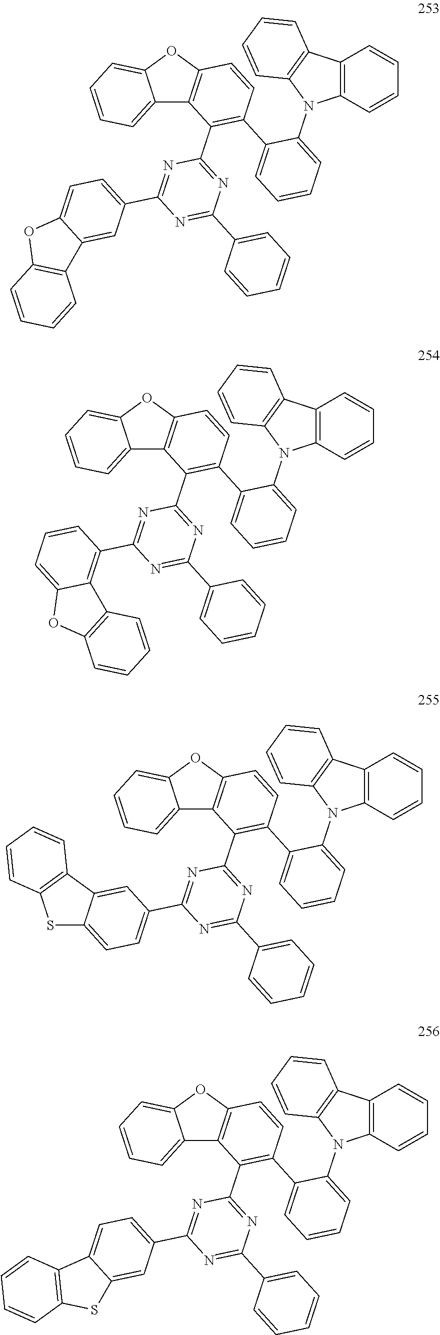 [SMILES: c1ccc(-c2nc(-c3ccc4oc5ccccc5c4c3)nc(-c3c(-c4ccccc4-n4c5ccccc5c5ccccc54)ccc4oc5ccccc5c34)n2)cc1 ; c1ccc(-c2nc(-c3cccc4oc5ccccc5c34)nc(-c3c(-c4ccccc4-n4c5ccccc5c5ccccc54)ccc4oc5ccccc5c34)n2)cc1 ; c1ccc(-c2nc(-c3ccc4sc5ccccc5c4c3)nc(-c3c(-c4ccccc4-n4c5ccccc5c5ccccc54)ccc4oc5ccccc5c34)n2)cc1 ; c1ccc(-c2nc(-c3ccc4c(c3)sc3ccccc34)nc(-c3c(-c4ccccc4-n4c5ccccc5c5ccccc54)ccc4oc5ccccc5c34)n2)cc1]

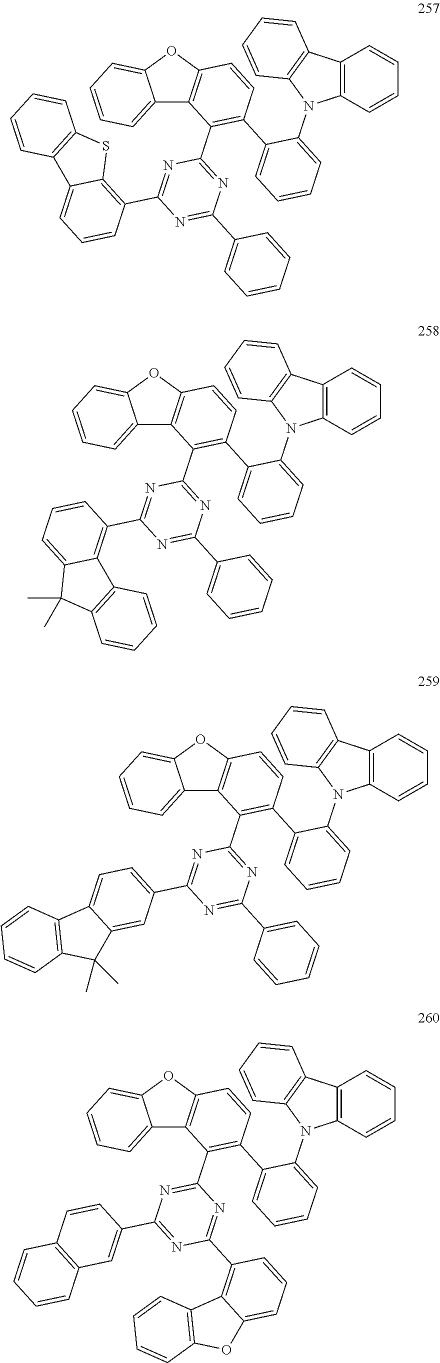

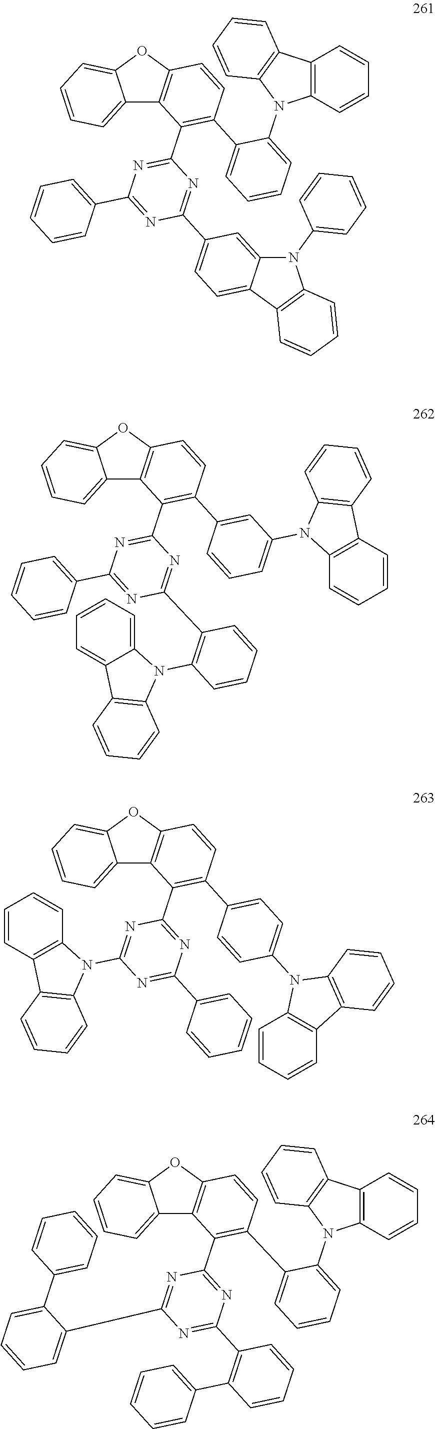 [SMILES: c1ccc(-c2nc(-c3ccc4c5ccccc5n(-c5ccccc5)c4c3)nc(-c3c(-c4ccccc4-n4c5ccccc5c5ccccc54)ccc4oc5ccccc5c34)n2)cc1 ; c1ccc(-c2nc(-c3ccccc3-n3c4ccccc4c4ccccc43)nc(-c3c(-c4cccc(-n5c6ccccc6c6ccccc65)c4)ccc4oc5ccccc5c34)n2)cc1 ; c1ccc(-c2nc(-c3c(-c4ccc(-n5c6ccccc6c6ccccc65)cc4)ccc4oc5ccccc5c34)nc(-n3c4ccccc4c4ccccc43)n2)cc1 ; c1ccc(-c2ccccc2-c2nc(-c3ccccc3-c3ccccc3)nc(-c3c(-c4ccccc4-n4c5ccccc5c5ccccc54)ccc4oc5ccccc5c34)n2)cc1]

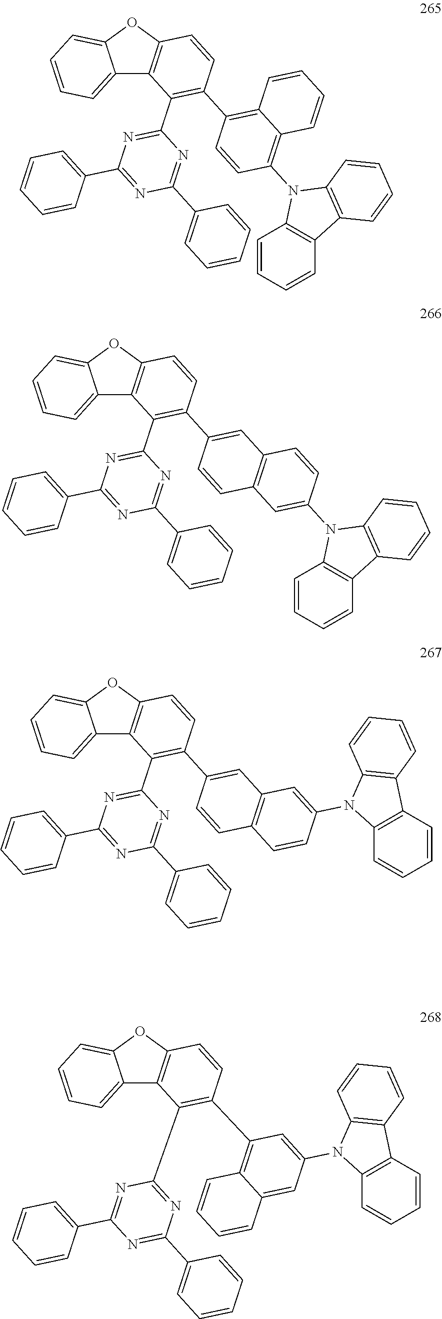 [SMILES: c1ccc(-c2nc(-c3ccccc3)nc(-c3c(-c4ccc(-n5c6ccccc6c6ccccc65)c5ccccc45)ccc4oc5ccccc5c34)n2)cc1 ; c1ccc(-c2nc(-c3ccccc3)nc(-c3c(-c4ccc5cc(-n6c7ccccc7c7ccccc76)ccc5c4)ccc4oc5ccccc5c34)n2)cc1 ; c1ccc(-c2nc(-c3ccccc3)nc(-c3c(-c4ccc5ccc(-n6c7ccccc7c7ccccc76)cc5c4)ccc4oc5ccccc5c34)n2)cc1 ; c1ccc(-c2nc(-c3ccccc3)nc(-c3c(-c4cc(-n5c6ccccc6c6ccccc65)cc5ccccc45)ccc4oc5ccccc5c34)n2)cc1]

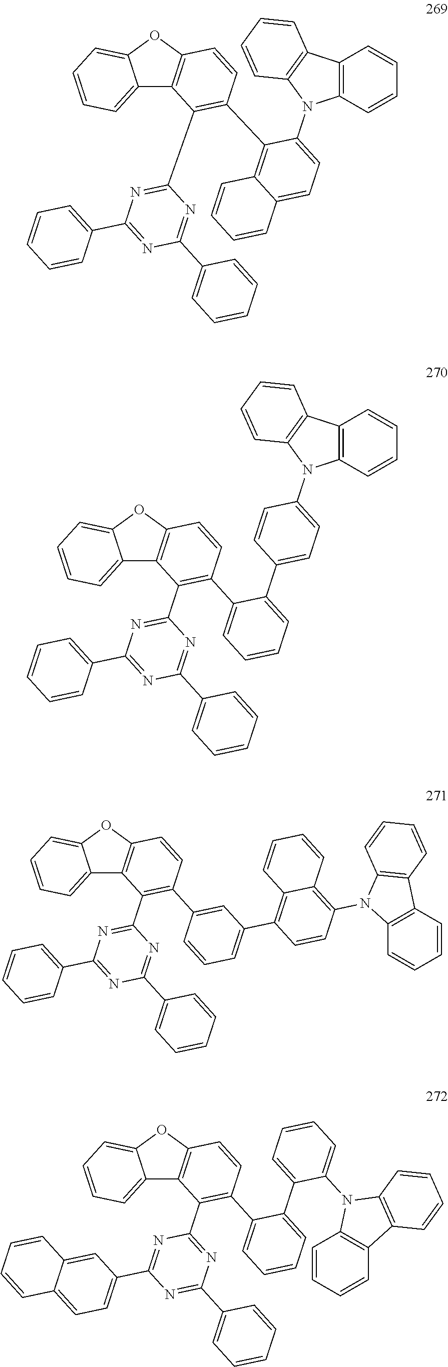 [SMILES: c1ccc(-c2nc(-c3ccccc3)nc(-c3c(-c4c(-n5c6ccccc6c6ccccc65)ccc5ccccc45)ccc4oc5ccccc5c34)n2)cc1 ; c1ccc(-c2nc(-c3ccccc3)nc(-c3c(-c4ccccc4-c4ccc(-n5c6ccccc6c6ccccc65)cc4)ccc4oc5ccccc5c34)n2)cc1 ; c1ccc(-c2nc(-c3ccccc3)nc(-c3c(-c4cccc(-c5ccc(-n6c7ccccc7c7ccccc76)c6ccccc56)c4)ccc4oc5ccccc5c34)n2)cc1 ; c1ccc(-c2nc(-c3ccc4ccccc4c3)nc(-c3c(-c4ccccc4-c4ccccc4-n4c5ccccc5c5ccccc54)ccc4oc5ccccc5c34)n2)cc1]

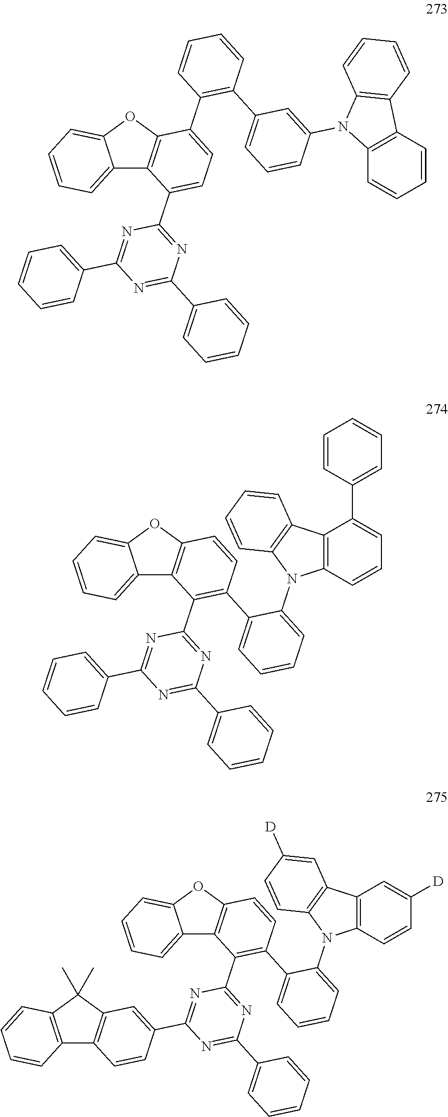 [SMILES: c1ccc(-c2nc(-c3ccccc3)nc(-c3ccc(-c4ccccc4-c4cccc(-n5c6ccccc6c6ccccc65)c4)c4oc5ccccc5c34)n2)cc1 ; c1ccc(-c2nc(-c3ccccc3)nc(-c3c(-c4ccccc4-n4c5ccccc5c5c(-c6ccccc6)cccc54)ccc4oc5ccccc5c34)n2)cc1 ; [2H]c1ccc2c(c1)c1cc([2H])ccc1n2-c1ccccc1-c1ccc2oc3ccccc3c2c1-c1nc(-c2ccccc2)nc(-c2ccc3c(c2)C(C)(C)c2ccccc2-3)n1]

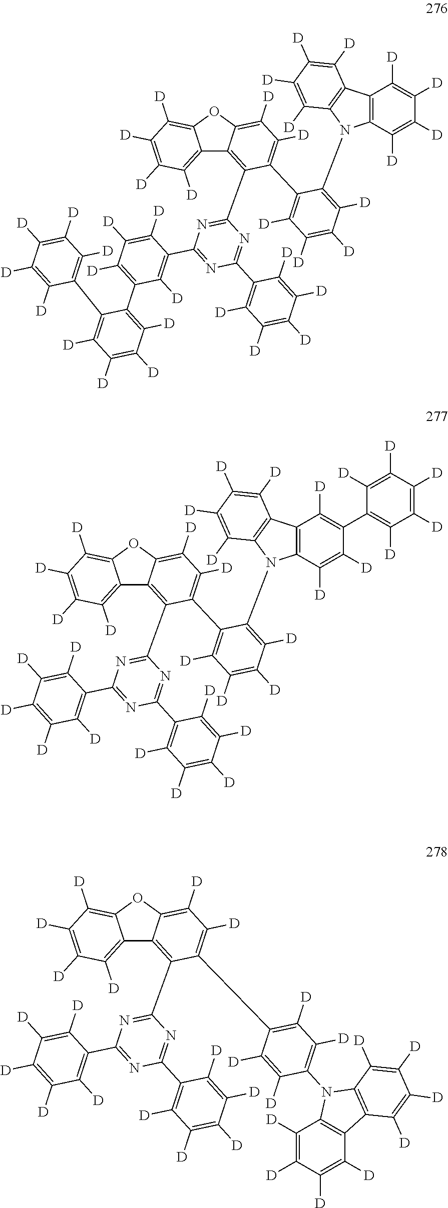

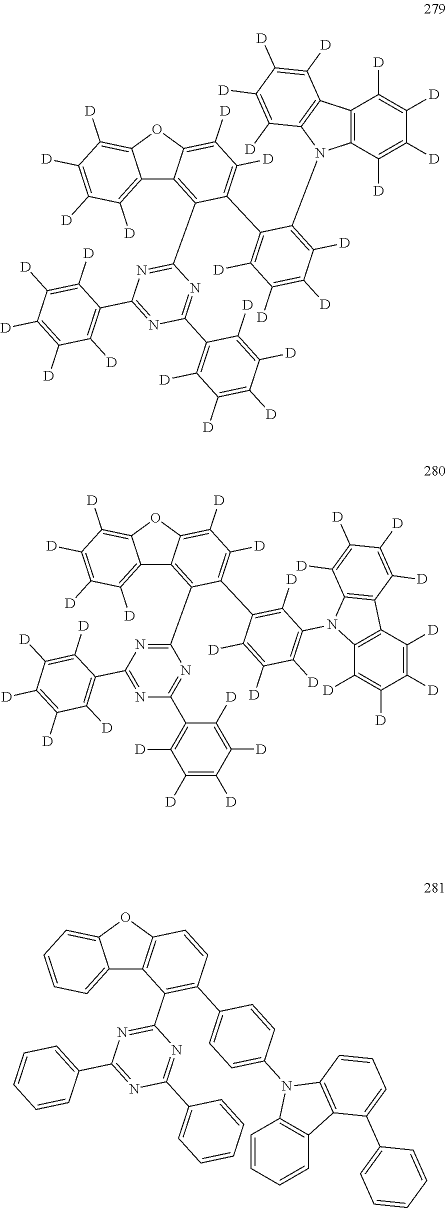

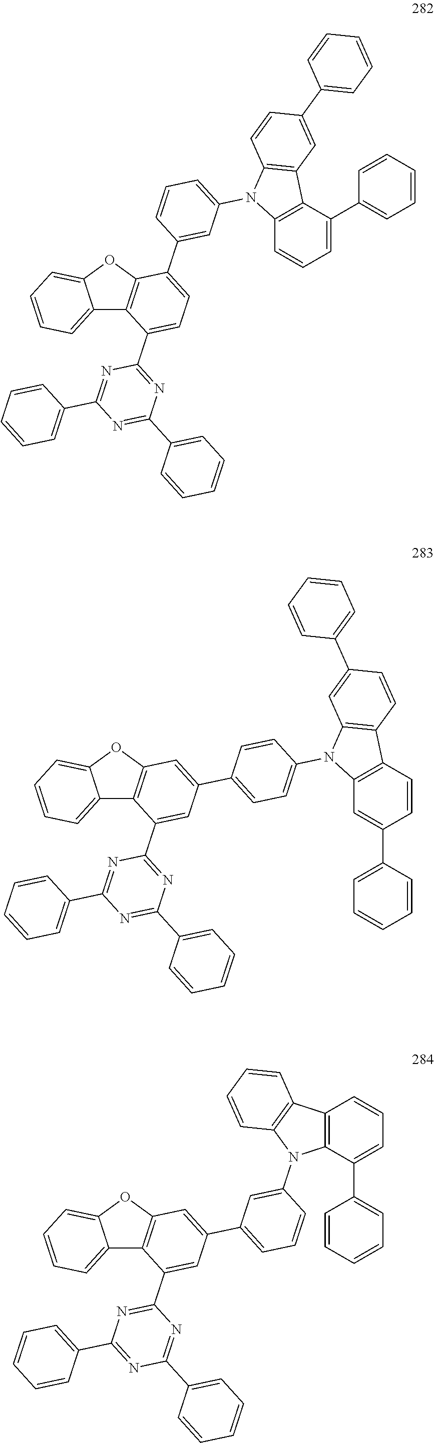 [SMILES: c1ccc(-c2ccc3c(c2)c2c(-c4ccccc4)cccc2n3-c2cccc(-c3ccc(-c4nc(-c5ccccc5)nc(-c5ccccc5)n4)c4c3oc3ccccc34)c2)cc1 ; c1ccc(-c2ccc3c4ccc(-c5ccccc5)cc4n(-c4ccc(-c5cc(-c6nc(-c7ccccc7)nc(-c7ccccc7)n6)c6c(c5)oc5ccccc56)cc4)c3c2)cc1 ; c1ccc(-c2nc(-c3ccccc3)nc(-c3cc(-c4cccc(-n5c6ccccc6c6cccc(-c7ccccc7)c65)c4)cc4oc5ccccc5c34)n2)cc1]

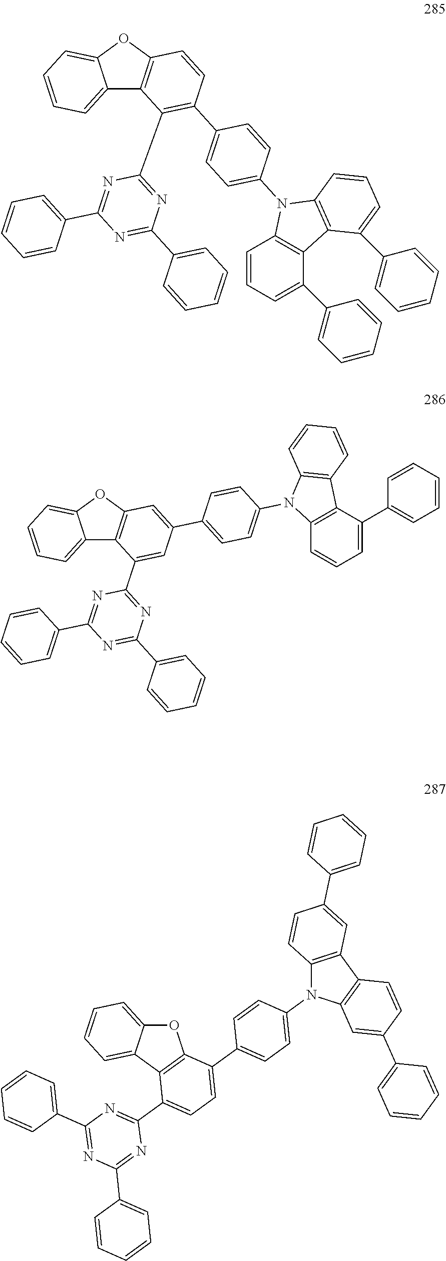 [SMILES: c1ccc(-c2nc(-c3ccccc3)nc(-c3c(-c4ccc(-n5c6cccc(-c7ccccc7)c6c6c(-c7ccccc7)cccc65)cc4)ccc4oc5ccccc5c34)n2)cc1 ; c1ccc(-c2nc(-c3ccccc3)nc(-c3cc(-c4ccc(-n5c6ccccc6c6c(-c7ccccc7)cccc65)cc4)cc4oc5ccccc5c34)n2)cc1 ; c1ccc(-c2ccc3c(c2)c2ccc(-c4ccccc4)cc2n3-c2ccc(-c3ccc(-c4nc(-c5ccccc5)nc(-c5ccccc5)n4)c4c3oc3ccccc34)cc2)cc1]

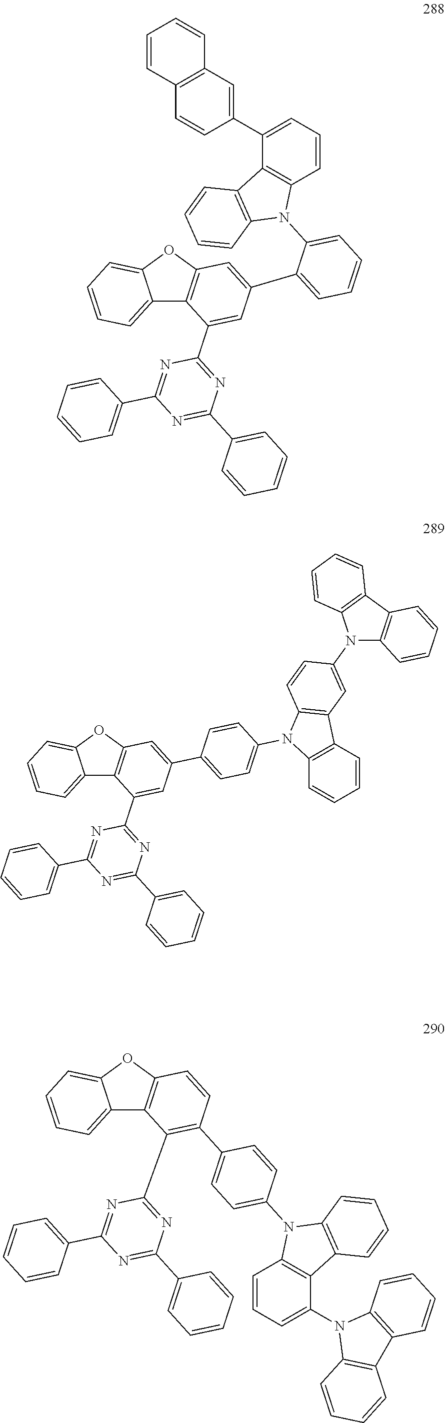 [SMILES: c1ccc(-c2nc(-c3ccccc3)nc(-c3cc(-c4ccccc4-n4c5ccccc5c5c(-c6ccc7ccccc7c6)cccc54)cc4oc5ccccc5c34)n2)cc1 ; c1ccc(-c2nc(-c3ccccc3)nc(-c3cc(-c4ccc(-n5c6ccccc6c6cc(-n7c8ccccc8c8ccccc87)ccc65)cc4)cc4oc5ccccc5c34)n2)cc1 ; c1ccc(-c2nc(-c3ccccc3)nc(-c3c(-c4ccc(-n5c6ccccc6c6c(-n7c8ccccc8c8ccccc87)cccc65)cc4)ccc4oc5ccccc5c34)n2)cc1]

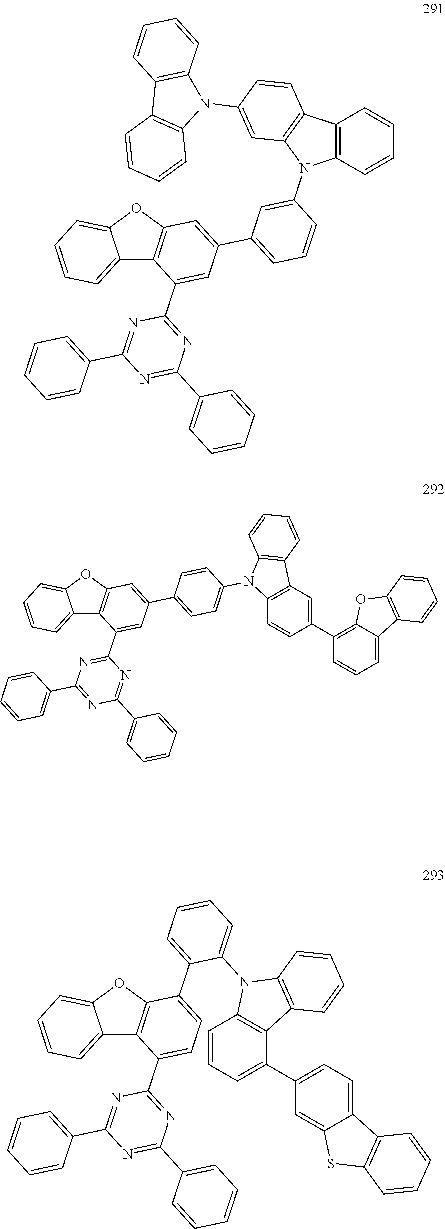 [SMILES: c1ccc(-c2nc(-c3ccccc3)nc(-c3cc(-c4cccc(-n5c6ccccc6c6ccc(-n7c8ccccc8c8ccccc87)cc65)c4)cc4oc5ccccc5c34)n2)cc1 ; c1ccc(-c2nc(-c3ccccc3)nc(-c3cc(-c4ccc(-n5c6ccccc6c6cc(-c7cccc8c7oc7ccccc78)ccc65)cc4)cc4oc5ccccc5c34)n2)cc1 ; c1ccc(-c2nc(-c3ccccc3)nc(-c3ccc(-c4ccccc4-n4c5ccccc5c5c(-c6ccc7c(c6)sc6ccccc67)cccc54)c4oc5ccccc5c34)n2)cc1]

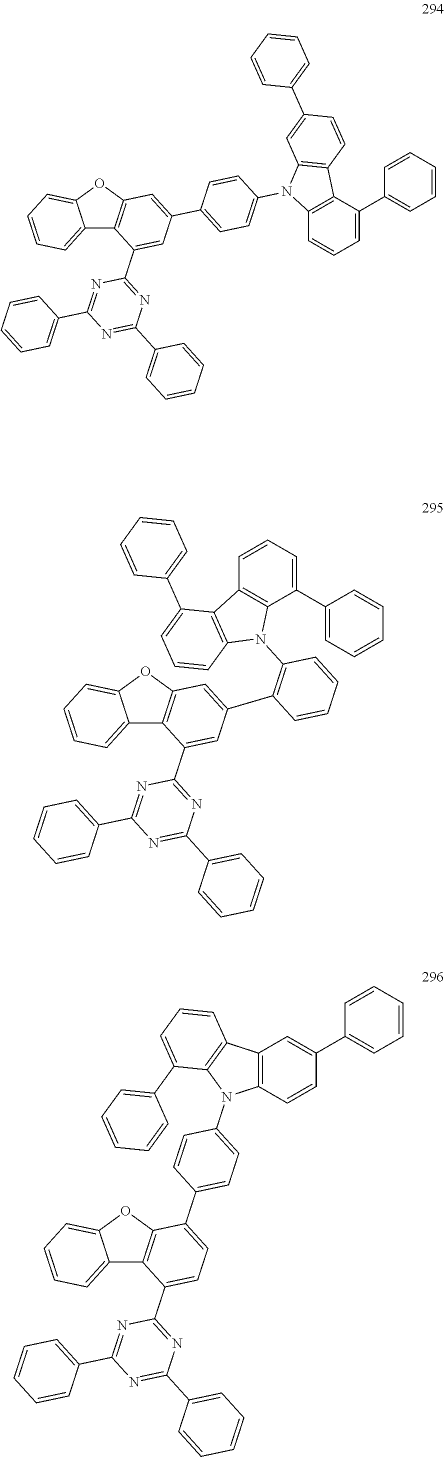 [SMILES: c1ccc(-c2ccc3c4c(-c5ccccc5)cccc4n(-c4ccc(-c5cc(-c6nc(-c7ccccc7)nc(-c7ccccc7)n6)c6c(c5)oc5ccccc56)cc4)c3c2)cc1 ; c1ccc(-c2nc(-c3ccccc3)nc(-c3cc(-c4ccccc4-n4c5cccc(-c6ccccc6)c5c5cccc(-c6ccccc6)c54)cc4oc5ccccc5c34)n2)cc1 ; c1ccc(-c2ccc3c(c2)c2cccc(-c4ccccc4)c2n3-c2ccc(-c3ccc(-c4nc(-c5ccccc5)nc(-c5ccccc5)n4)c4c3oc3ccccc34)cc2)cc1]

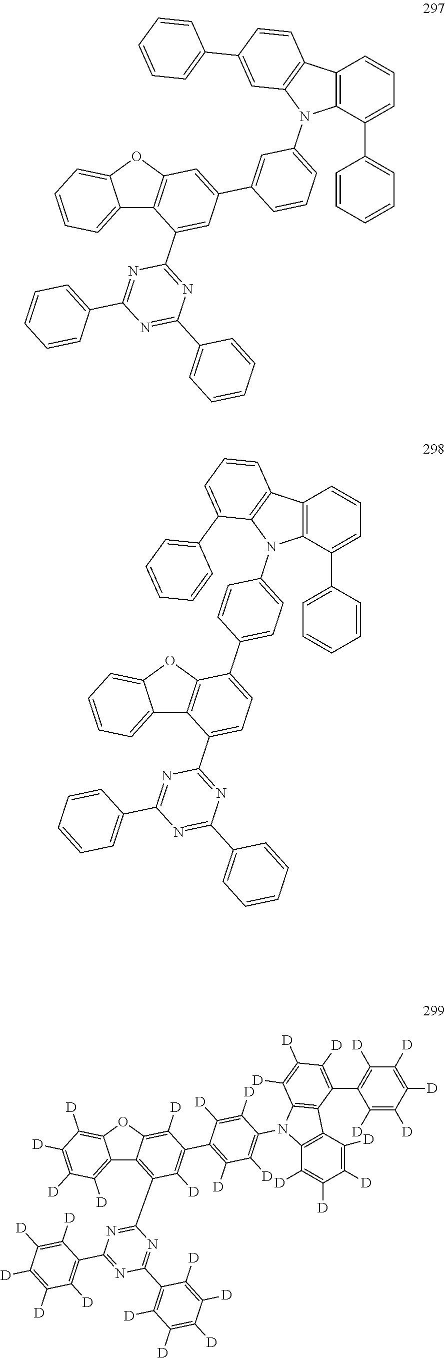 [SMILES: c1ccc(-c2ccc3c4cccc(-c5ccccc5)c4n(-c4cccc(-c5cc(-c6nc(-c7ccccc7)nc(-c7ccccc7)n6)c6c(c5)oc5ccccc56)c4)c3c2)cc1 ; c1ccc(-c2nc(-c3ccccc3)nc(-c3ccc(-c4ccc(-n5c6c(-c7ccccc7)cccc6c6cccc(-c7ccccc7)c65)cc4)c4oc5ccccc5c34)n2)cc1 ; [2H]c1c([2H])c([2H])c(-c2nc(-c3c([2H])c([2H])c([2H])c([2H])c3[2H])nc(-c3c([2H])c(-c4c([2H])c([2H])c(-n5c6c([2H])c([2H])c([2H])c([2H])c6c6c(-c7c([2H])c([2H])c([2H])c([2H])c7[2H])c([2H])c([2H])c([2H])c65)c([2H])c4[2H])c([2H])c4oc5c([2H])c([2H])c([2H])c([2H])c5c34)n2)c([2H])c1[2H]]

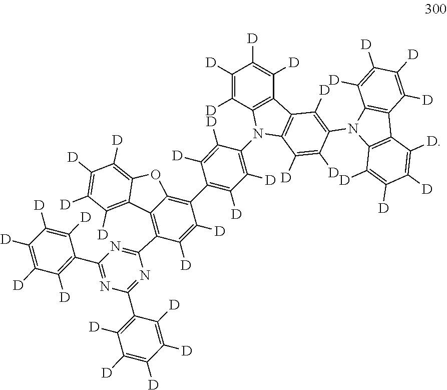

9. A light-emitting device comprising:
a first electrode;
a second electrode disposed on the first electrode; and
one or more organic layers interposed between the first electrode and the second electrode, at least one of the one or more organic layers comprising the heterocyclic compound according to claim 1.

10. The light-emitting device according to claim 9, wherein the organic layer comprises a light-emitting layer.

11. The light-emitting device according to claim 10, wherein the light-emitting layer comprises a dopant material and a host material, and the host material comprises the heterocyclic compound.

12. The light-emitting device according to claim 10, wherein the organic layer further comprises a hole transport layer disposed between the first electrode and the light-emitting layer, and an electron transport layer disposed between the light-emitting layer and the second electrode.

13. The light-emitting device according to claim 11, wherein the host material further comprises a compound represented by Formula 4 below or a compound represented by Formula 5: 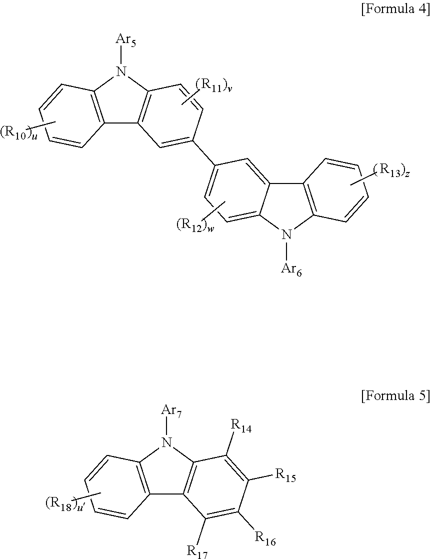
wherein, in Formula 4 and 5, two adjacent groups of R₁₄ to R₁₇ form a ring represented by 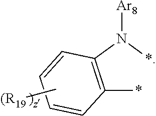
the remaining groups of R₁₀ to R₁₃, R₁₄ to R₁₇, and R₁₈ to R₁₉ that do not form a ring are each independently selected from the group consisting of hydrogen, deuterium, a halogen, a substituted or unsubstituted alkyl group having 1 to 60 carbon atoms, a substituted or unsubstituted alkenyl group having 2 to 60 carbon atoms, a substituted or unsubstituted alkynyl group having 2 to 60 carbon atoms, a substituted or unsubstituted alkoxy group having 1 to 60 carbon atoms, a substituted or unsubstituted cycloalkyl group having 3 to 60 carbon atoms, a substituted or unsubstituted heterocycloalkyl group having 2 to 60 carbon atoms, a substituted or unsubstituted aryl group having 6 to 60 carbon atoms, a substituted or unsubstituted heteroaryl group having 2 to 60 carbon atoms, —SiRR′R″, and —P(═O)RR′, wherein adjacent groups are linked to form a substituted or unsubstituted aryl group having 6 to 60 carbon atoms, a substituted or unsubstituted heterocycloalkyl group having 2 to 60 carbon atoms, or a substituted or unsubstituted heteroaryl group having 2 to 60 carbon atoms,
R, R′ and R″ are each independently hydrogen, deuterium, a cyano group (—CN), a substituted or unsubstituted alkyl group having 1 to 60 carbon atoms, a substituted or unsubstituted cycloalkyl group having 3 to 60 carbon atoms, a substituted or unsubstituted aryl group having 6 to 60 carbon atoms, or a substituted or unsubstituted heteroaryl group having 2 to 60 carbon atoms,
Ar₅ to Ar₈ are each independently a substituted or unsubstituted aryl group having 6 to 60 carbon atoms, or a substituted or unsubstituted heteroaryl group having 2 to 60 carbon atoms,
u, u′, z, and z′ are each independently 0 or an integer of 1 to 4,
v and w are each independently 0 or an integer of 1 to 3, and
* represents a bonding site.

14. The light-emitting device according to claim 13, wherein the compound represented by Formula 4 has a deuterium content of 0, or greater than 0 and not more than 100%, and
the compound represented by Formula 5 has a deuterium content of 0, or greater than 0 and not more than 100%.

15. The light-emitting device according to claim 13, wherein the compound represented by Formula 4 is represented by at least one selected from formulae below:

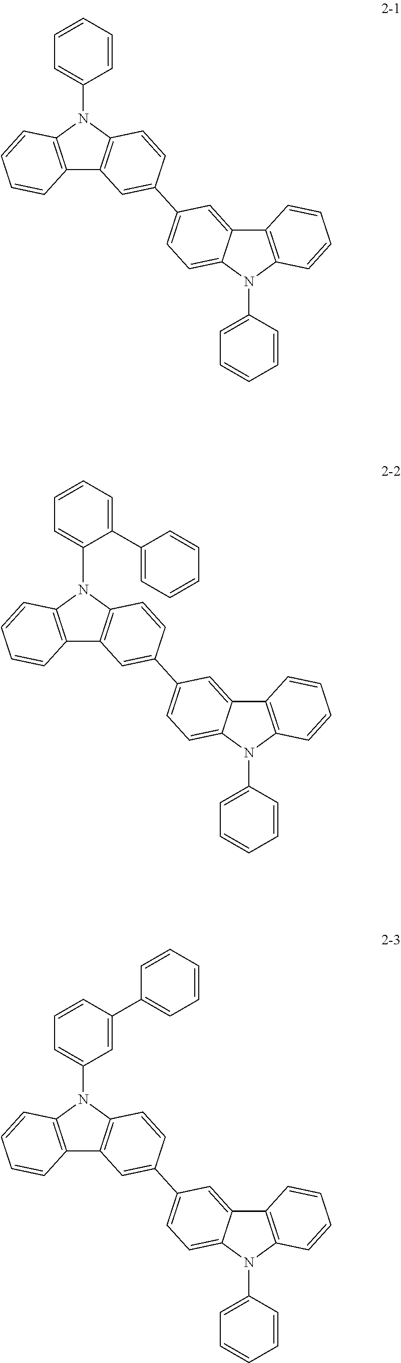 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1]

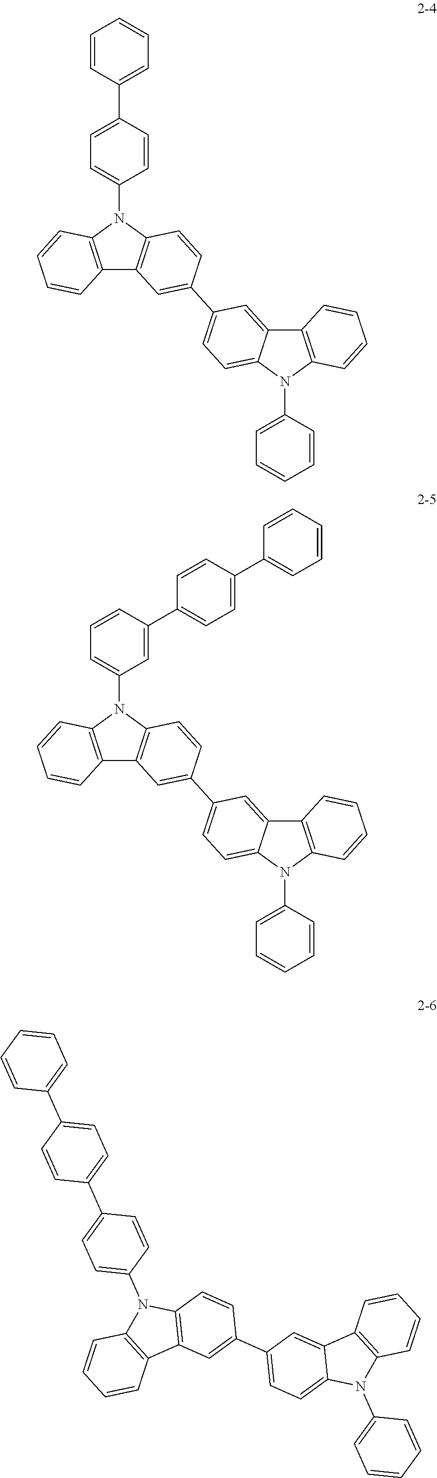

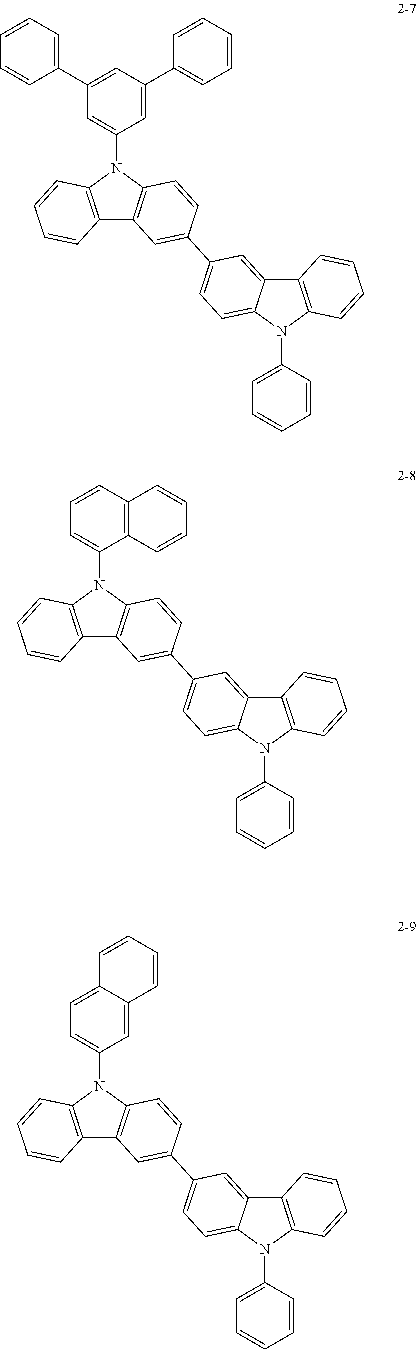 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1]

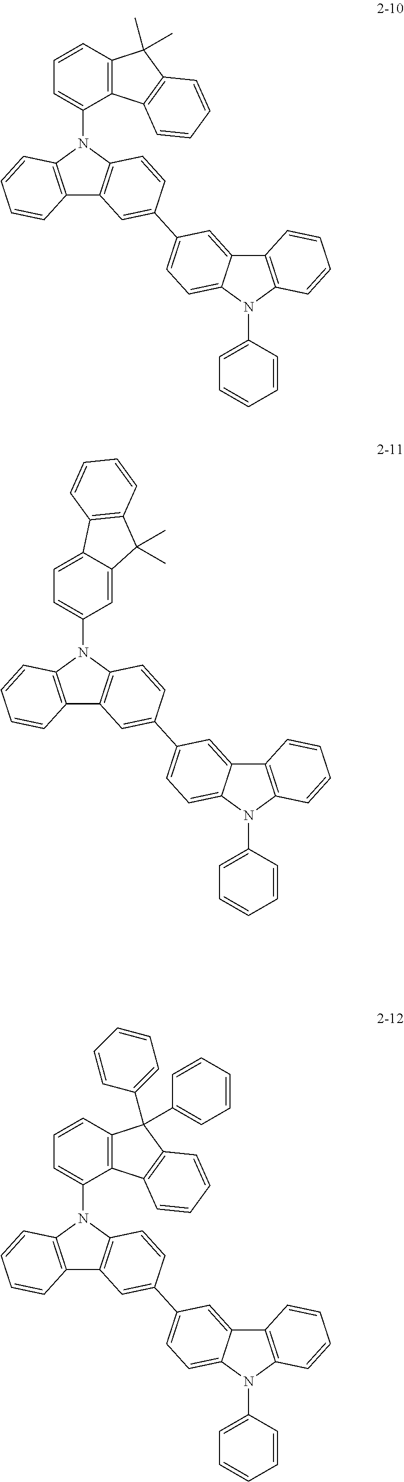 [SMILES: CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

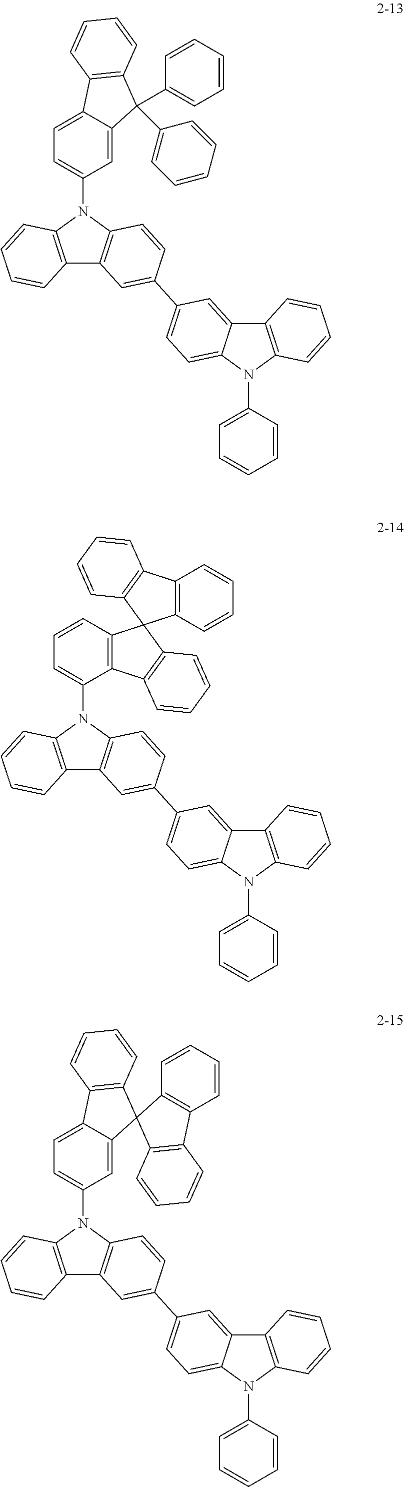 [SMILES: c1ccc(-n2c3ccccc3c3cc(-c4ccc5c(c4)c4ccccc4n5-c4ccc5c(c4)C(c4ccccc4)(c4ccccc4)c4ccccc4-5)ccc32)cc1 ; c1ccc(-n2c3ccccc3c3cc(-c4ccc5c(c4)c4ccccc4n5-c4cccc5c4-c4ccccc4C54c5ccccc5-c5ccccc54)ccc32)cc1 ; c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1]

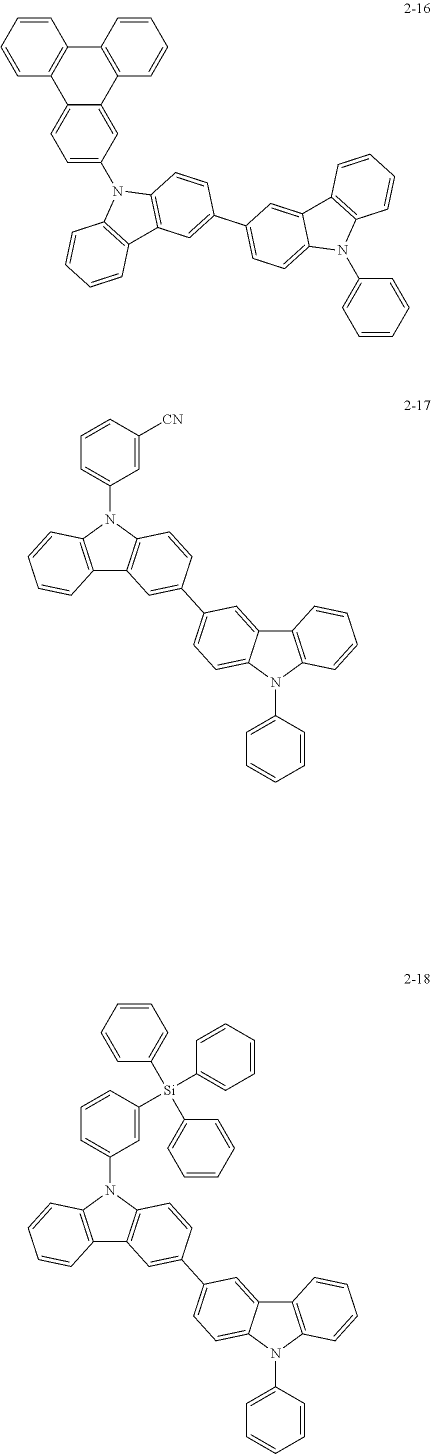 [SMILES: c1ccc(-n2c3ccccc3c3cc(-c4ccc5c(c4)c4ccccc4n5-c4ccc5c6ccccc6c6ccccc6c5c4)ccc32)cc1 ; N#Cc1cccc(-n2c3ccccc3c3cc(-c4ccc5c(c4)c4ccccc4n5-c4ccccc4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1]

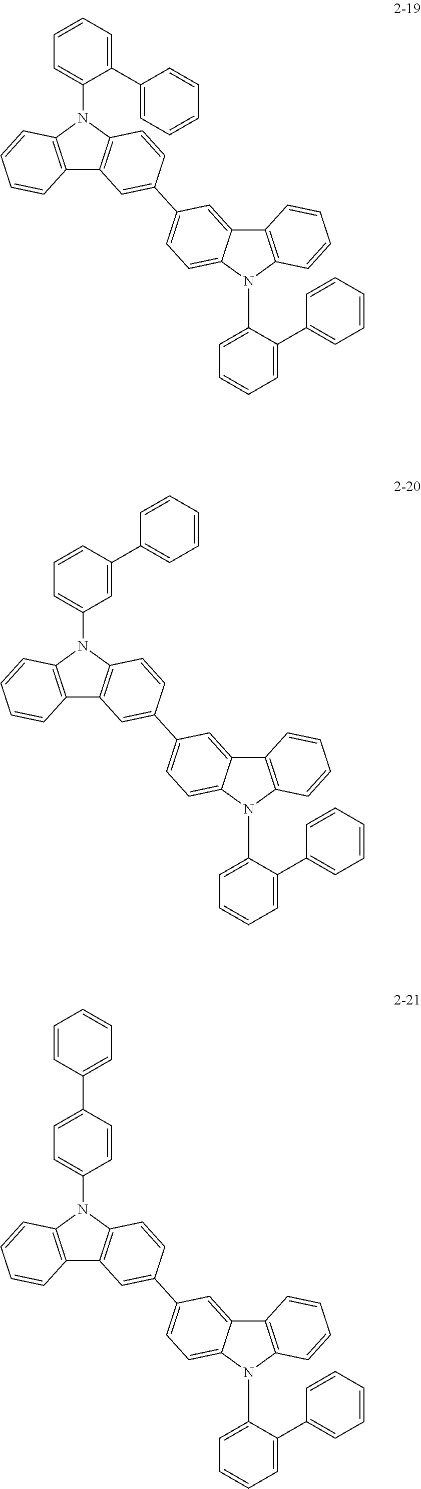 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5ccccc5-c5ccccc5)ccc43)cc2)cc1]

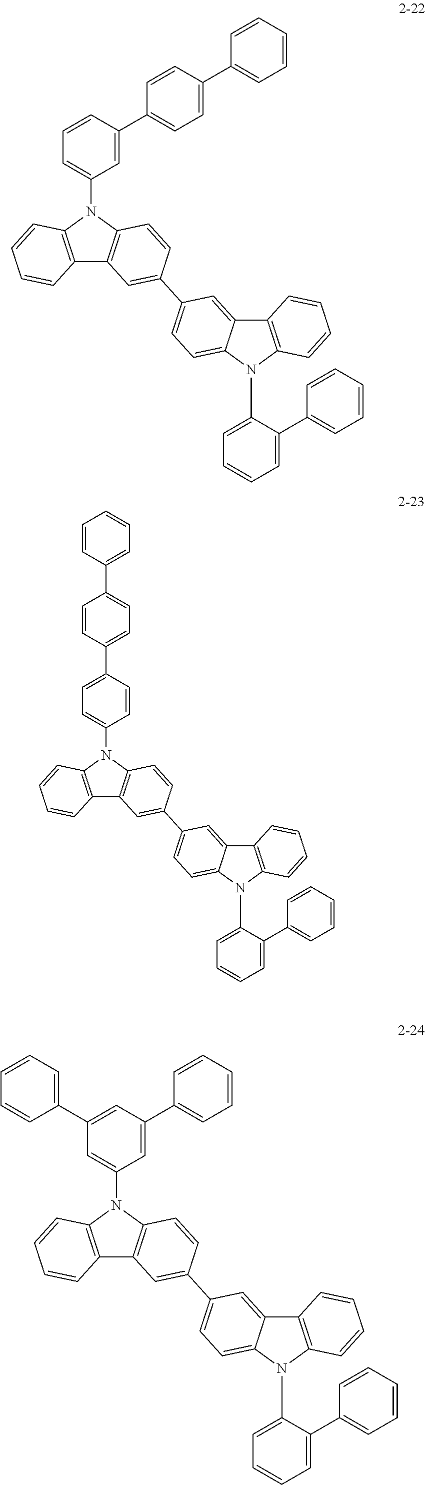 [SMILES: c1ccc(-c2ccc(-c3cccc(-n4c5ccccc5c5cc(-c6ccc7c(c6)c6ccccc6n7-c6ccccc6-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6-c6ccccc6)ccc54)cc3)cc2)cc1 ; c1ccc(-c2cc(-c3ccccc3)cc(-n3c4ccccc4c4cc(-c5ccc6c(c5)c5ccccc5n6-c5ccccc5-c5ccccc5)ccc43)c2)cc1]

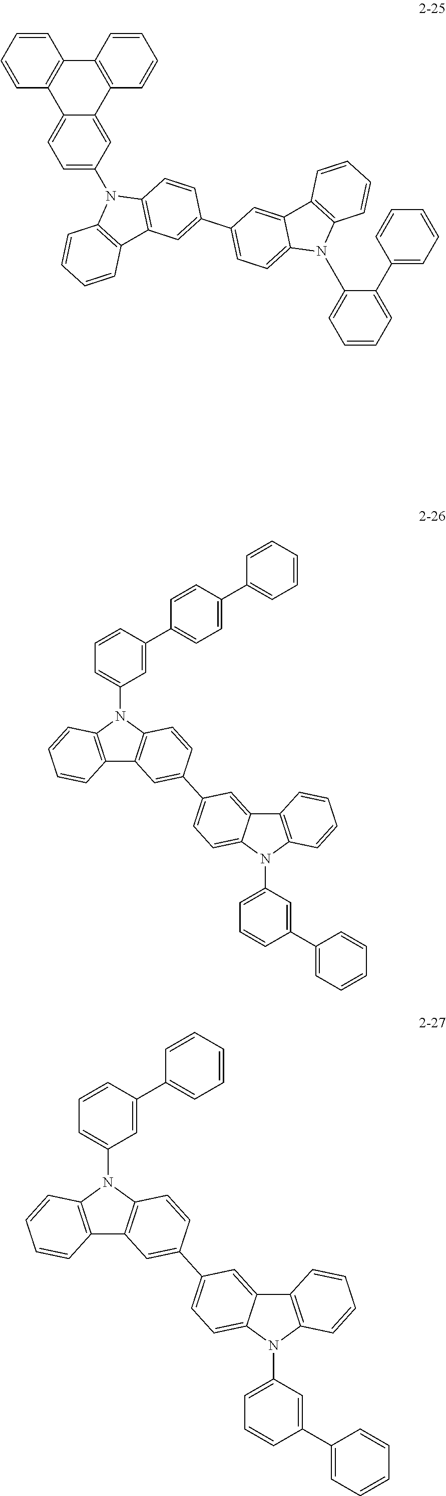 [SMILES: c1ccc(-c2ccccc2-n2c3ccccc3c3cc(-c4ccc5c(c4)c4ccccc4n5-c4ccc5c6ccccc6c6ccccc6c5c4)ccc32)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

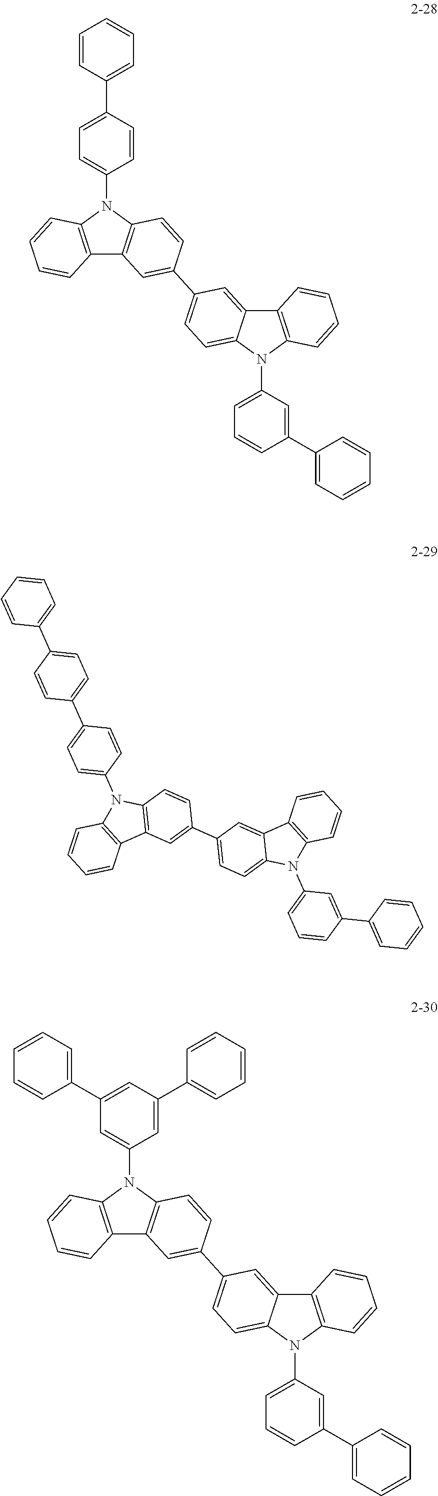 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

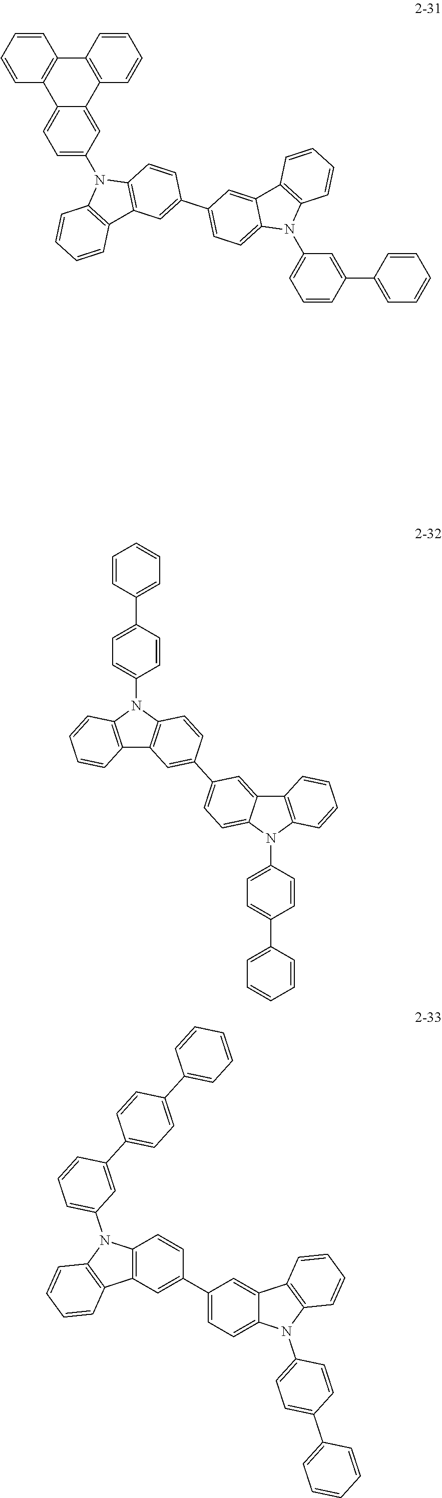 [SMILES: c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1]

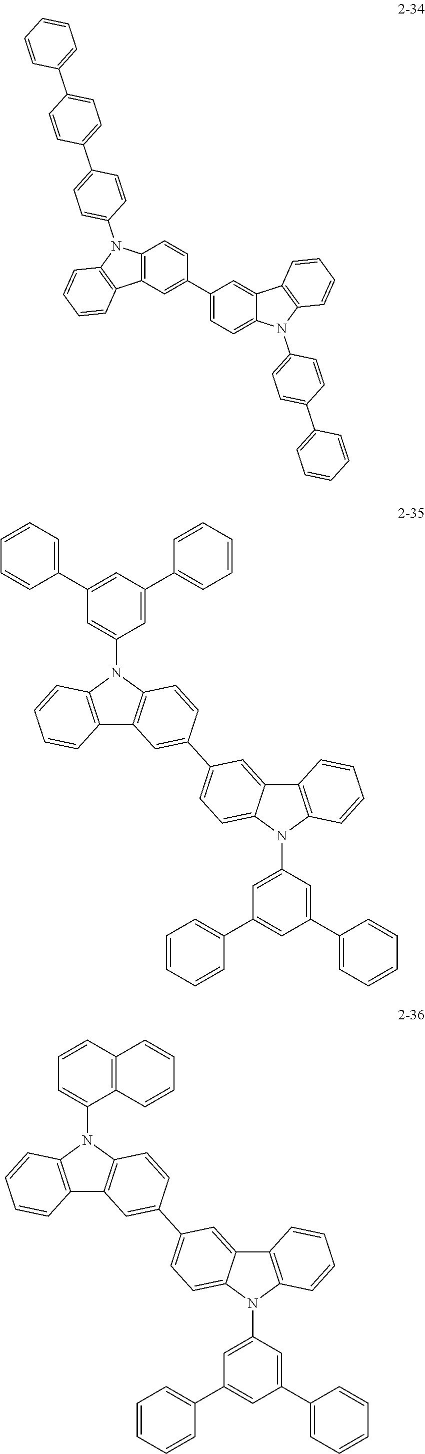 [SMILES: c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5cccc6ccccc56)ccc43)c2)cc1]

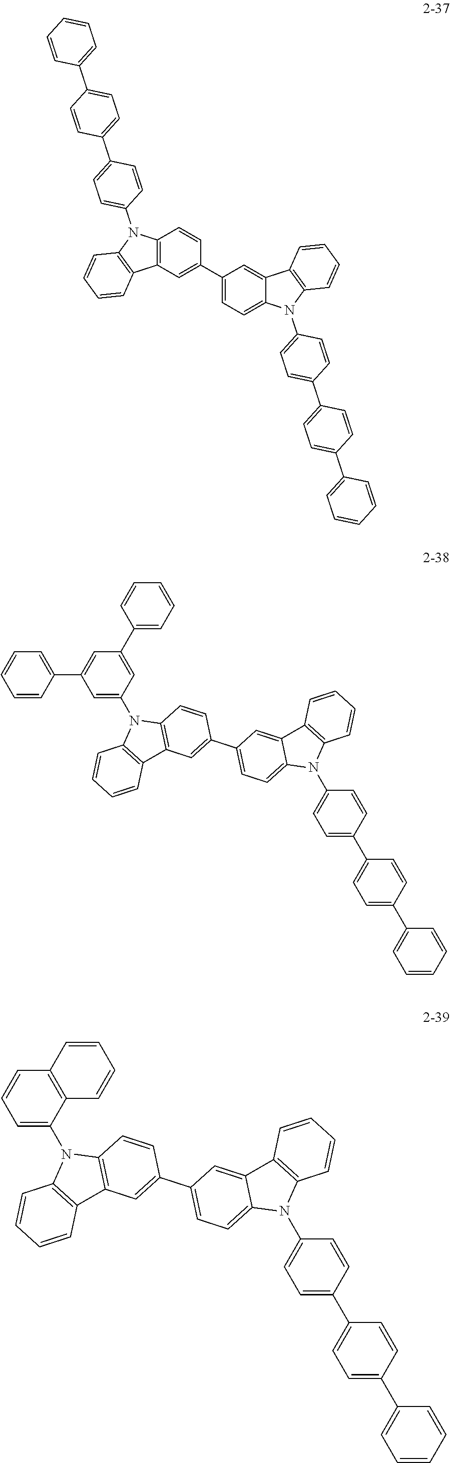 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1]

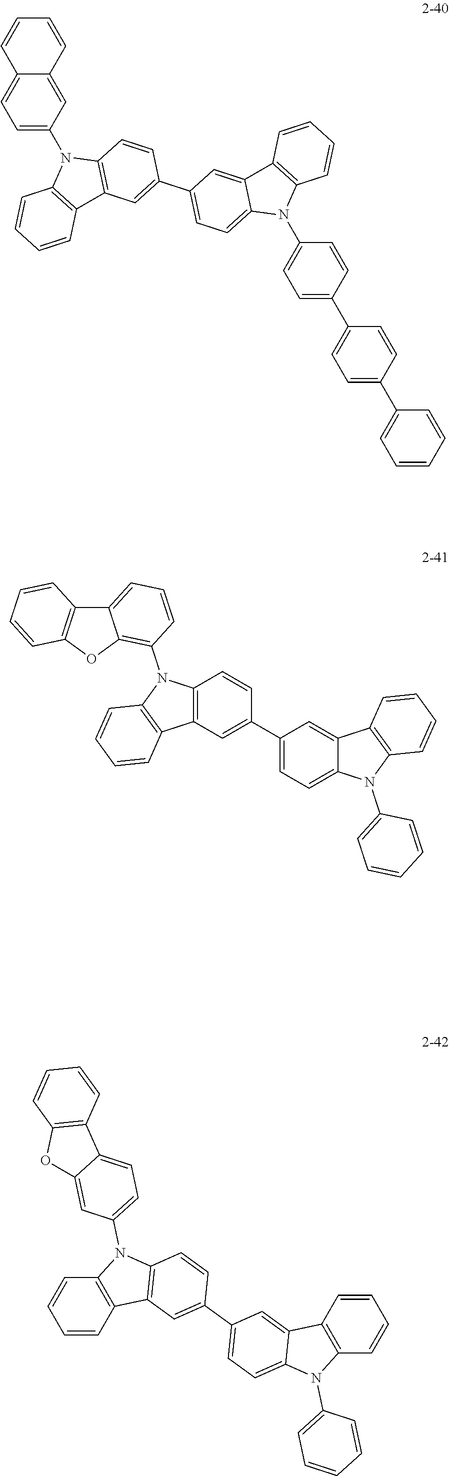 [SMILES: c1ccc(-c2ccc(-c3ccc(-n4c5ccccc5c5cc(-c6ccc7c(c6)c6ccccc6n7-c6ccc7ccccc7c6)ccc54)cc3)cc2)cc1 ; c1ccc(-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4ccc5c(c4)oc4ccccc45)ccc32)cc1]

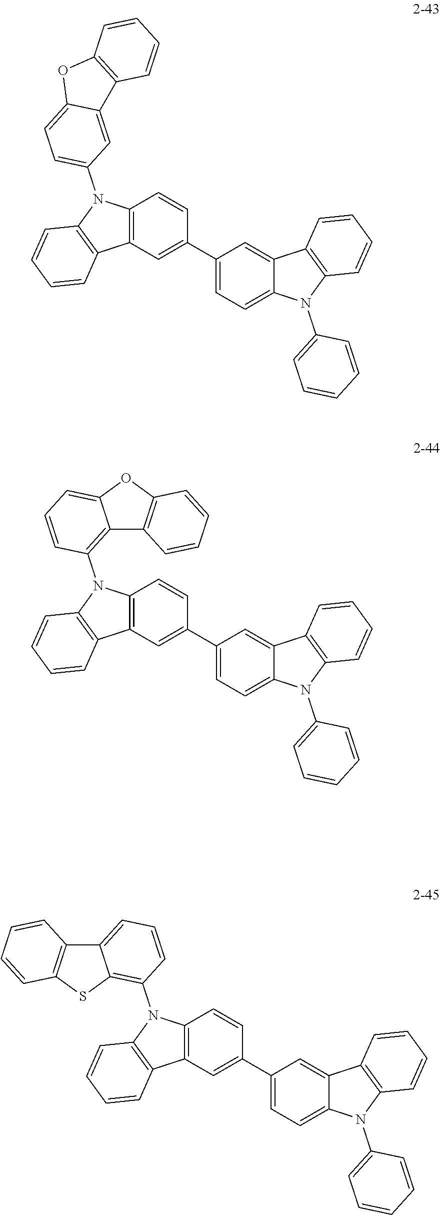 [SMILES: c1ccc(-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4cccc5oc6ccccc6c45)ccc32)cc1 ; c1ccc(-n2c3ccccc3c3cc(-c4ccc5c(c4)c4ccccc4n5-c4cccc5c4sc4ccccc45)ccc32)cc1]

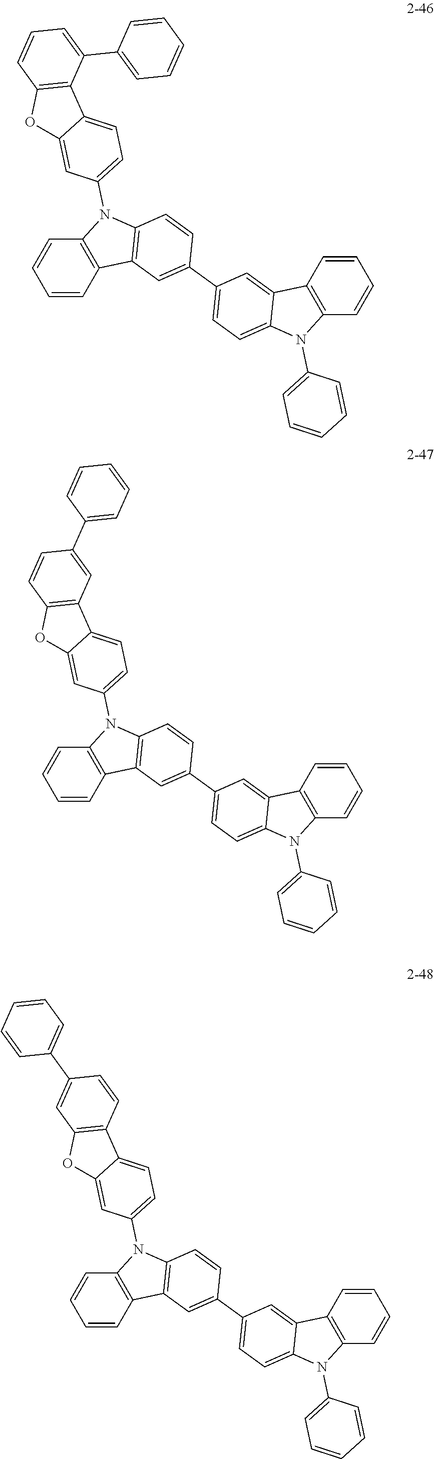 [SMILES: c1ccc(-c2cccc3oc4cc(-n5c6ccccc6c6cc(-c7ccc8c(c7)c7ccccc7n8-c7ccccc7)ccc65)ccc4c23)cc1 ; c1ccc(-c2ccc3oc4cc(-n5c6ccccc6c6cc(-c7ccc8c(c7)c7ccccc7n8-c7ccccc7)ccc65)ccc4c3c2)cc1 ; c1ccc(-c2ccc3c(c2)oc2cc(-n4c5ccccc5c5cc(-c6ccc7c(c6)c6ccccc6n7-c6ccccc6)ccc54)ccc23)cc1]

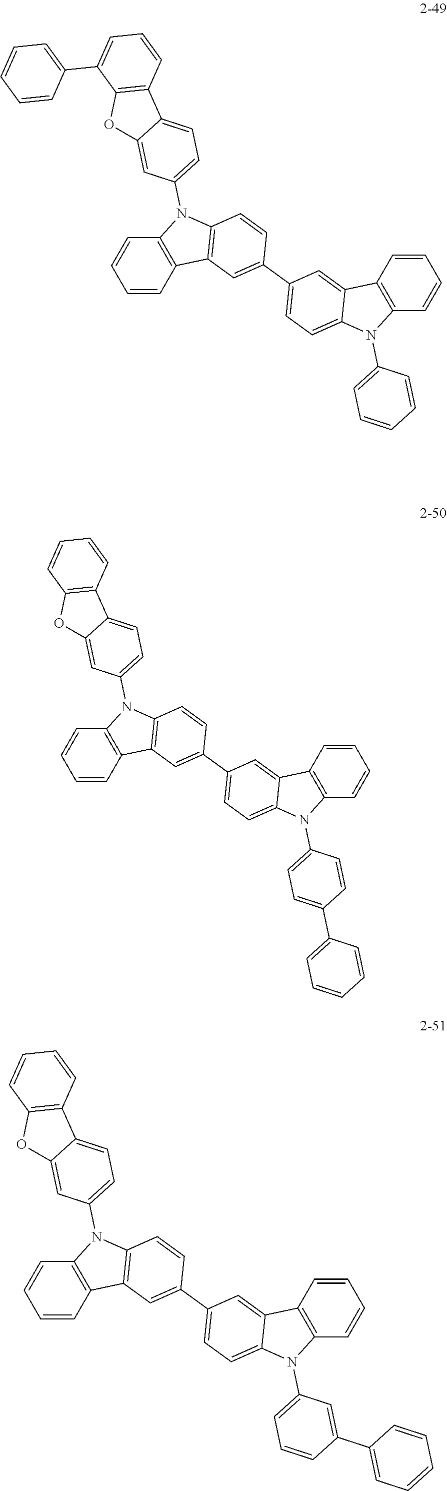 [SMILES: c1ccc(-c2cccc3c2oc2cc(-n4c5ccccc5c5cc(-c6ccc7c(c6)c6ccccc6n7-c6ccccc6)ccc54)ccc23)cc1 ; c1ccc(-c2ccc(-n3c4ccccc4c4cc(-c5ccc6c(c5)c5ccccc5n6-c5ccc6c(c5)oc5ccccc56)ccc43)cc2)cc1 ; c1ccc(-c2cccc(-n3c4ccccc4c4cc(-c5ccc6c(c5)c5ccccc5n6-c5ccc6c(c5)oc5ccccc56)ccc43)c2)cc1]

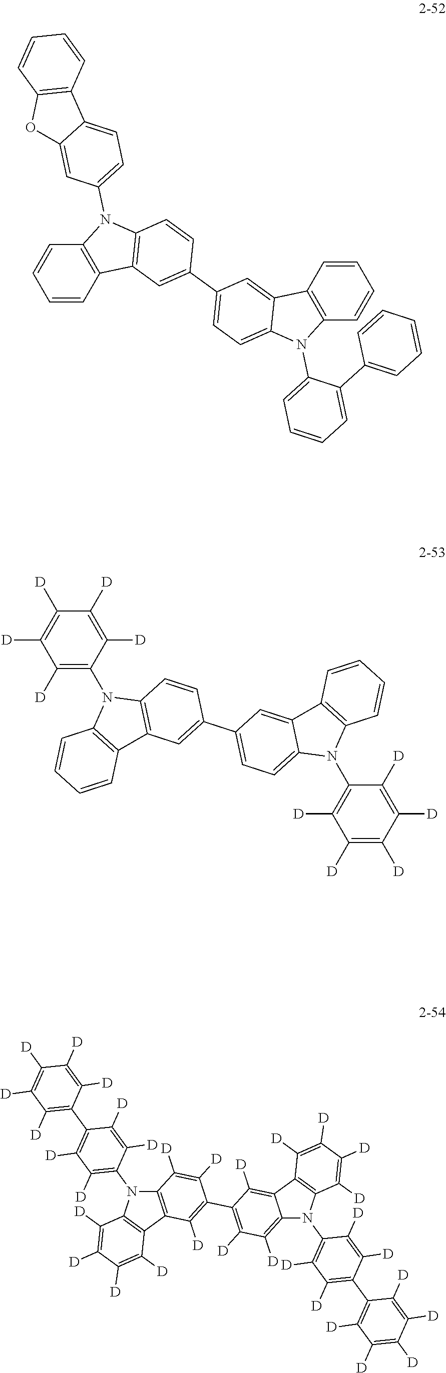 [SMILES: c1ccc(-c2ccccc2-n2c3ccccc3c3cc(-c4ccc5c(c4)c4ccccc4n5-c4ccc5c(c4)oc4ccccc45)ccc32)cc1 ; [2H]c1c([2H])c([2H])c(-n2c3ccccc3c3cc(-c4ccc5c(c4)c4ccccc4n5-c4c([2H])c([2H])c([2H])c([2H])c4[2H])ccc32)c([2H])c1[2H] ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c(-c6c([2H])c([2H])c([2H])c([2H])c6[2H])c([2H])c5[2H])c([2H])c([2H])c43)c([2H])c2[2H])c([2H])c1[2H]]

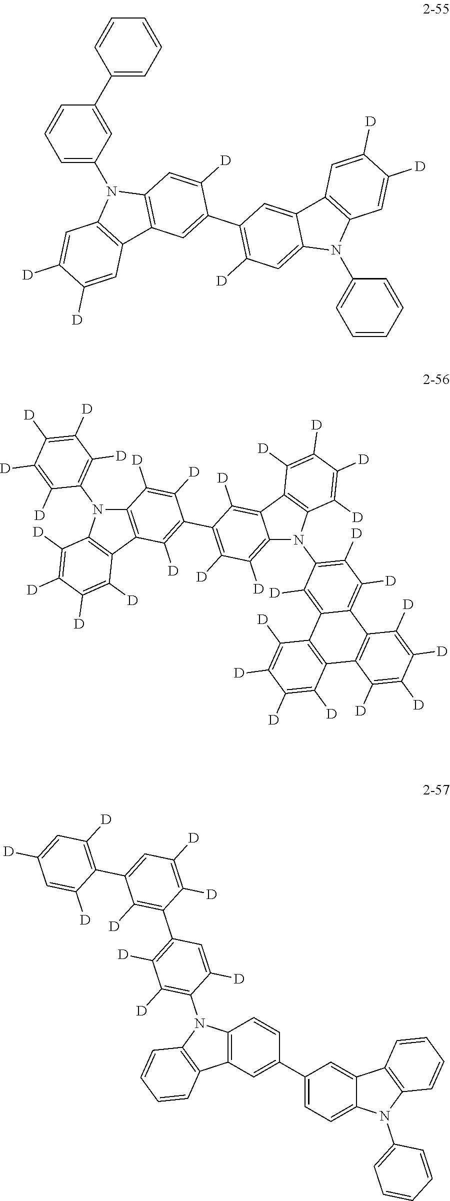 [SMILES: [2H]c1cc2c3cc(-c4cc5c6cc([2H])c([2H])cc6n(-c6cccc(-c7ccccc7)c6)c5cc4[2H])c([2H])cc3n(-c3ccccc3)c2cc1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c6c([2H])c([2H])c([2H])c([2H])c6c6c([2H])c([2H])c([2H])c([2H])c6c5c4[2H])c([2H])c([2H])c32)c([2H])c1[2H] ; [2H]c1cc([2H])c(-c2cc([2H])c([2H])c(-c3cc([2H])c(-n4c5ccccc5c5cc(-c6ccc7c(c6)c6ccccc6n7-c6ccccc6)ccc54)c([2H])c3[2H])c2[2H])c([2H])c1]

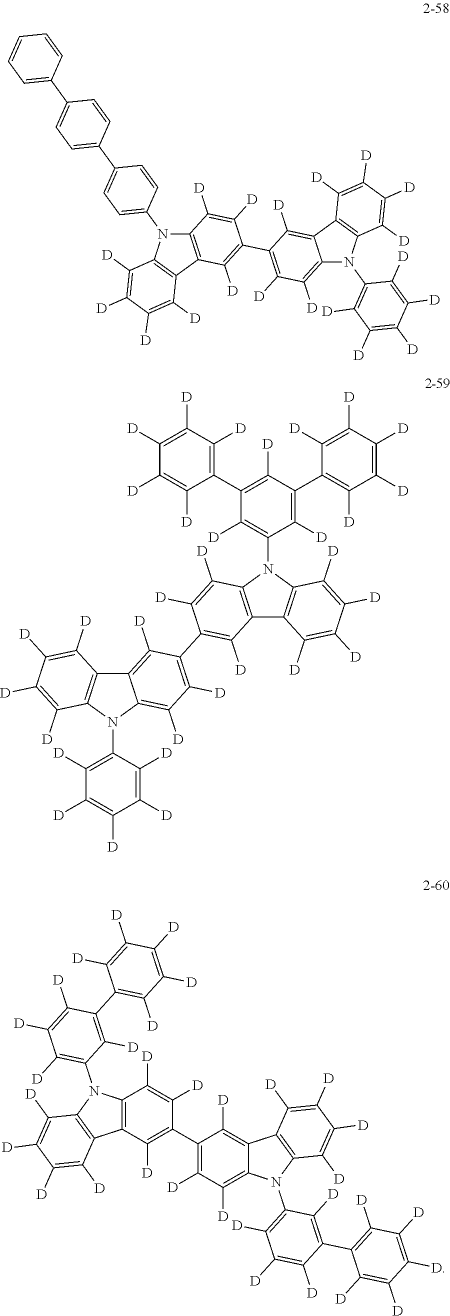

16. The light-emitting device according to claim 13, wherein the compound represented by Formula 5 is represented by at least one selected from formulae below:

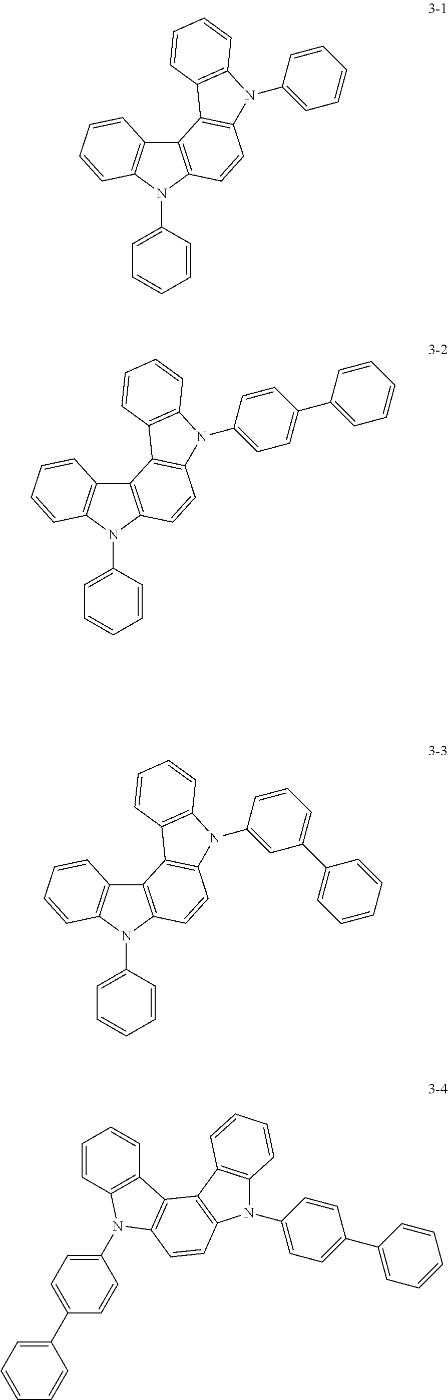 [SMILES: c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccc(-c7ccccc7)cc6)c5ccc43)cc2)cc1]

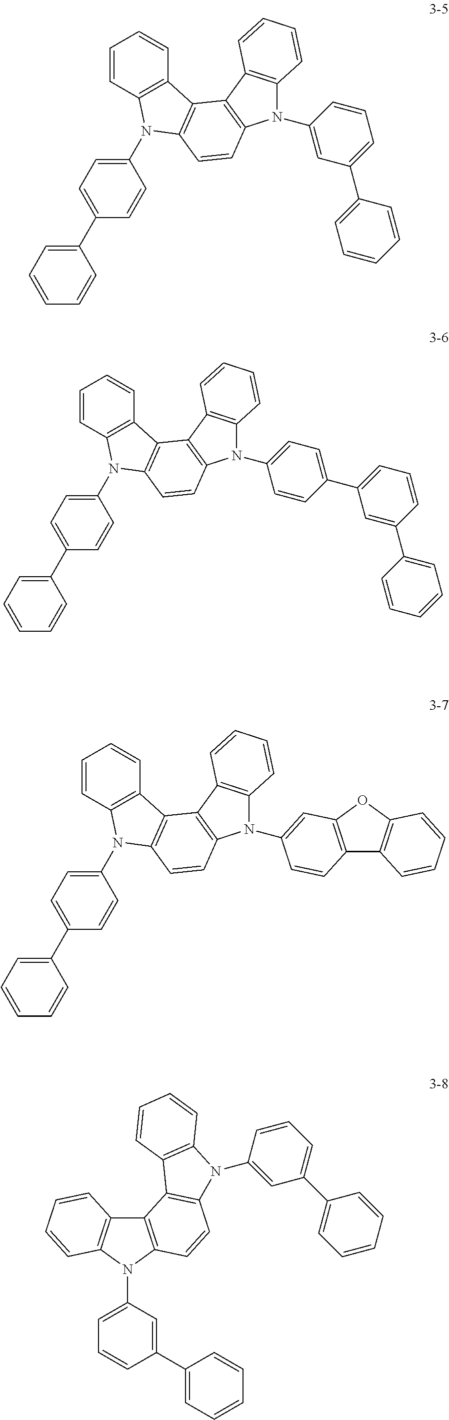 [SMILES: c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6ccc(-c7cccc(-c8ccccc8)c7)cc6)c5ccc43)cc2)cc1 ; c1ccc(-c2ccc(-n3c4ccccc4c4c5c6ccccc6n(-c6ccc7c(c6)oc6ccccc67)c5ccc43)cc2)cc1 ; c1ccc(-c2cccc(-n3c4ccccc4c4c5c6ccccc6n(-c6cccc(-c7ccccc7)c6)c5ccc43)c2)cc1]

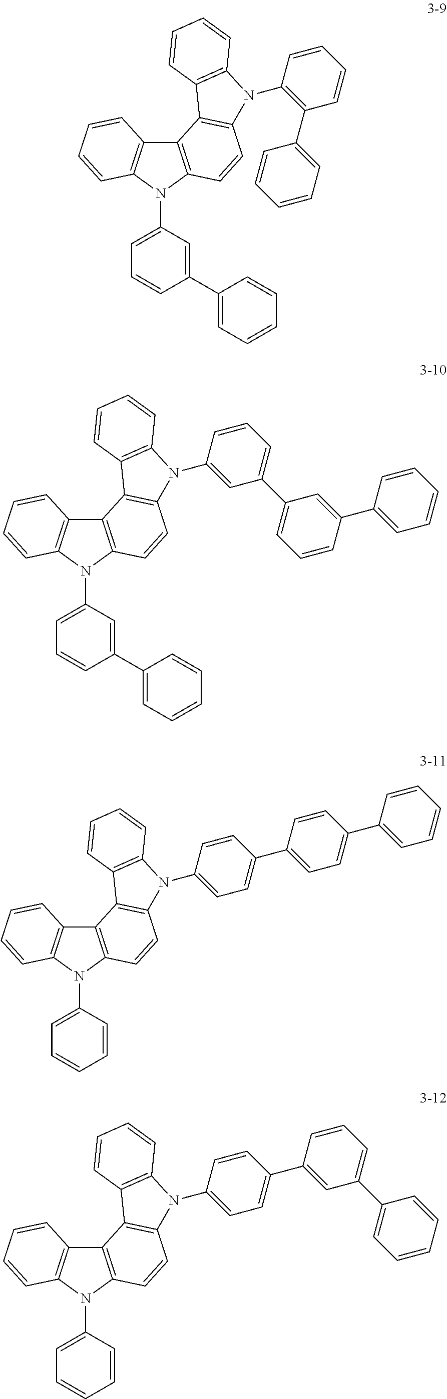 [SMILES: c1ccc(-c2cccc(-n3c4ccccc4c4c5c6ccccc6n(-c6ccccc6-c6ccccc6)c5ccc43)c2)cc1 ; c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2ccc(-c3ccc(-n4c5ccccc5c5c6c7ccccc7n(-c7ccccc7)c6ccc54)cc3)cc2)cc1 ; c1ccc(-c2cccc(-c3ccc(-n4c5ccccc5c5c6c7ccccc7n(-c7ccccc7)c6ccc54)cc3)c2)cc1]

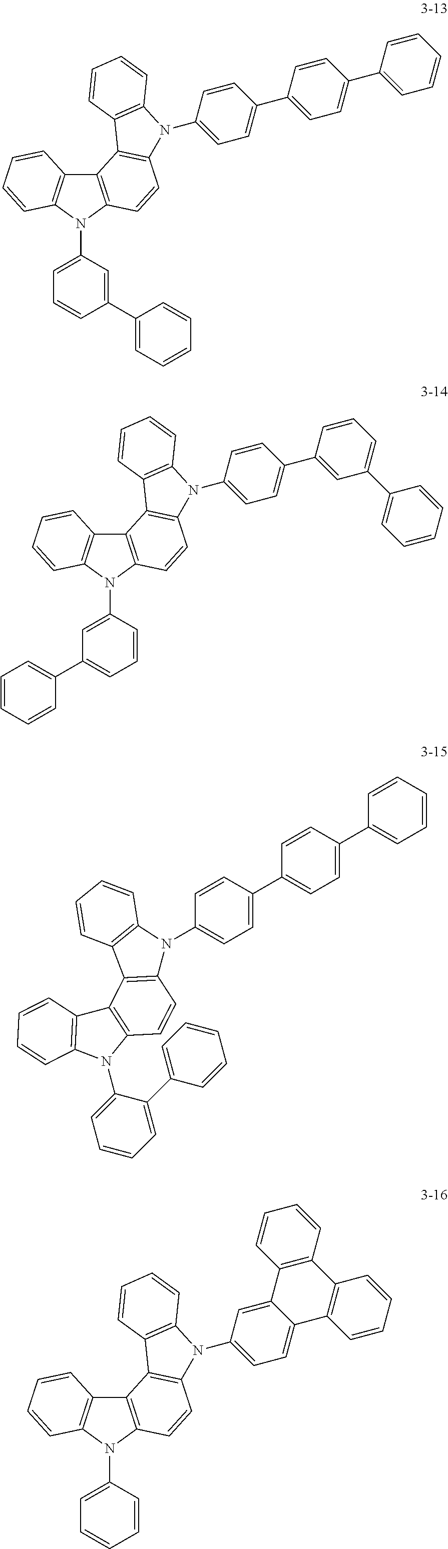 [SMILES: c1ccc(-c2ccc(-c3ccc(-n4c5ccccc5c5c6c7ccccc7n(-c7cccc(-c8ccccc8)c7)c6ccc54)cc3)cc2)cc1 ; c1ccc(-c2cccc(-c3ccc(-n4c5ccccc5c5c6c7ccccc7n(-c7cccc(-c8ccccc8)c7)c6ccc54)cc3)c2)cc1 ; c1ccc(-c2ccc(-c3ccc(-n4c5ccccc5c5c6c7ccccc7n(-c7ccccc7-c7ccccc7)c6ccc54)cc3)cc2)cc1 ; c1ccc(-n2c3ccccc3c3c4c5ccccc5n(-c5ccc6c7ccccc7c7ccccc7c6c5)c4ccc32)cc1]

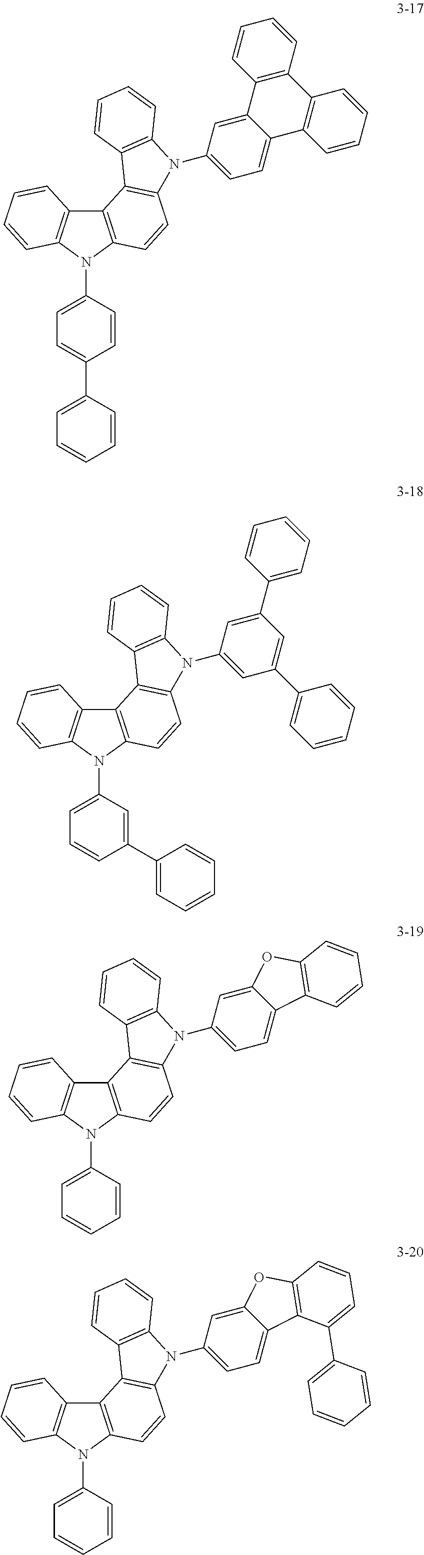 [SMILES: c1ccc(-c2ccc(-n3c4ccccc4c4c5c6ccccc6n(-c6ccc7c8ccccc8c8ccccc8c7c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1 ; c1ccc(-n2c3ccccc3c3c4c5ccccc5n(-c5ccc6c(c5)oc5ccccc56)c4ccc32)cc1 ; c1ccc(-c2cccc3oc4cc(-n5c6ccccc6c6c7c8ccccc8n(-c8ccccc8)c7ccc65)ccc4c23)cc1]

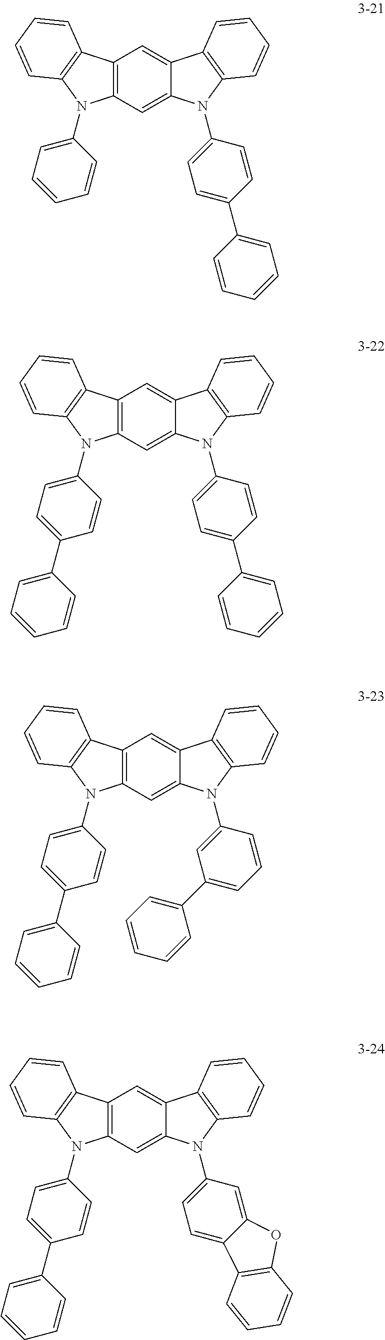 [SMILES: c1ccc(-c2ccc(-n3c4ccccc4c4cc5c6ccccc6n(-c6ccccc6)c5cc43)cc2)cc1 ; c1ccc(-c2ccc(-n3c4ccccc4c4cc5c6ccccc6n(-c6ccc(-c7ccccc7)cc6)c5cc43)cc2)cc1 ; c1ccc(-c2ccc(-n3c4ccccc4c4cc5c6ccccc6n(-c6cccc(-c7ccccc7)c6)c5cc43)cc2)cc1 ; c1ccc(-c2ccc(-n3c4ccccc4c4cc5c6ccccc6n(-c6ccc7c(c6)oc6ccccc67)c5cc43)cc2)cc1]

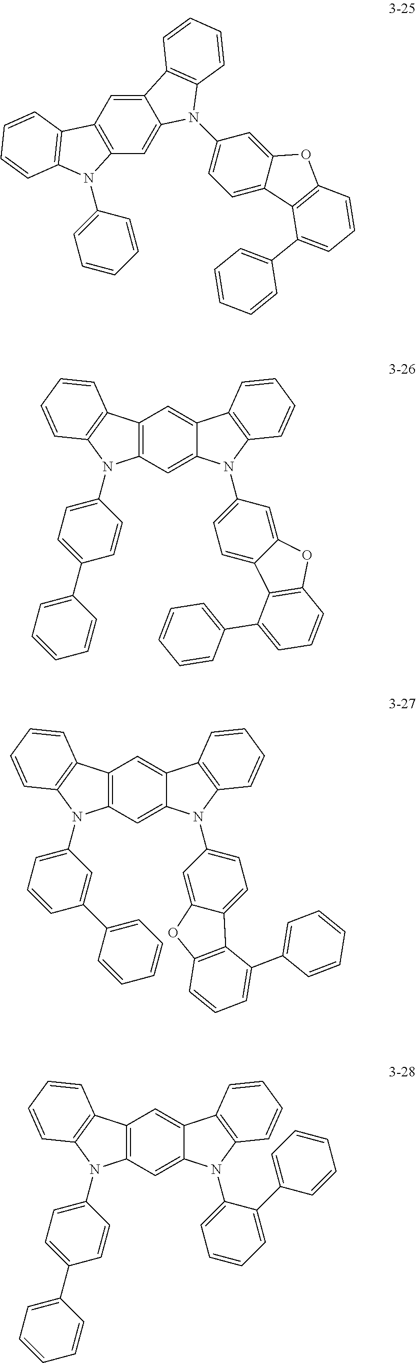 [SMILES: c1ccc(-c2cccc3oc4cc(-n5c6ccccc6c6cc7c8ccccc8n(-c8ccccc8)c7cc65)ccc4c23)cc1 ; c1ccc(-c2ccc(-n3c4ccccc4c4cc5c6ccccc6n(-c6ccc7c(c6)oc6cccc(-c8ccccc8)c67)c5cc43)cc2)cc1 ; c1ccc(-c2cccc(-n3c4ccccc4c4cc5c6ccccc6n(-c6ccc7c(c6)oc6cccc(-c8ccccc8)c67)c5cc43)c2)cc1 ; c1ccc(-c2ccc(-n3c4ccccc4c4cc5c6ccccc6n(-c6ccccc6-c6ccccc6)c5cc43)cc2)cc1]

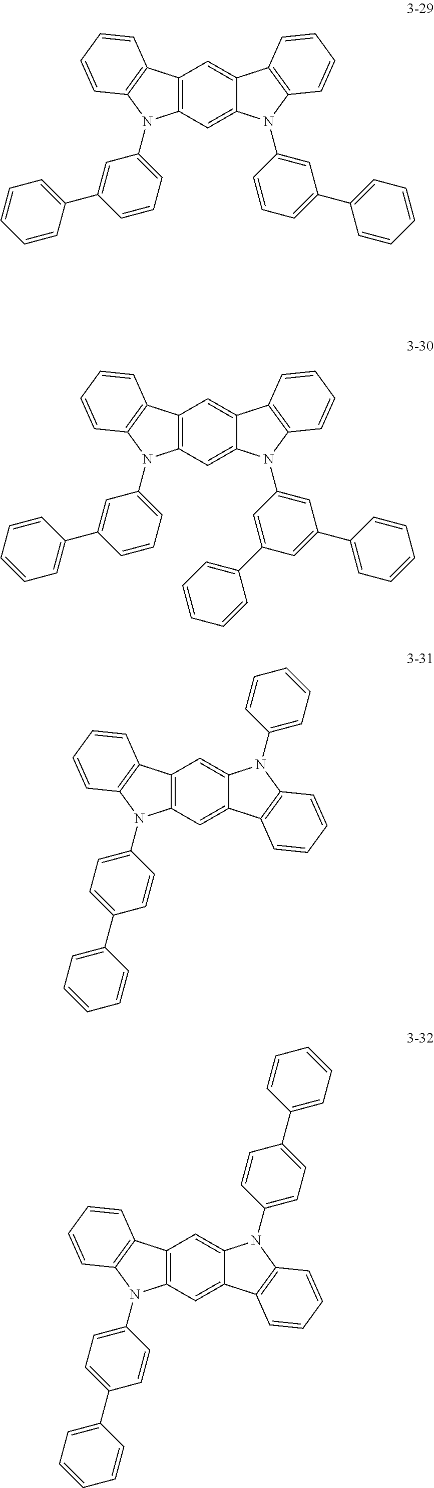 [SMILES: c1ccc(-c2cccc(-n3c4ccccc4c4cc5c6ccccc6n(-c6cccc(-c7ccccc7)c6)c5cc43)c2)cc1 ; c1ccc(-c2cccc(-n3c4ccccc4c4cc5c6ccccc6n(-c6cc(-c7ccccc7)cc(-c7ccccc7)c6)c5cc43)c2)cc1 ; c1ccc(-c2ccc(-n3c4ccccc4c4cc5c(cc43)c3ccccc3n5-c3ccccc3)cc2)cc1 ; c1ccc(-c2ccc(-n3c4ccccc4c4cc5c(cc43)c3ccccc3n5-c3ccc(-c4ccccc4)cc3)cc2)cc1]

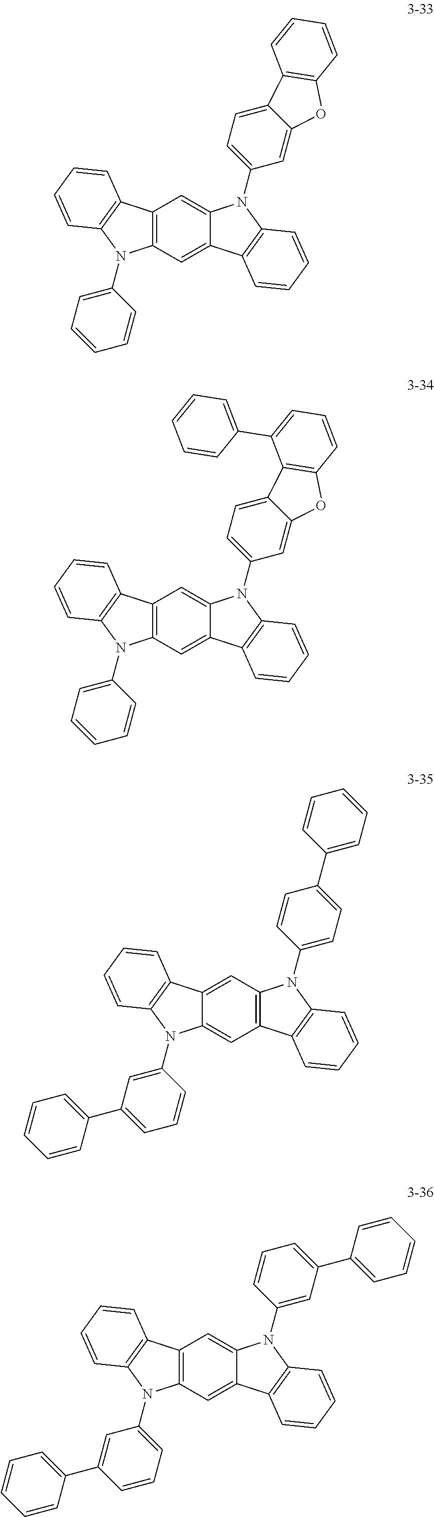 [SMILES: c1ccc(-n2c3ccccc3c3cc4c(cc32)c2ccccc2n4-c2ccc3c(c2)oc2ccccc23)cc1 ; c1ccc(-c2cccc3oc4cc(-n5c6ccccc6c6cc7c(cc65)c5ccccc5n7-c5ccccc5)ccc4c23)cc1 ; c1ccc(-c2ccc(-n3c4ccccc4c4cc5c(cc43)c3ccccc3n5-c3cccc(-c4ccccc4)c3)cc2)cc1 ; c1ccc(-c2cccc(-n3c4ccccc4c4cc5c(cc43)c3ccccc3n5-c3cccc(-c4ccccc4)c3)c2)cc1]

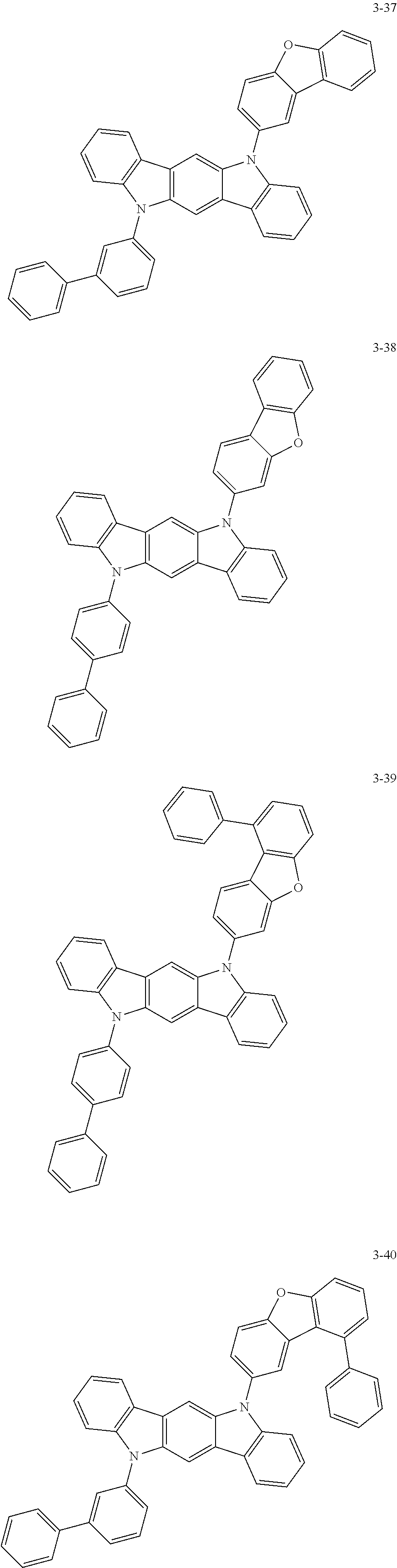 [SMILES: c1ccc(-c2cccc(-n3c4ccccc4c4cc5c(cc43)c3ccccc3n5-c3ccc4oc5ccccc5c4c3)c2)cc1 ; c1ccc(-c2ccc(-n3c4ccccc4c4cc5c(cc43)c3ccccc3n5-c3ccc4c(c3)oc3ccccc34)cc2)cc1 ; c1ccc(-c2ccc(-n3c4ccccc4c4cc5c(cc43)c3ccccc3n5-c3ccc4c(c3)oc3cccc(-c5ccccc5)c34)cc2)cc1 ; c1ccc(-c2cccc(-n3c4ccccc4c4cc5c(cc43)c3ccccc3n5-c3ccc4oc5cccc(-c6ccccc6)c5c4c3)c2)cc1]

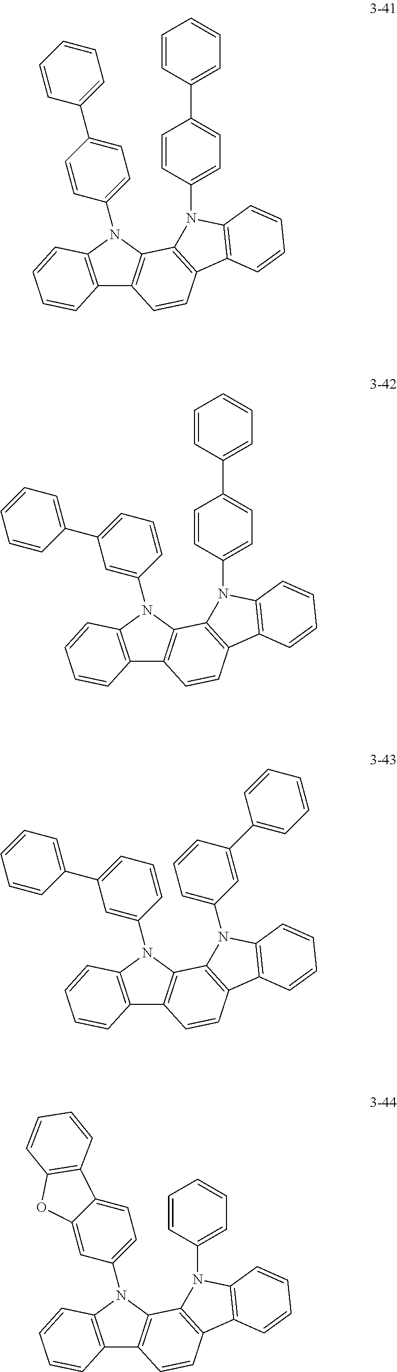 [SMILES: c1ccc(-c2ccc(-n3c4ccccc4c4ccc5c6ccccc6n(-c6ccc(-c7ccccc7)cc6)c5c43)cc2)cc1 ; c1ccc(-c2ccc(-n3c4ccccc4c4ccc5c6ccccc6n(-c6cccc(-c7ccccc7)c6)c5c43)cc2)cc1 ; c1ccc(-c2cccc(-n3c4ccccc4c4ccc5c6ccccc6n(-c6cccc(-c7ccccc7)c6)c5c43)c2)cc1 ; c1ccc(-n2c3ccccc3c3ccc4c5ccccc5n(-c5ccc6c(c5)oc5ccccc56)c4c32)cc1]

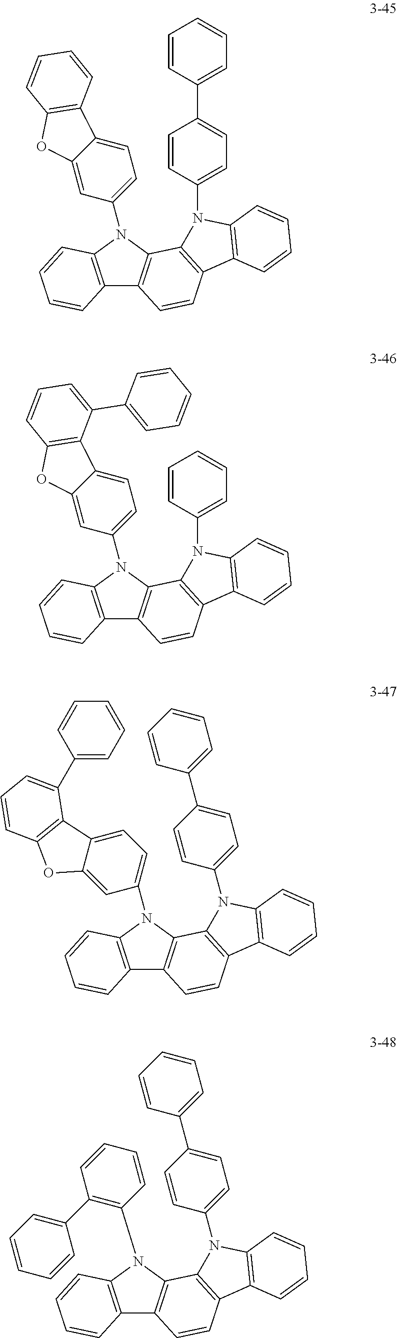 [SMILES: c1ccc(-c2ccc(-n3c4ccccc4c4ccc5c6ccccc6n(-c6ccc7c(c6)oc6ccccc67)c5c43)cc2)cc1 ; c1ccc(-c2cccc3oc4cc(-n5c6ccccc6c6ccc7c8ccccc8n(-c8ccccc8)c7c65)ccc4c23)cc1 ; c1ccc(-c2ccc(-n3c4ccccc4c4ccc5c6ccccc6n(-c6ccc7c(c6)oc6cccc(-c8ccccc8)c67)c5c43)cc2)cc1 ; c1ccc(-c2ccc(-n3c4ccccc4c4ccc5c6ccccc6n(-c6ccccc6-c6ccccc6)c5c43)cc2)cc1]

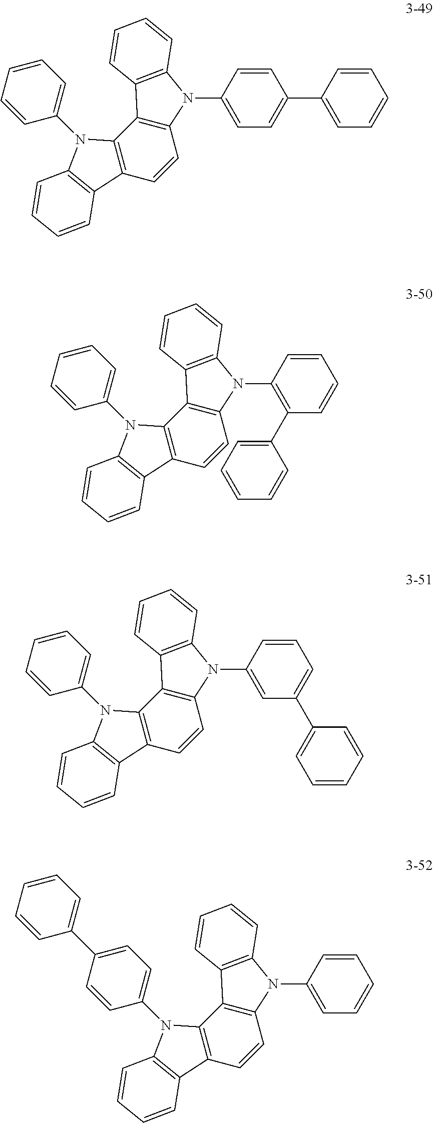 [SMILES: c1ccc(-c2ccc(-n3c4ccccc4c4c3ccc3c5ccccc5n(-c5ccccc5)c34)cc2)cc1 ; c1ccc(-c2ccccc2-n2c3ccccc3c3c2ccc2c4ccccc4n(-c4ccccc4)c23)cc1 ; c1ccc(-c2cccc(-n3c4ccccc4c4c3ccc3c5ccccc5n(-c5ccccc5)c34)c2)cc1 ; c1ccc(-c2ccc(-n3c4ccccc4c4ccc5c(c6ccccc6n5-c5ccccc5)c43)cc2)cc1]

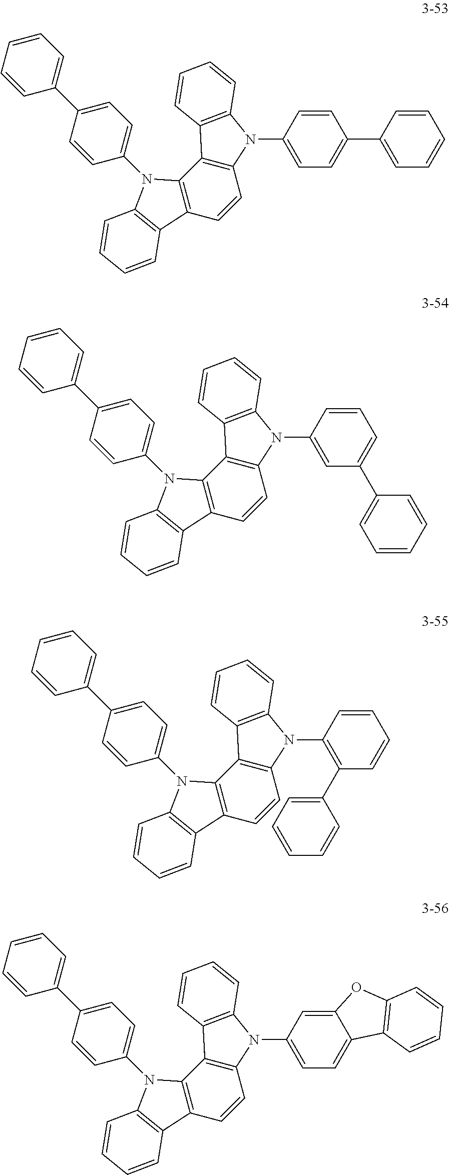 [SMILES: c1ccc(-c2ccc(-n3c4ccccc4c4c3ccc3c5ccccc5n(-c5ccc(-c6ccccc6)cc5)c34)cc2)cc1 ; c1ccc(-c2ccc(-n3c4ccccc4c4ccc5c(c6ccccc6n5-c5cccc(-c6ccccc6)c5)c43)cc2)cc1 ; c1ccc(-c2ccc(-n3c4ccccc4c4ccc5c(c6ccccc6n5-c5ccccc5-c5ccccc5)c43)cc2)cc1 ; c1ccc(-c2ccc(-n3c4ccccc4c4ccc5c(c6ccccc6n5-c5ccc6c(c5)oc5ccccc56)c43)cc2)cc1]

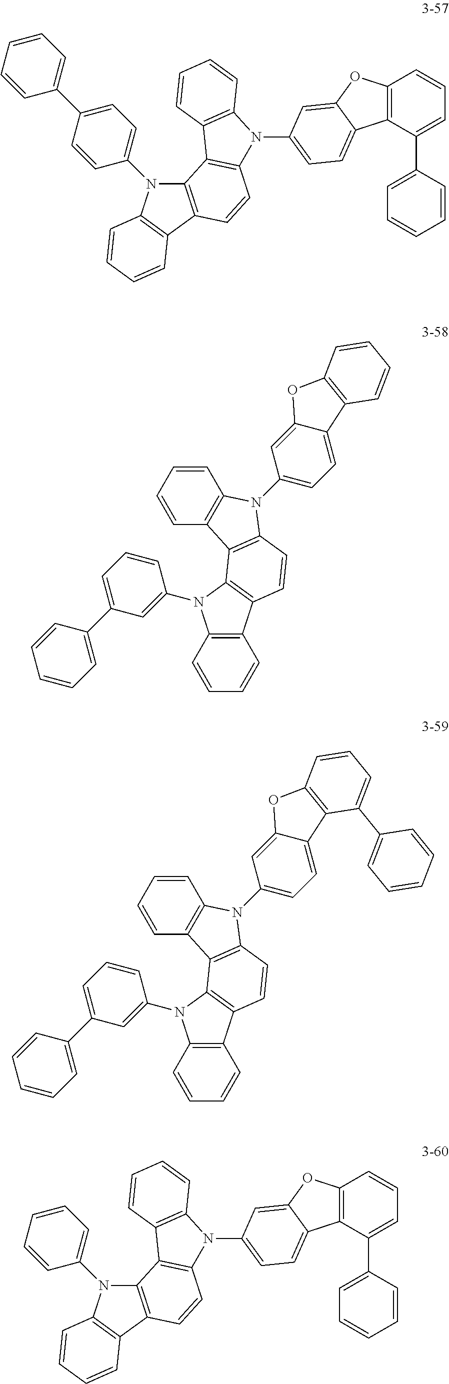 [SMILES: c1ccc(-c2ccc(-n3c4ccccc4c4ccc5c(c6ccccc6n5-c5ccc6c(c5)oc5cccc(-c7ccccc7)c56)c43)cc2)cc1 ; c1ccc(-c2cccc(-n3c4ccccc4c4ccc5c(c6ccccc6n5-c5ccc6c(c5)oc5ccccc56)c43)c2)cc1 ; c1ccc(-c2cccc(-n3c4ccccc4c4ccc5c(c6ccccc6n5-c5ccc6c(c5)oc5cccc(-c7ccccc7)c56)c43)c2)cc1 ; c1ccc(-c2cccc3oc4cc(-n5c6ccccc6c6c5ccc5c7ccccc7n(-c7ccccc7)c56)ccc4c23)cc1]

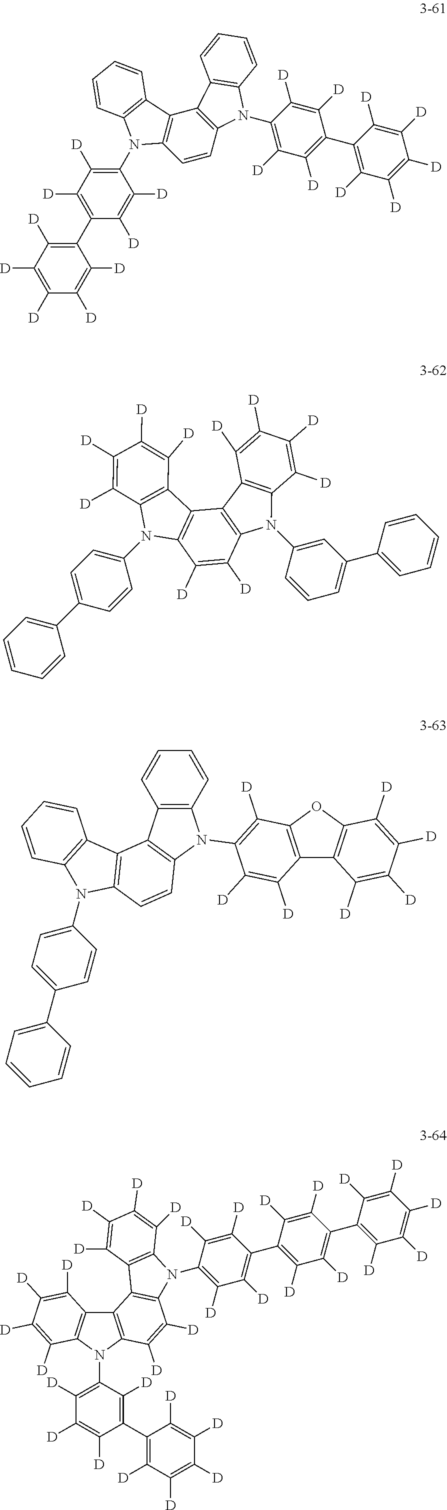

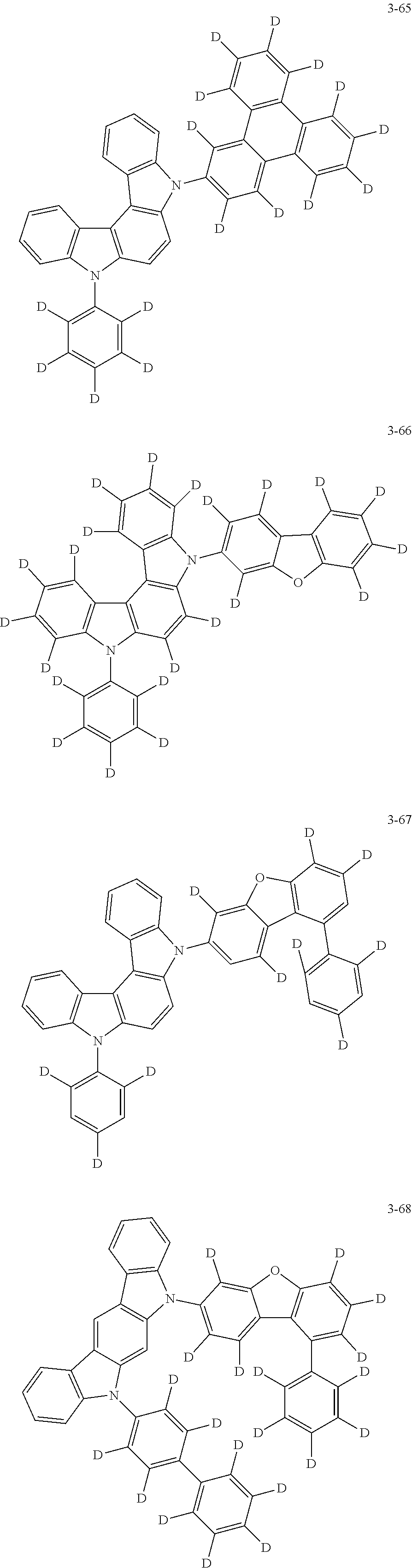

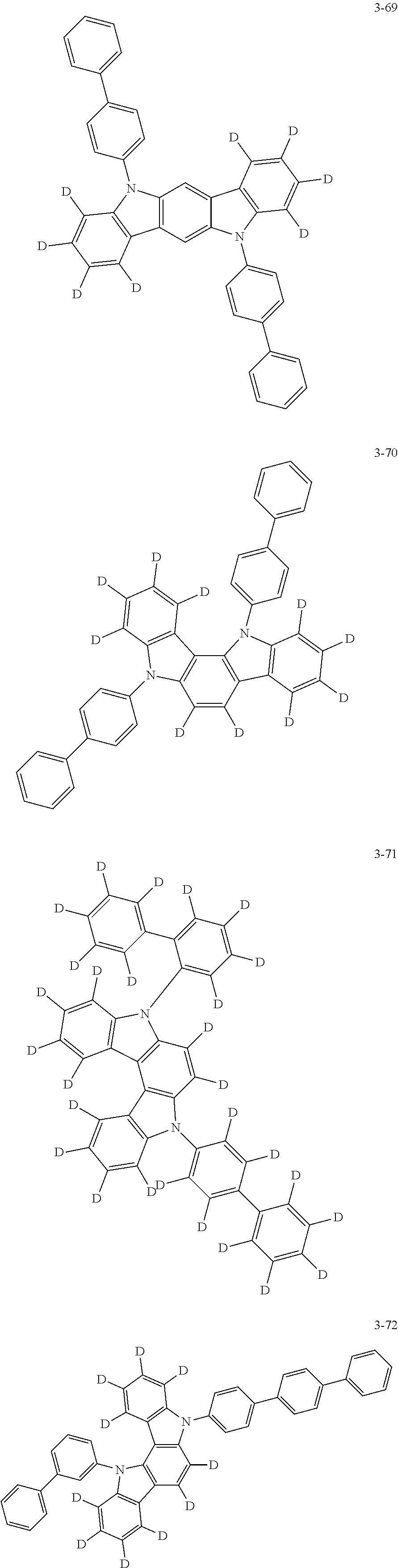

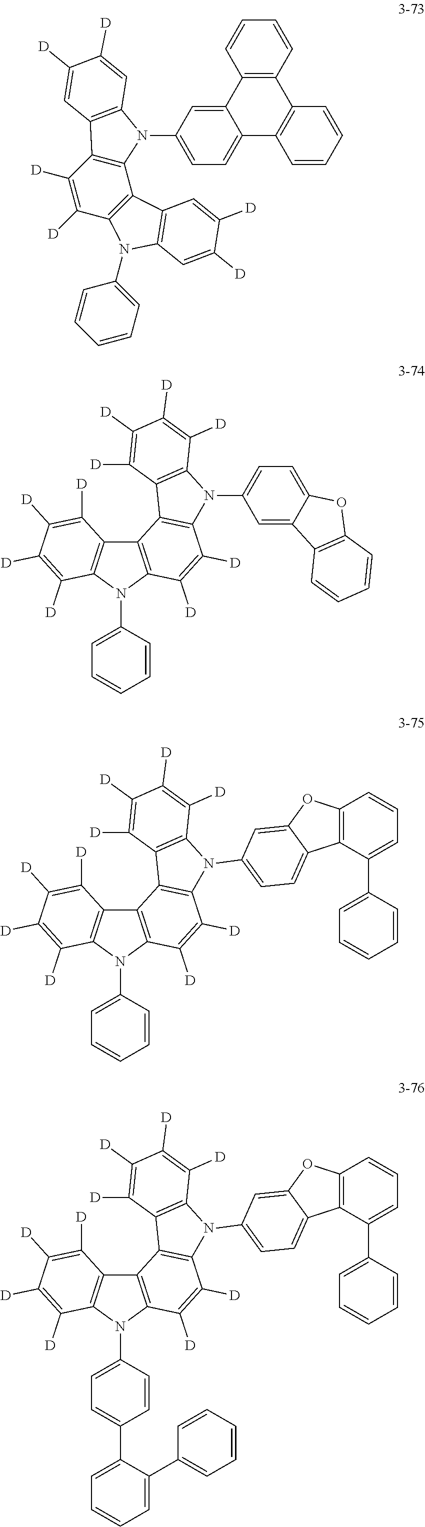

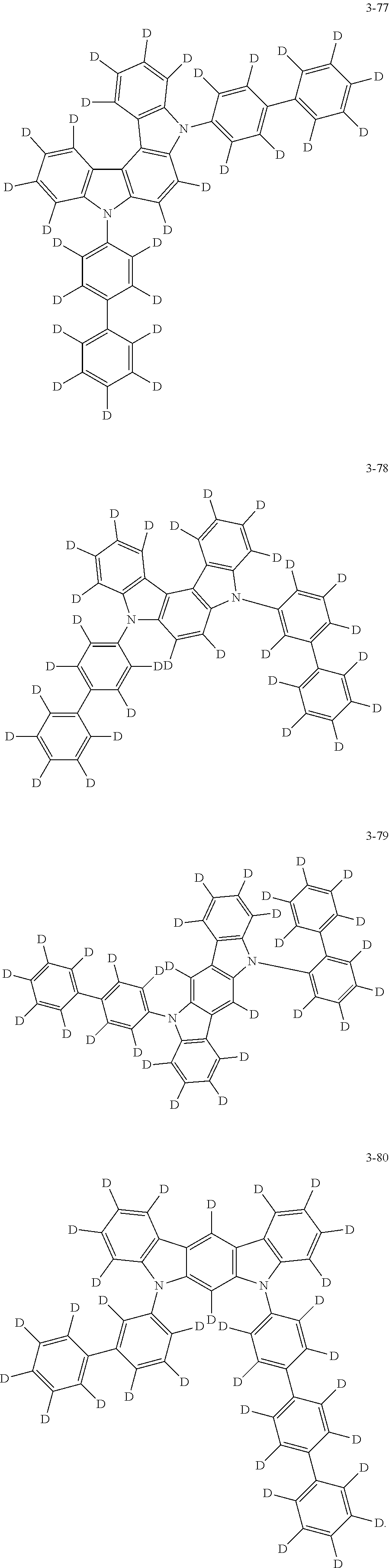

17. A composition for an organic layer of an organic light-emitting device comprising:
the heterocyclic compound represented by Formula 1 according to claim 1; and
a compound represented by Formula 4 below or a compound represented by Formula 5: 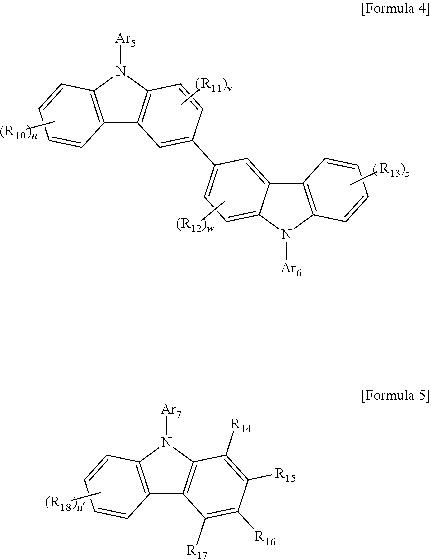
wherein, i Formula 4 and 5, two adjacent groups of R₁₄ to R₁₇ form a ring represented by 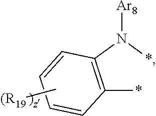
the remaining groups of R₁₀ to R₁₃, R₁₄ to R₁₇, and R₁₈ to R₁₉ that do not form a ring are each independently selected from the group consisting of hydrogen, deuterium, a halogen, a substituted or unsubstituted alkyl group having 1 to 60 carbon atoms, a substituted or unsubstituted alkenyl group having 2 to 60 carbon atoms, a substituted or unsubstituted alkynyl group having 2 to 60 carbon atoms, a substituted or unsubstituted alkoxy group having 1 to 60 carbon atoms, a substituted or unsubstituted cycloalkyl group having 3 to 60 carbon atoms, a substituted or unsubstituted heterocycloalkyl group having 2 to 60 carbon atoms, a substituted or unsubstituted aryl group having 6 to 60 carbon atoms, a substituted or unsubstituted heteroaryl group having 2 to 60 carbon atoms, —SiRR′R″, and —P(═O)RR′, wherein adjacent groups are linked to form a substituted or unsubstituted aryl group having 6 to 60 carbon atoms, a substituted or unsubstituted heterocycloalkyl group having 2 to 60 carbon atoms, or a substituted or unsubstituted heteroaryl group having 2 to 60 carbon atoms,
R, R′ and R″ are each independently hydrogen, deuterium, a cyano group (—CN), a substituted or unsubstituted alkyl group having 1 to 60 carbon atoms, a substituted or unsubstituted cycloalkyl group having 3 to 60 carbon atoms, a substituted or unsubstituted aryl group having 6 to 60 carbon atoms, or a substituted or unsubstituted heteroaryl group having 2 to 60 carbon atoms,
Ar₅ to Ar₈ are each independently a substituted or unsubstituted aryl group having 6 to 60 carbon atoms, or a substituted or unsubstituted heteroaryl group having 2 to 60 carbon atoms,
u, u′, z, and z′ are each independently 0 or an integer of 1 to 4,
v and w are each independently 0 or an integer of 1 to 3, and
* represents a bonding site.

18. The composition according to claim 17, wherein a ratio of the weight of the compound represented by Formula 4 or the compound represented by Formula 5 to the weight of the heterocyclic compound represented by Formula 1 in the total weight of the composition is 0.1 to 10.
